(19) United States
(12) Patent Application Publication
(10) Pub. No.: US 2025/0100993 A1
(43) Pub. Date: Mar. 27, 2025

(54) 2-SUBSTITUTED THIAZOLE HSD17B13 INHIBITORS AND USES THEREOF

(71) Applicant: Inipharm, Inc., Bellevue, WA (US)

(72) Inventors: Joshua ODINGO, Bothell, WA (US); Sampath Kumar ANANDAN, Fremont, CA (US); Heather Kay Webb HSU, Seattle, WA (US); Subramanyam Janardhan TANTRY, Karnataka (IN); Athisayamani Jeyaraj DURAISWAMY, Karnataka (IN); Bharathi Mohan KUPPUSAMY, Karnataka (IN)

(21) Appl. No.: 18/730,991

(22) PCT Filed: Jan. 25, 2023
(86) PCT No.: PCT/US2023/011520

§ 371 Date: Jul. 22, 2024

Related U.S. Application Data
(60) Provisional application No. 63/377,421, filed on Sep. 28, 2022.
(60) Provisional application No. 63/342,786, filed on May 17, 2022.

(30) Foreign Application Priority Data
Jan. 26, 2022 (IN) 202211004338

(51) Int. Cl. C07D 401/12; A61K 31/415; A61K 31/426; A61K 31/427; A61K 31/4439; A61K 31/444; A61K 31/517; A61K 31/53; A61K 31/5377; A61K 31/541; C07D 231/40; C07D 239/90; C07D 253/08; C07D 277/46; C07D 417/04; C07D 417/12; C07D 417/14

(52) U.S. Cl. CPC C07D 401/12; A61K 31/415; A61K 31/426; A61K 31/427; A61K 31/4439; A61K 31/444; A61K 31/517; A61K 31/53; A61K 31/5377; A61K 31/541; C07D 231/40; C07D 239/90; C07D 253/08; C07D 277/46; C07D 417/04; C07D 417/12; C07D 417/14

(57) ABSTRACT

Described herein are selective HSD17B13 inhibitors and pharmaceutical compositions comprising said inhibitors. The subject compounds and compositions are useful for the treatment of liver disease, metabolic disease, or cardiovascular disease, such as NAFLD or NASH, or drug induced liver injury (DILI).

### CROSS-REFERENCE

This application claims the benefit of Indian Application No. 202211004338 filed Jan. 26, 2022; U.S. Provisional Application Ser. No. 63/342,786 filed May 17, 2022; and U.S. Provisional Application Ser. No. 63/377,421 filed Sep. 28, 2022; which are hereby incorporated by reference in their entirety.

### BACKGROUND OF THE INVENTION

Nonalcoholic fatty liver diseases (NAFLDs) including NASH (nonalcoholic steatohepatitis) are considered to be hepatic manifestations of the metabolic syndrome and are characterized by the accumulation of triglycerides in the liver of patients without a history of excessive alcohol consumption. The majority of patients with NAFLD are obese or morbidly obese and have accompanying insulin resistance. The incidence of NAFLD/NASH has been rapidly increasing worldwide consistent with the increased prevalence of obesity, and it is currently the most common chronic liver disease.

NAFLD is classified into simple steatosis, in which only hepatic steatosis is observed, and NASH, in which intralobular inflammation and ballooning degeneration of hepatocytes is observed along with hepatic steatosis. The proportion of patients with NAFLD who have NASH is still not clear but might range from 20-40%. NASH is a progressive disease and may lead to liver cirrhosis and hepatocellular carcinoma. Twenty percent of NASH patients are reported to develop cirrhosis, and 30-40% of patients with NASH cirrhosis experience liver-related death. Recently, NASH has become the third most common indication for liver transplantation in the United States. Currently, the principal treatment for NAFLD/NASH is lifestyle modification by diet and exercise. However, pharmacological therapy is indispensable because obese patients with NAFLD often have difficulty maintaining improved lifestyles.

17β-Hydroxysteroid dehydrogenases (HSD17Bs) comprise a large family of 15 members some of which involved in sex hormone metabolism. Some HSD17Bs enzymes also play key roles in cholesterol and fatty acid metabolism. A recent study showed that hydroxysteroid 17β-dehydrogenase 13 (HSD17B13), an enzyme with unknown biological function, is a novel liver-specific lipid droplet (LD)-associated protein in mouse and humans. HSD17B13 expression is markedly upregulated in patients and mice with non-alcoholic fatty liver disease (NAFLD). Hepatic overexpression of HSD17B13 promotes lipid accumulation in the liver. HSD17B13 could also have potential as a biomarker of chronic liver disease, such as alcoholic liver disease (ALD), non-alcoholic fatty liver disease (NAFLD) (for example: steatosis, nonalcoholic steatohepatitis (NASH), NASH-fibrosis, or cirrhosis), steatohepatitis, and liver cancer.

### SUMMARY OF THE INVENTION

Provided herein are methods, compounds, and compositions useful for reducing expression or activity of HSD17B13 in a subject in need thereof. Also, provided herein are methods, compounds, and compositions comprising HSD17B13 specific inhibitors, which can be useful in reducing the morbidity of HSD17B13-related diseases or conditions in a subject in need thereof. Such methods, compounds, and compositions can be useful, for example, to treat, prevent, delay, or ameliorate liver disease, metabolic disease, or cardiovascular disease.

Disclosed herein is a compound of Formula (I), or a pharmaceutically acceptable salt, solvate, or stereoisomer thereof:

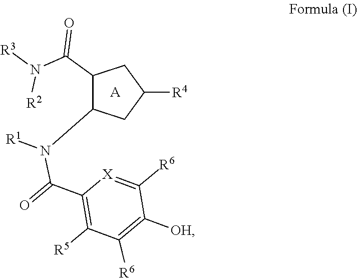

wherein:
Ring A is thiazolyl;
R¹ is hydrogen, C₁-C₆alkyl, C₁-C₆haloalkyl, C₁-C₆deuteroalkyl, C₁-C₆hydroxyalkyl, or C₁-C₆aminoalkyl;
R² is hydrogen or C₁-C₆alkyl;
R³ is C₁-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₂-C₁₀alkenyl, C₂-C₁₀alkynyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally and independently substituted with one or more R^{3a};
each R^{3a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{3a} on the same atom are taken together to form an oxo;
R⁴ is deuterium, halogen, C₁-C₆alkyl, C₂-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more with one or more R^{4a};
each R^{4a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally and independently substituted with one or more R;
or two R^{4a} on the same atom are taken together to form an oxo;
or two R^{4a} on the same carbon are taken together to form a cycloalkyl or heterocycloalkyl; each optionally substituted with one or more R;
or two R^{4a} on the different atoms are taken together to form a cycloalkyl, heterocycloalkyl, aryl, or heteroaryl; each optionally substituted with one or more R;
X is N or CR^{X};
R^{X} is hydrogen, deuterium, halogen, —CN, —OH, —OR^{a}, —NR^{c}R^{d}, C₁-C₆alkyl, C₁-C₆haloalkyl, or C₁-C₆deuteroalkyl;
R⁵ is hydrogen, deuterium, halogen, —CN, —OH, —OR^{a}, —NR^{c}R^{d}, C₁-C₆alkyl, C₁-C₆haloalkyl, or C₁-C₆deuteroalkyl;
each R⁶ is independently a halogen;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom form an oxo.

In some embodiments of a compound of Formula (I), the compound is of Formula (Ia):

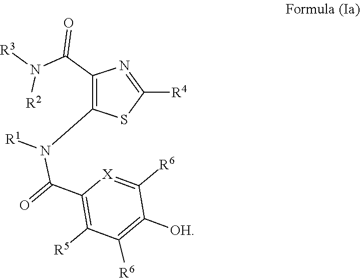

In some embodiments of a compound of Formula (I), the compound is of Formula (Ib):

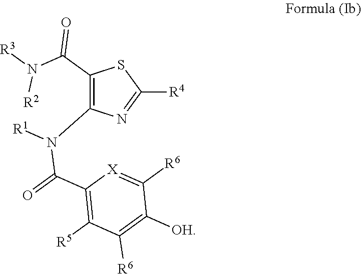

Also disclosed herein is a pharmaceutical composition comprising a compound disclosed herein, or a pharmaceutically acceptable salt, solvate, or stereoisomer thereof, and a pharmaceutically acceptable carrier.

Also disclosed herein is a method of treating a disease in a subject in need thereof, the method comprising administering a pharmaceutically effective amount of a compound disclosed herein, or a pharmaceutically acceptable salt, solvate, or stereoisomer thereof, or a pharmaceutical composition disclosed herein. In some embodiments of a method of treating a disease, the disease is a liver disease, a metabolic disease, or a cardiovascular disease. In some embodiments of a method of treating a disease, the disease is NAFLD. In some embodiments of a method of treating a disease, the disease is NASH. In some embodiments of a method of treating a disease, the disease is drug induced liver injury (DILI). In some embodiments of a method of treating a disease, the disease is associated with HSD17B13. In some embodiments of a method of treating a disease, the diseases is alcoholic liver disease. In some embodiments of a method of treating a disease, the disease is cirrhosis. In some embodiments of a method of treating a disease, the disease is decompensated portal hypertension. In some embodiments of a method of treating a disease, the disease is cholestatic liver disease.

Also disclosed herein is a method for selectively inhibiting HSD17B13, the method comprising administering a pharmaceutically effective amount of a compound disclosed herein, or a pharmaceutically acceptable salt, solvate, or stereoisomer thereof. In some embodiments, the compound selectively inhibit HSD17B13 over HSD17B2, HSD17B14, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl. In some embodiments, the alkyl is a C₁—C₆ alkyl. In some embodiments, the alkyl is a C₁-C₅ alkyl. In some embodiments, the alkyl is a C₁-C₄ alkyl. In some embodiments, the alkyl is a C₁-C₃ alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —Oalkyl where alkyl is defined as above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5-to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo [2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heteroalkyl” refers to an alkyl group in which one or more skeletal atoms of the alkyl are selected from an atom other than carbon, e.g., oxygen, nitrogen, sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen, sulfur, phosphorus, or combinations thereof wherein the heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or two atoms selected from the group consisting of oxygen, nitrogen, and sulfur wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-Cis heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₆ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). In some embodiments, the heterocycloalkyl is a 3- to 8-membered ring comprising 1, 2, or 3 heteroatoms selected from the group consisting of oxygen, nitrogen, or sulfur. In some embodiments, the heterocycloalkyl is a 3- to 6-membered ring comprising 1, 2, or 3 heteroatoms selected from the group consisting of oxygen, nitrogen, or sulfur. In some embodiments, the heterocycloalkyl is a 5- to 6-membered ring comprising 1, 2, or 3 heteroatoms selected from the group consisting of oxygen, nitrogen, or sulfur. Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. In some embodiments, the heteroaryl is a 5- to 6-membered ring comprising 1, 2, or 3 heteroatoms selected from the group consisting of oxygen, nitrogen, or sulfur. Examples include, but are not limited to, azepinyl, acridinyl, benzimidazolyl, benzothiazolyl, benzindolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, four, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a liver disease, e.g., NAFLD).

“Synergy” or “synergize” refers to an effect of a combination that is greater than additive of the effects of each component alone at the same doses.

“HSD17B13” means hydroxysteroid 17-beta dehydrogenase 13 and refers to any nucleic acid of HSD17B13. For example, in some embodiments, HSD17B13 includes a DNA sequence encoding HSD17B13, an RNA sequence transcribed from DNA encoding HSD17B13 (including genomic DNA comprising introns and exons). HSD17B13 can also refer to any amino acid sequence of HSD17B13 (may include secondary or tertiary structures of the protein molecule), encoded by a DNA sequence and/or RNA sequence. The target may be referred to in either upper or lower case.

### Compounds

Described herein are compounds of Formula (I), or a pharmaceutically acceptable salt, solvate, or stereoisomer thereof useful in the treatment of liver diseases. In some embodiments, the liver disease is NAFLD. In some embodiments, the compounds disclosed herein are selective HSD17B13 inhibitors.

Disclosed herein is a compound of Formula (I), or a pharmaceutically acceptable salt, solvate, or stereoisomer thereof:

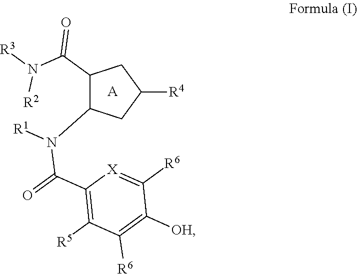

wherein:
Ring A is thiazolyl;
R¹ is hydrogen, C₁-C₆alkyl, C₁-C₆haloalkyl, C₁-C₆deuteroalkyl, C₁-C₆hydroxyalkyl, or C₁-C₆aminoalkyl;
R² is hydrogen or C₁-C₆alkyl;
R³ is C₁-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₂-C₁₀alkenyl, C₂-C₁₀alkynyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally and independently substituted with one or more R^{3a};
each R^{3a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NRbC(═O)NR^{c}R^{d}, —NR^{b}C(═O)R^{a}, —NR^{b}C(═O)OR, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{3a} on the same atom are taken together to form an oxo;
R⁴ is deuterium, halogen, C₁-C₆alkyl, C₂-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more with one or more R^{4a};
each R^{4a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally and independently substituted with one or more R;
or two R^{4a} on the same atom are taken together to form an oxo;
or two R^{4a} on the same carbon are taken together to form a cycloalkyl or heterocycloalkyl; each optionally substituted with one or more R;
or two R^{4a} on the different atoms are taken together to form a cycloalkyl, heterocycloalkyl, aryl, or heteroaryl; each optionally substituted with one or more R;
X is N or CR^{X};
R^{X} is hydrogen, deuterium, halogen, —CN, —OH, —OR^{a}, —NR^{c}R^{d}, C₁-C₆alkyl, C₁-C₆haloalkyl, or C₁-C₆deuteroalkyl;
R⁵ is hydrogen, deuterium, halogen, —CN, —OH, —OR^{a}, —NR^{c}R^{d}, C₁-C₆alkyl, C₁-C₆haloalkyl, or C₁-C₆deuteroalkyl;
each R⁶ is independently a halogen;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom form an oxo.

Disclosed herein is a compound of Formula (I), or a pharmaceutically acceptable salt, solvate, or stereoisomer thereof:

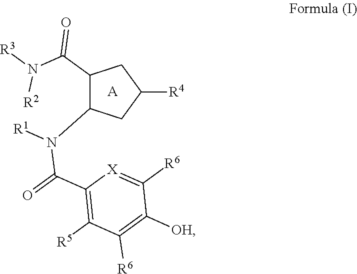

wherein:
Ring A is thiazolyl;
R¹ is hydrogen, C₁-C₆alkyl, C₁-C₆haloalkyl, C₁-C₆deuteroalkyl, C₁-C₆hydroxyalkyl, or C₁-C₆aminoalkyl;
R² is hydrogen or C₁-C₆alkyl;
R³ is C₁-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₂-C₁₀alkenyl, C₂-C₁₀alkynyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally and independently substituted with one or more R^{3a};
each R^{3a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{3a} on the same atom are taken together to form an oxo;
R⁴ is deuterium, halogen, C₁-C₆alkyl, C₂-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more with one or more R^{4a};
each R^{4a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{4a} on the same atom are taken together to form an oxo;
or two R^{4a} on the same carbon are taken together to form a cycloalkyl or heterocycloalkyl; each optionally substituted with one or more R;
or two R^{4a} on the different atoms are taken together to form a cycloalkyl, heterocycloalkyl, aryl, or heteroaryl; each optionally substituted with one or more R;
X is N or CR^{X};
R^{X} is hydrogen, deuterium, halogen, —CN, —OH, —OR^{a}, —NR^{c}R^{d}, C₁-C₆alkyl, C₁-C₆haloalkyl, or C₁-C₆deuteroalkyl;
R⁵ is hydrogen, deuterium, halogen, —CN, —OH, —OR^{a}, —NR^{c}R^{d}, C₁-C₆alkyl, C₁-C₆haloalkyl, or C₁-C₆deuteroalkyl;
each R⁶ is independently a halogen;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom form an oxo.

In some embodiments of a compound of Formula (I), the compound is of Formula (Ia):

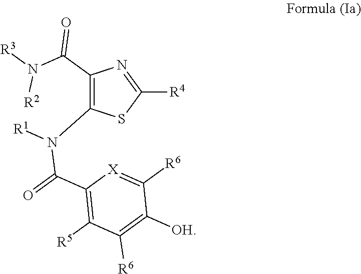

In some embodiments of a compound of Formula (I), the compound is of Formula (Ib):

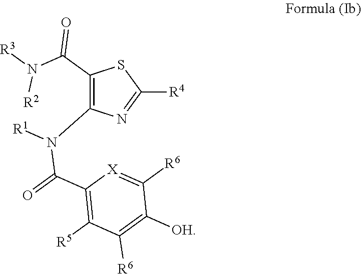

In some embodiments of a compound of Formula (I), (Ia), or (Ib), R¹ is hydrogen, C₁-C₆alkyl, or C₁-C₆haloalkyl. In some embodiments of a compound of Formula (I), (Ia), or (Ib), R¹ is hydrogen or C₁-C₆alkyl. In some embodiments of a compound of Formula (I), (Ia), or (Ib), R¹ is hydrogen.

In some embodiments of a compound of Formula (I), (Ia), or (Ib), R² is C₁-C₆alkyl. In some embodiments of a compound of Formula (I), (Ia), or (Ib), R² is hydrogen.

In some embodiments of a compound of Formula (I), (Ia), or (Ib), R³ is C₁-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}. In some embodiments of a compound of Formula (I), (Ia), or (Ib), R³ is C₄-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}. In some embodiments of a compound of Formula (I), (Ia), or (Ib), R³ is C₁-C₁₀alkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}. In some embodiments of a compound of Formula (I), (Ia), or (Ib), R³ is C₄-C₁₀alkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}. In some embodiments of a compound of Formula (I), (Ia), or (Ib), R³ is C₁-C₁₀alkyl or C₁-C₆alkylene(aryl); wherein the alkyl, alkylene, and aryl is optionally and independently substituted with one or more R^{3a}. In some embodiments of a compound of Formula (I), (Ia), or (Ib), R³ is C₄-C₁₀alkyl or C₂-C₆alkylene(aryl); wherein the alkyl, alkylene, and aryl is optionally and independently substituted with one or more R^{3a}. In some embodiments of a compound of Formula (I), (Ia), or (Ib), R³ is C₁-C₁₀alkyl optionally and independently substituted with one or more R^{3a}. In some embodiments of a compound of Formula (I), (Ia), or (Ib), R³ is C₄-C₁₀alkyl optionally and independently substituted with one or more R^{3a}. In some embodiments of a compound of Formula (I), (Ia), or (Ib), R³ is C₄-C₁₀alkyl. In some embodiments of a compound of Formula (I), (Ia), or (Ib), R³ is C₁-C₆alkylene(aryl); wherein the alkylene and aryl is optionally and independently substituted with one or more R^{3a}. In some embodiments of a compound of Formula (I), (Ia), or (Ib), R³ is C₂-C₆alkylene(aryl); wherein the alkylene and aryl is optionally and independently substituted with one or more R^{3a}. In some embodiments of a compound of Formula (I), (Ia), or (Ib), R³ is C₂-C₆alkylene(aryl); wherein the aryl is optionally and independently substituted with one or more R^{3a}.

In some embodiments of a compound of Formula (I), (Ia), or (Ib), each R^{3a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; or two R^{3a} on the same atom are taken together to form an oxo. In some embodiments of a compound of Formula (I), (Ia), or (Ib), each R^{3a} is independently deuterium, halogen, —OH, —OR^{a}, C₁-C₆alkyl, C₁-C₆haloalkyl, or C₁-C₆deuteroalkyl; or two R^{3a} on the same atom are taken together to form an oxo. In some embodiments of a compound of Formula (I), (Ia), or (Ib), each R^{3a} is independently halogen, —OR^{a}, or C₁-C₆haloalkyl. In some embodiments of a compound of Formula (I), (Ia), or (Ib), each R^{3a} is independently C₁-C₆haloalkyl.

In some embodiments of a compound of Formula (I), (Ia), or (Ib), X is N. In some embodiments of a compound of Formula (I), (Ia), or (Ib), X is CR^{X}.

In some embodiments of a compound of Formula (I), (Ia), or (Ib), R^{X} is hydrogen, halogen, C₁-C₆alkyl, or C₁-C₆haloalkyl. In some embodiments of a compound of Formula (I), (Ia), or (Ib), R^{X} is hydrogen or C₁-C₆alkyl. In some embodiments of a compound of Formula (I), (Ia), or (Ib), R^{X} is hydrogen.

In some embodiments of a compound of Formula (I), (Ia), or (Ib), R is hydrogen, halogen, C₁-C₆alkyl, or C₁-C₆haloalkyl. In some embodiments of a compound of Formula (I), (Ia), or (Ib), R⁵ is hydrogen or C₁-C₆alkyl. In some embodiments of a compound of Formula (I), (Ia), or (Ib), R⁵ is hydrogen.

In some embodiments of a compound of Formula (I), (Ia), or (Ib), each R⁶ is chloro. In some embodiments of a compound of Formula (I), (Ia), or (Ib), each R⁶ is fluoro. In some embodiments of a compound of Formula (I), (Ia), or (Ib), one R⁶ is fluoro and the other R⁶ is chloro.

In some embodiments of a compound of Formula (I), (Ia), or (Ib),

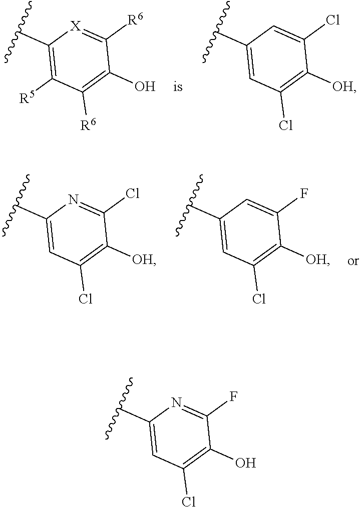

In some embodiments of a compound of Formula (I), (Ia), or (Ib),

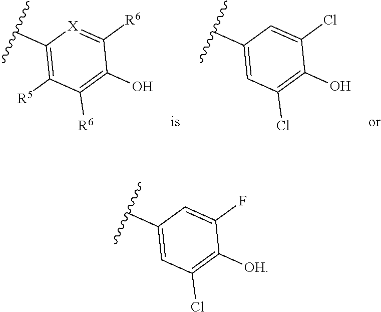

In some embodiments of a compound of Formula (I), (Ia), or (Ib),

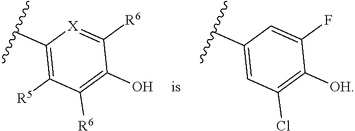

In some embodiments of a compound of Formula (I), (Ia), or (Ib),

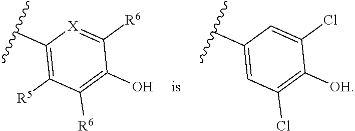

In some embodiments of a compound of Formula (I), (Ia), or (Ib), R⁴ is cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more with one or more R^{4a}.

In some embodiments of a compound of Formula (I), (Ia), or (Ib), R⁴ is cycloalkyl, heterocycloalkyl, aryl, or heteroaryl; wherein the cycloalkyl, heterocycloalkyl, aryl, and heteroaryl is optionally substituted with one or more with one or more R^{4a}.

In some embodiments of a compound of Formula (I), (Ia), or (Ib), R⁴ is cycloalkyl optionally substituted with one or more with one or more R^{4a}.

In some embodiments of a compound of Formula (I), (Ia), or (Ib), R⁴ is heterocycloalkyl optionally substituted with one or more with one or more R^{4a}.

In some embodiments of a compound of Formula (I), (Ia), or (Ib), R⁴ is aryl or heteroaryl; wherein the aryl and heteroaryl is optionally substituted with one or more with one or more R^{4a}.

In some embodiments of a compound of Formula (I), (Ia), or (Ib), R⁴ is aryl optionally substituted with one or more with one or more R^{4a}.

In some embodiments of a compound of Formula (I), (Ia), or (Ib), R⁴ is heteroaryl optionally substituted with one or more with one or more R^{4a}.

In some embodiments of a compound of Formula (I), (Ia), or (Ib), each R^{4a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein the alkyl is optionally and independently substituted with one or more R; or two R^{4a} on the same atom are taken together to form an oxo.

In some embodiments of a compound of Formula (I), (Ia), or (Ib), each R^{4a} is independently deuterium, halogen, —CN, —OH, —OR^{a}, —S(═O)R^{a}, —NR^{c}R^{d}, C₁-C₆alkyl, or C₁-C₆haloalkyl; wherein the alkyl is optionally and independently substituted with one or more R; or two R^{4a} on the same atom are taken together to form an oxo.

In some embodiments of a compound of Formula (I), (Ia), or (Ib), each R^{4a} is independently deuterium, halogen, —CN, —S(═O)R^{a}, or C₁-C₆alkyl optionally and independently substituted with one or more R; or two R^{4a} on the same atom are taken together to form an oxo.

In some embodiments of a compound of Formula (I), (Ia), or (Ib), each R^{4a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R^{4a} on the same atom are taken together to form an oxo.

In some embodiments of a compound of Formula (I), (Ia), or (Ib), each R^{4a} is independently deuterium, halogen, —CN, —OH, —OR^{a}, —S(═O)R^{a}, —NR^{c}R^{d}, C₁-C₆alkyl, C₁-C₆haloalkyl, C₁-C₆deuteroalkyl, cycloalkyl, or heterocycloalkyl; or two R^{4a} on the same atom are taken together to form an oxo.

In some embodiments of a compound of Formula (I), (Ia), or (Ib), each R^{4a} is independently halogen, —CN, —OH, —OR^{a}, —S(═O)R^{a}, —NR^{c}R^{d}, C₁-C₆alkyl, or C₁-C₆haloalkyl; or two R^{4a} on the same atom are taken together to form an oxo.

In some embodiments of a compound of Formula (I), (Ia), or (Ib), each R^{4a} is independently halogen, —S(═O)R^{a}, C₁-C₆alkyl, or C₁-C₆haloalkyl; or two R^{4a} on the same atom are taken together to form an oxo.

In some embodiments of a compound of Formula (I), (Ia), or (Ib), each R^{4a} is independently halogen, —S(═O)R^{a}, C₁-C₆alkyl, or C₁-C₆haloalkyl.

In some embodiments of a compound of Formula (I), (Ia), or (Ib), each R^{4a} is independently halogen.

In some embodiments of a compound of Formula (I), (Ia), or (Ib), two R^{4a} on the same carbon are taken together to form a cycloalkyl or heterocycloalkyl; each optionally substituted with one or more R. In some embodiments of a compound of Formula (I), (Ia), or (Ib), two R^{4a} on the same carbon are taken together to form a cycloalkyl optionally substituted with one or more R. In some embodiments of a compound of Formula (I), (Ia), or (Ib), two R^{4a} on the same carbon are taken together to form a heterocycloalkyl; each optionally substituted with one or more R.

In some embodiments of a compound of Formula (I), (Ia), or (Ib), two R^{4a} on the different atoms are taken together to form a cycloalkyl optionally substituted with one or more R. In some embodiments of a compound of Formula (I), (Ia), or (Ib), two R^{4a} on the different atoms are taken together to form a heterocycloalkyl optionally substituted with one or more R. In some embodiments of a compound of Formula (I), (Ia), or (Ib), two R^{4a} on the different atoms are taken together to form a aryl optionally substituted with one or more R. In some embodiments of a compound of Formula (I), (Ia), or (Ib), two R^{4a} on the different atoms are taken together to form a heteroaryl optionally substituted with one or more R.

In some embodiments of a compound of Formula (I),

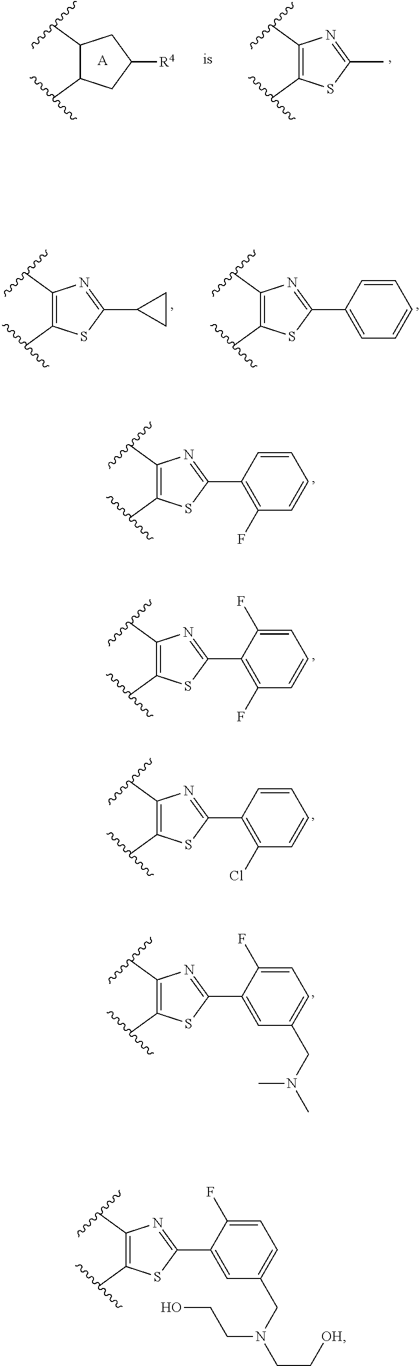

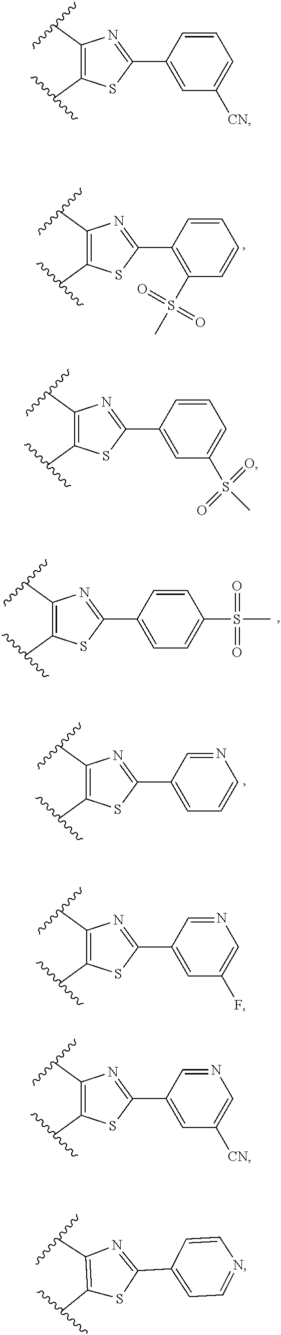

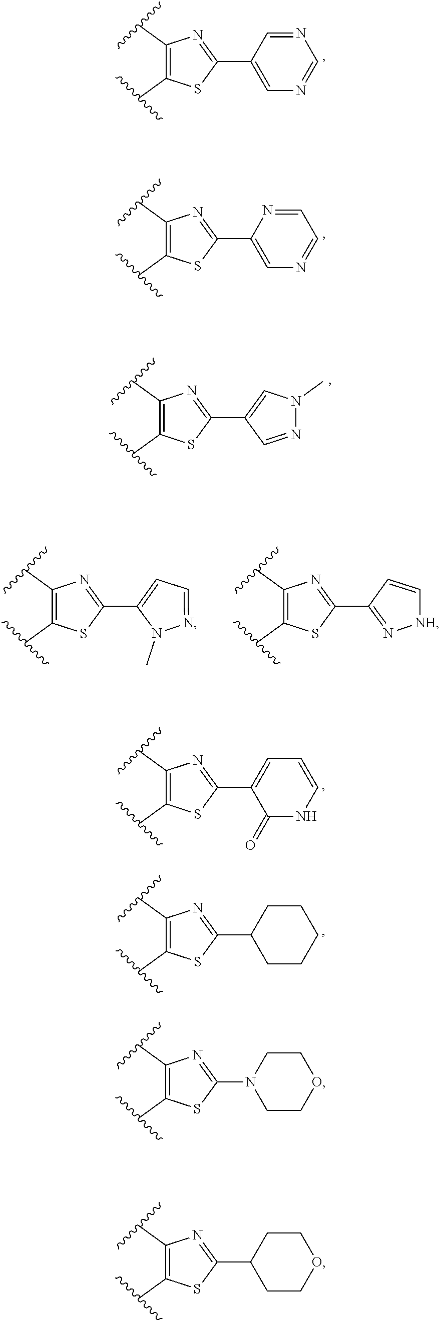

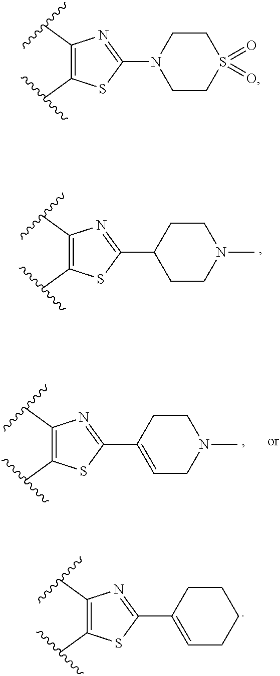 [SMILES: *c1nc(N2CCS(=O)(=O)CC2)sc1* ; *c1nc(C2CCN(C)CC2)sc1* ; *c1nc(C2=CCN(C)CC2)sc1* ; *c1nc(C2=CCCCC2)sc1*]

In some embodiments of a compound of Formula (I),

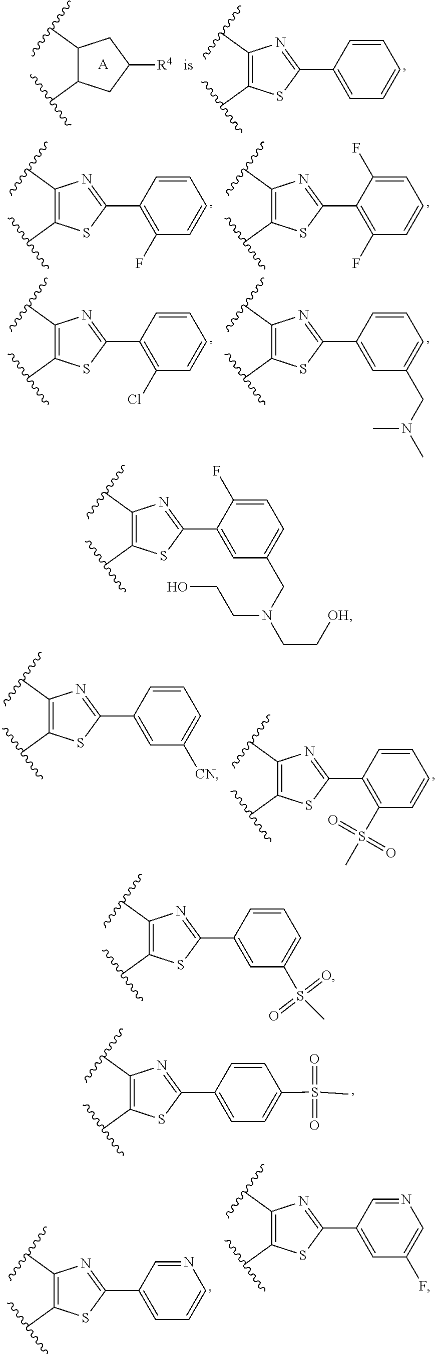

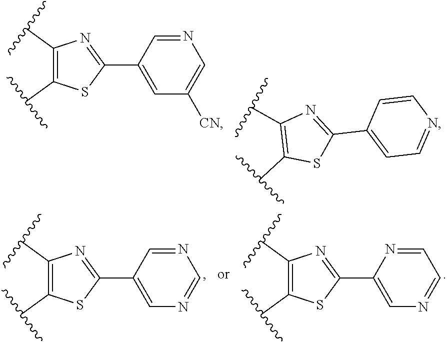 [SMILES: *c1nc(-c2cncc(C#N)c2)sc1* ; *c1nc(-c2ccncc2)sc1* ; *c1nc(-c2cncnc2)sc1* ; *c1nc(-c2cnccn2)sc1*]

In some embodiments of a compound of Formula (I)

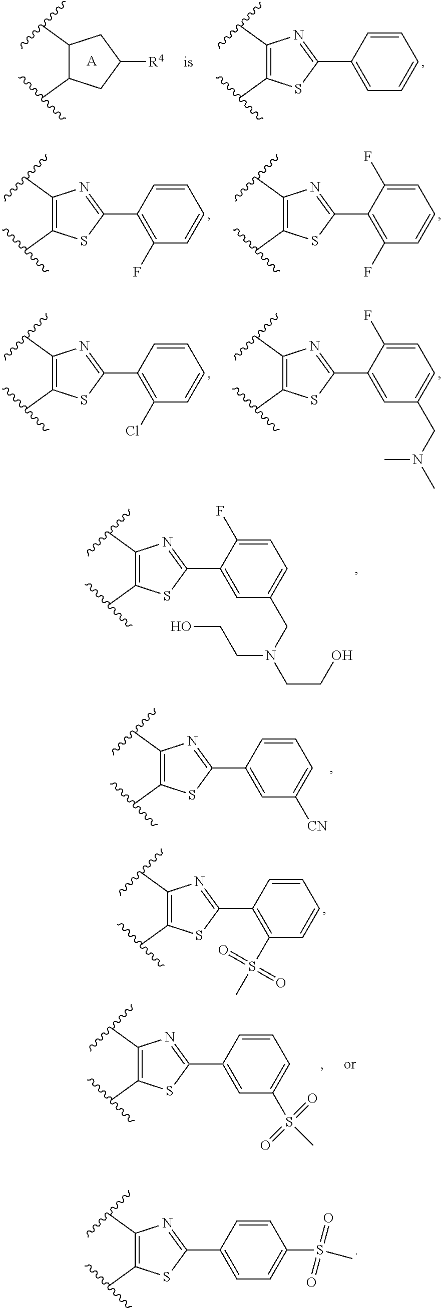

In some embodiments of a compound of Formula (I),

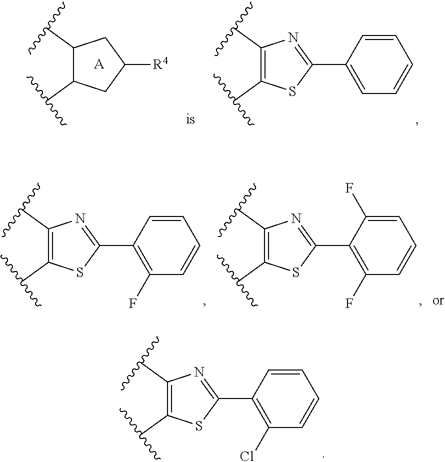

In some embodiments of a compound of Formula (I),

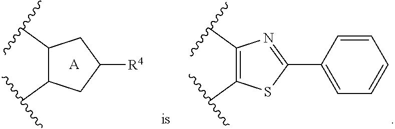

In some embodiments of a compound of Formula (I),

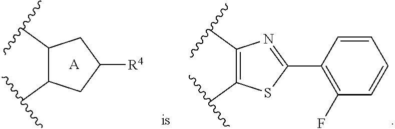

In some embodiments of a compound of Formula (I),

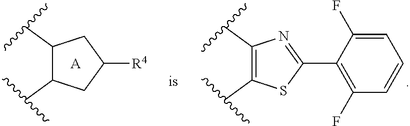

In some embodiments of a compound of Formula (I),

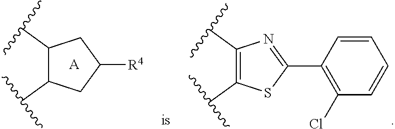

In some embodiments of a compound of Formula (I),

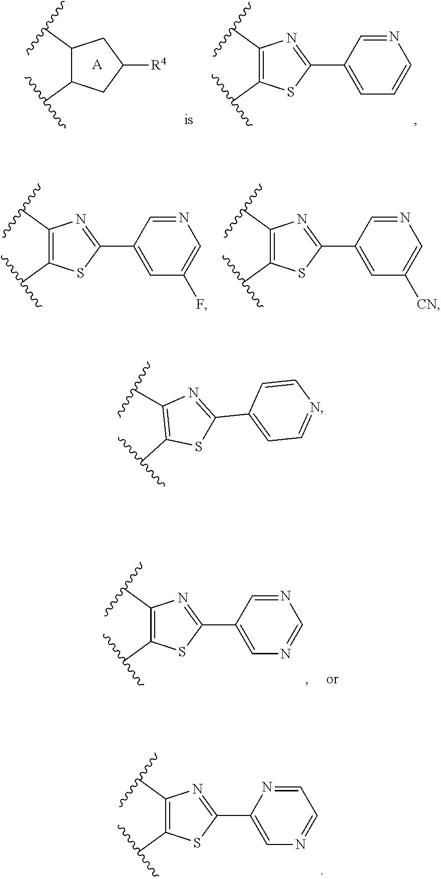

In some embodiments of a compound of Formula (I),

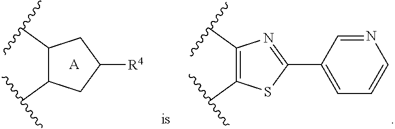

In some embodiments of a compound of Formula (I), is

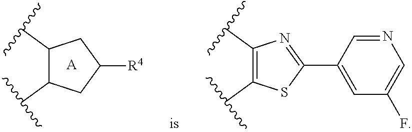

In some embodiments of a compound of Formula (I), is

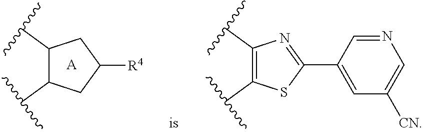

In some embodiments of a compound of Formula (I) is

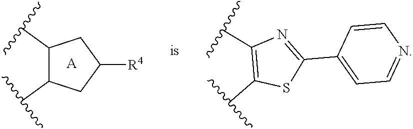

In some embodiments of a compound of Formula (I), is

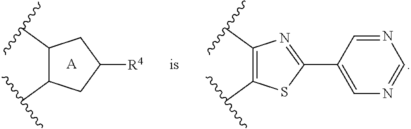

In some embodiments of a compound of Formula (I), is

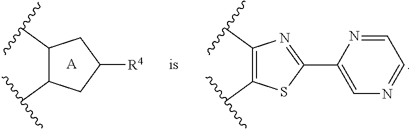

Also disclosed herein is a compound of Formula (II), or a pharmaceutically acceptable salt, solvate, or stereoisomer thereof:

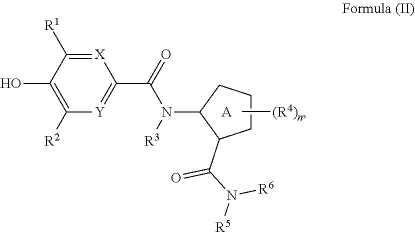

wherein:
R¹ is halogen;
R² is halogen;
X is N or CR^{X};
R^{X} is hydrogen, deuterium, halogen, —CN, —OH, —OR^{a}, —NR^{c}R^{d}, C₁-C₆alkyl, C₁-C₆haloalkyl, or C₁-C₆deuteroalkyl;
Y is N or CR^{Y};
R^{Y} is hydrogen, deuterium, halogen, —CN, —OH, —OR^{a}, —NR^{c}R^{d}, C₁-C₆alkyl, C₁-C₆haloalkyl, or C₁-C₆deuteroalkyl;
R³ is hydrogen, C₁-C₆alkyl, C₁-C₆haloalkyl, or C₁-C₆deuteroalkyl;
Ring A is a pyrazole;
each R⁴ is independently deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
n is 0-2;
R⁵ is hydrogen, C₁-C₆alkyl, C₁-C₆haloalkyl, or C₁-C₆deuteroalkyl;
R⁶ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally substituted with one or more R^{6a};
each R^{6a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R^{6b};
or two R^{6a} on the same atom are taken together to form an oxo;
each R^{6b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{6b} on the same atom are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently deuterium, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R on the same atom are taken together to form an oxo.

In some embodiments of a compound of Formula (II), the compound is of Formula (IIa), or a pharmaceutically acceptable salt, solvate, or stereoisomer thereof:

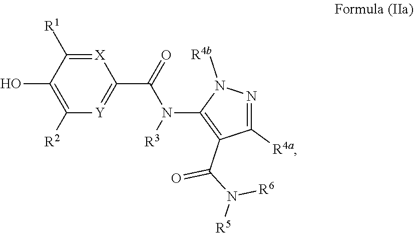

wherein:
R^{4a} is hydrogen or R⁴; and
R^{4b} is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R.

In some embodiments, of a compound of Formula (II), the compound is of Formula (IIb), or a pharmaceutically acceptable salt, solvate, or stereoisomer thereof:

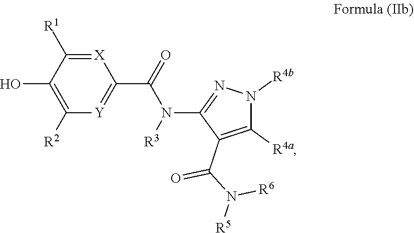

wherein:
R^{4a} is hydrogen or R⁴; and
R^{4b} is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R.

In some embodiments, of a compound of Formula (II), the compound is of Formula (IIc), or a pharmaceutically acceptable salt, solvate, or stereoisomer thereof:

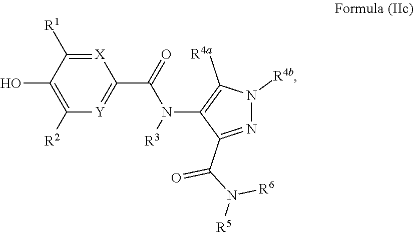

wherein:
R^{4a} is hydrogen or R⁴; and
R^{4b} is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R.

In some embodiments, of a compound of Formula (II), the compound is of Formula (IId), or a pharmaceutically acceptable salt, solvate, or stereoisomer thereof:

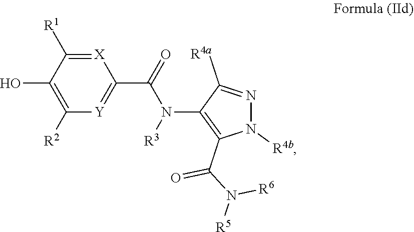

wherein:
R^{4a} is hydrogen or R⁴; and
R^{4b} is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R.

In some embodiments of a compound of Formula (IIa)-(IId), R^{4a} is hydrogen, halogen, or C₁-C₆alkyl. In some embodiments of a compound of Formula (IIa)-(IId), R^{4a} is hydrogen.

In some embodiments of a compound of Formula (II) or (IIa)-(IId), R¹ is fluoro or chloro. In some embodiments of a compound of Formula (II) or (IIa)-(IId), R¹ is chloro.

In some embodiments of a compound of Formula (II) or (IIa)-(IId), R² is fluoro or chloro. In some embodiments of a compound of Formula (II) or (IIa)-(IId), R² is chloro.

In some embodiments of a compound of Formula (II) or (IIa)-(IId), X is CR^{X}. In some embodiments of a compound of Formula (II) or (IIa)-(IId), X is N.

In some embodiments of a compound of Formula (II) or (IIa)-(IId), R^{X} is hydrogen, deuterium, halogen, or C₁-C₆alkyl. In some embodiments of a compound of Formula (II) or (IIa)-(IId), R^{X} is hydrogen or halogen. In some embodiments of a compound of Formula (II) or (IIa)-(IId), R^{X} is hydrogen.

In some embodiments of a compound of Formula (II) or (IIa)-(IId), Y is CR^{Y}. In some embodiments of a compound of Formula (II) or (IIa)-(IId), Y is N.

In some embodiments of a compound of Formula (II) or (IIa)-(IId), R^{Y} is hydrogen, deuterium, halogen, or C₁-C₆alkyl. In some embodiments of a compound of Formula (II) or (IIa)-(IId), R is hydrogen or halogen. In some embodiments of a compound of Formula (II) or (IIa)-(IId), R^{Y} is hydrogen.

In some embodiments of a compound of Formula (II) or (IIa)-(IId), R³ is hydrogen or C₁-C₆alkyl. In some embodiments of a compound of Formula (II) or (IIa)-(IId), R³ is hydrogen.

In some embodiments of a compound of Formula (II) or (IIa)-(IId), each R⁴ is independently deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R. In some embodiments of a compound of Formula (II) or (IIa)-(IId), each R⁴ is independently deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is optionally and independently substituted with one or more R. In some embodiments of a compound of Formula (II) or (IIa)-(IId), each R⁴ is independently deuterium, halogen, C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is optionally and independently substituted with one or more R. In some embodiments of a compound of Formula (II) or (IIa)-(IId), each R⁴ is independently C₁-C₆alkyl, C₁-C₆haloalkyl, cycloalkyl, or aryl; wherein each alkyl, cycloalkyl, and aryl is optionally and independently substituted with one or more R. In some embodiments of a compound of Formula (II) or (IIa)-(IId), each R⁴ is independently C₁-C₆alkyl, C₁-C₆haloalkyl, cycloalkyl, or aryl. In some embodiments of a compound of Formula (II) or (IIa)-(IId), each R⁴ is independently C₁-C₆alkyl or C₁-C₆haloalkyl. In some embodiments of a compound of Formula (II) or (IIa)-(IId), each R⁴ is independently aryl.

In some embodiments of a compound of Formula (II) or (IIa)-(IId), n is 0 or 1. In some embodiments of a compound of Formula (II) or (IIa)-(IId), n is 0. In some embodiments of a compound of Formula (II) or (IIa)-(IId), n is 1. In some embodiments of a compound of Formula (II) or (IIa)-(IId), n is 2.

In some embodiments of a compound of Formula (IIa)-(IId), R^{4b} is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R. In some embodiments of a compound of Formula (IIa)-(IId), R^{4b} is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is optionally and independently substituted with one or more R. In some embodiments of a compound of Formula (IIa)-(IId), R^{4b} is C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is optionally and independently substituted with one or more R. In some embodiments of a compound of Formula (IIa)-(IId), R^{4b} is C₁-C₆alkyl, C₁-C₆haloalkyl, cycloalkyl, or aryl; wherein each alkyl, cycloalkyl, and aryl is optionally and independently substituted with one or more R. In some embodiments of a compound of Formula (IIa)-(IId), R^{4b} is C₁-C₆alkyl, C₁-C₆haloalkyl, cycloalkyl, or aryl. In some embodiments of a compound of Formula (IIa)-(IId), R^{4b} is C₁-C₆alkyl or C₁-C₆haloalkyl. In some embodiments of a compound of Formula (IIa)-(IId), R^{4b} is aryl.

In some embodiments of a compound of Formula (II) or (IIa)-(IId), R⁵ is hydrogen or C₁-C₆alkyl. In some embodiments of a compound of Formula (II) or (IIa)-(IId), R⁵ is hydrogen.

In some embodiments of a compound of Formula (II) or (IIa)-(IId), R⁶ is C₁-C₆alkyl, C₁-C₆haloalkyl, C₁-C₆deuteroalkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally substituted with one or more R^{6a}. In some embodiments of a compound of Formula (II) or (IIa)-(IId), R⁶ is C₁-C₆alkyl optionally substituted with one or more R^{6a}. In some embodiments of a compound of Formula (II) or (IIa)-(IId), R⁶ is C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more R^{6a}. In some embodiments of a compound of Formula (II) or (IIa)-(IId), R⁶ is C₁-C₆alkylene(aryl) or C₁-C₆alkylene(heteroaryl); wherein the alkylene, aryl, and heteroaryl is optionally substituted with one or more R^{6a}. In some embodiments of a compound of Formula (II) or (IIa)-(IId), R⁶ is C₁-C₆alkylene(aryl); wherein the alkylene and aryl is optionally substituted with one or more R^{6a}.

In some embodiments of a compound of Formula (II) or (IIa)-(IId), each R^{6a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is optionally and independently substituted with one or more R^{6b}.

In some embodiments of a compound of Formula (II) or (IIa)-(IId), each R^{6a} is independently deuterium, halogen, —CN, —OH, —OR^{a}, —NR^{c}R^{d}, C₁-C₆alkyl, C₁-C₆haloalkyl, or C₁-C₆deuteroalkyl; wherein each alkyl is optionally and independently substituted with one or more R^{6b}. In some embodiments of a compound of Formula (II) or (IIa)-(IId), each R^{6a} is independently —OR^{a}, C₁-C₆alkyl, or C₁-C₆haloalkyl.

In some embodiments of a compound of Formula (II) or (IIa)-(IId), each R^{6b} is independently deuterium, halogen, —CN, —OH, —OR^{a}, —NR^{c}R^{d}, C₁-C₆alkyl, or C₁-C₆haloalkyl. In some embodiments of a compound of Formula (II) or (IIa)-(IId), each R^{6b} is independently deuterium, halogen, C₁-C₆alkyl, or C₁-C₆haloalkyl.

Also disclosed herein is a compound of Formula (III), or a pharmaceutically acceptable salt, solvate, or stereoisomer thereof:

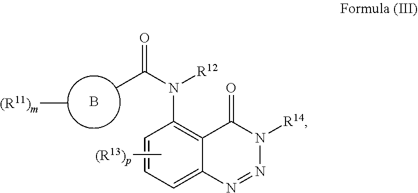

wherein:
Ring B is cycloalkyl, heterocycloalkyl, aryl, or heteroaryl;
each R¹¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
m is 1-5;
R¹² is hydrogen, C₁-C₆alkyl, C₁-C₆haloalkyl, or C₁-C₆deuteroalkyl;
each R¹³ is independently deuterium, halogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0-3;
R¹⁴ is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is optionally and independently substituted with one or more R^{14a}.
each R^{14a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R^{14b};
or two R^{14a} on the same atom are taken together to form an oxo;
each R^{14b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{14b} on the same atom are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently deuterium, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R on the same atom are taken together to form an oxo.

In some embodiments of a compound of Formula (II), Ring B is aryl or heteroaryl. In some embodiments of a compound of Formula (II), Ring B is aryl. In some embodiments of a compound of Formula (II), Ring B is heteroaryl. In some embodiments of a compound of Formula (II), Ring B is 5- or 6-membered heteroaryl. In some embodiments of a compound of Formula (II), Ring B is phenyl or pyridyl. In some embodiments of a compound of Formula (II), Ring B is phenyl. In some embodiments of a compound of Formula (II), Ring B is pyridyl.

In some embodiments of a compound of Formula (II), each R¹¹ is independently deuterium, halogen, —CN, —OH, —OR^{a}, —NR^{c}R^{d}, C₁-C₆alkyl, or C₁-C₆haloalkyl.

In some embodiments of a compound of Formula (II), each R¹¹ is independently halogen, —OH, C₁-C₆alkyl, or C₁-C₆haloalkyl. In some embodiments of a compound of Formula (II), each R¹¹ is independently halogen or —OH.

In some embodiments of a compound of Formula (III), m is 1-3. In some embodiments of a compound of Formula (III), m is 2 or 3. In some embodiments of a compound of Formula (III), m is 2. In some embodiments of a compound of Formula (III), m is 3.

In some embodiments, of a compound of Formula (III), the compound is of Formula (IIIa), or a pharmaceutically acceptable salt, solvate, or stereoisomer thereof:

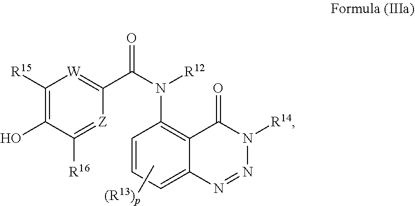

wherein:
R¹⁵ is halogen;
R¹⁶ is halogen;
W is N or CR^{W};
R^{W} is hydrogen, deuterium, halogen, —CN, —OH, —OR^{a}, —NR^{c}R^{d}, C₁-C₆alkyl, C₁-C₆haloalkyl, or C₁-C₆deuteroalkyl;
Z is N or CR^{Z};
R^{Z} is hydrogen, deuterium, halogen, —CN, —OH, —OR^{a}, —NR^{c}R^{d}, C₁-C₆alkyl, C₁-C₆haloalkyl, or C₁-C₆deuteroalkyl.

In some embodiments of a compound of Formula (IIIa), R¹⁵ is fluoro or chloro. In some embodiments of a compound of Formula (IIIa), R⁵ is chloro.

In some embodiments of a compound of Formula (IIIa), R¹⁶ is fluoro or chloro. In some embodiments of a compound of Formula (IIIa), R¹⁶ is chloro.

In some embodiments of a compound of Formula (IIIa), W is CR^{W}. In some embodiments of a compound of Formula (IIIa), W is N.

In some embodiments of a compound of Formula (IIIa), R^{W} is hydrogen, deuterium, halogen, or C₁-C₆alkyl. In some embodiments of a compound of Formula (IIIa), R^{W} is hydrogen or halogen. In some embodiments of a compound of Formula (IIIa), R^{W} is hydrogen.

In some embodiments of a compound of Formula (IIIa), Z is CR^{Z}. In some embodiments of a compound of Formula (IIIa), Z is N.

In some embodiments of a compound of Formula (IIIa), R^{Z} is hydrogen, deuterium, halogen, or C₁-C₆alkyl. In some embodiments of a compound of Formula (IIIa), R^{Z} is hydrogen or halogen. In some embodiments of a compound of Formula (IIIa), R^{Z} is hydrogen.

In some embodiments of a compound of Formula (III) or (IIIa), R¹² is hydrogen or C₁-C₆alkyl. In some embodiments of a compound of Formula (III) or (IIIa), R¹² is hydrogen.

In some embodiments of a compound of Formula (III) or (IIIa), each R¹³ is independently deuterium, halogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein each alkyl, cycloalkyl, heterocycloalkyl, aryl, and heteroaryl is optionally and independently substituted with one or more R. In some embodiments of a compound of Formula (III) or (IIIa), each R¹³ is independently halogen, C₁-C₆alkyl, or C₁-C₆haloalkyl. In some embodiments of a compound of Formula (III) or (IIIa), each R¹³ is independently halogen or C₁-C₆alkyl. In some embodiments of a compound of Formula (III) or (IIIa), each R¹³ is independently halogen. In some embodiments of a compound of Formula (III) or (IIIa), each R¹³ is independently C₁-C₆alkyl.

In some embodiments of a compound of Formula (III) or (IIIa), p is 0-2. In some embodiments of a compound of Formula (III) or (IIIa), p is 0 or 1. In some embodiments of a compound of Formula (III) or (IIIa), p is 0. In some embodiments of a compound of Formula (III) or (IIIa), p is 1. In some embodiments of a compound of Formula (III) or (IIIa), p is 2.

In some embodiments of a compound of Formula (III) or (IIIa), R¹⁴ is C₁-C₆alkyl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, cycloalkyl, heterocycloalkyl, aryl, and heteroaryl is optionally and independently substituted with one or more R^{14a}. In some embodiments of a compound of Formula (III) or (IIIa), R¹⁴ is C₁-C₆alkyl optionally substituted with one or more R^{14a}. In some embodiments of a compound of Formula (III) or (IIIa), R¹⁴ is C₁-C₆alkylene(aryl) or C₁-C₆alkylene(heteroaryl); wherein each alkylene, aryl, and heteroaryl is optionally and independently substituted with one or more R^{14a}. In some embodiments of a compound of Formula (III) or (IIIa), R¹⁴ is C₁-C₆alkylene(aryl); wherein each alkylene and aryl is optionally and independently substituted with one or more R^{14a}.

In some embodiments of a compound of Formula (III) or (IIIa), each R^{14a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is optionally and independently substituted with one or more R^{14b}.

In some embodiments of a compound of Formula (III) or (IIIa), each R^{14a} is independently deuterium, halogen, —CN, —OH, —OR^{a}, —NR^{c}R^{d}, C₁-C₆alkyl, C₁-C₆haloalkyl, or C₁-C₆deuteroalkyl; wherein each alkyl is optionally and independently substituted with one or more R^{14b}.

In some embodiments of a compound of Formula (III) or (IIIa), each R^{14a} is independently —OR^{a}, C₁-C₆alkyl, or C₁-C₆haloalkyl. In some embodiments of a compound of Formula (III) or (IIIa), each R^{14a} is independently C₁-C₆haloalkyl.

In some embodiments of a compound of Formula (III) or (IIIa), each R^{14b} is independently deuterium, halogen, —CN, —OH, —OR^{a}, —NR^{c}R^{d}, C₁-C₆alkyl, or C₁-C₆haloalkyl. In some embodiments of a compound of Formula (III) or (IIIa), each R^{14b} is independently deuterium, halogen, C₁-C₆alkyl, or C₁-C₆haloalkyl.

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein each alkyl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein each alkyl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is independently hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein each alkyl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are independently hydrogen. In some embodiments of a compound disclosed herein, each R_{c} and R^{d} are independently C₁-C₆alkyl.

In some embodiments of a compound disclosed herein, R_{c} and R^{d} are taken together with the atom to which they are attached to form a 5- or 6-membered heterocycloalkyl optionally substituted with one or more R. In some embodiments of a compound disclosed herein, R^{c} and R^{d} are taken together with the atom to which they are attached to form a 5-membered heterocycloalkyl optionally substituted with one or more R. In some embodiments of a compound disclosed herein, R^{c} and R^{d} are taken together with the atom to which they are attached to form a 6-membered heterocycloalkyl optionally substituted with one or more R.

In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl; or two R on the same atom form an oxo. In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl; or two R on the same atom form an oxo. In some embodiments of a compound disclosed herein, each R is independently halogen, —CN, —OH, —OC₁-C₃alkyl, —OC₁-C₃haloalkyl, —NH₂, C₁-C₃alkyl, C₁-C₃haloalkyl, C₁-C₃deuteroalkyl, or C₃-C₆cycloalkyl; or two R on the same atom form an oxo. In some embodiments of a compound disclosed herein, each R is independently halogen, C₁-C₃alkyl, or C₁-C₃haloalkyl; or two R on the same atom form an oxo. In some embodiments of a compound disclosed herein, each R is independently halogen, C₁-C₃alkyl, or C₁-C₃haloalkyl. In some embodiments of a compound disclosed herein, each R is independently halogen or C₁-C₃alkyl. In some embodiments of a compound disclosed herein, each R is independently halogen.

Any combination of the groups described above for the various variables is contemplated herein. Throughout the specification, groups and substituents thereof are chosen by one skilled in the field to provide stable moieties and compounds.

Described herein is a compound, or a pharmaceutically acceptable salt, solvate, or stereoisomer thereof, selected from a compound in Table 1a.

TABLE 1a

| Example | Structure |
|---|---|
| 1 | 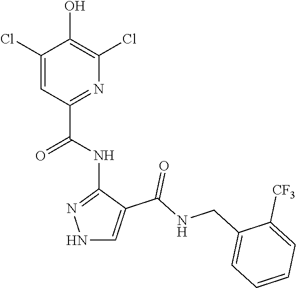 [SMILES: O=C(Nc1n[nH]cc1C(=O)NCc1ccccc1C(F)(F)F)c1cc(Cl)c(O)c(Cl)n1] |
| 2 | 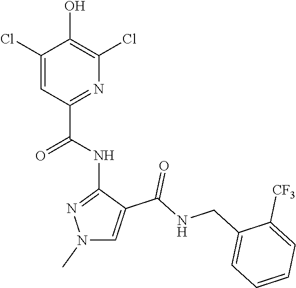 [SMILES: Cn1cc(C(=O)NCc2ccccc2C(F)(F)F)c(NC(=O)c2cc(Cl)c(O)c(Cl)n2)n1] |
| 3 | 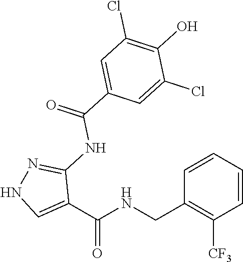 [SMILES: O=C(Nc1n[nH]cc1C(=O)NCc1ccccc1C(F)(F)F)c1cc(Cl)c(O)c(Cl)c1] |
| 4 | 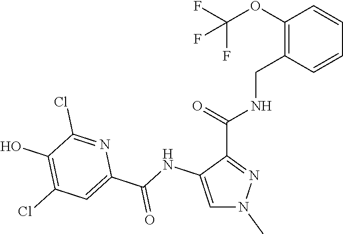 [SMILES: Cn1cc(NC(=O)c2cc(Cl)c(O)c(Cl)n2)c(C(=O)NCc2ccccc2OC(F)(F)F)n1] |
| 5 | 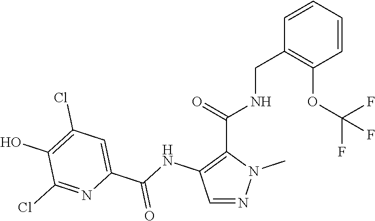 [SMILES: Cn1ncc(NC(=O)c2cc(Cl)c(O)c(Cl)n2)c1C(=O)NCc1ccccc1OC(F)(F)F] |
| 6 | 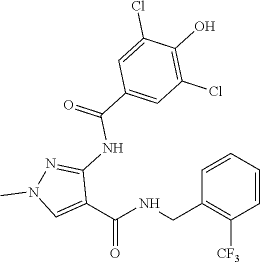 [SMILES: Cn1cc(C(=O)NCc2ccccc2C(F)(F)F)c(NC(=O)c2cc(Cl)c(O)c(Cl)c2)n1] |
| 7 | 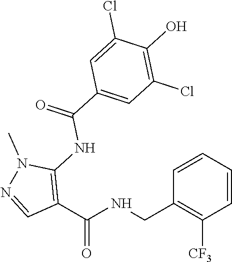 [SMILES: Cn1ncc(C(=O)NCc2ccccc2C(F)(F)F)c1NC(=O)c1cc(Cl)c(O)c(Cl)c1] |
| 8 | 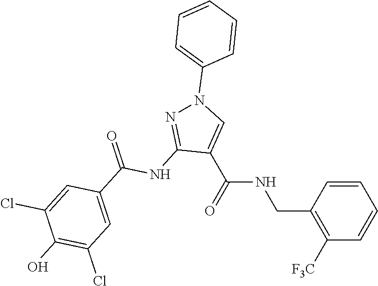 [SMILES: O=C(Nc1nn(-c2ccccc2)cc1C(=O)NCc1ccccc1C(F)(F)F)c1cc(Cl)c(O)c(Cl)c1] |
| 9 | 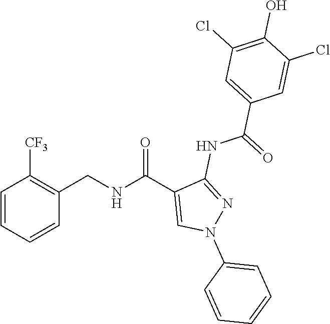 [SMILES: O=C(Nc1nn(-c2ccccc2)cc1C(=O)NCc1ccccc1C(F)(F)F)c1cc(Cl)c(O)c(Cl)c1] |
| 10 | 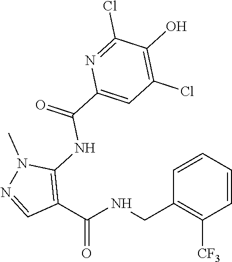 [SMILES: Cn1ncc(C(=O)NCc2ccccc2C(F)(F)F)c1NC(=O)c1cc(Cl)c(O)c(Cl)n1] |
| 11 | 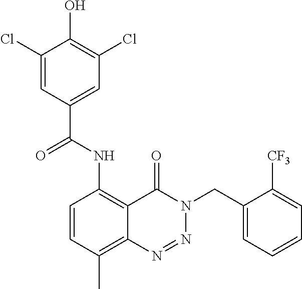 [SMILES: Cc1ccc(NC(=O)c2cc(Cl)c(O)c(Cl)c2)c2c(=O)n(Cc3ccccc3C(F)(F)F)nnc12] |
| 12 | 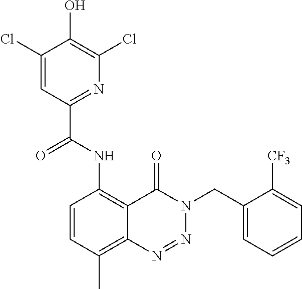 [SMILES: Cc1ccc(NC(=O)c2cc(Cl)c(O)c(Cl)n2)c2c(=O)n(Cc3ccccc3C(F)(F)F)nnc12] |
| 13 | 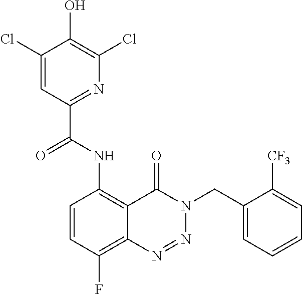 [SMILES: O=C(Nc1ccc(F)c2nnn(Cc3ccccc3C(F)(F)F)c(=O)c12)c1cc(Cl)c(O)c(Cl)n1] |
| 14 | 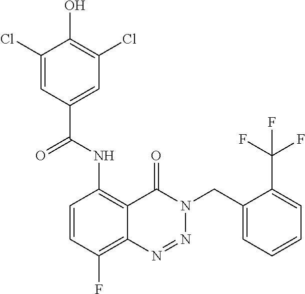 [SMILES: O=C(Nc1ccc(F)c2nnn(Cc3ccccc3C(F)(F)F)c(=O)c12)c1cc(Cl)c(O)c(Cl)c1] |
| 15 | 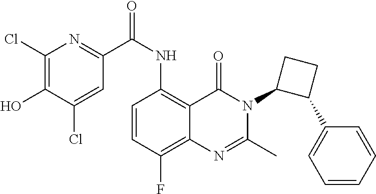 [SMILES: Cc1nc2c(F)ccc(NC(=O)c3cc(Cl)c(O)c(Cl)n3)c2c(=O)n1[C@H]1CC[C@@H]1c1ccccc1] |
| 16 | 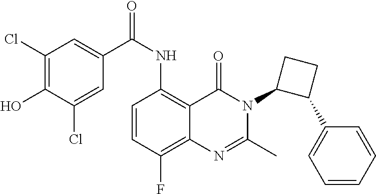 [SMILES: Cc1nc2c(F)ccc(NC(=O)c3cc(Cl)c(O)c(Cl)c3)c2c(=O)n1[C@H]1CC[C@@H]1c1ccccc1] |
| 17 | 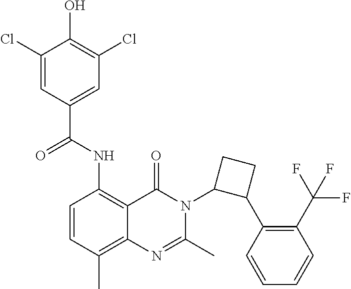 [SMILES: Cc1ccc(NC(=O)c2cc(Cl)c(O)c(Cl)c2)c2c(=O)n(C3CCC3c3ccccc3C(F)(F)F)c(C)nc12] |
| 18 | 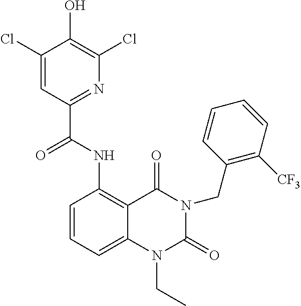 [SMILES: CCn1c(=O)n(Cc2ccccc2C(F)(F)F)c(=O)c2c(NC(=O)c3cc(Cl)c(O)c(Cl)n3)cccc21] |
| 19 | 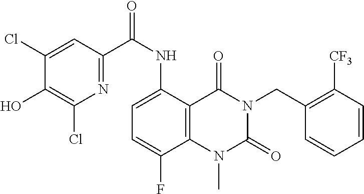 [SMILES: Cn1c(=O)n(Cc2ccccc2C(F)(F)F)c(=O)c2c(NC(=O)c3cc(Cl)c(O)c(Cl)n3)ccc(F)c21] |
| 20 | 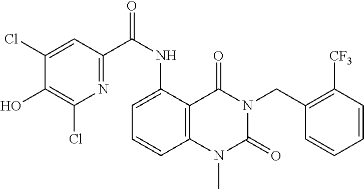 [SMILES: Cn1c(=O)n(Cc2ccccc2C(F)(F)F)c(=O)c2c(NC(=O)c3cc(Cl)c(O)c(Cl)n3)cccc21] |
| 21 | 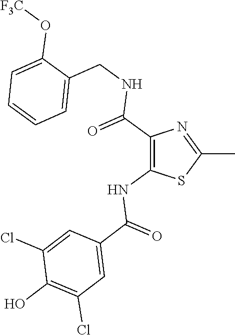 [SMILES: Cc1nc(C(=O)NCc2ccccc2OC(F)(F)F)c(NC(=O)c2cc(Cl)c(O)c(Cl)c2)s1] |
| 22 | 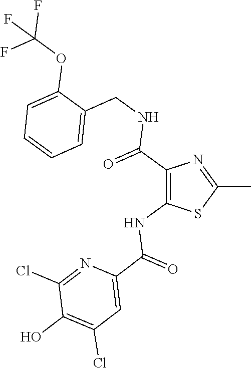 [SMILES: Cc1nc(C(=O)NCc2ccccc2OC(F)(F)F)c(NC(=O)c2cc(Cl)c(O)c(Cl)n2)s1] |
| 23 | 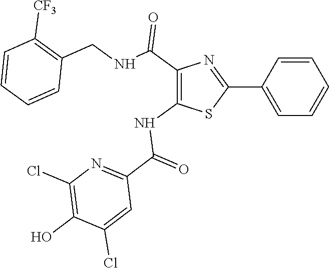 [SMILES: O=C(Nc1sc(-c2ccccc2)nc1C(=O)NCc1ccccc1C(F)(F)F)c1cc(Cl)c(O)c(Cl)n1] |
| 24 | 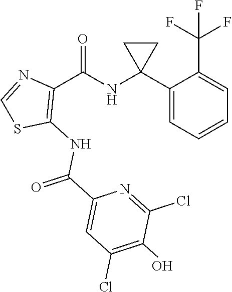 [SMILES: O=C(Nc1scnc1C(=O)NC1(c2ccccc2C(F)(F)F)CC1)c1cc(Cl)c(O)c(Cl)n1] |
| 25 | 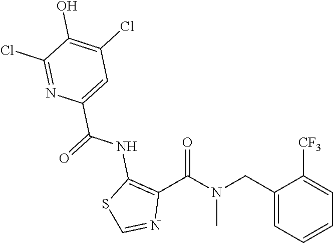 [SMILES: CN(Cc1ccccc1C(F)(F)F)C(=O)c1ncsc1NC(=O)c1cc(Cl)c(O)c(Cl)n1] |
| 26 | 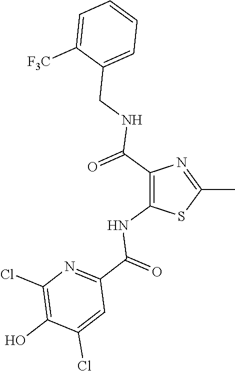 [SMILES: Cc1nc(C(=O)NCc2ccccc2C(F)(F)F)c(NC(=O)c2cc(Cl)c(O)c(Cl)n2)s1] |
| 27 | 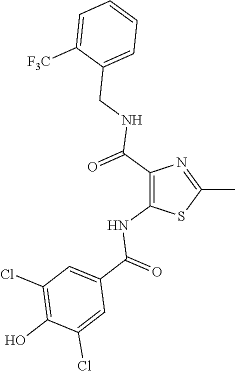 [SMILES: Cc1nc(C(=O)NCc2ccccc2C(F)(F)F)c(NC(=O)c2cc(Cl)c(O)c(Cl)c2)s1] |
| 28 | 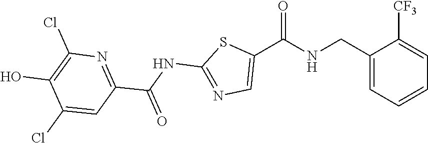 [SMILES: O=C(Nc1ncc(C(=O)NCc2ccccc2C(F)(F)F)s1)c1cc(Cl)c(O)c(Cl)n1] |
| 29 | 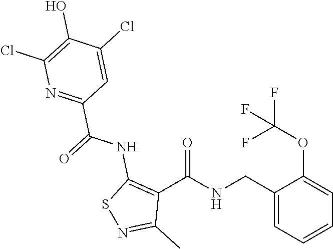 [SMILES: Cc1nsc(NC(=O)c2cc(Cl)c(O)c(Cl)n2)c1C(=O)NCc1ccccc1OC(F)(F)F] |
| 30 | 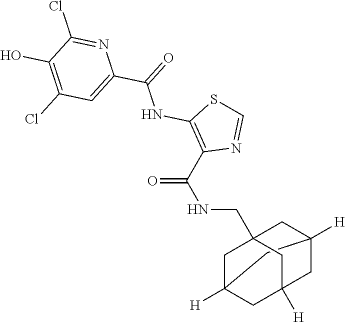 [SMILES: O=C(Nc1scnc1C(=O)NCC12CC3CC(CC(C3)C1)C2)c1cc(Cl)c(O)c(Cl)n1] |
| 31 | 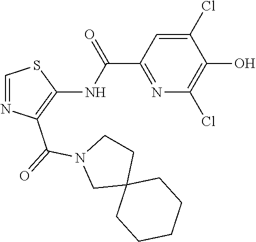 [SMILES: O=C(Nc1scnc1C(=O)N1CCC2(CCCCC2)C1)c1cc(Cl)c(O)c(Cl)n1] |
| 32 | 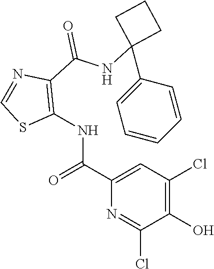 [SMILES: O=C(Nc1scnc1C(=O)NC1(c2ccccc2)CCC1)c1cc(Cl)c(O)c(Cl)n1] |
| 33 | 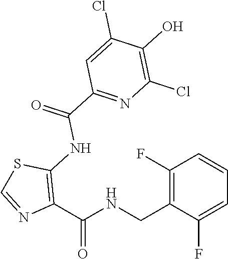 [SMILES: O=C(Nc1scnc1C(=O)NCc1c(F)cccc1F)c1cc(Cl)c(O)c(Cl)n1] |
| 34 | 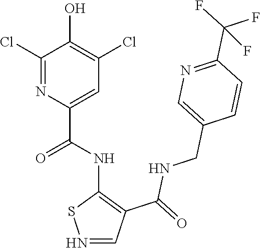 [SMILES: O=C(Nc1sncc1C(=O)NCc1ccc(C(F)(F)F)nc1)c1cc(Cl)c(O)c(Cl)n1] |
| 35 | 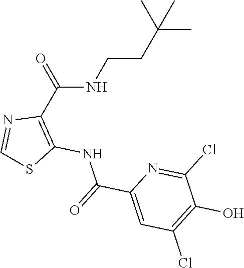 [SMILES: CC(C)(C)CCNC(=O)c1ncsc1NC(=O)c1cc(Cl)c(O)c(Cl)n1] |
| 36 | 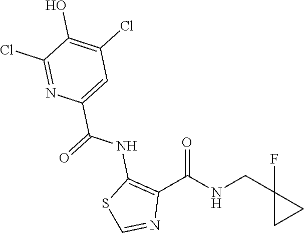 [SMILES: O=C(Nc1scnc1C(=O)NCC1(F)CC1)c1cc(Cl)c(O)c(Cl)n1] |
| 37 | 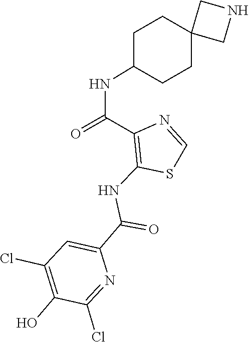 [SMILES: O=C(Nc1scnc1C(=O)NC1CCC2(CC1)CNC2)c1cc(Cl)c(O)c(Cl)n1] |
| 38 | 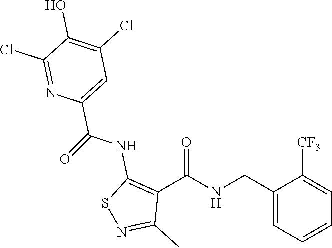 [SMILES: Cc1nsc(NC(=O)c2cc(Cl)c(O)c(Cl)n2)c1C(=O)NCc1ccccc1C(F)(F)F] |
| 39 | 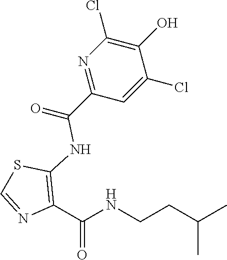 [SMILES: CC(C)CCNC(=O)c1ncsc1NC(=O)c1cc(Cl)c(O)c(Cl)n1] |
| 40 | 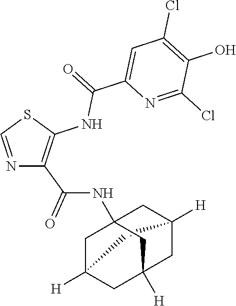 [SMILES: O=C(Nc1scnc1C(=O)NC12C[C@H]3C[C@@H](C1)C[C@@H](C2)C3)c1cc(Cl)c(O)c(Cl)n1] |
| 41 | 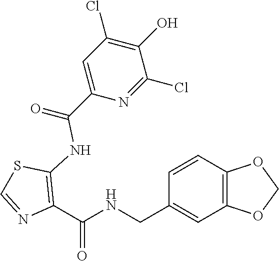 [SMILES: O=C(Nc1scnc1C(=O)NCc1ccc2c(c1)OCO2)c1cc(Cl)c(O)c(Cl)n1] |
| 42 | 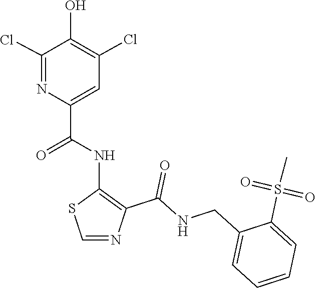 [SMILES: CS(=O)(=O)c1ccccc1CNC(=O)c1ncsc1NC(=O)c1cc(Cl)c(O)c(Cl)n1] |
| 43 | 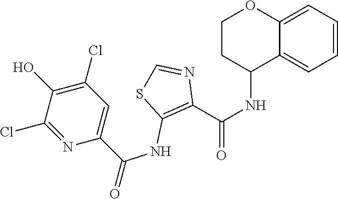 [SMILES: O=C(Nc1scnc1C(=O)NC1CCOc2ccccc21)c1cc(Cl)c(O)c(Cl)n1] |
| 44 | 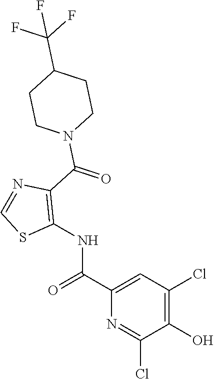 [SMILES: O=C(Nc1scnc1C(=O)N1CCC(C(F)(F)F)CC1)c1cc(Cl)c(O)c(Cl)n1] |
| 46 | 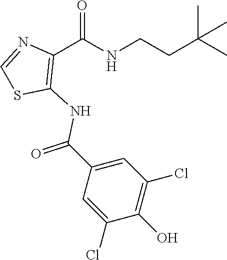 [SMILES: CC(C)(C)CCNC(=O)c1ncsc1NC(=O)c1cc(Cl)c(O)c(Cl)c1] |
| 48 | 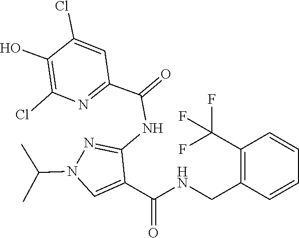 [SMILES: CC(C)n1cc(C(=O)NCc2ccccc2C(F)(F)F)c(NC(=O)c2cc(Cl)c(O)c(Cl)n2)n1] |
| 49 | 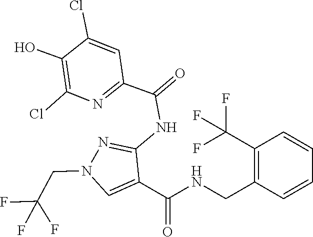 [SMILES: O=C(Nc1nn(CC(F)(F)F)cc1C(=O)NCc1ccccc1C(F)(F)F)c1cc(Cl)c(O)c(Cl)n1] |
| 50 | 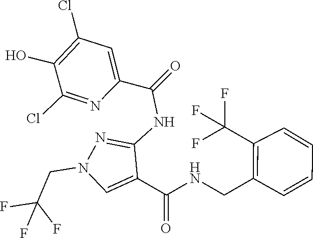 [SMILES: O=C(Nc1nn(CC(F)(F)F)cc1C(=O)NCc1ccccc1C(F)(F)F)c1cc(Cl)c(O)c(Cl)n1] |
| 51 | 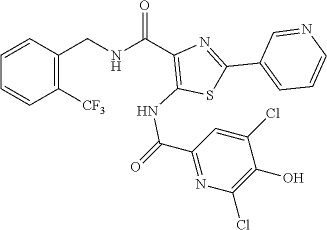 [SMILES: O=C(Nc1sc(-c2cccnc2)nc1C(=O)NCc1ccccc1C(F)(F)F)c1cc(Cl)c(O)c(Cl)n1] |
| 52 | 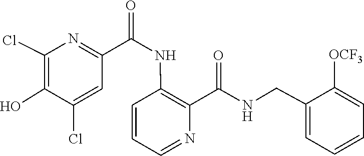 [SMILES: O=C(Nc1cccnc1C(=O)NCc1ccccc1OC(F)(F)F)c1cc(Cl)c(O)c(Cl)n1] |
| 53 | 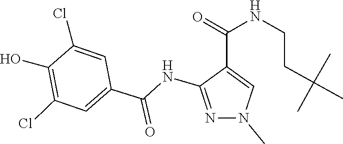 [SMILES: Cn1cc(C(=O)NCCC(C)(C)C)c(NC(=O)c2cc(Cl)c(O)c(Cl)c2)n1] |
| 54 | 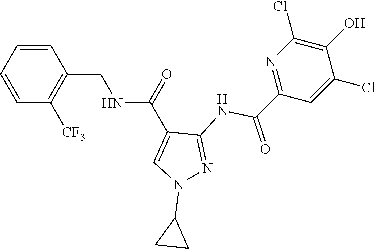 [SMILES: O=C(Nc1nn(C2CC2)cc1C(=O)NCc1ccccc1C(F)(F)F)c1cc(Cl)c(O)c(Cl)n1] |
| 55 | 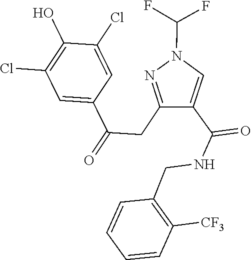 [SMILES: O=C(Cc1nn(C(F)F)cc1C(=O)NCc1ccccc1C(F)(F)F)c1cc(Cl)c(O)c(Cl)c1] |
| 56 | 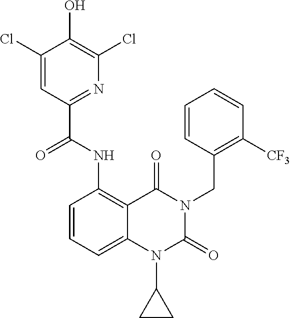 [SMILES: O=C(Nc1cccc2c1c(=O)n(Cc1ccccc1C(F)(F)F)c(=O)n2C1CC1)c1cc(Cl)c(O)c(Cl)n1] |
| 57 | 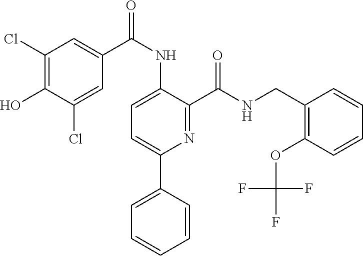 [SMILES: O=C(Nc1ccc(-c2ccccc2)nc1C(=O)NCc1ccccc1OC(F)(F)F)c1cc(Cl)c(O)c(Cl)c1] |
| 58 | 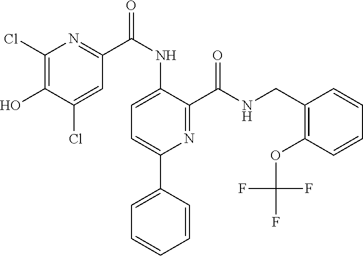 [SMILES: O=C(Nc1ccc(-c2ccccc2)nc1C(=O)NCc1ccccc1OC(F)(F)F)c1cc(Cl)c(O)c(Cl)n1] |
| 59 | 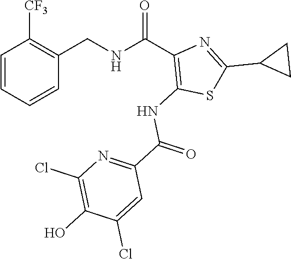 [SMILES: O=C(Nc1sc(C2CC2)nc1C(=O)NCc1ccccc1C(F)(F)F)c1cc(Cl)c(O)c(Cl)n1] |
| 60 | 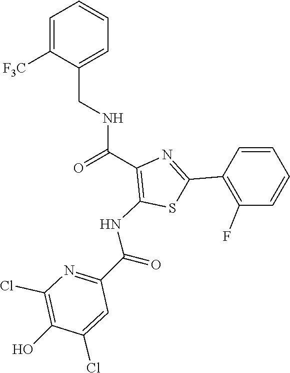 [SMILES: O=C(Nc1sc(-c2ccccc2F)nc1C(=O)NCc1ccccc1C(F)(F)F)c1cc(Cl)c(O)c(Cl)n1] |
| 61 | 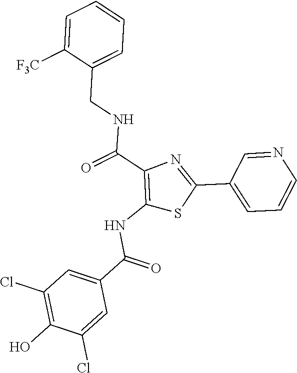 [SMILES: O=C(Nc1sc(-c2cccnc2)nc1C(=O)NCc1ccccc1C(F)(F)F)c1cc(Cl)c(O)c(Cl)c1] |
| 62 | 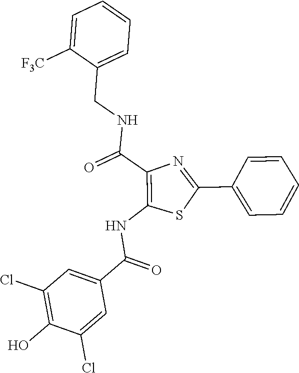 [SMILES: O=C(Nc1sc(-c2ccccc2)nc1C(=O)NCc1ccccc1C(F)(F)F)c1cc(Cl)c(O)c(Cl)c1] |
| 63 | 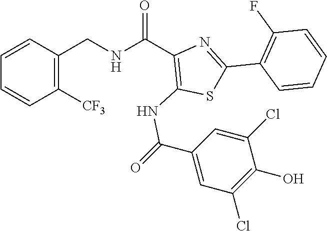 [SMILES: O=C(Nc1sc(-c2ccccc2F)nc1C(=O)NCc1ccccc1C(F)(F)F)c1cc(Cl)c(O)c(Cl)c1] |
| 64 | 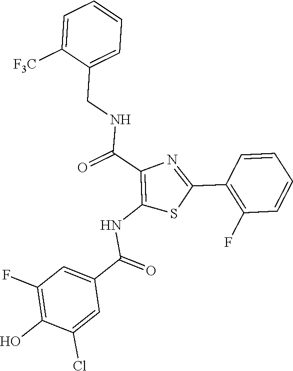 [SMILES: O=C(Nc1sc(-c2ccccc2F)nc1C(=O)NCc1ccccc1C(F)(F)F)c1cc(F)c(O)c(Cl)c1] |
| 65 | 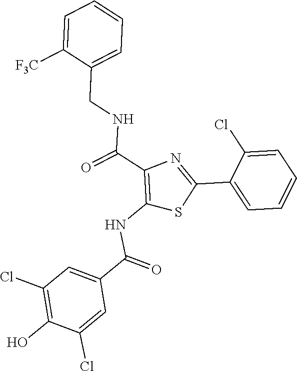 [SMILES: O=C(Nc1sc(-c2ccccc2Cl)nc1C(=O)NCc1ccccc1C(F)(F)F)c1cc(Cl)c(O)c(Cl)c1] |
| 66 | 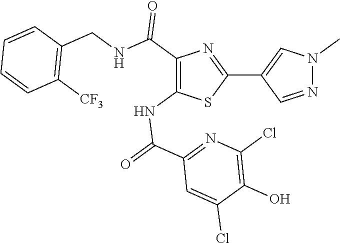 [SMILES: Cn1cc(-c2nc(C(=O)NCc3ccccc3C(F)(F)F)c(NC(=O)c3cc(Cl)c(O)c(Cl)n3)s2)cn1] |
| 67 | 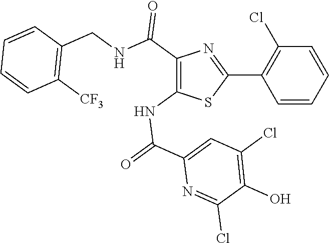 [SMILES: O=C(Nc1sc(-c2ccccc2Cl)nc1C(=O)NCc1ccccc1C(F)(F)F)c1cc(Cl)c(O)c(Cl)n1] |
| 68 | 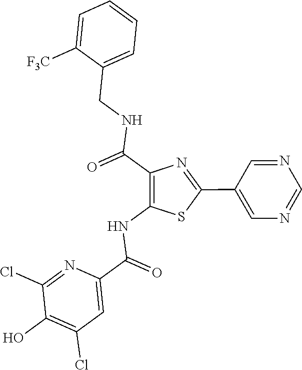 [SMILES: O=C(Nc1sc(-c2cncnc2)nc1C(=O)NCc1ccccc1C(F)(F)F)c1cc(Cl)c(O)c(Cl)n1] |
| 69 | 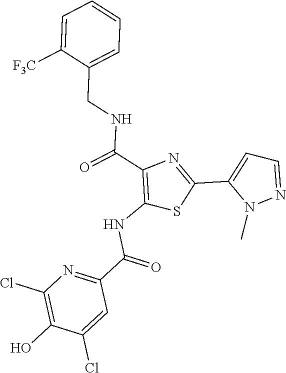 [SMILES: Cn1nccc1-c1nc(C(=O)NCc2ccccc2C(F)(F)F)c(NC(=O)c2cc(Cl)c(O)c(Cl)n2)s1] |
| 70 | 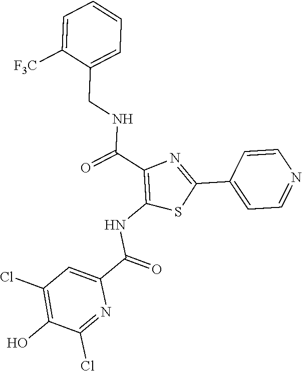 [SMILES: O=C(Nc1sc(-c2ccncc2)nc1C(=O)NCc1ccccc1C(F)(F)F)c1cc(Cl)c(O)c(Cl)n1] |
| 71 | 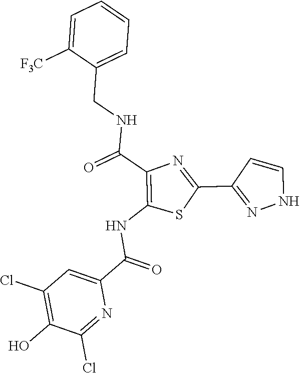 [SMILES: O=C(Nc1sc(-c2cc[nH]n2)nc1C(=O)NCc1ccccc1C(F)(F)F)c1cc(Cl)c(O)c(Cl)n1] |
| 72 | 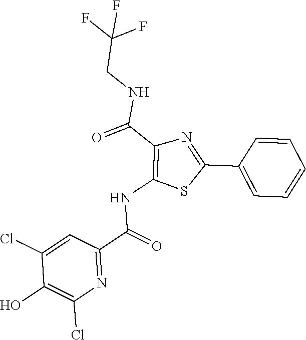 [SMILES: O=C(Nc1sc(-c2ccccc2)nc1C(=O)NCC(F)(F)F)c1cc(Cl)c(O)c(Cl)n1] |
| 73 | 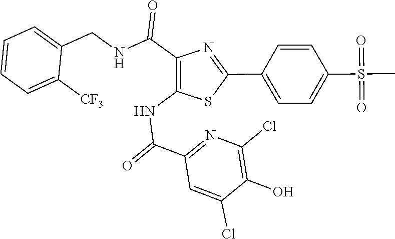 [SMILES: CS(=O)(=O)c1ccc(-c2nc(C(=O)NCc3ccccc3C(F)(F)F)c(NC(=O)c3cc(Cl)c(O)c(Cl)n3)s2)cc1] |
| 74 | 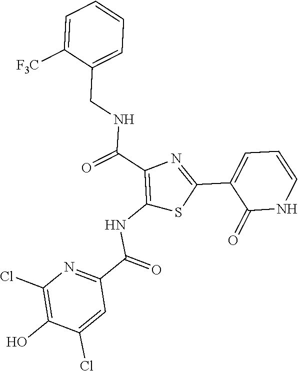 [SMILES: O=C(Nc1sc(-c2ccc[nH]c2=O)nc1C(=O)NCc1ccccc1C(F)(F)F)c1cc(Cl)c(O)c(Cl)n1] |
| 75 | 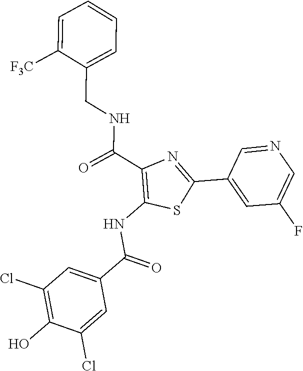 [SMILES: O=C(Nc1sc(-c2cncc(F)c2)nc1C(=O)NCc1ccccc1C(F)(F)F)c1cc(Cl)c(O)c(Cl)c1] |
| 76 | 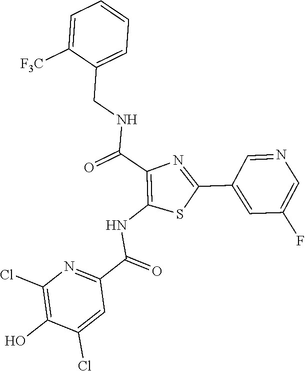 [SMILES: O=C(Nc1sc(-c2cncc(F)c2)nc1C(=O)NCc1ccccc1C(F)(F)F)c1cc(Cl)c(O)c(Cl)n1] |
| 77 | 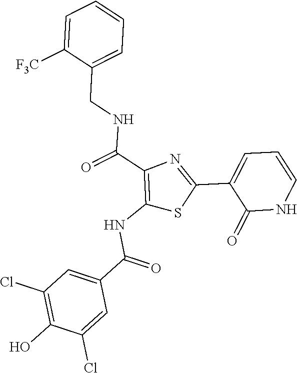 [SMILES: O=C(Nc1sc(-c2ccc[nH]c2=O)nc1C(=O)NCc1ccccc1C(F)(F)F)c1cc(Cl)c(O)c(Cl)c1] |
| 78 | 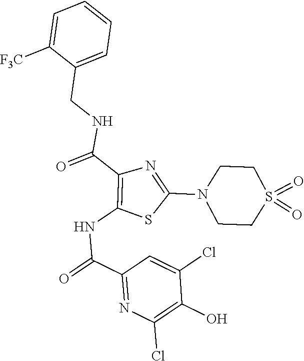 [SMILES: O=C(Nc1sc(N2CCS(=O)(=O)CC2)nc1C(=O)NCc1ccccc1C(F)(F)F)c1cc(Cl)c(O)c(Cl)n1] |
| 79 | 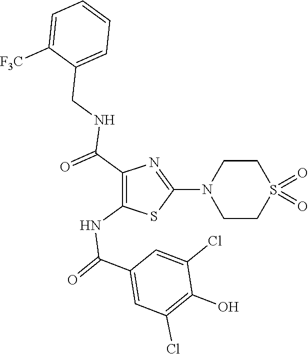 [SMILES: O=C(Nc1sc(N2CCS(=O)(=O)CC2)nc1C(=O)NCc1ccccc1C(F)(F)F)c1cc(Cl)c(O)c(Cl)c1] |
| 80 | 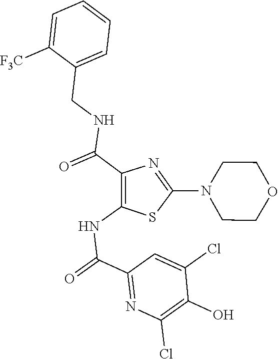 [SMILES: O=C(Nc1sc(N2CCOCC2)nc1C(=O)NCc1ccccc1C(F)(F)F)c1cc(Cl)c(O)c(Cl)n1] |
| 81 | 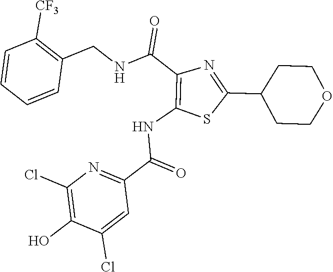 [SMILES: O=C(Nc1sc(C2CCOCC2)nc1C(=O)NCc1ccccc1C(F)(F)F)c1cc(Cl)c(O)c(Cl)n1] |
| 82 | 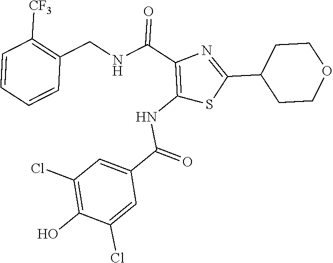 [SMILES: O=C(Nc1sc(C2CCOCC2)nc1C(=O)NCc1ccccc1C(F)(F)F)c1cc(Cl)c(O)c(Cl)c1] |
| 83 | 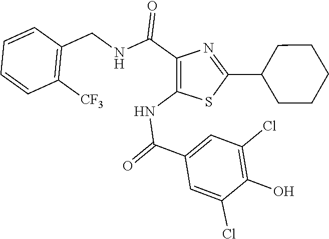 [SMILES: O=C(Nc1sc(C2CCCCC2)nc1C(=O)NCc1ccccc1C(F)(F)F)c1cc(Cl)c(O)c(Cl)c1] |
| 84 | 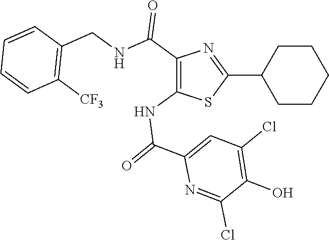 [SMILES: O=C(Nc1sc(C2CCCCC2)nc1C(=O)NCc1ccccc1C(F)(F)F)c1cc(Cl)c(O)c(Cl)n1] |
| 85 | 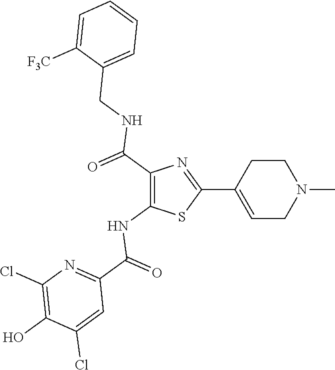 [SMILES: CN1CC=C(c2nc(C(=O)NCc3ccccc3C(F)(F)F)c(NC(=O)c3cc(Cl)c(O)c(Cl)n3)s2)CC1] |
| 86 | 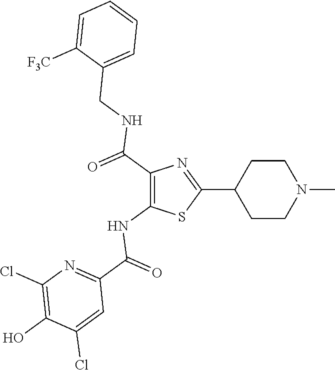 [SMILES: CN1CCC(c2nc(C(=O)NCc3ccccc3C(F)(F)F)c(NC(=O)c3cc(Cl)c(O)c(Cl)n3)s2)CC1] |
| 87 | 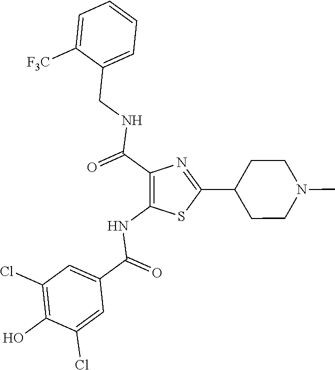 [SMILES: CN1CCC(c2nc(C(=O)NCc3ccccc3C(F)(F)F)c(NC(=O)c3cc(Cl)c(O)c(Cl)c3)s2)CC1] |
| 88 | 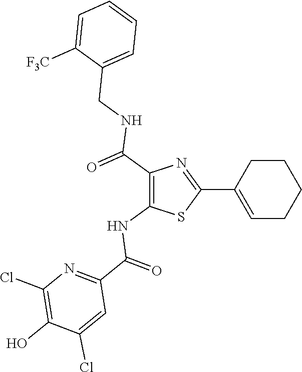 [SMILES: O=C(Nc1sc(C2=CCCCC2)nc1C(=O)NCc1ccccc1C(F)(F)F)c1cc(Cl)c(O)c(Cl)n1] |
| 89 | 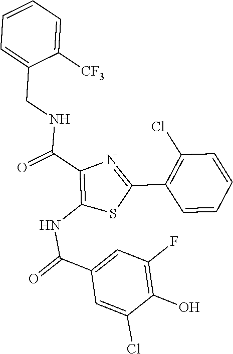 [SMILES: O=C(Nc1sc(-c2ccccc2Cl)nc1C(=O)NCc1ccccc1C(F)(F)F)c1cc(F)c(O)c(Cl)c1] |
| 90 | 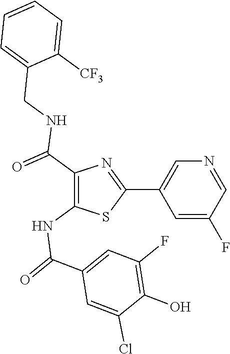 [SMILES: O=C(Nc1sc(-c2cncc(F)c2)nc1C(=O)NCc1ccccc1C(F)(F)F)c1cc(F)c(O)c(Cl)c1] |
| 91 | 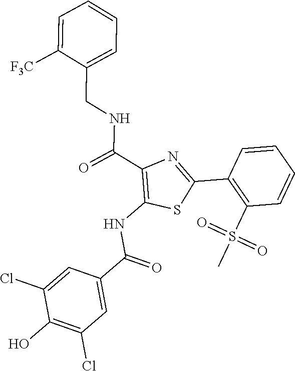 [SMILES: CS(=O)(=O)c1ccccc1-c1nc(C(=O)NCc2ccccc2C(F)(F)F)c(NC(=O)c2cc(Cl)c(O)c(Cl)c2)s1] |
| 92 | 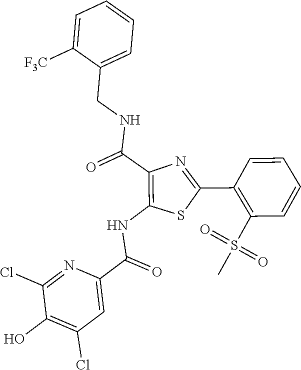 [SMILES: CS(=O)(=O)c1ccccc1-c1nc(C(=O)NCc2ccccc2C(F)(F)F)c(NC(=O)c2cc(Cl)c(O)c(Cl)n2)s1] |
| 93 | 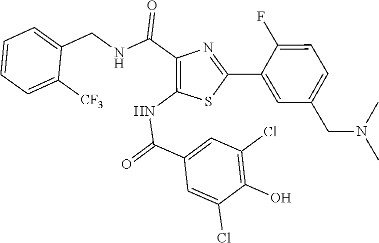 [SMILES: CN(C)Cc1ccc(F)c(-c2nc(C(=O)NCc3ccccc3C(F)(F)F)c(NC(=O)c3cc(Cl)c(O)c(Cl)c3)s2)c1] |
| 94 | 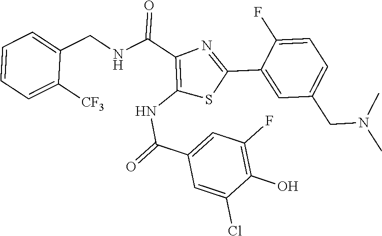 [SMILES: CN(C)Cc1ccc(F)c(-c2nc(C(=O)NCc3ccccc3C(F)(F)F)c(NC(=O)c3cc(F)c(O)c(Cl)c3)s2)c1] |
| 95 | 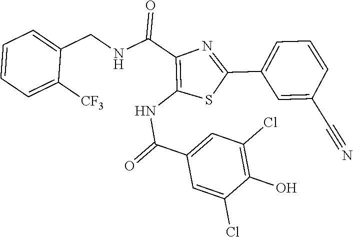 [SMILES: N#Cc1cccc(-c2nc(C(=O)NCc3ccccc3C(F)(F)F)c(NC(=O)c3cc(Cl)c(O)c(Cl)c3)s2)c1] |
| 96 | 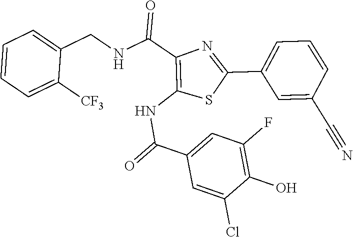 [SMILES: N#Cc1cccc(-c2nc(C(=O)NCc3ccccc3C(F)(F)F)c(NC(=O)c3cc(F)c(O)c(Cl)c3)s2)c1] |
| 97 | 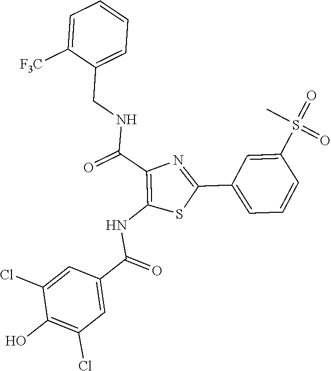 [SMILES: CS(=O)(=O)c1cccc(-c2nc(C(=O)NCc3ccccc3C(F)(F)F)c(NC(=O)c3cc(Cl)c(O)c(Cl)c3)s2)c1] |
| 98 | 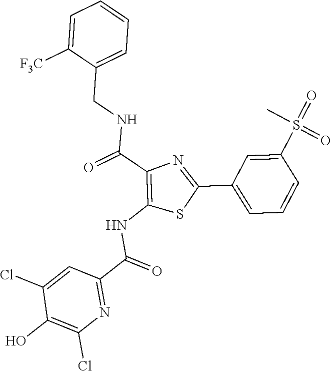 [SMILES: CS(=O)(=O)c1cccc(-c2nc(C(=O)NCc3ccccc3C(F)(F)F)c(NC(=O)c3cc(Cl)c(O)c(Cl)n3)s2)c1] |
| 99 | 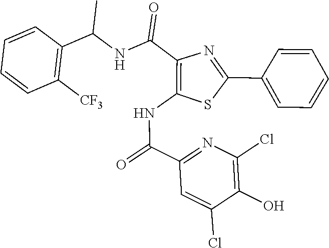 [SMILES: CC(NC(=O)c1nc(-c2ccccc2)sc1NC(=O)c1cc(Cl)c(O)c(Cl)n1)c1ccccc1C(F)(F)F] |
| 100 | 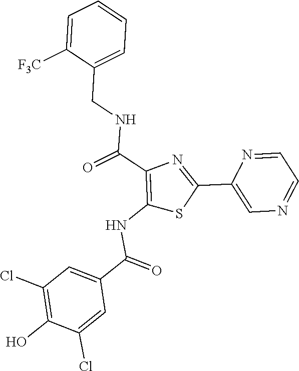 [SMILES: O=C(Nc1sc(-c2cnccn2)nc1C(=O)NCc1ccccc1C(F)(F)F)c1cc(Cl)c(O)c(Cl)c1] |
| 101 | 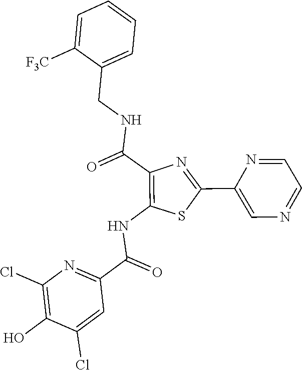 [SMILES: O=C(Nc1sc(-c2cnccn2)nc1C(=O)NCc1ccccc1C(F)(F)F)c1cc(Cl)c(O)c(Cl)n1] |
| 102 | 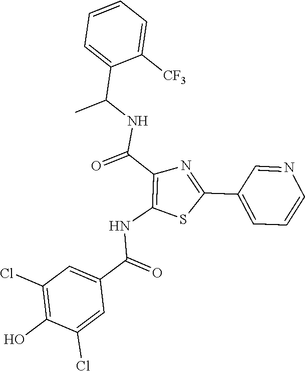 [SMILES: CC(NC(=O)c1nc(-c2cccnc2)sc1NC(=O)c1cc(Cl)c(O)c(Cl)c1)c1ccccc1C(F)(F)F] |
| 103 | 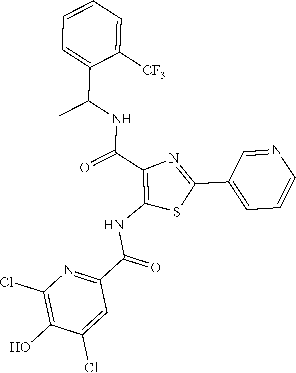 [SMILES: CC(NC(=O)c1nc(-c2cccnc2)sc1NC(=O)c1cc(Cl)c(O)c(Cl)n1)c1ccccc1C(F)(F)F] |
| 104 | 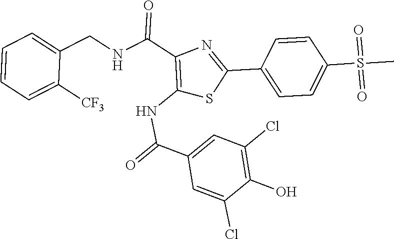 [SMILES: CS(=O)(=O)c1ccc(-c2nc(C(=O)NCc3ccccc3C(F)(F)F)c(NC(=O)c3cc(Cl)c(O)c(Cl)c3)s2)cc1] |
| 105 | 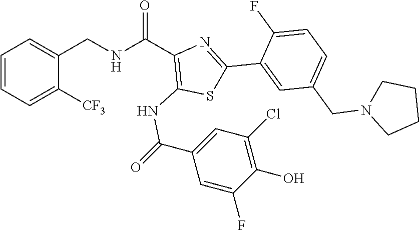 [SMILES: O=C(Nc1sc(-c2cc(CN3CCCC3)ccc2F)nc1C(=O)NCc1ccccc1C(F)(F)F)c1cc(F)c(O)c(Cl)c1] |
| 106 | 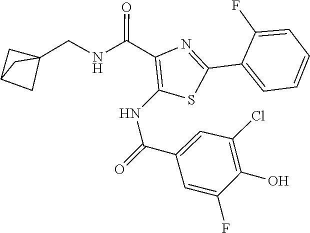 [SMILES: O=C(Nc1sc(-c2ccccc2F)nc1C(=O)NCC12CC(C1)C2)c1cc(F)c(O)c(Cl)c1] |
| 107 | 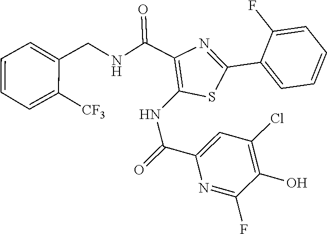 [SMILES: O=C(Nc1sc(-c2ccccc2F)nc1C(=O)NCc1ccccc1C(F)(F)F)c1cc(Cl)c(O)c(F)n1] |

Described herein is a compound, or a pharmaceutically acceptable salt, solvate, or stereoisomer thereof, selected from a compound in Table 1b.

TABLE 1b
Structure

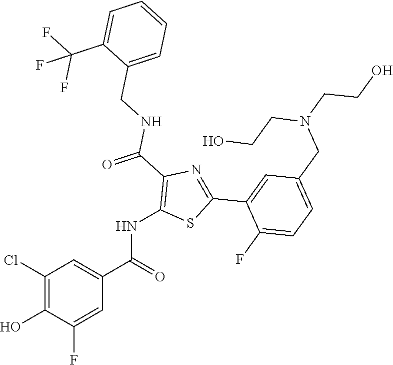 [SMILES: O=C(Nc1sc(-c2cc(CN(CCO)CCO)ccc2F)nc1C(=O)NCc1ccccc1C(F)(F)F)c1cc(F)c(O)c(Cl)c1]

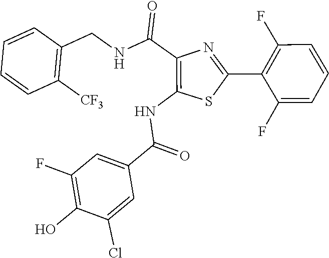 [SMILES: O=C(Nc1sc(-c2c(F)cccc2F)nc1C(=O)NCc1ccccc1C(F)(F)F)c1cc(F)c(O)c(Cl)c1]

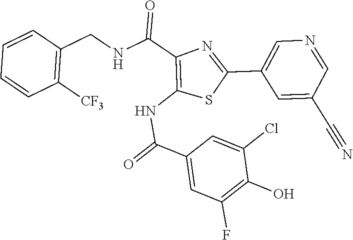 [SMILES: N#Cc1cncc(-c2nc(C(=O)NCc3ccccc3C(F)(F)F)c(NC(=O)c3cc(F)c(O)c(Cl)c3)s2)c1]

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

In some embodiments, the labeled compounds described herein are used for measuring in vitro and in vivo binding of unlabeled HSD17B13 inhibitors.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Provided herein are methods of inhibiting HSD17B13 expression or activity, which can be useful for treating, preventing, or ameliorating a disease associated with HSD17B13 in a subject in need thereof, such as NAFLD or NASH, by administration of a compound that targets HSD17B13, or a pharmaceutically acceptable salt, solvate, or stereoisomer thereof.

Provided herein are methods of inhibiting expression or activity of HSD17B13 in a cell comprising contacting the cell with a HSD17B13 inhibitor disclosed or a pharmaceutically acceptable salt, solvate, or stereoisomer thereof, thereby inhibiting expression or activity of HSD17B13 in the cell. In some embodiments, the cell is a hepatocyte cell. In some embodiments, the cell is in the liver. In some embodiments, the cell is in the liver of a subject who has, or is at risk of having a disease, disorder, condition, symptom, or physiological marker associated with a liver disease, metabolic disease, or cardiovascular disease or disorder. In some embodiments, the cells are the adipocytes or monocytes from a subject who has or is at risk of having a disease. In some embodiments, the cells are the lymphocytes from a subject who has or is at risk of having a disease. In some embodiments, the liver disease, metabolic disease, or cardiovascular disease or disorder is metabolic syndrome, fatty liver disease, chronic liver disease, liver cirrhosis, hepatic steatosis, steatohepatitis, nonalcoholic fatty liver disease (NAFLD), alcoholic liver disease, nonalcoholic steatohepatitis (NASH), fulminant Wilson's disease, rapidly fibrosing hepatitis C viral injury, and decompensated portal vein hypertension. In some embodiments, the liver disease, metabolic disease, or cardiovascular disease or disorder is NASH. In some embodiments, the liver disease, metabolic disease, or cardiovascular disease or disorder is cholestatic liver disease.

In some embodiments, the liver disease is primary biliary cirrhosis or primary sclerosing cholangitis.

Provided herein are methods of treating, preventing, delaying the onset, slowing the progression, or ameliorating one or more diseases, disorders, conditions, symptoms, or physiological markers associated with HSD17B13 comprising administering to a subject in need thereof a compound disclosed herein, or a pharmaceutically acceptable salt, solvate, or stereoisomer thereof. In some embodiments, the subject in need thereof is identified as having, or at risk of having, the disease, disorder, condition, symptom, or physiological marker. In some embodiments, the liver disease, metabolic disease, or cardiovascular disease or disorder is metabolic syndrome, liver disease, fatty liver disease, chronic liver disease, liver cirrhosis, hepatic steatosis, steatohepatitis, alcoholic liver disease, nonalcoholic fatty liver disease (NAFLD), and nonalcoholic steatohepatitis (NASH). In some embodiments, the liver disease, metabolic disease, or cardiovascular disease or disorder is NASH.

Provided herein are methods of reducing, improving, or regulating hepatic steatosis, liver fibrosis, triglyceride synthesis, lipid levels, hepatic lipids, ALT levels, NAFLD Activity Score (NAS), cholesterol levels, or triglyceride levels, or a combination thereof, in a subject in need thereof comprising administering to the subject a compound disclosed herein, or a pharmaceutically acceptable salt, solvate, or stereoisomer thereof. In some embodiments, the compound disclosed herein, or a pharmaceutically acceptable salt, solvate, or stereoisomer thereof, is provided for use in reducing, improving, or regulating hepatic steatosis in the individual. In some embodiments, the compound disclosed herein, or a pharmaceutically acceptable salt, solvate, or stereoisomer thereof, is provided for use in reducing, improving, or regulating liver fibrosis in the individual. In some embodiments, the compound disclosed herein, or a pharmaceutically acceptable salt, solvate, or stereoisomer thereof, is provided for use in reducing, improving, or regulating triglyceride synthesis in the individual. In some embodiments, the compound disclosed herein, or a pharmaceutically acceptable salt, solvate, or stereoisomer thereof, is provided for use in reducing, improving, or regulating lipid levels in the individual. In some embodiments, the compound disclosed herein, or a pharmaceutically acceptable salt, solvate, or stereoisomer thereof, is provided for use in reducing, improving, or regulating hepatic lipids in the individual. In some embodiments, the compound disclosed herein, or a pharmaceutically acceptable salt, solvate, or stereoisomer thereof, is provided for use in reducing, improving, or regulating ALT levels in the individual. In some embodiments, the compound disclosed herein, or a pharmaceutically acceptable salt, solvate, or stereoisomer thereof, is provided for use in reducing, improving, or regulating NAFLD Activity Score in the individual. In some embodiments, the compound disclosed herein, or a pharmaceutically acceptable salt, solvate, or stereoisomer thereof, is provided for use in reducing, improving, or regulating cholesterol levels in the individual. In some embodiments, the compound disclosed herein, or a pharmaceutically acceptable salt, solvate, or stereoisomer thereof, is provided for use in reducing, improving, or regulating triglyceride levels in the individual. In some embodiments, the subject is identified as having, or at risk of having a disease, disorder, condition, symptom, or physiological marker associated with a liver disease, metabolic disease, or cardiovascular disease or disorder. In some embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In some embodiments, the liver disease, metabolic disease, or cardiovascular disease or disorder is NASH.

Provided herein are methods for treating, preventing, or delaying onset drug induced liver injury (DILI) in a subject in need thereof, comprising administering to the subject a compound disclosed herein, or a pharmaceutically acceptable salt, solvate, or stereoisomer thereof. In some embodiments, the liver injury is steatohepatitis. Also provided herein are methods for treating, preventing, or delaying onset drug induced steatohepatitis (DISH) in a subject in need thereof. In some embodiments, the subject in need thereof is receiving chemotherapy for treating cancer. In some embodiments, the subject in need thereof is receiving a treatment for a cardiovascular disease. In some embodiments, the subject in need thereof is receiving treatment for a psychiatric disease/condition. In some embodiments, the subject in need thereof is receiving treatment for pain. In some embodiments, the subject in need thereof is receiving treatment for arthritis. In some embodiments, the chemotherapy is tamoxifen, toremifene, irinotecan, methotrexate, fluorouracil (5-FU), or any combination thereof. In some embodiments, the subject in need thereof is receiving amiodarone, perhexiline, propranolol, or any combination thereof. In some embodiments, the subject in need thereof is receiving amitriptyline, clozapine, or any combination thereof. In some embodiments, the subject in need thereof is receiving methotrexate, pirprofen, or any combinations thereof.

### Cholestatic Diseases

Provided herein is a method of treating a cholestatic disease in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

In some embodiments, inhibiting HSD17B13 improves bile flow. In some embodiments, inhibition of HSD17B13 is used to treat a cholestatic disease. Cholestatic diseases include primary sclerosis cholangitis (PSC), primary biliary cholangitis (PBC), Alagille Syndrome, biliary atresia, liver injury in cystic fibrosis patients and progressive familial intrahepatic cholestasis (PFIC).

HSD17B13 protein is very highly expressed in liver and gallbladder. The gallbladder is developmentally downstream of hepatocytes through the biliary tree. Inactive HSD17B13 has been associated with increases in liver and plasma phosphatidylcholine. Phosphatidylcholine is essential for bile flow. Hepatic phosphatidylcholine is secreted into the bile at a rate equivalent to the total liver levels of phosphatidylcholine being secreted within a day along with bile acids (BAs) and cholesterol. In some embodiments, inactive HSD17B13 is associated with increased plasma levels of VLDL-cholesterol. Meaning that there is a greater secretion of cholesterol out of the liver and not catabolized to bile acids for secretion in bile. In some embodiments, inhibition of HSD17B13 improves bile flow through increased phosphatidylcholine. In some embodiments, HSD17B13 is protecting the biliary tree by preventing inflammation. In some embodiments, HSD17B13 is protecting the biliary tree by preventing the cytotoxic bile acids from injuring the biliary tree.

In some embodiments, the cholestatic disease is primary sclerosis cholangitis (PSC), primary biliary cholangitis (PBC), Alagille Syndrome, biliary atresia, liver injury in a cystic fibrosis patient, progressive familial intrahepatic cholestasis (PFIC), intrahepatic cholestasis of pregnancy; drug-induced cholestasis, AIDS cholangiopathy, IG4-associated cholangitis, biliary stricture, or low phospholipid-associated cholestasis.

In some embodiments, the cholestatic disease is primary sclerosis cholangitis (PSC), primary biliary cholangitis (PBC), Alagille Syndrome, biliary atresia, liver injury in a cystic fibrosis patient, or progressive familial intrahepatic cholestasis (PFIC).

In some embodiments, the cholestatic disease is primary sclerosis cholangitis (PSC).

In some embodiments, the PSC is accompanied by inflammatory bowel disease (IBD). In some embodiments, the PSC is accompanied by elevated levels of lipopolysaccharide (LPS) (endotoxemia). In some embodiments, the elevated levels of LPS is in the blood. In some embodiments, the elevated levels of LPS is in the liver. In some embodiments, the elevated levels of LPS is in the biliary tree. In some embodiments, the cholestatic disease is primary biliary cholangitis (PBC). In some embodiments, the cholestatic disease is Alagille Syndrome. In some embodiments, the cholestatic disease is biliary atresia. In some embodiments, the cholestatic disease is liver injury in a cystic fibrosis patient. In some embodiments, the cholestatic disease is progressive familial intrahepatic cholestasis (PFIC). In some embodiments, the PFIC is PFIC-3 type. In some embodiments, the PFIC-3 type is due to a mutation in ABCB4 which requires phosphatidylcholine for bile acid transport.

In some embodiments, the cholestatic disease is treated by improving bile flow in the subject in need thereof. In some embodiments, the cholestatic disease is treated by improving cholesterol secretion out of the liver in the subject in need thereof.

In some embodiments, inactive HSD17B13 is associated to lower cytokines and inflammatory gene expression. In some embodiments, there is an improvement in the hepatocyte response to LPS in hepatocytes with inactive HSD17B13. In some embodiments, inactive HSD17B13 is associated with improved autophagy in response to LPS (see FIG. 1A, FIG. 1B, and FIG. 1C). In some embodiment, increases in LC3B-11 combined with increases in p62 indicate accumulation of autophagosomes and defective autophagy.

### Liver Injury Due to Protein Accumulation

Disclosed herein is a method of treating liver injury due to protein accumulation in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the protein that accumulates is alpha 1-antitrypsin.

Alpha 1-antitrypsin is encoded by the gene SERPINA1. The normal allele is referred to as M, while common SERPINA1 alleles that have been associated with liver disease have amino acid changes Glu264Val, referred to as S and Glu342Lys, referred to as Z. Individuals that are homozygotes for the Z (designated PiZZ) allele, have pulmonary manifestations of their A1AT deficiency and can be treated by supplementation of the enzyme. However, individuals that are heterozygotes for the Z allele either with the M (designated PiMZ) or with the S allele (designated PiSZ) have liver disease manifestations. The protein resulting from translation of the Z and S alleles is a misfolded protein. This misfolded protein has been found to polymerize and lead to cellular injury due to accumulation of misfolded and polymerized protein. Protection against the injury due to accumulated misfolded protein has been observed by increasing autophagy.

In some embodiments, HSD17B13 plays a role in autophagy. In some embodiments, autophagy is important for eliminating misfolded proteins and has been implicated in liver injury due to alpha 1-antitrypsin deficiency. In some embodiments, inhibition of HSD17B13 improves autophagy and thus improve clearance of misfolded proteins and thus, improve liver health. In some embodiments, liver disease associated with alpha 1-antitrypsin deficiency include inflammation and decreases in platelets. In some embodiments, inactive HSD17B13 is associated with lower inflammation, decreased inflammatory genes including NF-kB and TGF-β as well as increases in platelets.

In some embodiments, the protein accumulation is cleared via autophagy. In some embodiments, the protein is a misfolded protein. In some embodiments, the liver injury is due to alpha 1-antitrypsin deficiency. In some embodiments, the alpha 1-antitrypsin deficiency results in a protein that does not get fully processed and accumulates as a mis-folded protein in the liver. In some embodiments, the liver disease associated with alpha 1-antitrypsin deficiency include inflammation. In some embodiments, the liver disease associated with alpha 1-antitrypsin deficiency include decreases in platelets.

### Treatment of Viral-Induced Injuries

Disclosed herein is a method of treating a viral infection-induced liver injury in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

Also disclosed herein is a method of decreasing the severity of inflammation in a subject with a viral infection, the method comprising administering a therapeutically effective amount of a hydroxysteroid 17-beta dehydrogenase 13 (HSD17B13) inhibitor to the subject.

Also disclosed herein is a method of decreasing the severity of acute immune response in a subject with a viral infection, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

Viral infections are often associated with dysregulated liver inflammatory response. In some embodiments, inactive HSD17B13 is associated with lower levels of genes of activation of immune response including innate immune response, cytokines. Inflammation is a key component of viral-driven injury. In some embodiments, inactive HSD17B13 protects against rapidly progressing fibrosis in HCV patients.

AAV-driven gene therapies are aided by autophagy for incorporating into liver cells so an HSD17B13 inhibitor that improves autophagy and decreases inflammation can both enhance efficacy and decrease side effects.

In some embodiments, the viral infection is hepatitis A. In some embodiments, the viral infection is hepatitis B. In some embodiments, the viral infection is hepatitis C.

In some embodiments, the viral infection is SARS-Cov-2.

### SARS-Cov-2

Liver injury and elevations of liver transaminases are a common feature in patients with severe SARS-Cov-2 infection. There is no apparent increased risk of COVID-19 associated with liver disease, therefore, 2-11% of patients with COVID-19 have underlying liver comorbidities. In contrast, a much larger portion of patients (14-53%) have elevated liver transaminases. Patients suspected of having COVID-19 that test positive for the SARS-CoV-2 RNA had elevations in ALT, AST and decreases in albumin when compared to patients that tested negative for the viral RNA consistent with liver injury. In patients that died from COVID-19, 58% and 78% of patients had liver transaminase elevations. The liver transaminase elevations have been especially notable in patients in intensive care units at 62% compared to 25% of patients not requiring ICU care. Patients with a CT-scan confirmed diagnosis of COVID-19 in the subclinical stage have a lower incidence of liver transaminase elevations when compared to patients diagnosed after the onset of symptoms. While there may be a role of the virus causing direct liver injury as evidenced by the observation of SARS-CoV-2 RNA in blood and feces and 2-10% of patients that have diarrhea, the liver injury may also arise from secondary inflammatory events or the use of many different drugs during critical care.

In addition to liver injury in patients with severe COVID-19, there is an increase in cytokines, or incidence of cytokine storm that is a cause of multiple organ failure and death. To prevent or treat the cytokine storm would therefore be important to treating the patients with the most severe disease. Analysis of COVID-19 patient's blood has shown in general decreases in CD4 and CD8 cells but an increase in Th17 cells. Similarly, in a cohort of patients with severe disease there was an increase in IL-6, IL-10, IL-2 and IFNγ. On the whole, liver injury is seen in COVID-19 patients who are experiencing a broader and more severe disease where multiple organs, beyond the lungs, have become involved. Among the possible reasons for this could be that through an oral route that is then taken up through a more permeable intestinal barrier in patients with co-morbidities such as diabetes. The liver then is exposed to the virus and as a critical mediator for inflammatory responses, could thus mediate either a resolving inflammatory process or mediate an accelerating inflammatory process that leads to increased cytokine release and further organ damage.

The genetic evidence for the gene for the enzyme HSD17B13 association with liver transaminase levels and liver diseases has grown rapidly. At this writing, the genetic evidence includes many studies with genetic and disease information from more than 200,000 individuals. The lack of HSD17B13 activity has been shown to be protective against liver disease caused by over nutrition, excess and chronic alcohol intake, toxins such as copper and viruses such as HCV. The highly reproducible association of HSD17B13 with liver disease, liver transaminase elevations and liver injury in multiple forms has inspired efforts to develop inhibitors of this enzyme to protect against and reverse liver damage. The variety of toxicants and injuries against which HSD17B13 loss of function protects is such that it is proposed here to protect against liver injury in severe COVID-19.

HSD17B13 activity has been shown to be involved in inflammation and inflammatory response. HSD17B13 loss of function has recently been associated with decreased gene expression and protein levels of immune response genes involved in adverse outcomes including IL-6, IL-10 and IL-1β. Enzymatically inactive polymorphs of HSD17B13 are associated with lower severity of histopathological endpoints of inflammation in addition to lower ALT levels in patients with nonalcoholic fatty liver disease.

In some embodiments, HSD17B13 inhibitors prevent and treat liver injury and decrease the severity of inflammation and acute immune response in patients with COVID-19.

In some embodiments, the viral infection is associated with a dysregulated liver inflammatory response. In some embodiments, the administration results in lowering the levels of immune response activation genes. In some embodiments, the administration results in decreased inflammation. In some embodiments, the subject in need thereof has elevated levels of alanine aminotransferase (ALT). In some embodiments, the subject in need thereof has elevated levels of aspartate aminotransferase (AST). In some embodiments, the subject in need thereof has decreased levels of albumin.

### Malignancies

Disclosed herein is a method of treating a malignancy selected from hepatocellular carcinoma (HCC), cholangioadenoma, cholangiocarcinoma, gallbladder adenocarcinoma, and malignancy of bile duct, in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

Inactive HSD17B13 is associated with lower rates of HCC. However, HSD17B13 expression is low in tumor tissue but normal in the peritumoral space. High protein levels in the peritumoral space is associated with improved survival and tumor free survival. This apparent contradiction supports the use of small molecules to inhibit HSD17B13 but keep the protein available to support autophagic activity. Autophagy is important to maintaining a killer T cell response and use the body's own immune system to fight the tumor. By inhibiting HSD17B13, the surrounding tissue will be capable of killer T cell response due to effective autophagy.

HSD17B13 is highly expressed in normal gallbladder. Inactive HSD17B13 is associated with increased phospotidylcholine which is essential for sequestering cytotoxic bile acids for excretion and bile flow. For these reasons and the additional improved killer T cell response and decreased inflammation and fibrosis are the mechanism by which an inhibitor of HSD17B13 can be used to treat cholangioadenoma and gallbladder adenocarcinoma.

In some embodiments, the malignancy is hepatocellular carcinoma (HCC). In some embodiments, the malignancy is cholangioadenoma. In some embodiments, the malignancy is cholangiocarcinoma. In some embodiments, the malignancy is gallbladder adenocarcinoma. In some embodiments, the HSD17B13 receptor is expressed in the peritumoral space.

### Hemochromatosis

A method of treating hemochromatosis in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the hemochromatosis is primary hemochromatosis. In some embodiments, the hemochromatosis is secondary hemochromatosis. In some embodiments, the hemochromatosis is caused by a liver disease. In some embodiments, the hemochromatosis is drug-induced hemochromatosis.

### Lysosomal Acid Lipase Deficiency (LAL-D)

Disclosed herein is a method of treating lysosomal acid lipase deficiency (LAL-D) in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the lysosomal acid lipase deficiency (LAL-D) is caused by mutations in the LIPA gene. In some embodiments, the lysosomal acid lipase deficiency (LAL-D) causes Wolman disease. In some embodiments, the lysosomal acid lipase deficiency (LAL-D) causes Cholesteryl ester storage disease. In some embodiments, the lysosomal acid lipase deficiency (LAL-D) is caused by mutations in the LIPA gene. In some embodiments, the lysosomal acid lipase deficiency (LAL-D) causes a buildup of fatty substances in the body's cells and tissues.

### Bleeding Disorders/Coagulation Factor Disorders

Disclosed herein is a method of treating a bleeding disorder in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the bleeding disorder is hemophilia A or hemophilia B.

Disclosed herein is a method of treating a coagulation factor disorder in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the coagulation factor disorder is Von Willebrand disease.

In some embodiments, the disease to be treated by inhibition of hydroxysteroid 17-beta dehydroenase 13 (HSD17B13) is listed in the table below.

| | |
|---|---|
| AAV-induced liver injury | Hepatic adenoma |
| AAV injury | Hepatic encephalopathy |
| Alagille Syndrome | Hepatitis A |
| Alcohol-related liver disease | Hepatitis B |
| Alpha-1 Antitrypsin deficiency | Hepatitis C |
| Autoimmune hepatitis | Hepatorenal syndrome |
| Biliary atresia | Intrahepatic cholestasis of |
| | pregnancy (ICP) |
| Cholangiocarcinoma | Liver cancer |
| Covid 19 related | LAL-D |
| Crigler-Najjar Syndrome | Newborn jaundice |
| Cystic fibrosis - related | NAFLD |
| Drug induced liver injury | PBC |
| Galactosemia | PSC |
| Gallbladder cancer | Progressive familial intrahepatic |
| | cholestasis |
| Gilbert syndrome | Reye syndrome |
| HCC | Type I glycogen storage disease |
| Hemochromatosis | Wilson disease |

Also disclosed herein is a method for selectively inhibiting HSD17B13, the method comprising administering a pharmaceutically effective amount of a compound disclosed herein, or a pharmaceutically acceptable salt, solvate, or stereoisomer thereof. In some embodiments, the compound selectively inhibit HSD17B13 over HSD171B2, HSD17B14, or any combination thereof. In some embodiments, inhibition of HSD17B2 is associated with disruption of normal endocrine function in multiple tissues. In some embodiments, inhibition of HSD17B2 is associated with weight loss. In some embodiments, inhibition of HSD17B2 is associated with muscle loss. In some embodiments, inhibition of HSD17B14 is associated with disruption of normal endocrine function in multiple tissues.

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition. In one aspect, prophylactic treatments include administering to a mammal having patatin-like phospholipase domain-containing 3 (PNPLA3) polymorphism, a pharmaceutical composition comprising a compound described herein, or a pharmaceutically acceptable salt thereof, in order to prevent liver damages. The 148 Isoleucine to Methionine protein variant (I148M) of patatin-like phospholipase domain-containing 3 (PNPLA3), a protein is expressed in the liver and is involved in lipid metabolism, has recently been identified as a major determinant of liver fat content. Several studies confirmed that the I148M variant predisposes towards the full spectrum of liver damage associated with fatty liver: from simple steatosis to steatohepatitis and progressive fibrosis. Furthermore, the I148M variant represents a major determinant of progression of alcohol related steatohepatitis to cirrhosis, and to influence fibrogenesis and related clinical outcomes in chronic hepatitis C virus hepatitis, and possibly chronic hepatitis B virus hepatitis, hereditary hemochromatosis and primary sclerosing cholangitis. In some embodiments, PNPLA3 polymorphism is used to predict liver disease progression.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal, and topical routes of administration.

In another aspect, provided herein are pharmaceutical compositions comprising a compound described herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid catrer. In some embodiments, the liquid camer is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are method of treating a liver disease, metabolic disease, or cardiovascular disease using a compound disclosed herein, or a pharmaceutically acceptable salt, solvate, or stereoisomer thereof, in combination with an additional therapeutic agent.

In some embodiments, the additional therapeutic agent is used for the treatment of diabetes or diabetes related disorder or conditions.

In some instances, the additional therapeutic agent comprises a statin, an insulin sensitizing drug, an insulin secretagogue, an alpha-glucosidase inhibitor, a GLP agonist, a GIP agonist, a THR beta agonist, a PDE inhibitor, a DPP-4 inhibitor (such as sitagliptin, vildagliptin, saxagliptin, linagliptin, anagliptin, teneligliptin, alogliptin, gemigliptin, or dutogliptin), a catecholamine (such as epinephrine, norepinephrine, or dopamine), peroxisome proliferator-activated receptor (PPAR)-gamma agonist (e.g., a thiazolidinedione (TZD) [such as pioglitazone, rosiglitazone, rivoglitazone, or troglitazone], aleglitazar, farglitazar, muraglitazar, or tesaglitazar), peroxisome proliferator-activated receptor (PPAR)-alpha agonist, peroxisome proliferator-activated receptor (PPAR)-delta agonist, a farnesoid X receptor (FXR) agonist (e.g., obeticholic acid), or a combination thereof. In some cases, the statin is a HMG-CoA reductase inhibitor. In other instances, additional therapeutic agents include fish oil, fibrate, vitamins such as niacin, retinoic acid (e.g., 9 cis-retinoic acid), nicotinamide ribonucleoside or its analogs thereof, or combinations thereof. In other instances, additional therapeutic agents include ACC inhibitors, FGF19 and FGF21 mimics, CCR2/CCR5 antagonists, or combinations thereof.

In some embodiments, the additional therapeutic agent is vivitrol.

In some embodiments, the additional therapeutic agent is a statin such as a HMG-CoA reductase inhibitor, fish oil, fibrate, niacin, or a combination thereof. In other instances, the additional therapeutic agent is a dyslipidemia drug that prevent lipid absorption such as orlistat.

In some embodiments, the additional therapeutic agent is a vitamin such as retinoic acid or tocopheryl acetate for the treatment of diabetes and diabetes related disorder or condition such as lowering elevated body weight and/or lowering elevated blood glucose from food intake.

In some embodiments, the additional therapeutic agent is a glucose-lowering agent. In some embodiments, the additional therapeutic agent is an anti-obesity agent. In some embodiments, the additional therapeutic agent is selected from among a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a glucophage, a human amylin analog, a biguanide, an alpha-glucosidase inhibitor, a meglitinide, a thiazolidinedione, and sulfonylurea. In some embodiments, the additional therapeutic agent is metformin, sitagliptin, saxaglitpin, repaglinide, nateglinide, exenatide, liraglutide, insulin lispro, insulin aspart, insulin glargine, insulin detemir, insulin isophane, and glucagon-like peptide 1, or any combination thereof. In some embodiments, the additional therapeutic agent is a lipid-lowering agent.

In some embodiments, the additional therapeutic agent is an antioxidant, corticosteroid such as budesonide, anti-tumor necrosis factor (TNF), or a combination thereof.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

### EXAMPLE

### Example 1: Synthesis of 4,6-dichloro-5-hydroxy-N-(4-((2-(trifluoromethyl)benzyl) carbamoyl)-1H-pyrazol-3-yl)picolinamide

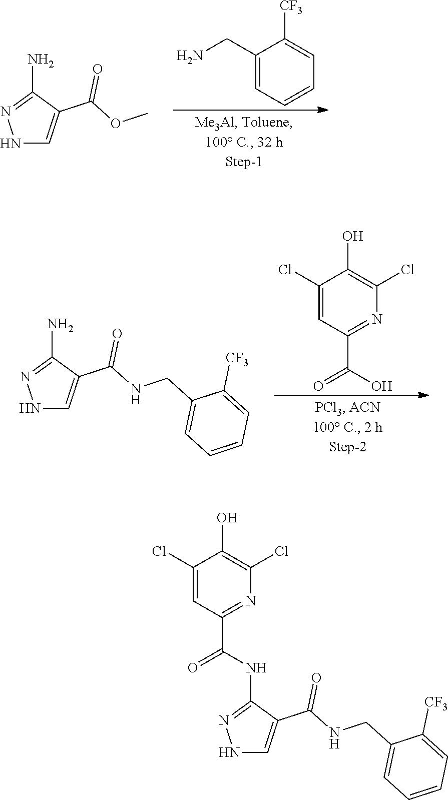

### Step 1: Synthesis of 3-amino-N-(2-(trifluoromethyl)benzyl)-1H-pyrazole-4-carboxamide

To a stirred solution of methyl 3-amino-1H-pyrazole-4-carboxylate (0.6 g, 4.25 mmol) and 1-[2-(trifluoromethyl)phenyl]methanamine (0.745 g, 4.25 mmol) in Toluene (3 mL) was added 2M trimethylaluminium in toluene (2.04 mL, 21.3 mmol) at 0° C. The resulting reaction mixture was stirred at 100° C. for 32 h. The reaction mixture was cooled to ambient temperature and quenched with ice-cold water (10 mL) and extracted with Ethyl acetate (15×2 mL). The combined organic phase was washed with brine solution (15 mL) and dried over anhydrous sodium sulphate, filtered, and evaporated in vacuo to obtain crude. The crude was purified through flash column chromatography to afford 3-amino-N-(2-(trifluoromethyl)benzyl)-1H-pyrazole-4-carboxamide (0.520 g, 43%) as a brown solid. LCMS (ES): m/z calculated for C12H11F3N4O, 284.24; found, 285.1 (M+H).

### Step 2: Synthesis of 4,6-dichloro-5-hydroxy-N-(4-((2-(trifluoromethyl)benzyl) carbamoyl)-1H-pyrazol-3-yl)picolinamide

To a stirred solution of 3-amino-N-{[2-(trifluoromethyl)phenyl]methyl}-1H-pyrazole-4-carboxamide (0.5 g, 1.76 mmol) and 4,6-dichloro-5-hydroxypyridine-2-carboxylic acid (0.439 g, 2.11 mmol) in Acetonitrile (5 mL, 76.6 mmol) was added Phosphorous trichloride (0.164 mL, 1.76 mmol) at ambient temperature. The resulting reaction mixture was stirred at 100° C. for 2 h. The reaction mixture was cooled to ambient temperature and quenched with ice-cold water (20 mL). The precipitated solid was filtered and dried under reduced pressure to afford crude solid. The resulting crude product was purified by reverse phase prep-HPLC to afford 4,6-dichloro-5-hydroxy-N-(4-((2-(trifluoromethyl)benzyl)carbamoyl)-1H-pyrazol-3-yl)picolinamide (0.093 g, 11%) as an off-white solid. LCMS (ES): m/z calculated for C18H12C12F3N5O3; 473.03 found, 472.1 (M−H); 1H NMR (400 MHz, DMSO d6): δ 11.71 (s, 1H), 8.83 (t, J=5.2 Hz, 1H), 8.21-8.16 (m, 1H), 8.10 (s, 1H), 7.75 (d, J=7.6 Hz, 1H), 7.70-7.66 (m, 1H), 7.55-7.48 (m, 2H), 4.73-4.63 (m, 2H).

### Example 2: Synthesis of 4,6-dichloro-5-hydroxy-N-(1-methyl-4-((2-(trifluoromethyl) benzyl)carbamoyl)-1H-pyrazol-3-yl)picolinamide

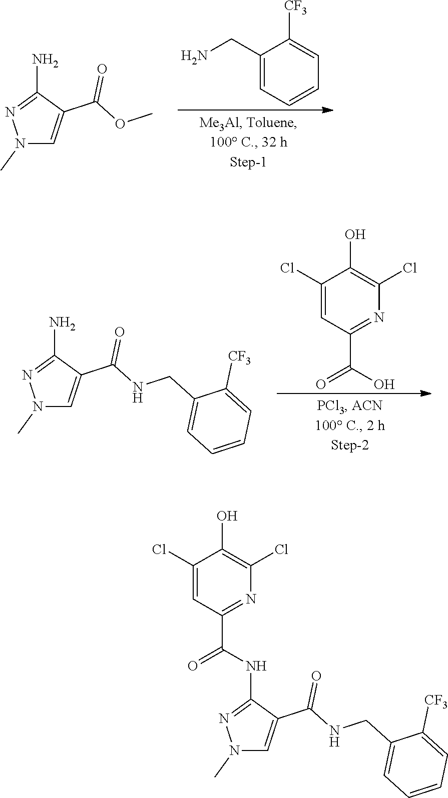

### Step 1: Synthesis of 3-amino-1-methyl-N-(2-(trifluoromethyl)benzyl)-1H-pyrazole-4-carboxamide

To a stirred solution of methyl 3-amino-1-methyl-1H-pyrazole-4-carboxylate (0.4 g, 2.58 mmol) and 1-[2-(trifluoromethyl)phenyl]methanamine (0.45 g, 2.58 mmol) in toluene (4 mL) was added 2 M trimethylaluminium in toluene (2.47 mL, 25.8 mmol) at 0° C. The resulting reaction mixture was stirred at 100° C. for 32 h. The reaction mixture was cooled to ambient temperature and quenched with ice-cold water (10 mL) and extracted with Ethyl acetate (15×2 mL). The combined organic phase was washed with brine solution (15 mL) and dried over anhydrous sodium sulphate, filtered, and evaporated in vacuo to obtain crude. The crude was purified through flash column chromatography to afford 3-amino-1-methyl-N-{[2-(trifluoromethyl)phenyl]methyl}-1H-pyrazole-4-carboxamide (0.5 g, 65.05%) as a brown solid. LCMS (ES): m/z calculated for C13H13F3N4O, 298.27; found, 299.1 (M+H).

### Step 2: Synthesis of 4,6-dichloro-5-hydroxy-N-(1-methyl-4-((2-(trifluoromethyl)benzyl) carbamoyl)-1H-pyrazol-3-yl)picolinamide

To a stirred solution of 3-amino-1-methyl-N-{[2-(trifluoromethyl)phenyl]methyl}-1H-pyrazole-4-carboxamide (0.5 g, 1.68 mmol) and 4,6-dichloro-5-hydroxypyridine-2-carboxylic acid (0.42 g, 2.01 mmol) in Acetonitrile (5 mL) was added Phosphorous trichloride (0.157 mL, 1.68 mmol) at ambient temperature. The resulting reaction mixture was stirred at 100° C. for 2 h. The reaction mixture was cooled to ambient temperature and quenched with ice-cold water (20 mL). The precipitated solid was filtered and dried under reduced pressure to afford crude. The resulting crude product was purified by reverse phase prep-HPLC to afford 4,6-dichloro-5-hydroxy-N-[1-methyl-4-({[2-(trifluoromethyl)phenyl]methyl}carbamoyl)-1H-pyrazol-3-yl]pyridine-2-carboxamide (0.073 g, 9%) as a pale yellow solid. LCMS (ES): m/z calculated for C19H14C12F3N5O3, 487.04; found, 486.1 (M−H); 1H NMR (400 MHz, DMSO-d6): δ 11.47 s, 1H), 8.77 (t, J=6.0 Hz, 1H), 8.27 (s, 1H), 8.05 (s, 1H), 7.75 (d, J=8.0 Hz, 1H), 7.95-7.68 (m, 1H), 7.54-7.47 (m, 2H), 4.68-4.58 (m, 2H), 3.86 (s, 3H).

### Example 3: Synthesis of 3-(3,5-dichloro-4-hydroxybenzamido)-N-(2-(trifluoromethyl) benzyl)-1H-pyrazole-4-carboxamide

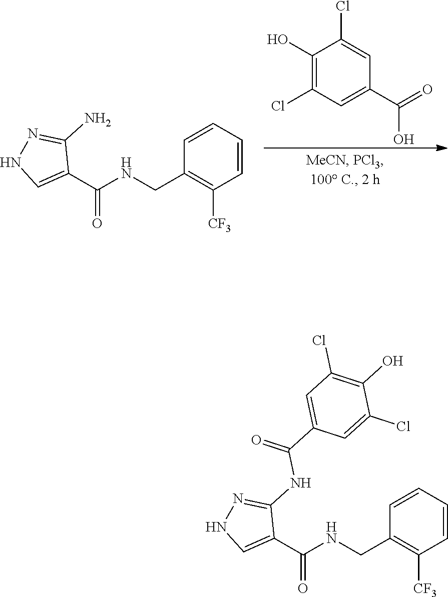 [SMILES: Nc1n[nH]cc1C(=O)NCc1ccccc1C(F)(F)F ; O=C(O)c1cc(Cl)c(O)c(Cl)c1 ; O=C(Nc1n[nH]cc1C(=O)NCc1ccccc1C(F)(F)F)c1cc(Cl)c(O)c(Cl)c1]

To a stirred solution of 3-amino-N-{[2-(trifluoromethyl)phenyl]methyl}-1H-pyrazole-4-carboxamide (0.15 g, 0.528 mmol) and 3,5-dichloro-4-hydroxybenzoic acid (0.12 g, 0.580 mmol) in Acetonitrile (3 mL) was added Phosphorus trichloride (0.013 mL, 0.158 mmol) dropwise at room temperature. The resulting reaction mixture was heated to 100° C. for 2 h. The reaction mixture was cooled to ambient temperature and quenched with ice-cold water (15 mL), obtained solid was collected through filtration, washed with diethyl ether (5 mL) and dried under reduced vacuum to afford crude as off white solid. The resulting crude product was purified by prep-HPLC (Column: XBridge C18 (250 mm×4.6 mm×5 μm); Mobile phase (A): 0.1% TFA in Water; Mobile phase (B): Acetonitrile; Flow rate:1.0 mL/min) to afford 3-(3,5-dichloro-4-hydroxybenzamido)-N-(2-(trifluoromethyl)benzyl)-1H-pyrazole-4-carboxamide (0.058 g, 23%) as white solid. LCMS (ES) m/z calculated for C19H13C12F3N4O3 is 472.03; found, 473.1 (M+H), 98.63% at 254 nm. ¹H NMR (400 MHz, DMSO d6 at 90° C.) δ 8.4 (bs, 1H), 8.13 (bs, 1H), 7.85 (s, 2H), 7.70 (d, J=7.6 Hz, 1H), 7.62-7.57 (m, 2H), 7.49-7.47 (m, 1H), 4.63 (d, J=24.8 Hz, 2H).

### Example 4: Synthesis of 4,6-dichloro-5-hydroxy-N-(1-methyl-3-((2-(trifluoromethoxy) benzyl)carbamoyl)-1H-pyrazol-4-yl)picolinamide

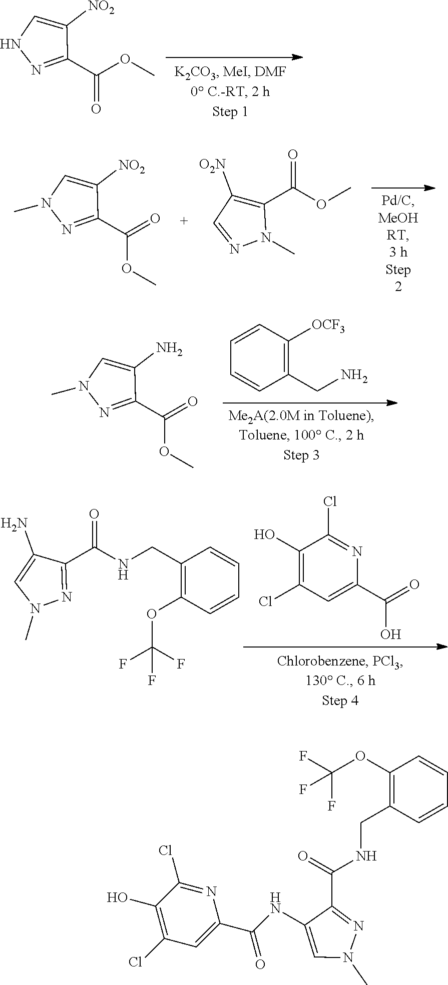

### Step 1: Synthesis of methyl 1-methyl-4-nitro-1H-pyrazole-3-carboxylate

To a stirred solution of methyl 4-nitro-1H-pyrazole-3-carboxylate (3 g, 17.5 mmol) in anhydrous dimethylformamide (25 mL) was added potassium carbonate (2.91 g, 21 mmol) at 0° C. followed by iodomethane (2.99 g, 21 mmol) was added slowly over the period of 15 min. The resulting reaction mixture was stirred at room temperature for 2 h. The reaction mixture was quenched with water (30 mL) and extracted into ethyl acetate (20 mL×2 times). The combined organic layer was washed with water (30 mL×2 times), brine (20 mL×2 times), dried over sodium sulfate and evaporated under reduced pressure to result in crude. The crude product was purified by flash chromatography to afford methyl 1-methyl-4-nitro-1H-pyrazole-3-carboxylate (1.87 g, 58%) as off-white solid and methyl 1-methyl-4-nitro-1H-pyrazole-5-carboxylate (0.92 g, 28%) as yellow viscous solid respectively. LCMS (ES) m/z calculated for C6H7N3O4 is 185.04; found, 186.1 (M+H).

### Step 2: Synthesis of methyl 4-amino-1-methyl-1H-pyrazole-3-carboxylate

To a stirred solution of methyl 1-methyl-4-nitro-1H-pyrazole-3-carboxylate (1.87 g, 10.1 mmol) in methanol (30 mL) was added Pd/C (0.45 g, 2.11 mmol) and then stirred at room temperature under hydrogen atmosphere for 3 h. The reaction mixture was filtered through pad of celite and washed with methanol (10 mL). The filtrate was concentrated under reduced pressure to result in crude compound which was triturated with n-pentane (10 mL), diethyl ether (10 ml) to afford methyl 4-amino-1-methyl-1H-pyrazole-3-carboxylate (1.2 g, 77%) as a black color solid.

### Step 3: Synthesis of 4-amino-1-methyl-N-(2-(trifluoromethoxy)benzyl)-1H-pyrazole-3-carboxamide

To a stirred solution of methyl 4-amino-1-methyl-1H-pyrazole-3-carboxylate (0.25 g, 1.61 mmol) and 1-[2-(trifluoromethoxy)phenyl]methanamine (0.30 g, 1.61 mmol) in Toluene (5 mL) was added 2M trimethylaluminium in toluene (0.46 mL, 4.83 mmol) at 0° C. The resulting reaction mixture was stirred at 100° C. for 2 h. The reaction mixture was cooled to ambient temperature and quenched with ice-cold water (10 mL) and extracted with Ethyl acetate (15×2 mL). The combined organic phase was washed with brine solution (15 mL) and dried over anhydrous sodium sulphate, filtered, and evaporated in vacuo to obtain crude. The crude was purified through flash column chromatography to afford 4-amino-1-methyl-N-(2-(trifluoromethoxy)benzyl)-1H-pyrazole-3-carboxamide (0.17 g, 35%) as pale yellow liquid. LCMS (ES) m/z calculated for C13H13F3N4O2 is 314.10; found, 315.1 (M+H).

### Step 4: Synthesis of 4,6-dichloro-5-hydroxy-N-(1-methyl-3-((2-(trifluoromethoxy) benzyl)carbamoyl)-1H-pyrazol-4-yl)picolinamide

To a stirred solution of 4-amino-1-methyl-N-(2-(trifluoromethoxy)benzyl)-1H-pyrazole-3-carboxamide (0.14 g, 0.445 mmol) and 4,6-dichloro-5-hydroxypyridine-2-carboxylic acid (0.1 g, 0.490 mmol) in chlorobenzene (0.8 mL) was added Phosphorus trichloride (0.011 mL, 0.134 mmol) dropwise to at room temperature. The resulting reaction mixture was heated to 100° C. for 6 h. The reaction mixture was cooled to ambient temperature and quenched with ice-cold water (15 mL), obtained solid was collected through filtration, washed with diethyl ether (5 mL) and dried under reduced vacuum to afford crude as off white solid. The resulting crude product was purified by prep-HPLC to afford 4,6-dichloro-5-hydroxy-N-(1-methyl-3-((2-(trifluoromethoxy)benzyl)carbamoyl)-1H-pyrazol-4-yl)picolinamide (0.067 g, 30%) as off-white solid. LCMS (ES) m/z calculated for C19H14C12F3N5O4 is 503.04; found, 504.1 (M+H). 1H NMR (400 MHz, DMSO d6) δ 11.13 (s, 1H), 8.98 (t, J=6.0 Hz, 1H), 8.43 (s, 1H), 8.07 (s, 1H), 7.43-7.36 (m, 4H), 4.55 (d, J=6.0 Hz, 2H), 3.96 (s, 3H).

### Example 5: Synthesis of 4,6-dichloro-5-hydroxy-N-(1-methyl-5-((2-(trifluoromethoxy)benzyl)carbamoyl)-1H-pyrazol-4-yl)picolinamide

The title compound was synthesized as described above. LCMS (ES) m/z calculated for C19H14C12F3N5O4 is 503.04; found, 504.1 (M+H). 1H NMR (400 MHz, DMSO d6) δ 11.13 (s, 1H), 8.94 (t, J=6.0 Hz, 1H), 8.43 (s, 1H), 8.07 (s, 1H), 7.43-7.36 (m, 4H) 4.55 (d, J=6.0 Hz, 2H), 3.96 (s, 3H).

### Example 6: Synthesis of 3-(3,5-dichloro-4-hydroxybenzamido)-1-methyl-N-(2-(trifluoromethyl)benzyl)-1H-pyrazole-4-carboxamide

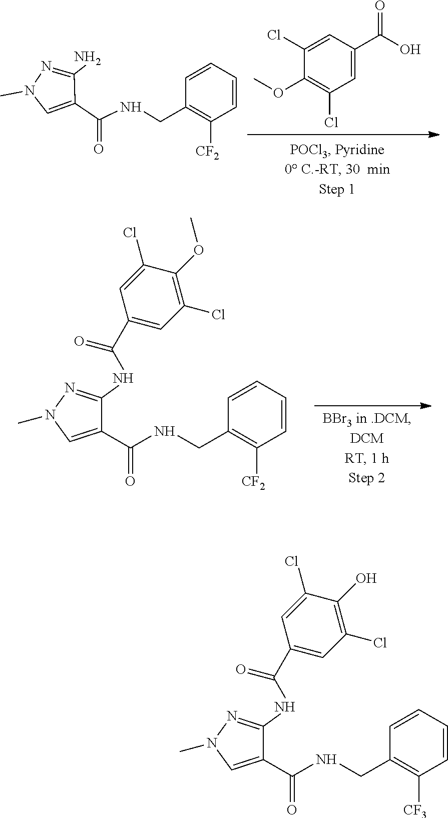

### Step 1: Synthesis of 3-(3,5-dichloro-4-methoxybenzamido)-1-methyl-N-(2-(trifluoromethyl)benzyl)-1H-pyrazole-4-carboxamide

To a stirred solution of 3,5-dichloro-4-methoxybenzoic acid (0.22 g, 1.01 mmol) in pyridine (2 mL) was added phosphoroyl trichloride (0.094 mL, 1.01 mmol) at 0° C. and allowed to stir at room temperature for 10 minutes. Then added 3-amino-1-methyl-N-{[2-(trifluoromethyl)phenyl]methyl}-1H-pyrazole-4-carboxamide (0.3 g, 1.01 mmol) and stirred for 30 minutes. Progress of the reaction was monitored by TLC and LCMS. The reaction mixture was quenched with ice-cold water, extracted with ethyl acetate (3 mL×2). The combined organic layer was washed with water (2 mL×2), brine (2 mL×2), dried over sodium sulfate to afford crude compound which was purified through flash column chromatography to afford 3-(3,5-dichloro-4-methoxybenzamido)-1-methyl-N-{[2-(trifluoromethyl)phenyl]methyl}-1H-pyrazole-4-carboxamide (0.2 g, 40%) as white solid. LCMS (ES) m/z calcd, For C21H17C12F3N4O3, 500.06; found, 501.1 (M+H).

### Step 2: Synthesis of 3-(3,5-dichloro-4-hydroxybenzamido)-1-methyl-N-(2-(trifluoromethyl)benzyl)-1H-pyrazole-4-carboxamide

To a stirred solution of 3-(3,5-dichloro-4-methoxybenzamido)-1-methyl-N—{[2-(trifluoromethyl)phenyl]methyl}-1H-pyrazole-4-carboxamide (0.110 g, 0.219 mmol) in dichloromethane (0.5 mL) was added boranetribromide in dichloromethane (0.167 mL, 1.76 mmol) was added at 0° C. and the reaction was allowed to stir at room temperature for 1 hour. Progress of the reaction was monitored by TLC and LCMS. After completion of the reaction, reaction mixture was quenched with ice-cold water and then extracted with DCM (3×5 mL). The combined organic layer was washed with water (3 mL×2), brine (2 mL×2), dried over sodium sulfate to afford crude compound which was purified flash column chromatography to afford off white solid. The solid material was further purified by reverse phase prep-HPLC to afford pure 3-(3,5-dichloro-4-hydroxybenzamido)-1-methyl-N-{[2-(trifluoromethyl)phenyl]methyl}-1H-pyrazole-4-carboxamide as a white solid. (31 mg, 29%). LCMS(ES) m/z calculated for C20H15Cl2F3N4O3, 486.05; found, 487.1 (M+H), ¹H NMR (400 MHz, DMSO-d6) δ 10.98 (s, 1H), 10.51 (s, 1H), 8.51 (t, J=5.6 Hz, 1H), 8.22 (s, 1H), 7.83 (s, 2H), 7.70 (d, J=7.6 Hz, 1H), 7.61 (t, J=7.6 Hz, 1H), 7.53 (d, J=7.6 Hz, 1H), 7.46 (t, J=7.6 Hz, 1H) 4.58 (d, J=5.6 Hz, 2H), 3.85 (s, 3H).

### Example 7: Synthesis of 5-(3,5-dichloro-4-hydroxybenzamido)-1-methyl-N-(2-(trifluoromethyl)benzyl)-1H-pyrazole-4-carboxamide

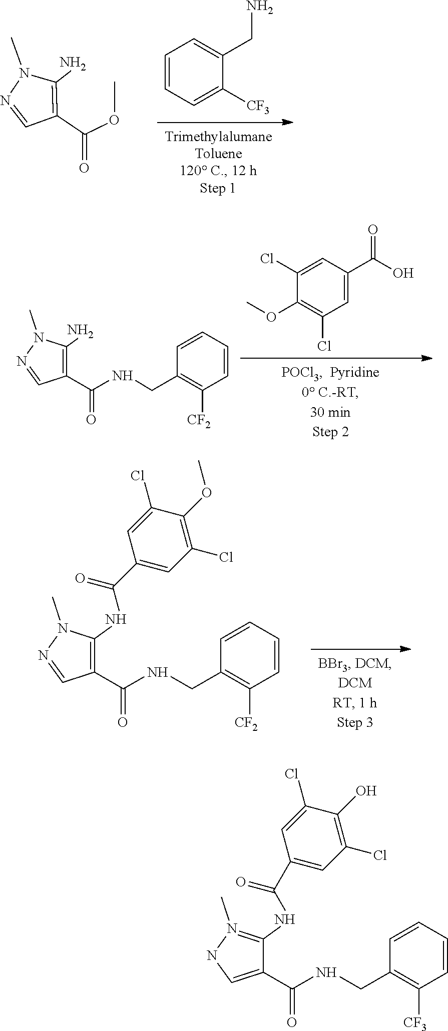

### Step 1: Synthesis of 5-amino-1-methyl-N-(2-(trifluoromethyl)benzyl)-1H-pyrazole-4-carboxamide

To a solution of methyl 5-amino-1-methyl-1H-pyrazole-4-carboxylate (0.4 g, 2.58 mmol) and 1-[2-(trifluoromethyl)phenyl]methanamine (0.358 mL, 2.58 mmol) in toluene (5 mL, 42.3 mmol) was added 2M trimethylaluminium in toluene (0.741 mL, 3 eq., 7.73 mmol) at 0° C. Then the reaction mixture was allowed to stir at 120° C. for 12 h. The reaction mixture was cooled to ambient temperature and poured into cold water, obtained solid material was filtered off and filtrate was extracted with DCM (3 mL×2). The combined organic phase was washed with water (2 mL×2), brine (2 mL×2), dried over sodium sulfate and evaporated under reduced pressure to afford 5-amino-1-methyl-N-{[2-(trifluoromethyl)phenyl]methyl}-1H-pyrazole-4-carboxamide (0.77 g, quantitative). LCMS (ES) m/z calcd, For C13H13F3N4O, 298.10; found, 299.1 (M+H).

### Step 2: Synthesis of 5-(3,5-dichloro-4-methoxybenzamido)-1-methyl-N-(2-(trifluoromethyl)benzyl)-1H-pyrazole-4-carboxamide

To a stirred solution of 3,5-dichloro-4-methoxybenzoic acid (0.0593 g, 0.268 mmol) in pyridine (1 mL) was added phosphoroyl trichloride (0.025 mL, 0.268 mmol) at 0° C. and allowed to stirred at room temperature for 10 minutes. To this stirred solution was added 5-amino-1-methyl-N-{[2-(trifluoromethyl)phenyl]methyl}-1H-pyrazole-4-carboxamide (0.08 g, 0.268 mmol) and stirred for another 30 minutes. The reaction mixture was quenched with ice-cold water, extracted with ethyl acetate (3 mL×2). The combined organic layer was washed with water (2 mL×2), brine (2 mL×2), dried over sodium sulfate to afford crude compound which was purified through flash column chromatography to afford 5-(3,5-dichloro-4-methoxybenzamido)-1-methyl-N-{[2-(trifluoromethyl)phenyl]methyl}-1H-pyrazole-4-carboxamide as a white solid (0.11 g, 82%). LCMS (ES) m/z calcd, For C21H17C12F3N4O3, 500.06; found, 501.0 (M+H).

### Step 3: Synthesis of 5-(3,5-dichloro-4-hydroxybenzamido)-1-methyl-N-(2-(trifluoromethyl)benzyl)-1H-pyrazole-4-carboxamide

To a stirred solution of 5-(3,5-dichloro-4-methoxybenzamido)-1-methyl-N-{[2-(trifluoromethyl)phenyl]methyl}-1H-pyrazole-4-carboxamide (0.1 g, 0.199 mmol) in dichloromethane (0.556 mL) was added (0.151 mL, 1.6 mmol) was added at 0° C. and the reaction was allowed to stir at room temperature for 1 h. Progress of the reaction was monitored by TLC and LCMS. After completion of the reaction, reaction mixture was quenched with ice-cold water, extracted using DCM (3×5 mL). The combined organic layer was washed with water (3 mL×2), brine (2 mL×2), dried over sodium sulfate to afford crude compound which was purified through flash column chromatography to give an off white solid, which was further purified by reverse phase prep-HPLC to afford 5-(3,5-dichloro-4-hydroxybenzamido)-1-methyl-N-{[2-(trifluoromethyl)phenyl]methyl}-1H-pyrazole-4-carboxamide (5 mg, 5%) as white solid. LCMS(ES) m/z calculated for C20H15Cl2F3N4O3, 486.05; found, 487.1 (M+H); ¹H NMR (400 MHz, DMSO-d6) δ 11.01 (s, 1H), 10.31 (bs, 1H), 8.55 (t, J=5.6 Hz, 1H), 8.00 (d, J=6.4 Hz, 3H), 7.70 (d, J=8 Hz, 1H), 7.61 (t, J=7.6 Hz, 1H), 7.52-7.43 (m, 2H), 4.57 (d, J=5.6 Hz, 2H), 3.65 (s, 3H).

### Example 8: Synthesis of 4,6-dichloro-5-hydroxy-N-(1-phenyl-4-((2-(trifluoromethyl) benzyl)carbamoyl)-1H-pyrazol-3-yl)picolinamide

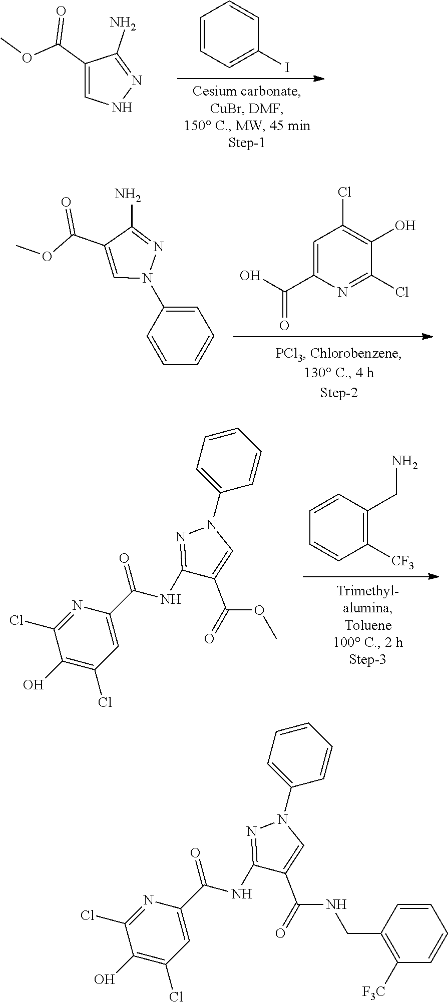

### Step 1: Synthesis of methyl 3-amino-1-phenyl-1H-pyrazole-4-carboxylate

A stirred solution of methyl 3-amino-1H-pyrazole-4-carboxylate (0.3 g, 2.13 mmol) in DMF (10 mL) was added iodobenzene (0.35 mL, 3.19 mmol), cesium carbonate (693 mg, 2.13 mmol) and copper bromide (30.5 mg, 0.213 mmol) and heated the reaction mass to 150° C. in MW for 45 min. After completion of reaction, reaction mass was quenched with water (20 mL) and extracted with ethyl acetate (20 mL), organic layer was collected and washed with water (20 mL), brine solution (20 mL), organic layer was dried over sodium sulphate and filtered and evaporated to get crude product which was purified by flash column chromatography to get pure methyl 3-amino-1-phenyl-1H-pyrazole-4-carboxylate (0.3 g, 65%) as off-white solid. LCMS (ES) m/z calculated for C11H11N3O2, 217; found, 218 (M+H).

### Step 2: Synthesis of methyl 3-(4,6-dichloro-5-hydroxypicolinamido)-1-phenyl-1H-pyrazole-4-carboxylate

A stirred solution of methyl 3-amino-1-phenyl-1H-pyrazole-4-carboxylate (250 mg, 1.15 mmol) in chlorobenzene (5 mL) was added 4,6-dichloro-5-hydroxypicolinic acid (311 mg, 1.5 mmol), PCl₃ (158 mg, 1.15 mmol) and heated the reaction mass to 130° C. for 4 h. After completion of reaction, reaction mass was cooled to room temperature and quenched with ice water (10 mL) and extract with ethyl acetate (10 mL), organic layer was collected and washed with water (10 mL), brine solution (10 mL). Organic layer was dried over sodium sulphate and filter and dried to get crude product, which was purified by flash column chromatography to get title product methyl 3-(4,6-dichloro-5-hydroxypicolinamido)-1-phenyl-1H-pyrazole-4-carboxylate (250 mg, 53%) as off-white solid. LCMS (ES) m/z calculated for C17H12C12N4O4, 406; found, 407 (M+H).

### Step 3: Synthesis of 4,6-dichloro-5-hydroxy-N-(1-phenyl-4-((2-(trifluoromethyl) benzyl)carbamoyl)-1H-pyrazol-3-yl)picolinamide

A stirred solution of methyl 3-(4,6-dichloro-5-hydroxypicolinamido)-1-phenyl-1H-pyrazole-4-carboxylate (220 mg, 0.540 mmol) in toluene (5 mL) was added (2-(trifluoromethyl)phenyl)methanamine (0.076 mL, 0.540 mmol) and cooled the reaction mass to 0° C. and added trimethylaluminium 2.0 M in toluene (0.81 mL, 1.62 mmol) and heated the reaction mass to 100° C. for 2 h. Progress of the reaction was monitored by TLC and LCMS, after completion of reaction, reaction mass was cooled to room temperature and quenched with ice water (10 mL) and extract with ethyl acetate (20 mL) and dried over sodium sulphate to get crude product which was purified by flash column chromatography, fractions were collected and dried to get crude product which was further purified by prep-HPLC. LCMS (ES) m/z calculated for C24H16C12F3N5O3, 549.0; found, 550 (M+H). ¹H NMR (400 MHz, DMSO-d₆): δ 10.53 (s, 1H), 8.70-8.62 (m, 1H), 8.28 (s, 1H), 7.91 (s, 1H), 7.71 (d, J=8.0 Hz, 1H), 7.63-7.60 (m, 1H), 7.56-7.53 (m, 3H), 7.49-7.45 (m, 3H), 7.40-7.38 (m, 1H), 4.60 (d, J=5.2 Hz, 2H).

### Example 9: Synthesis of 3-(3,5-dichloro-4-hydroxybenzamido)-1-phenyl-N-(2-(trifluoromethyl)benzyl)-1H-pyrazole-4-carboxamide

The title compound was synthesized as described above. LCMS (ES): m/z calculated for C25H17C12F3N4O3, 548.0; found, 549 (M+H). ¹H NMR (400 MHz, DMSO-d₆): δ 11.00 (bs, 1H), 10.73 (s, 1H), 9.01 (s, 1H), 8.66 (t, J=5.6 Hz, 1H), 7.94 (s, 2H), 7.87-7.80 (m, 2H), 7.73-7.68 (m, 1H), 7.64-7.62 (m, 2H), 7.61-7.57 (m, 2H), 7.50-7.47 (m, 1H), 7.40-7.37 (m, 1H), 4.63 (d, J=5.2 Hz, 2H).

### Example 10: Synthesis of 4,6-dichloro-5-hydroxy-N-(1-methyl-4-((2-(trifluoromethyl) benzyl)carbamoyl)-1H-pyrazol-5-yl)picolinamide

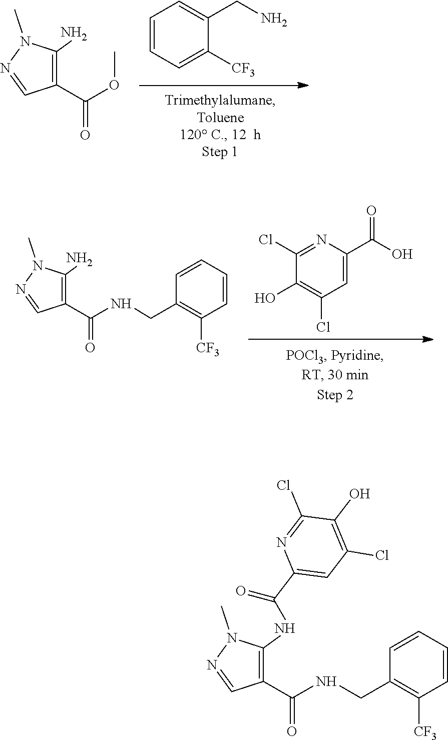

### Step 1: Synthesis of 5-amino-1-methyl-N-(2-(trifluoromethyl)benzyl)-1H-pyrazole-4-carboxamide

To a solution of methyl 5-amino-1-methyl-1H-pyrazole-4-carboxylate (0.4 g, 2.58 mmol) and 1-[2-(trifluoromethyl)phenyl]methanamine (0.358 mL, 2.58 mmol) in toluene (5 mL, 42.3 mmol), trimethylalumane (0.741 mL, 3 eq., 7.73 mmol) was added dropwise at 0° C. under nitrogen and stirred for some time. Then the reaction mixture was allowed to stir at 120° C. for 12 h. Progress of the reaction was monitored by TLC and LCMS. After reaction completion, the reaction mixture was quenched with ice chunks and the solid formed was filtered off. The aqueous layer was extracted with DCM (3 mL×2). The combined organic layer was washed with water (2 mL×2), brine (2 mL×2), dried over sodium sulfate and evaporated under reduced pressure to resulted in 5-amino-1-methyl-N-(2-(trifluoromethyl)benzyl)-1H-pyrazole-4-carboxamide (0.77 g, 2.58 mmol). LCMS (ES) m/z calcd, For C13H13F3N4O, 298.10; found, 299.1 (M+H).

### Step 2: Synthesis of 4,6-dichloro-5-hydroxy-N-(1-methyl-4-((2-(trifluoromethyl)benzyl) carbamoyl)-1H-pyrazol-5-yl)picolinamide

To a stirred solution of 4,6-dichloro-5-hydroxypyridine-2-carboxylic acid (0.174 g, 0.838 mmol) in pyridine (2 mL) was added PCl₃ (0.157 mL, 1.68 mmol) at 0° C. and allowed to stirred at room temperature for 15 min. After that 5-amino-1-methyl-N-(2-(trifluoromethyl)benzyl)-1H-pyrazole-4-carboxamide (0.25 g, 0.838 mmol) was added to it and stirred for 30 min. Progress of the reaction was monitored by TLC and LCMS. After reaction completion, the reaction mass was quenched with ice-cold water, extracted with ethyl acetate (5 mL×2). The combined organic layer was washed with water (3 mL×2), brine (4 mL×2), dried over sodium sulfate to afford crude compound which was purified by Combi flash chromatography. Pure fractions were collected and concentrated to afford title compound which was further purified by reverse phase prep-HPLC. Pure fractions were collected and concentrated to afford pure 4,6-dichloro-5-hydroxy-N-(1-methyl-4-((2-(trifluoromethyl)benzyl) carbamoyl)-1H-pyrazol-5-yl)picolinamide (0.012 g, 0.0246 mmol) as white solid. LCMS(ES) m/z calculated for C19H14C12F3N5O3, 487.04; found, 488.00 (M+H), 99.6% at 254 nm. ¹H NMR (400 MHz, DMSO-d₆) δ 10.41 (s, 1H), 8.59-8.57 (m, 1H), 7.99 (d, J=17.6 Hz, 2H), 7.67 (d, J=8 Hz, 1H), 7.56-7.47 (m, 1H), 7.48-7.42 (m, 2H), 4.54 (d, J=4.4 Hz, 2H), 3.66 (s, 3H).

### Example 11: Synthesis of 3,5-dichloro-4-hydroxy-N-(8-methyl-4-oxo-3-(2-(trifluoromethyl) benzyl)-3,4-dihydrobenzo[d][1,2,3]triazin-5-yl)benzamide

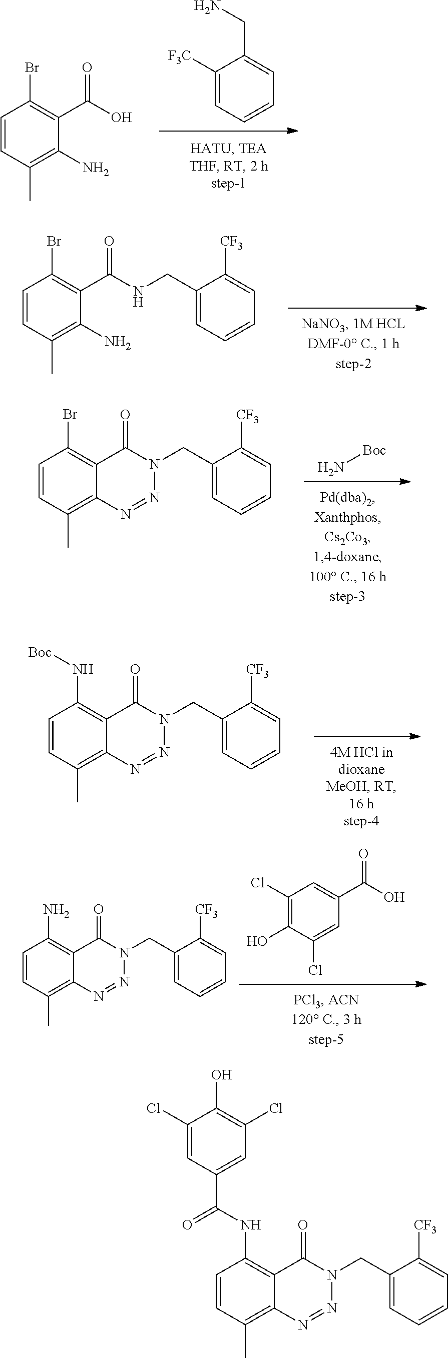

### Step 1: Synthesis of 2-amino-6-bromo-3-methyl-N-(2-(trifluoromethyl)benzyl)benzamide

To a stirred solution of 2-amino-6-bromo-3-methylbenzoic acid (2 g, 8.69 mmol) in N,N-dimethylformamide (10 mL) was added by 1-[2-(trifluoromethyl)phenyl]methanamine (1.23 mL, 8.69 mmol), triethylamine (4.24 mL, 30.4 mmol), HATU (3.97 g, 10.4 mmol) and N,N-dimethylpyridin-4-amine (0.106 g, 0.869 mmol) and allowed to stirred at room temperature for 2 h. The reaction was monitored by TLC. The reaction mixture was poured into water (40 mL) and extracted with ethyl acetate (2×75 mL). The combined organic phase was washed with water, brine and dried over anhydrous sodium sulfate, filtered, and concentrated in vacuo. The crude material was purified by flash column chromatography to afford 2-amino-6-bromo-3-methyl-N-(2-(trifluoromethyl)benzyl)benzamide (2 g, 59%) as off white solid. LCMS (ES): m/z calculated for C16H14BrF3N2O, 386.02; found, 387.2.

### Step 2: Synthesis of 5-bromo-8-methyl-3-(2-(trifluoromethyl)benzyl)benzo[d][1,2,3]triazin-4(3H)-one

To a stirred solution of 1M hydrogen chloride (72 mL) was added sodium nitrite (2.89 g, 41.8 mmol) portion wise 0° C. and stirred for 20 min, then 2-amino-6-bromo-3-methyl-N-(2-(trifluoromethyl)benzyl)benzamide (5.4 g, 13.9 mmol) in NN-dimethylformamide (4 mL) was added at 0° C. and allowed to stirred for 1 h. The reaction was monitored by TLC. After adjusting the pH to about 9 with concentrated ammonia, reaction mixture was stirred for 20 minutes and then adjust the pH to about 2 with concentrated hydrochloric acid, and continued stirring for another 20 min. The precipitated solid was filtered and washed with hexane (25 mL), dried to obtain 5-bromo-8-methyl-3-(2-(trifluoromethyl)benzyl)benzo[d][1,2,3]triazin-4(3H)-one (3.4 g, 61%) as orange solid. LCMS (ES): m/z calculated for C16H11BrF3N3O, 397.18; found, 398.

### Step 3: Synthesis of tert-butyl (8-methyl-4-oxo-3-(2-(trifluoromethyl)benzyl)-3,4-dihydrobenzo[d][1,2,3]triazin-5-yl)carbamate

To a suspension of 5-bromo-8-methyl-3-(2-(trifluoromethyl)benzyl)benzo[d][1,2,3]triazin-4(3H)-one (3.4 g, 8.54 mmol), tert-butyl carbamate (1.10 g, 9.39 mmol) and cesium carbonate (8.35 g, 25.6 mmol) in 1,4-dioxane (80 mL, 1.04 mmol) was degassed with nitrogen and added Xanthphos (0.49 g, 0.854 mmol) and Pd(dba)₂ (0.491 g, 0.854 mmol) at room temperature and stirred at 100° C. for 16 h in a sealed tube. The progress of the reaction was monitored by LCMS and TLC. After completion, reaction mixture was quenched with water and compound was extracted with ethyl acetate. Organic layer was washed with brine solution, dried over anhydrous sodium sulphate, filtered, and evaporated under vacuum to obtain crude solid. The crude was purified through flash column chromatography to afford tert-butyl (8-methyl-4-oxo-3-(2-(trifluoromethyl)benzyl)-3,4-dihydrobenzo[d][1,2,3]triazin-5-yl)carbamate (2.1 g, 56%) as off white solid. LCMS (ES): m/z calcd. C21H21F3N403, 434.42; found, 435.1.

### Step 4: Synthesis of 5-amino-8-methyl-3-(2-(trifluoromethyl) benzyl) benzo[d][1,2,3]triazin-4(3H)-one

To a suspension of tert-butyl (8-methyl-4-oxo-3-(2-(trifluoromethyl)benzyl)-3,4-dihydrobenzo[d][1,2,3]triazin-5-yl)carbamate (2 g, 4.76 mmol) in methanol (3.12 mL, 77.1 mmol) was added 4M HCl in dioxane (0.826 mL, 23.8 mmol) at room temperature. The resulting reaction mixture was stirred at ambient temperature for 16 h. The progress of the reaction was monitored by TLC. The reaction mixture was poured into water (40 mL) and extracted with ethyl acetate (3×50 mL). The organic phase was washed with water, brine and dried over anhydrous sodium sulfate, filtered, and concentrated in vacuo to afford 5-amino-8-methyl-3-(2-(trifluoromethyl) benzyl) benzo[d][1,2,3]triazin-4(3H)-one (1.4 g, 92%) as a yellow solid. LCMS (ES): m/z calculated for C16H13F3N40, 334.30; found, 335.1.

### Step 5: Synthesis of 3,5-dichloro-4-hydroxy-N-(8-methyl-4-oxo-3-(2-(trifluoromethyl)benzyl)-3,4-dihydrobenzo[d][1,2,3]triazin-5-yl)benzamide

To a suspension of 5-amino-8-methyl-3-(2-(trifluoromethyl) benzyl) benzo[d][1,2,3]triazin-4(3H)-one (0.350 g, 1.05 mmol) in Acetonitrile (5 mL) was added 3,5-dichloro-4-hydroxybenzoic acid (0.238 g, 1.15 mmol) and Phosphorus trichloride (0.916 mL, 1.05 mmol) at ambient temperature. The resulting reaction mixture was heated to 130° C. for 3 h. The reaction mixture was cooled to ambient temperature and then poured into ice cold water (20 mL) and extracted with ethyl acetate (10 mL x 3). The combined organic phase was washed with water, brine and dried over anhydrous sodium sulfate, filtered, and concentrated in vacuo. The crude product was purified through prep-HPLC to afford 3,5-dichloro-4-hydroxy-N-(8-methyl-4-oxo-3-(2-(trifluoromethyl)benzyl)-3,4-dihydrobenzo [d][1,2,3]triazin-5-yl)benzamide (0.010 g, 2%) as white solid. LCMS (ES): m/z calculated for C23H15C12F3N4O3, 522.05; found 521.1 (M−H); ¹H NMR (400 MHz, DMSO-d6): δ 12.29 (s, 1H), 11.15 (bs, 1H), 8.86 (d, J=8.4 Hz, 1H), 8.00 (d, J=8.4 Hz, 1H), 7.84-7.80 (m, 3H), 7.62-7.52 (m, 2H), 7.31 (d, J=8 Hz, 1H), 5.78 (s, 2H), 2.72 (s, 3H).

### Example 12: Synthesis of 4,6-dichloro-5-hydroxy-N-(8-methyl-4-oxo-3-(2-(trifluoromethyl)benzyl)-3,4-dihydrobenzo[d][1,2,3]triazin-5-yl)picolinamide

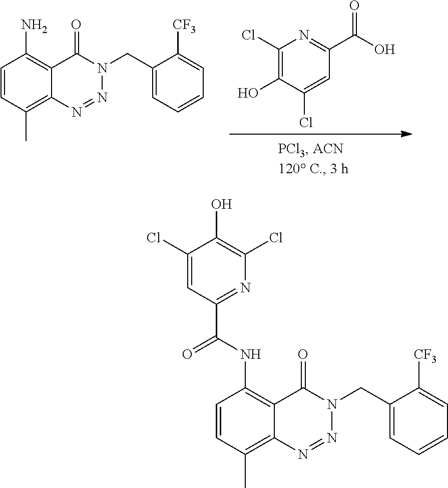 [SMILES: Cc1ccc(N)c2c(=O)n(Cc3ccccc3C(F)(F)F)nnc12 ; O=C(O)c1cc(Cl)c(O)c(Cl)n1 ; Cc1ccc(NC(=O)c2cc(Cl)c(O)c(Cl)n2)c2c(=O)n(Cc3ccccc3C(F)(F)F)nnc12]

To a suspension of 5-amino-8-methyl-3-(2-(trifluoromethyl) benzyl) benzo[d][1,2,3]triazin-4(3H)-one (0.35 g, 1.05 mmol) in acetonitrile (5 mL) was added 4,6-dichloro-5-hydroxypyridine-2-carboxylic acid (0.240 g, 1.15 mmol) and Phosphorus trichloride (0.09 mL, 1.05 mmol) at ambient temperature. The resulting reaction mixture was heated to 130° C. for 3 h. The reaction mixture was cooled to ambient temperature and then poured into ice cold water (20 mL) and extracted with ethyl acetate (10 mL x 3). The combined organic phase was washed with water, brine and dried over anhydrous sodium sulfate, filtered, and concentrated in vacuo. The crude product was purified through prep-HPLC to afford 4,6-dichloro-5-hydroxy-N-(8-methyl-4-oxo-3-(2-(trifluoromethyl) benzyl)-3,4-dihydrobenzo [d][1,2,3]triazin-5-yl)picolinamide (0.006 g, 1.8%) as white solid. LCMS (ES): m/z calculated for C22H14C12F3N5O3, 523.04; found 524.1 (M+H); ¹H NMR (400 MHz, DMSO-d6): δ 13.05 (s, 1H), 9.0 (d, J=8.4 Hz, 1H), 8.09 (s, 1H), 7.97 (d, J=8.8 Hz, 1H), 7.82 (t, J=7.6 Hz, 1H), 7.61-7.51 (m, 2H), 7.30 (d, J=8 Hz, 1H), 5.76 (s, 2H), 2.71 (s, 3H).

### Example 13: Synthesis of 4,6-dichloro-N-(8-fluoro-4-oxo-3-(2-(trifluoromethyl) benzyl)-3,4-dihydrobenzo[d][1,2,3]triazin-5-yl)-5-hydroxypicolinamide

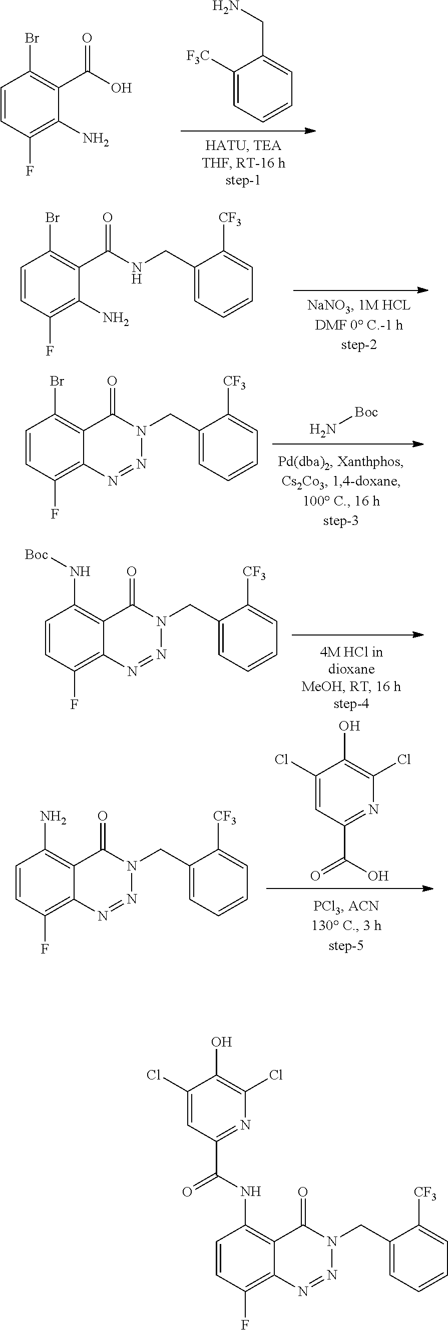

### Step 1: Synthesis of 2-amino-6-bromo-3-fluoro-N-(2-(trifluoromethyl)benzyl) benzamide

To a stirred solution of 2-amino-6-bromo-3-fluorobenzoic acid (1 g, 4.27 mmol) in DMF (15 mL) was added 1-[2-(trifluoromethyl) phenyl]methanamine (0.604 mL, 4.27 mmol), triethylamine (2.08 mL, 15 mmol), HATU (1.95 g, 5.13 mmol), N,N-dimethylpyridin-4-amine (0.052 g, 0.427 mmol) and allowed to stirred at room temperature for 16 h. The reaction was monitored by TLC. The reaction mixture was poured into water (40 mL) and extracted with ethyl acetate (150 mL). The organic phase was washed with water, brine and dried over anhydrous sodium sulfate, filtered, and concentrated in vacuo. The crude material was purified by flash column chromatography to afford 2-amino-6-bromo-3-fluoro-N-(2-(trifluoromethyl)benzyl)benzamide (1.5 g, 89.74%) as off white solid. LCMS (ES): m/z calculated for C15H11BrF4N2O, 390.0; found, 391.0.

### Step 2: Synthesis of 5-bromo-8-fluoro-3-(2-(trifluoromethyl) benzyl) benzo[d][1,2,3]triazin-4(3H)-one

To a stirred solution of sodium nitrite (1.06 g, 15.3 mmol), add 1M solution of HCL (25 mL) dropwise at 0° C. and stir for 20 min, then was added by 2-amino-6-bromo-3-fluoro-N-{[2-(trifluoromethyl)phenyl]methyl}benzamide (2 g, 5.11 mmol) dissolved in DMF (10 mL) and allowed to stirred at 0° C. for 1 h. The reaction was monitored by TLC. Then adjust the pH to about 9 with concentrated ammonia, stir for 20 minutes and then adjust the pH to about 2 with concentrated hydrochloric acid, and continue stirring for 20 minutes. After the reaction is completed, suction filtration is performed, the filter cake is washed with a small amount of Hexane, and dried to obtain 5-bromo-8-fluoro-3-(2-(trifluoromethyl) benzyl) benzo[d][1,2,3]triazin-4(3H)-one (0.6 g, 29%) as orange solid. LCMS (ES): m/z calculated for C15H8BrF4N3O, 400.98; found, 402.0.

### Step 3: Preparation of tert-butyl (8-fluoro-4-oxo-3-(2-(trifluoromethyl) benzyl)-3,4-dihydrobenzo[d][1,2,3]triazin-5-yl)carbamate

To a suspension of 5-bromo-8-fluoro-3-(2-(trifluoromethyl) benzyl) benzo[d][1,2,3]triazin-4(3H)-one (0.2 g, 0.497 mmol), tert-butyl carbamate (0.117 g, 0.995 mmol) and cesium carbonate (0.486 g, 1.49 mmol) in 1,4-dioxane (5.15 mL, 60.4 mmol) was degassed with nitrogen and added Xantphos (0.0288 g, 0.0497 mmol), Pd(dba)2 (0.0286 g, 0.0497 mmol) at room temperature and stirred at 100° C. for 16 h in a sealed tube. The progress of the reaction was monitored by LCMS and TLC. The reaction mixture was poured into water (20 mL) and extracted with ethyl acetate (20 mL×3). The organic phase was washed with water (10 mL), brine (3 ml) and dried over anhydrous sodium sulfate, filtered, and concentrated in vacuo. The crude material was purified by flash column chromatography to afford tert-butyl (8-fluoro-4-oxo-3-(2-(trifluoromethyl)benzyl)-3,4-dihydrobenzo[d][1,2,3]triazin-5-yl)carbamate (0.130 g, 59.6%) as colorless liquid. LCMS (ES): m/z calcd. C20H18F4N4O3, 438.34; found, 439.1.

### Step 4: Synthesis of 5-amino-8-fluoro-3-(2-(trifluoromethyl) benzyl) benzo[d][1,2,3]triazin-4(3H)-one

To a suspension of tert-butyl (8-fluoro-4-oxo-3-(2-(trifluoromethyl)benzyl)-3,4-dihydrobenzo[d][1,2,3]triazin-5-yl)carbamate (0.130 g, 0.297 mmol) in methanol (5 ml) and was added 4M HCl in dioxane (0.0515 mL, 1.48 mmol) was allowed to stirred at RT for 16 h. The reaction mixture was poured into water (20 mL) and extracted with ethyl acetate (20 mL×3). The organic phase was washed with water (10 ml), brine (5 ml) and dried over anhydrous sodium sulfate, filtered, and concentrated in vacuo. The crude material was purified by flash column chromatography to afford 5-amino-8-fluoro-3-(2-(trifluoromethyl)benzyl)benzo[d][1,2,3]triazin-4(3H)-one (0.095 g, 94%). LCMS (ES): m/z calculated for C15H10F4N4O, 338.27; found, 339.1.

### Step 5: Synthesis of 4,6-dichloro-5-hydroxy-N-(8-methyl-4-oxo-3-(2-(trifluoromethyl) benzyl)-3,4-dihydrobenzo[d][1,2,3]triazin-5-yl)picolinamide

To a suspension of 5-amino-8-fluoro-3-(2-(trifluoromethyl) benzyl) benzo[d][1,2,3]triazin-4(3H)-one (0.130 g, 0.384 mmol) in acetonitrile (5 ml) was added 4,6-dichloro-5-hydroxypyridine-2-carboxylic acid (0.0879 g, 0.423 mmol) and phosphorous trichloride (33.6 μL, 0.384 mmol) was allowed to stirred at 120° C. for 3 h. The reaction was monitored by TLC. The reaction mixture was poured into water (20 mL) and extracted with ethyl acetate (10 mL×3). The organic phase was washed with water (10 ml), brine (5 ml) and dried over anhydrous sodium sulfate, filtered, and concentrated in vacuo. The crude material was purified by Prep HPLC. Pure fractions were collected and concentrated to afford 4,6-dichloro-N-(8-fluoro-4-oxo-3-(2-(trifluoromethyl)benzyl)-3,4-dihydrobenzo[d][1,2,3]triazin-5-yl)-5-hydroxypicolinamide (0.007 g, 3.45%) as white solid. ¹H NMR (400 MHz, DMSO-d₆): δ 12.90 (s, 1H), 9.14-9.10 (m, 1H), 8.10-8.04 (m, 2H), 7.86 (d, J=7.6 Hz, 1H), 7.64-7.54 (m, 2H), 7.38 (d, J=8 Hz, 1H), 5.80 (s, 2H). LCMS (ES) m/z calculated for C21H11C12F4N5O3, 527.02; found 528.1 (M+H).

### Example 14: Synthesis of 3,5-dichloro-N-(8-fluoro-4-oxo-3-(2-(trifluoromethyl)benzyl)-3,4-dihydrobenzo[d][1,2,3]triazin-5-yl)-4-hydroxybenzamide

The title compound was synthesized as described above. LCMS (ES) m/z calculated for C22H12C12F4N4O3, 526.02; found, 527.0 (M+H); ¹H NMR (400 MHz, DMSO-d₆) δ 12.03 (s, 1H), 11.22 (s, 1H), 8.90 (d, J=9.2 Hz, 1H), 8.07 (t, J=9.6 Hz, 1H), 7.85-7.81 (m, 3H), 7.61-7.51 (m, 2H), 7.36 (d, J=8 Hz, 1H), 5.78 (s, 2H).

### Example 15: Synthesis of 4,6-dichloro-N-(8-fluoro-2-methyl-4-oxo-3-((1S,2R)-2-phenylcyclobutyl)-3,4-dihydroquinazolin-5-yl)-5-hydroxypicolinamide

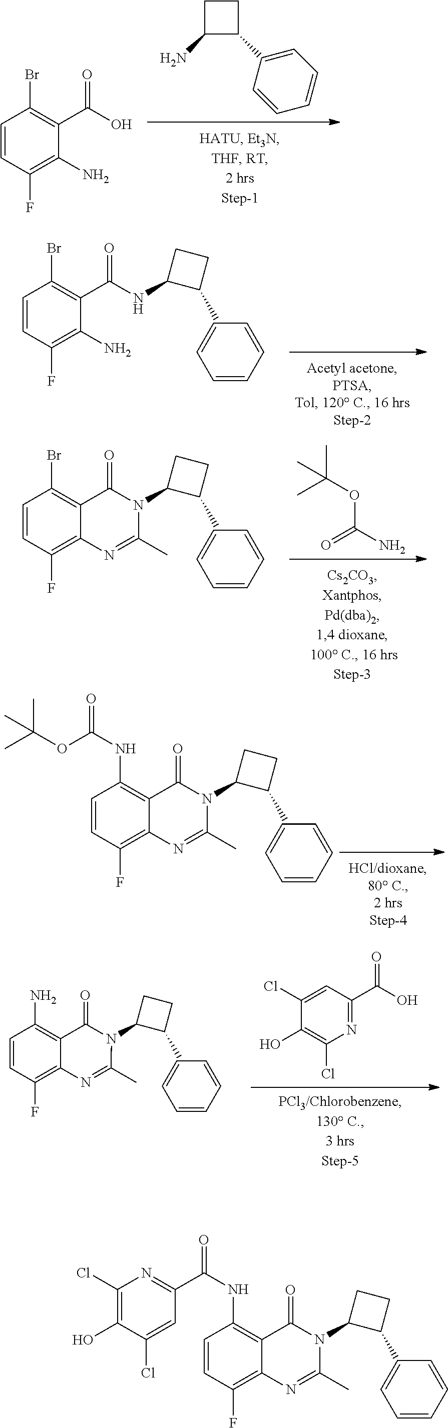

### Step 1: Synthesis of 2-amino-6-bromo-3-fluoro-N-((1S,2R)-2-phenylcyclobutyl) benzamide

To a stirred solution of 2-amino-6-bromo-3-fluorobenzoic acid (0.5 g, 2.14 mmol) in tetrahydrofuran (5 mL) was added triethylamine (0.89 mL, 6.41 mmol), HATU (0.75 g, 3.2 mmol) and (1S,2R)-2-phenylcyclobutan-1-amine (315 mg, 2.14 mmol) at room temperature. The resulting reaction mixture was stirred at room temperature for 2 h. The reaction mixture was poured into water (25 mL) and extracted into ethyl acetate (2×50 mL). The combined organics were washed with water (10 mL) and brine solution (10 mL), dried over anhydrous sodium sulphate, filtered and concentrated under reduced vacuum to afford crude which was purified through flash column chromatography to afford 2-amino-6-bromo-3-fluoro-N-((1S,2R)-2-phenylcyclobutyl)benzamide (0.62 g, 80%) as an off white solid. LCMS (ES) m/z calculated for C17H16BrFN2O, 362.04; found, 363.0 (M+2H).

### Step 2: Synthesis of 5-bromo-8-fluoro-2-methyl-3-((1S,2R)-2-phenylcyclobutyl) quinazolin-4(3H)-one

To a stirred solution of 2-amino-6-bromo-3-fluoro-N-[(1S,2R)-2-phenylcyclobutyl]benzamide (619 mg, 1.7 mmol) in toluene (6 mL) was added p-TSA (58.7 mg, 0.341 mmol) and pentane-2,4-dione (0.192 mL, 1.87 mmol) at room temperature. The resulting reaction mixture was heated to 120° C. for 16 hours in a sealed tube. After completion, the reaction mixture was quenched with water (25 mL) and extracted into ethyl acetate (2×50 mL). The combined organics were washed with water (20 mL) and brine solution (20 mL), dried over anhydrous sodium sulfate, filtered, and evaporated under reduced vacuum to afford the crude 5-bromo-8-fluoro-2-methyl-3-((1S,2R)-2-phenylcyclobutyl)quinazolin-4(3H)-one (0.62 g, 94%) as a brown solid. LCMS (ES) m/z calculated for C19H16BrFN2O, 387.05; found, 389.1 (M+2H).

### Step 3: Synthesis of tert-butyl (8-fluoro-2-methyl-4-oxo-3-((1S,2R)-2-phenylcyclobutyl)-3,4-dihydroquinazolin-5-yl)carbamate

To a stirred solution of 5-bromo-8-fluoro-2-methyl-3-[(1S,2R)-2-phenylcyclobutyl]-3,4-dihydroquinazolin-4-one (0.5 g, 1.29 mmol) in 1,4-dioxane (3 mL) was added tert-butyl carbamate (454 mg, 3.87 mmol) and cesium carbonate (1.26 g, 3.87 mmol). The mixture was purged with N2 for 10 min followed by Xanthphos (0.15 g, 0.258 mmol) and Pd(dba)₂ (74.2 mg, 0.129 mmol) was added and heated to 100° C. for 16 hr. The reaction mixture was filtered through celite pad, washed with Ethyl acetate (30 mL), the filtrate was evaporated to give crude which was purified through flash column chromatography to afford tert-butyl (8-fluoro-2-methyl-4-oxo-3-((1S,2R)-2-phenylcyclobutyl)-3,4-dihydroquinazolin-5-yl)carbamate (0.33 g, 60%) as an off white solid. LCMS (ES) m/z calculated for C24H26FN3O3, 423.20; found, 424.2 (M+H).

### Step 4: Synthesis of 5-amino-8-fluoro-2-methyl-3-((1S,2R)-2-phenylcyclobutyl) quinazolin-4(3H)-one

To a solid tert-butyl N-{8-fluoro-2-methyl-4-oxo-3-[(1S,2R)-2-phenylcyclobutyl]-3,4-dihydroquinazolin-5-yl}carbamate (0.33 g, 0.779 mmol) was added 4M HCl in Dioxane (1.95 mL) at 0° C. under nitrogen atmosphere. The reaction mixture was heated to 80° C. for 2 hours. After completion, the reaction mixture was evaporated to dryness under reduced pressure to get solid, which was further triturated with n-pentane (20 mL) and dried to afford 5-amino-8-fluoro-2-methyl-3-((1S,2R)-2-phenylcyclobutyl)quinazolin-4(3H)-one (0.2 g, 79%) as a white solid. LCMS (ES) m/z calculated for C19H18FN3O, 323.14; found, 324.1 (M+H).

### Step 5: Synthesis of 4,6-dichloro-N-(8-fluoro-2-methyl-4-oxo-3-((1S,2R)-2-phenylcyclobutyl)-3,4-dihydroquinazolin-5-yl)-5-hydroxypicolinamide

To a stirred solution of hydroxypyridine 5-amino-8-fluoro-2-methyl-3-[(1S,2R)-2-phenylcyclobutyl]-3,4-dihydroquinazolin-4-one (150 mg, 0.464 mmol) in acetonitrile (2 mL) was added 4,6-dichloro-5-2-carboxylic acid (0.092 g, 0.18 mmol) and Phosphorus trichloride (0.041 mL, 0.464 mmol). The resulting reaction mixture was heated at 100° C. for 3 hours in a sealed tube. After completion, reaction mixture was quenched with ice cold water (15 mL) and stirred for 5 minutes, obtained solid was collected through filtration. The solid material was washed with n-pentane (10 mL) and Acetonitrile (10 mL), dried under reduced pressure to afford 4,6-dichloro-N-(8-fluoro-2-methyl-4-oxo-3-((1S,2R)-2-phenylcyclobutyl)-3,4-dihydroquinazolin-5-yl)-5-hydroxypicolinamide (0.092 g, 39%) as an off white solid. LCMS (ES) m/z calculated for C25H19C12FN4O3, 512.08; found, 513.1 (M+H). HNMR (400 MHz, DMSO-d6) δ 13.54 (s, 1H), 8.72-8.68 (m, 2H), 8.12 (s, 1H), 7.70-7.65 (m, 1H), 7.35-7.28 (m, 4H), 7.22-7.19 (m, 1H), 4.95-4.90 (m, 1H), 4.81-4.76 (m, 1H), 2.40 (s, 3H), 2.36-2.25 (m, 2H), 2.01-1.96 (m, 2H).

### Example 16: Synthesis of 3,5-dichloro-N-(8-fluoro-2-methyl-4-oxo-3-((1S,2R)-2-phenylcyclobutyl)-3,4-dihydroquinazolin-5-yl)-4-hydroxybenzamide

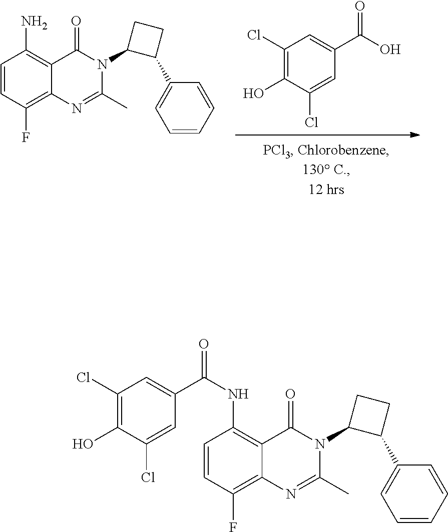 [SMILES: Cc1nc2c(F)ccc(N)c2c(=O)n1[C@H]1CC[C@@H]1c1ccccc1 ; O=C(O)c1cc(Cl)c(O)c(Cl)c1 ; Cc1nc2c(F)ccc(NC(=O)c3cc(Cl)c(O)c(Cl)c3)c2c(=O)n1[C@H]1CC[C@@H]1c1ccccc1]

To a stirred solution of 5-amino-8-fluoro-2-methyl-3-[(1S,2R)-2-phenylcyclobutyl]-3,4-dihydroquinazolin-4-one (90 mg, 0.278 mmol) and 3,5-dichloro-4-hydroxybenzoic acid (69.1 mg, 0.334 mmol) in chlorobenzene (3.0 mL) was added Phosphorus trichloride (0.024 mL, 0.278 mmol) at 0° C. under nitrogen atmosphere. The resulting reaction mass was heated to 130° C. for 12 hours. The reaction mixture was quenched with ice cold water (10 mL) and stirred for 5 minutes, obtained solid was collected through filtration. The solid was washed with n-pentane (10 ml) and dried under reduced vacuum to afford crude as a pale yellow solid. The crude material was purified through Prep-HPLC to afford 3,5-dichloro-N-(8-fluoro-2-methyl-4-oxo-3-((1S,2R)-2-phenylcyclobutyl)-3,4-dihydroquinazolin-5-yl)-4-hydroxybenzamide (0.030 g, 21%) as an off white solid. LCMS (ES) m/z calculated for C26H20Cl2FN3O3, 511.09; found, 512.1 (M+H). HNMR (400 MHz, DMSO-d6) δ 12.73 (s, 1H), 11.17 (s, 1H), 8.56-8.53 (m, 1H), 7.93 (s, 2H), 7.70-7.65 (m, 1H), 7.34-7.18 (m, 5H), 4.97-4.93 (m, 1H), 4.76-4.69 (m, 1H), 3.25-3.15 (m, 2H), 2.42 (s, 3H), 2.38-2.25 (m, 2H), 1.99-1.94 (m, 1H).

### Example 17: Preparation 3,5-dichloro-N-(2,8-dimethyl-4-oxo-3-(2-(2-(trifluoromethyl) phenyl) cyclobutyl)-3,4-dihydroquinazolin-5-yl)-4-hydroxybenzamide

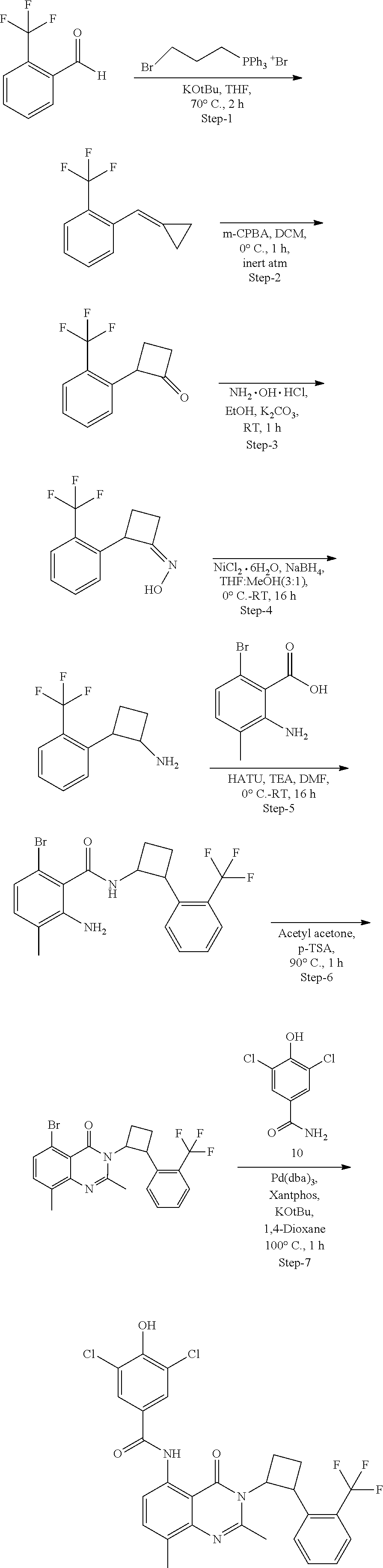

### Step 1: Preparation of 1-(cyclopropylidenemethyl)-2-(trifluoromethyl) benzene

To a stirred solution of 2-(trifluoromethyl) benzaldehyde (10 g, 57.4 mmol) (1) in tetrahydrofuran (150 mL) was added (3-bromopropyl) triphenyl phosphonium bromide (29.3 g, 63.2 mmol) followed by Potassium tert-butoxide (19.3 g, 172 mmol) and then stirred at 70° C. for 2 h. The reaction mixture was cooled to ambient temperature and poured into cold water (50 mL), extracted with ethyl acetate (3×45 mL). The combined organic layer was dried over sodium sulphate, filtered, and concentrated to afford crude. The crude was purified through flash column chromatography to afford 1-(cyclopropylidenemethyl)-2-(trifluoromethyl) benzene as yellowish oil (2.5 g, 22%). 1H NMR (400 MHz, CDCl₃): δ 7.96 (d, J=8.0 Hz, 1H), 7.63 (d, J=8.0 Hz, 1H), 7.48 (t, J=7.6 Hz, 1H), 7.32-7.25 (m, 1H), 7.12 (s, 1H), 1.2-1.5 (m, 4H).

### Step 2: Synthesis of 2-(2-(trifluoromethyl) phenyl) cyclobutan-1-one

To a stirred solution of 1-(cyclopropylidenemethyl)-2-(trifluoromethyl) benzene (1.2 g, 6.05 mmol in dichloromethane (10 mL) was added m-CPBA (1.25 g, 7.27 mmol) at 0° C. After completion of addition, the reaction mixture was stirred at room temperature for 1 hour. The reaction mixture was quenched with cold water (25 mL) and extracted with ethyl acetate (3×25 mL). The combined organic layer was washed with aqueous saturated sodium bicarbonate solution (25 mL) and dried over sodium sulphate, filtered and concentrated to afford crude. The crude was purified through flash column chromatography to afford 2-(2-(trifluoromethyl) phenyl) cyclobutan-1-one as yellowish oil (0.5 g, yield: 39%). 1H NMR (400 MHz, CDCl3): δ7.67 (d, J=8.0 Hz, 1H), 7.43-7.58 (m, 2H), 7.38 (t, J=7.6 Hz, 1H), 4.90 (t, J=8.8 Hz, 1H), 3.25-3.4 (m, 1H), 3.12-3.18 (m, 1H), 2.6-2.7 (m, 1H), 2.08-2.2 (m, 1H).

### Step 3: Synthesis of 2-(2-(trifluoromethyl) phenyl) cyclobutan-1-one oxime

To a stirred solution of 2-(2-(trifluoromethyl) phenyl) cyclobutan-1-one (4) (0.365 g, 1.7 mmol) in ethanol (10 mL) was added hydroxylamine hydrogen chloride (0.355 g, 5.11 mmol) and potassium carbonate (0.707 g, 5.11 mmol). The resulting reaction mixture was stirred at room temperature for 16 h. the reaction mixture was quenched with cold water (30 mL) and extracted with ethyl acetate (3×25 mL), combined organic layer was dried over sodium sulphate, filtered, and concentrated to afford 2-(2-(trifluoromethyl) phenyl) cyclobutan-1-one oxime (0.4 g, yield: 102%) as crude yellow solid. LCMS (ES) m/z calculated for C11H10F3NO, 229.07; found 230.1 (M+H);

### Step 4: Synthesis of 2-(2-(trifluoromethyl) phenyl) cyclobutan-1-amine

To a stirred solution of 2-(2-(trifluoromethyl) phenyl) cyclobutan-1-one oxime (0.4 g, 1.75 mmol) in THF:MeOH (4:1) (12 mL) was added nickel chloride hexahydrate (0.083 mg, 0.349 mmol) followed by sodium borohydride (0.330 mg, 8.73 mmol) addition portion wise at 0° C. under inert atmosphere, after complete addition the reaction was stirred at ambient temperature for 1 h. the reaction mixture was quenched with cold water (30 mL) and extracted with ethyl acetate (3×25 mL), combined organic layer was dried over sodium sulphate, filtered and concentrated to afford 2-(2-(trifluoromethyl) phenyl) cyclobutan-1-amine (6) as a yellow viscous solid (0.215 g, 57%). LCMS (ES) m/z calculated for C11H12F3N, 215.09; found 216.1 (M+H).

### Step 5: Synthesis of 2-amino-6-bromo-3-methyl-N-(2-(2-(trifluoromethyl) phenyl) cyclobutyl) benzamide

To a stirred solution of 2-amino-6-bromo-3-methylbenzoic acid (7) (0.2 g, 0.869 mmol) in N, N-dimethylformamide (5 mL) was added triethylamine (0.422 mL, 3.04 mmol) followed by HATU (0.661 g, 1.74 mmol) at 0° C. under inert atmosphere and stirred for 5 min, then to this solution was added 2-(2-(trifluoromethyl) phenyl) cyclobutan-1-amine (6) (0.187 g, 0.869 mmol) and then stirred at room temperature for 16 h. the reaction mixture was quenched with cold water (30 mL) and extracted with ethyl acetate (3×25 mL), combined organic layer was dried over sodium sulphate, filtered and concentrated to afford crude. The crude was purified through flash column chromatography to afford 2-amino-6-bromo-3-methyl-N-(2-(2-(trifluoromethyl) phenyl) cyclobutyl) benzamide as a yellow viscous solid (0.1 g, 26.92%). LCMS (ES) m/z calculated for C19H18BrF3N2O, 426.06; found 427.0 (M+H).

### Step 6: Synthesis of 5-bromo-2,8-dimethyl-3-(2-(2-(trifluoromethyl) phenyl) cyclobutyl) quinazolin-4(3H)-one

To a stirred solution of 2-amino-6-bromo-3-methyl-N-(2-(2-(trifluoromethyl) phenyl) cyclobutyl) benzamide (0.090 g, 0.211 mmol) in toluene (1 mL) was added pentane-2,4-dione (0.031 g, 0.316 mmol) and p-TSA (0.008 g, 0.042 mmol). The resulting reaction mixture was stirred at 100° C. for 1 h. The reaction mixture was concentrated under reduced pressure to get residue. The residue was dissolved in ethyl acetate (25 mL) and washed with sat. sodium bicarbonate solution (15 mL), water (15 mL) and then organic layer was dried over sodium sulphate, filtered, and concentrated to afford crude 5-bromo-2,8-dimethyl-3-(2-(2-(trifluoromethyl) phenyl) cyclobutyl) quinazolin-4(3H)-one (0.090 g, yield: 95%) as a yellow viscous solid. LCMS (ES) m/z calculated for C21H18BrF3N2O, 450.06; found 451.1 (M+H).

### Step 7: Preparation 3,5-dichloro-N-(2,8-dimethyl-4-oxo-3-(2-(2-(trifluoromethyl) phenyl)cyclobutyl)-3,4-dihydroquinazolin-5-yl)-4-hydroxybenzamide

To a stirred solution of 5-bromo-2,8-dimethyl-3-(2-(2-(trifluoromethyl) phenyl) cyclobutyl) quinazolin-4(3H)-one (9) (0.075 g, 0.166 mmol) in 1,4-dioxane (7.5 mL) was added 3,5-dichloro-4-hydroxybenzamide (10) (0.034 mg, 0.166 mmol) and potassium t-butoxide (0.056 g, 0.499 mmol), the reaction mixture was degassed for 10 min using argon gas, then to this solution was added Xantphos (0.0192 g, 0.033 mmol) and Bis(dibenzylideneacetone)palladium(0) (0.0191 g, 0.033 mmol) at room temperature. The resulting reaction mixture was heated to 100° C. for 1 h, the reaction mixture was cooled to room temperature and to it was added ice cold water (15 mL) and extracted with ethyl acetate (3×25 mL). The combined organic layer was dried over sodium sulphate, filtered, and concentrated to afford crude. The crude was purified through flash column chromatography to afford 3,5-dichloro-N-(2,8-dimethyl-4-oxo-3-(2-(2-(trifluoromethyl) phenyl) cyclobutyl)-3,4-dihydroquinazolin-5-yl)-4-hydroxybenzamide as off-white solid (0.008 g, 8%). LCMS (ES) m/z calculated for C28H22C12F3N3O3, 575.10; found 576.2 (M+H); 1H NMR (400 MHz, DMSO-d6) δ 12.41 (s, 1H), 11.1 (s, 1H), 8.36 (d, J=8.4 Hz, 1H), 7.65-7.8 (m, 2H), 7.53 (d. J=8.4 Hz, 1H), 7.37 (d, J=8.0 Hz, 2H), 7.26 (t, J=7.2 Hz, 1H), 7.16 (t, J=7.6 Hz, 1H), 5.41-5.45 (m, 1H), 4.60-4.64 (m, 1H), 3.0-3.10 (m, 1H), 2.65-2.8 (m, 1H), 2.57 (s, 3H), 2.37 (s, 3H), 1.2-1.3 (m, 1H), 1.15 (t, J=7.2 Hz, 1H).

### Example 18: Synthesis of 4,6-dichloro-N-(1-ethyl-2,4-dioxo-3-(2-(trifluoromethyl)benzyl)-1,2,3,4-tetrahydroquinazolin-5-yl)-5-hydroxypicolinamide

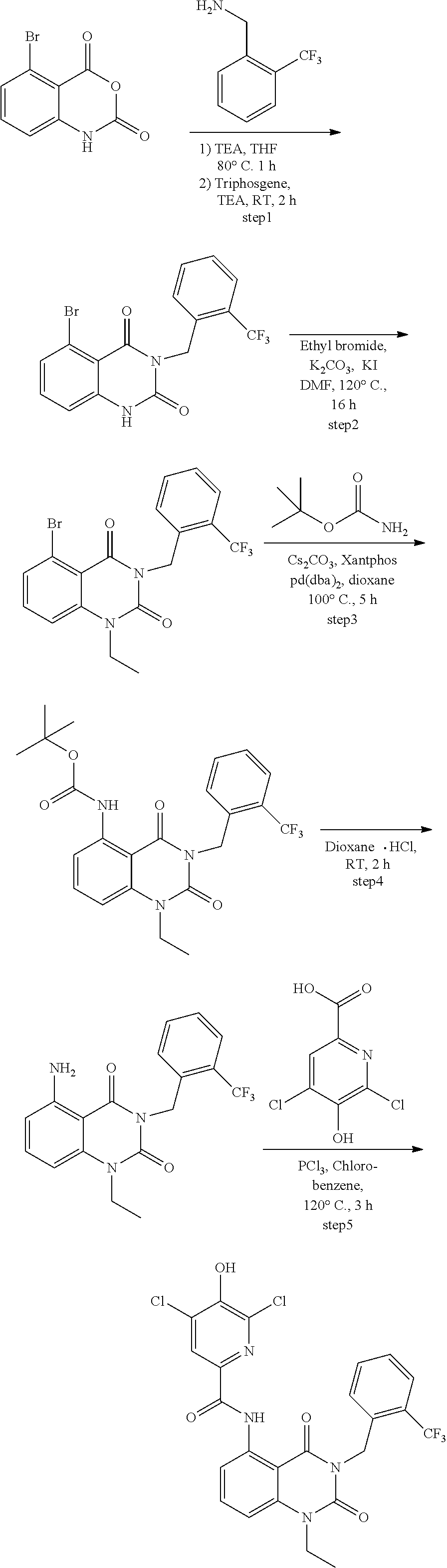

### Step1: Synthesis of 5-bromo-3-{[2-(trifluoromethyl)phenyl]methyl}-1,2,3,4-tetrahydro quinazoline-2,4-dione

To a solution of 5-bromo-2H-benzo[d][1,3]oxazine-2,4(1H)-dione (5 g, 20.7 mmol) in THF (40 mL) was added (2-(trifluoromethyl)phenyl)methanamine (3.62 g, 20.7 mmol) and triethylamine (6.7 mL, 2.5 eq., 51.6 mmol) at ambient temperature. The resulting reaction mixture was heated to 80° C. for 1 h, cooled to room temperature and diluted with ethyl acetate (50 mL) and water (30 mL) and extracted into ethyl acetate. The organic layer was dried over Na₂SO₄ and concentrated to obtain a brown oil. The viscous compound was dissolved in THF (10 mL) cooled to 0° C., then Triphosgene (3.07 g, 0.5 eq., 10.3 mmol) was added and stirred at ambient temperature for 30 min, then triethylamine was added to the stirred solution and continued the stirring for another 2 h, the solid material was removed from the reaction mixture by filtration. The filtrate was concentrated and dried to give brown solid which was triturated with diethyl ether (10 mL) to get 5-bromo-3-(2-(trifluoromethyl)benzyl)quinazoline-2,4(1H,3H)-dione as off white solid (5 g, 60% yield). LCMS (ES) m/z calculated for C16H10BrF3N2O2 397.99 found, 399.0.

### Step 2: Synthesis of 5-bromo-1-ethyl-3-(2-(trifluoromethyl)benzyl)quinazoline-2,4(1H,3H)-dione

To a solution of 5-bromo-3-(2-(trifluoromethyl)benzyl)quinazoline-2,4(1H,3H)-dione (0.5 g, 1.25 mmol) in 5 mL of dimethylformamide was added potassium carbonate (0.26 g, 1.88 mmol), potassium iodide (0.05 g, 0.301 mmol) and bromoethane (0.13 g, 1.25 mmol). The resulting reaction mixture was heated at 120° C. for 16 h, cooled to room temperature and diluted with ethyl acetate (30 mL) and water (20 mL) and extracted into ethyl acetate (2×20 mL). The organic layer was dried over Na₂SO₄ and concentrated to a obtain residue which was triturated with diethyl ether (20 mL) to afford pure 5-bromo-1-ethyl-3-(2-(trifluoromethyl) benzyl)quinazoline-2,4(1H,3H)-dione (0.5 g, 93%) as an off white solid. LCMS (ES) m/z calculated for C18H14BrF3N2O2 426.0; found, 427.0.

### Step 3: Synthesis of tert-butyl (1-ethyl-2,4-dioxo-3-(2-(trifluoromethyl)benzyl)-1,2,3,4-tetrahydroquinazolin-5-yl)carbamate

To a stirred solution of 5-bromo-1-ethyl-3-(2-(trifluoromethyl) benzyl)quinazoline-2,4(1H,3H)-dione (1 g, 2.34 mmol) and tert-butyl carbamate (0.27 g, 2.34 mmol) in 1,4-dioxane (5 mL) and cesium carbonate (1.53 g, 4.68 mmol) was degassed with nitrogen for 10 min, then added Pd(dba)2 (0.067 g, 0.117 mmol) and Xanthphos (0.13 g, 0.234 mmol). The resulting reaction mixture was stirred at 100° C. for 5 h. Progress of the reaction was monitored by LCMS. After the completion of the reaction, reaction mixture was filtered through celite and filtrate was diluted with ethyl acetate (50 mL) and washed with water (30 mL) and the organic layer was dried over Na₂SO₄ and concentrated to get residue which was triturated with diethyl ether (20 mL) to get pure tert-butyl (1-ethyl-2,4-dioxo-3-(2-(trifluoromethyl)benzyl)-1,2,3,4-tetrahydroquinazolin-5-yl)carbamate (0.6 g, 55%). LCMS (ES) m/z calculated for C23H24F3N3O4 463.17; found, 364.1 (M-Boc).

### Step 4: Synthesis of 5-amino-1-ethyl-3-(2-(trifluoromethyl)benzyl)quinazoline-2,4(1H,3H)-dione

To a stirred solution of tert-butyl (1-ethyl-2,4-dioxo-3-(2-(trifluoromethyl)benzyl)-1,2,3,4-tetrahydroquinazolin-5-yl)carbamate (0.6 g, 1.29 mmol) in dichloromethane (5 mL) was cooled to 0° C. and added HCl in dioxane (5 mL) and the reaction was stirred at ambient temperature for 2 h. Progress of the reaction was monitored by TLC/LCMS. After completion of reaction, reaction mixture was concentrated and triturated with diethyl ether (10 mL) to get pure 5-amino-1-ethyl-3-(2-(trifluoromethyl)benzyl)quinazoline-2,4(1H,3H)-dione (450 mg, 95% yield) as off white solid. LCMS (ES) m/z calculated for C18H16F3N3O2 363.2; found, 364.1 (M+H).

### Step 5: Synthesis of 4,6-dichloro-N-(1-ethyl-2,4-dioxo-3-(2-(trifluoromethyl)benzyl)-1,2,3,4-tetrahydroquinazolin-5-yl)-5-hydroxypicolinamide

To a solution of 5-amino-1-ethyl-3-(2-(trifluoromethyl)benzyl)quinazoline-2,4(1H,3H)-dione (0.2 g, 0.55 mmol) and 4,6-dichloro-5-hydroxypyridine-2-carboxylic acid (0.11 g, 0.55 mmol) in chlorobenzene (2 mL) was added PCl₃ (0.075 g, 0.550 mmol). The reaction mixture was heated at 120° C. for 3 h. The progress of the reaction was monitored by TLC/LCMS. After the completion of the reaction, reaction mixture was diluted with ethyl acetate (10 mL) and washed with water (10 mL×2), aqueous bicarbonate solution (10 mL) and the combined organic layer was concentrated to get residue which was purified by reverse phase prep-HPLC. Pure fractions were collected and concentrated to afford 4,6-dichloro-N-(1-ethyl-2,4-dioxo-3-(2-(trifluoromethyl)benzyl)-1,2,3,4-tetrahydroquinazolin-5-yl)-5-hydroxypicolinamide (54 mg, 18% yield) as white solid. LCMS (ES) m/z calculated for C24H17C12F3N4O4 553.36; found, 553.1 (M+H). ¹H NMR (400 MHz, DMSO-d₆) δ 13.54 (s, 1H), 8.64 (d, J=8 Hz, 1H), 8.08 (s, 1H), 7.82-7.7.5 (m, 2H), 7.53 (t, J=7.6 Hz, 1H), 7.45 (t, J=8 Hz, 1H), 7.30-7.22 (m, 1H), 7.21 (d, J=9.2 Hz, 1H), 5.33 (s, 2H), 4.17 (q, J=6.8 Hz, 2H), 1.23 (t, J=6.8 Hz, 3H).

### Example 19: Synthesis of 4,6-dichloro-N-(8-fluoro-1-methyl-2,4-dioxo-3-(2-(trifluoromethyl) benzyl)-1,2,3,4-tetrahydroquinazolin-5-yl)-5-hydroxypicolinamide

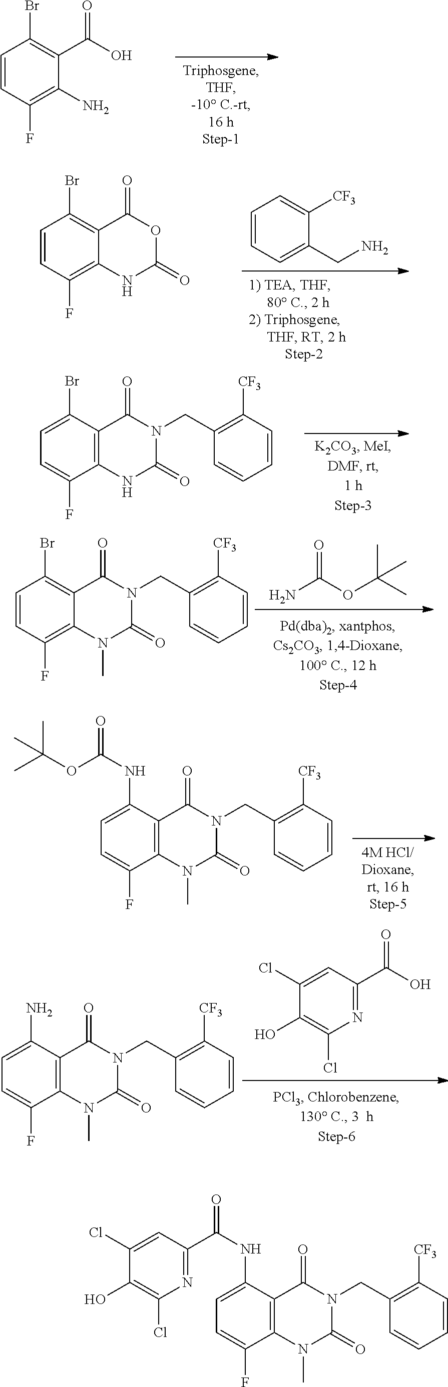

### Step 1: Synthesis of 5-bromo-8-fluoro-2H-benzo[d][1,3]oxazine-2,4(1H)-dione

A stirred solution of 2-amino-6-bromo-3-fluorobenzoic acid (0.5 g, 2.14 mmol) in THF (6 mL) was added triphosgene (0.63 g, 2.14 mmol) at −10° C. for 30 min, then it was stirred for 1 h at −10 to −5° C., followed by 16 hours at room temperature. After completion, the reaction mixture was evaporated under reduced vacuum to afford the residue to which diethyl ether (3×50 mL) was added and stirred for 5 min, obtained solid was collected through filtration, the solid was triturated with diethyl ether (25 mL) and evaporated to dryness to afford 5-bromo-8-fluoro-2H-benzo[d][1,3]oxazine-2,4(1H)-dione (0.4 g, 72%) as light yellow colored solid. LCMS (ES) m/z calcd, For C8H3BrFNO3, 258.93; found, 259.9 (M+H).

### Step 2: Synthesis of 5-bromo-8-fluoro-3-(2-(trifluoromethyl) benzyl) quinazoline-2,4(1H,3H)-dione

To a solution of 5-bromo-8-fluoro-2H-benzo[d][1,3]oxazine-2,4(1H)-dione (0.4 g, 1.54 mmol) in tetrahydrofuran (10 mL) was added with 1-[2-(trifluoromethyl)phenyl]methanamine (0.21 mL, 1.54 mmol) and triethylamine (0.24 mL) dropwise. The reaction mixture was heated at 80° C. for 2 h. After completion, reaction mixture was cooled to room temperature and quenched with water (50 mL) and extracted into ethyl acetate (3×50 mL). The combined organics were washed with water (10 mL) and brine solution (10 mL) dried over Na₂SO₄, filtered, and concentrated under reduced vacuum to afford a brown oil. The oil was dissolved in Tetrahydrofuran (10 mL) cooled to 0° C., then Triphosgene (0.22 g, 0.76 mmol) was added. The reaction mixture was stirred at room temperature for 30 min then was added with triethylamine (0.24 mL). The reaction mixture was stirred at room temperature for 2 h and filtered. The filtrate was concentrated and dried to give 5-bromo-8-fluoro-3-(2-(trifluoromethyl) benzyl) quinazoline-2,4(1H,3H)-dione (0.6 g, 93.5%) as yellow solid. LCMS (ES) m/z calcd, For C16H9BrF4N2O2, 415.98; found, 417.0 (M+H).

### Step 3: Synthesis of 5-bromo-8-fluoro-1-methyl-3-(2-(trifluoromethyl) benzyl) quinazoline-2,4(1H,3H)-dione

To a stirred solution of 5-bromo-8-fluoro-3-(2-(trifluoromethyl) benzyl) quinazoline-2,4(1H,3H)-dione (0.7 g, 1.68 mmol) in N, N-dimethylformamide (7 mL) was added with potassium carbonate (0.69 g, 5.03 mmol) and stirred at room temperature for 15 min. Then iodomethane (0.15 mL, 2.52 mmol) was added to the reaction mixture and was stirred at room temperature for 1 h. After completion, water (25 mL) was added and it was extracted with ethyl acetate (3×25 mL). The combined organics were washed with brine solution (15 mL) and dried over anhydrous sodium sulfate, filtered and dried under reduced vacuum to afford 5-bromo-8-fluoro-1-methyl-3-(2-(trifluoromethyl) benzyl) quinazoline-2,4(1H,3H)-dione 5 (0.5 g, 69.1%) as off white solid. LCMS (ES) m/z calcd, For C17H11BrF4N2O2, 431.2; found, 432.0 (M+H).

### Step 4: Synthesis of tert-butyl (8-fluoro-1-methyl-2,4-dioxo-3-(2-(trifluoromethyl)benzyl)-1,2,3,4-tetrahydroquinazolin-5-yl)carbamate

To a stirred solution of 5-bromo-8-fluoro-1-methyl-3-(2-(trifluoromethyl) benzyl) quinazoline-2,4(1H,3H)-dione (0.2 g, 0.464 mmol) and tert-butyl carbamate (0.16 g, 1.39 mmol) in 1,4-dioxane (3 mL) was added with cesium carbonate (0.45 g, 1.39 mmol). The reaction mixture was degassed with nitrogen and was added with Xantphos (0.053 g, 0.92 mmol) and Pd(dba)2 (0.026 g, 0.46 mmol). The reaction mixture was heated to 100° C. for 12 h. The reaction mixture was filtered through celite pad, washed with ethyl acetate (2×50 mL), the filtrate was evaporated under reduced pressure to afford crude tert-butyl (8-fluoro-1-methyl-2,4-dioxo-3-(2-(trifluoromethyl)benzyl)-1,2,3,4-tetrahydroquinazolin-5-yl)carbamate (0.5 g, 92%) as off white solid. LCMS (ES) m/z calcd, For C22H21F4N3O4, 467.15; found, 368.1 (M− Boc H).

### Step 5: Synthesis of 5-amino-8-fluoro-1-methyl-3-(2 (trifluoromethyl) benzyl) quinazoline-2,4(1H,3H)-dione

To a stirred solution of tert-butyl (8-fluoro-1-methyl-2,4-dioxo-3-(2-(trifluoromethyl)benzyl)-1,2,3,4-tetrahydroquinazolin-5-yl)carbamate (0.5 g, 1.07 mmol) in 1,4-dioxane was added 4M HCl in 1,4-Dioxane (5 mL) at room temperature and then stirred at same temperature for 16 h. After completion of the reaction, the reaction mixture was evaporated under reduced vacuum to afford yellow colored solid, the solid was triturated with diethyl ether (25 mL) and evaporated to dryness to afford 5-amino-8-fluoro-1-methyl-3-(2-(trifluoromethyl)benzyl)quinazoline-2,4(1H,3H)-dione (0.3 g, 76%) as light yellow colored solid. LCMS (ES) m/z calcd, For C₁₇H13F4N302, 367.09; found, 368.1 (M+H).

### Step 6: Synthesis of 4,6-dichloro-N-(8-fluoro-1-methyl-2,4-dioxo-3-(2-(trifluoromethyl) benzyl)-1,2,3,4-tetrahydroquinazolin-5-yl)-5-hydroxypicolinamide

To a stirred solution of 5-amino-8-fluoro-1-methyl-3-(2-(trifluoromethyl)benzyl)quinazoline-2,4(1H,3H)-dione (0.15 g, 0.408 mmol) and 4,6-dichloro-5-hydroxypyridine-2-carboxylic acid (0.084 mg, 0.40 mmol) in chlorobenzene (2 mL) was added with trichlorophosphane (0.025 mL, 0.286 mmol) at 0° C. under nitrogen atmosphere and was heated to 130° C. for 3 h. After completion, the reaction mass was quenched with ice cold water (15 mL) and stirred for 5 min, obtained solid was collected through filtration, washed with diethyl ether (2×15 mL) and dried under reduced vacuum to afford crude as a yellow colored solid. The crude solid material was purified through prep-purification. Collected fractions were lyophilized to afford 4,6-dichloro-N-(8-fluoro-1-methyl-2,4-dioxo-3-(2-(trifluoromethyl)benzyl)-1,2,3,4-tetrahydroquinazolin-5-yl)-5-hydroxypicolinamide (0.045 g, 20%) as an off white solid. LCMS (ES) m/z calcd, For C23H14C12F4N4O4, 556.03; found, 555.1 (M−H); 1H NMR (400 MHz, DMSO-d6) δ13.42 (s, 1H), 8.65-8.61 (m, 1H), 8.07 (s, 1H), 7.77-7.72 (m, 2H), 7.56-7.52 (m, 1H), 7.48-7.44 (m, 1H), 7.31-7.29 (m, 1H), 5.31 (s, 2H), 3.68 (d, J=9.2 Hz, 3H).

### Example 20: Synthesis of 4,6-dichloro-5-hydroxy-N-(1-methyl-2,4-dioxo-3-(2-(trifluoromethyl)benzyl)-1,2,3,4-tetrahydroquinazolin-5-yl)picolinamide

The title compound was synthesized as described above. LCMS (ES) m/z calculated for C23H15C12F3N4O4 calculated for 538.04; found, 539.1 (M+H). ¹H NMR (400 MHz, DMSO-d₆) δ 13.52 (s, 1H), 8.65 (d, J=8.4 Hz, 1H), 8.08 (s, 1H), 7.83-7.75 (m, 2H), 7.55-7.40 (m, 2H), 7.25-7.22 (m, 2H), 5.33 (s, 2H), 3.54 (s, 3H).

### Example 21: Synthesis of 5-(3,5-dichloro-4-hydroxybenzamido)-2-methyl-N-(2-(trifluoromethoxy)benzyl)thiazole-4-carboxamide

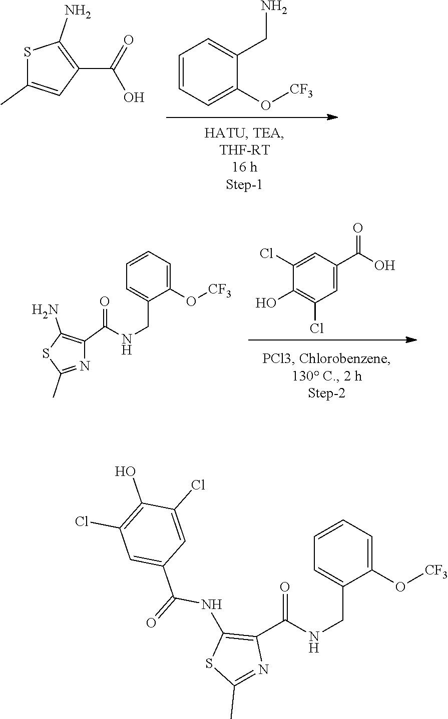

### Step 1: Synthesis of 5-amino-2-methyl-N-(2-(trifluoromethoxy)benzyl)thiazole-4-carboxamide

To a stirred solution of 5-amino-2-methylthiazole-4-carboxylic acid (0.5 g, 3.16 mmol) in tetrahydrofuran (5 mL) was added HATU (2.4 g, 6.32 mmol), triethylamine (2.2 mL, 15.8 mmol) and (2-(trifluoromethoxy)phenyl)methanamine (0.6 g, 3.16 mmol) at ambient temperature. The resulting reaction mixture was stirred for 16 h at ambient temperature. Progress of the reaction was monitored by TLC/LCMS. After completion of the reaction the reaction mixture was quenched with ice cold water (20 mL) and extracted with ethyl acetate (3×10 ml). The combined organic layer was washed with water (10 mL), brine (10 mL), dried over sodium sulfate and evaporated under reduced pressure to result in crude compound which was purified through flash column chromatography to afford 5-amino-2-methyl-N-(2-(trifluoromethoxy)benzyl)thiazole-4-carboxamide as a yellow liquid (0.88 g, 84%). LCMS (ES) m/z calcd. C13H12F3N3O2S, 331.0; found, 332.1 (M+1).

### Step 2: Synthesis of 5-(3,5-dichloro-4-hydroxybenzamido)-2-methyl-N-(2-(trifluoromethoxy)benzyl)thiazole-4-carboxamide

To a stirred solution of 5-amino-2-methyl-N-(2-(trifluoromethoxy)benzyl)thiazole-4-carboxamide (0.4 g, 1.21 mmol) in Chlorobenzene (5 mL) was added phosphorus trichloride (52.8 μL, 0.604 mmol) and 3,5-dichloro-4-hydroxybenzoic acid (275 mg, 1.33 mmol) and allowed to stirred at 130° C. for 2 h. Progress of the reaction was monitored by TLC/LCMS. After completion of the reaction, the reaction mixture was quenched with ice cold water (20 mL) and filtered to result in crude compound which was purified through Flash column chromatography to get off-white solid. The solid was further triturated with hot methanol (10 mL) to afford 5-(3,5-dichloro-4-hydroxybenzamido)-2-methyl-N-(2-(trifluoromethoxy)benzyl)thiazole-4-carboxamide as a white solid (0.05 g, 8%). LCMS (ES) m/z calcd. C20H14Cl2F3N3O4S, 519.0; found, 520.1 (M+1). 1H NMR (400 MHz, DMSO d6) δ 12.31 (s, 1H), 11.32 (s, 1H), 9.11 (t, J=8.0 Hz, 1H), 7.82 (s, 2H), 7.45-7.35 (m, 4H), 4.59 (d, J=4.0 Hz, 2H), 2.65 (s, 3H).

### Example 22: Synthesis of 5-(4,6-dichloro-5-hydroxypicolinamido)-2-methyl-N-(2-(trifluoromethoxy) benzyl)thiazole-4-carboxamide

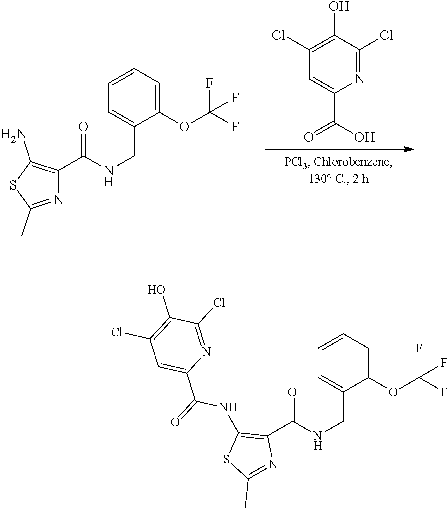 [SMILES: Cc1nc(C(=O)NCc2ccccc2OC(F)(F)F)c(N)s1 ; O=C(O)c1cc(Cl)c(O)c(Cl)n1 ; Cc1nc(C(=O)NCc2ccccc2OC(F)(F)F)c(NC(=O)c2cc(Cl)c(O)c(Cl)n2)s1]

To a stirred solution of 5-amino-2-methyl-N-(2-(trifluoromethoxy)benzyl)thiazole-4-carboxamide (0.4 g, 1.21 mmol) in Chlorobenzene (5 mL) was added Phosphorus trichloride (52.8 μL, 0.604 mmol) and 4,6-dichloro-5-hydroxypicolinic acid (0.27 g, 1.33 mmol) at ambient temperature. The resulting reaction mixture was heated to 130° C. for 2 h. Progress of the reaction was monitored by TLC/LCMS. After completion of the reaction, the reaction mixture was quenched with ice water (20 mL). Precipitated solid was filtered and dried to get off white solid. The solid material was purified through Flash column chromatography to afford 5-(4,6-dichloro-5-hydroxypicolinamido)-2-methyl-N-(2-(trifluoromethoxy)benzyl)thiazole-4-carboxamide as a white solid (0.051 g, 8%). LCMS (ES) m/z calcd. C19H13C12F3N4O4S, 520.00; found, 521.1 (M+1); 1H NMR (400 MHz, DMSO d6) δ 12.64 (s, 1H), 9.0 (t, J=4 Hz, 1H), 8.09 (s, 1H), 7.40-7.32 (m, 4H), 4.55 (d, J=8.0 Hz, 2H), 2.62 (s, 3H).

### Example 23: Synthesis of 5-(4,6-dichloro-5-hydroxypicolinamido)-2-phenyl-N-(2 (trifluoromethyl)benzyl)thiazole-4-carboxamide

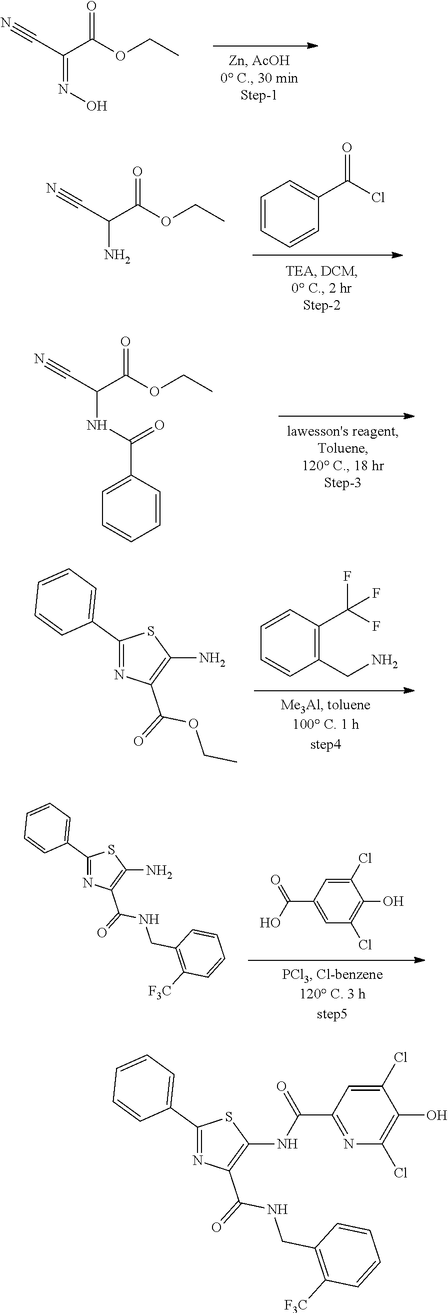

### Step1: Synthesis of ethyl 2-amino-2-cyanoacetate

To a stirred solution of (E)-(ethyl cyano(hydroxyimino)formate) (5 g, 35.2 mmol) in acetic acid (50 mL) was added zinc (11.5 g, 5 eq., 176 mmol) portion-wise at ambient temperature. The reaction mixture was stirred at ambient temperature for 30 minutes. The reaction progress was monitored by TLC. After completion of the reaction, the mixture was filtered through celite, washed with dichloromethane (200 mL). The filtrate was evaporated under reduced pressure to afford ethyl 2-amino-2-cyanoacetate (4 g) as an orange viscous solid. The crude compound was used for the next step without purification.

### Step2: Synthesis of ethyl 2-cyano-2-(phenylformamido)acetate

To a stirred solution of ethyl 2-amino-2-cyanoacetate (4 g, 31.2 mmol) in dichloromethane (50 mL) was added benzoyl chloride (4.83 g, 34 mmol) and triethylamine (7.9 g, 78 mmol) at 0° C. The reaction was allowed to stir for 2 h at ambient temperature. The reaction progress was monitored by TLC. After completion of the reaction, the mixture was diluted with dichloromethane (50 mL), washed with water (100 mL) and brine solution (100 mL). The organic phase was dried over anhydrous sodium sulfate, filtered, and evaporated to get crude product. Purification by flash chromatography afforded ethyl 2-cyano-2-(phenylformamido) acetate as yellow solid (1 g, 13% yield). LCMS (ES) m/z calculated for C₁₂H₁₂N₂O₃ 232.08; found, 233.1.

### Step3: Synthesis of ethyl 5-amino-2-phenyl-1,3-thiazole-4-carboxylate

To a stirred solution of ethyl 2-cyano-2-(phenylformamido)acetate (1 g, 4.31 mmol) in toluene (20 mL) was added Lawesson's reagent (5.22 g, 3 eq., 12.9 mmol). The resulting reaction mixture was heated to reflux for 18 hr. The reaction progress was monitored by TLC and LCMS. After completion, reaction mixture was diluted with Ethyl acetate (50 mL), washed with saturated sodium bicarbonate solution (3×50 mL). The organic phase was dried over anhydrous sodium sulfate, filtered, and evaporated to get a residue. Which was purified by flash chromatography to afford as ethyl 5-amino-2-phenyl-1,3-thiazole-4-carboxylate as a yellow solid (400 mg, 37%). LCMS (ES) m/z calculated for C12H12N2O2S 248.06; found, 249.1.

### Step 5: Synthesis of 5-amino-2-phenyl-N—{[2-(trifluoromethyl)phenyl]methyl}-1,3-thiazole-4-carboxamide

To a stirred solution of ethyl 5-amino-2-phenyl-1,3-thiazole-4-carboxylate (0.25 g, 1.01 mmol) and 1-[2-(trifluoromethyl)phenyl]methanamine (0.21 g, 1.21 mmol) in Toluene (1 mL) was added 2 M trimethylaluminium in toluene (2 mL, 4 mmol). The reaction mixture was heated at 100° C. for 1 h. The reaction mixture was cooled to ambient temperature and quenched with ice cold water (15 mL) and extracted with Ethyl acetate (25×2 mL). The combined organic phase was washed with brine solution (15 mL) and dried over anhydrous sodium sulphate, filtered, and evaporated in vacuo to obtain crude brown solid which was triturated with diethyl ether (10 mL) to afford 5-amino-2-phenyl-N-{[2-(trifluoromethyl)phenyl]methyl}-1,3-thiazole-4-carboxamide (0.2 g, 52% yield). LCMS (ES) m/z calculated for C18H14F3N3OS 377.3; found, 378.1.

### Step 6: Synthesis of Synthesis of 5-(4,6-dichloro-5-hydroxypicolinamido)-2-phenyl-N-(2 (trifluoromethyl)benzyl)thiazole-4-carboxamide

To a stirred solution of 5-amino-2-phenyl-N-{[2-(trifluoromethyl)phenyl]methyl}-1,3-thiazole-4-carboxamide (0.2 g, 0.53 mmol) and 4,6-dichloro-5-hydroxypyridine-2-carboxylic acid (110 mg, 0.53 mmol) in Chlorobenzene (2 mL) was added Phosphorous trichloride (72.8 mg, 0.53 mmol) at ambient temperature. The resulting reaction mixture was heated at 120° C. for 3 h. The progress of reaction was monitored by LCMS. The reaction mixture was cooled to ambient temperature and diluted with ethyl acetate (25 mL) and washed with water (10 mL×2), aqueous bicarbonate solution (10 mL). The organic phase was dried over Na2SO4, concentrated under reduced pressure to get crude residue which was purified by reverse phase prep-HPLC to afford 4,6-dichloro-5-hydroxy-N-[2-phenyl-4-({[2-(trifluoromethyl)phenyl]methyl}carbamoyl)-1,3-thiazol-5-yl]pyridine-2-carboxamide as a pale yellow solid (0.07 g, 22% yield). LCMS (ES) m/z calculated for C24H15C12F3N4O3S 566.02 found, 565.1 (M−H). 1H NMR (400 MHz, DMSO d6) δ 12.73 (s, 1H), 9.25 (d, J=6 Hz, 1H), 8.13 (s, 1H), 8.04 (d, J=7.2 Hz, 2H), 7.74 (d, J=8 Hz, 1H), 7.66-6.63 (m, 1H), 7.54-7.47 (m, 5H), 4.75 (d, J=4.8 Hz, 2H).

### Example 24: 5-(4,6-dichloro-5-hydroxypicolinamido)-N-(1-(2-(trifluoromethyl)phenyl) cyclopropyl)thiazole-4-carboxamide

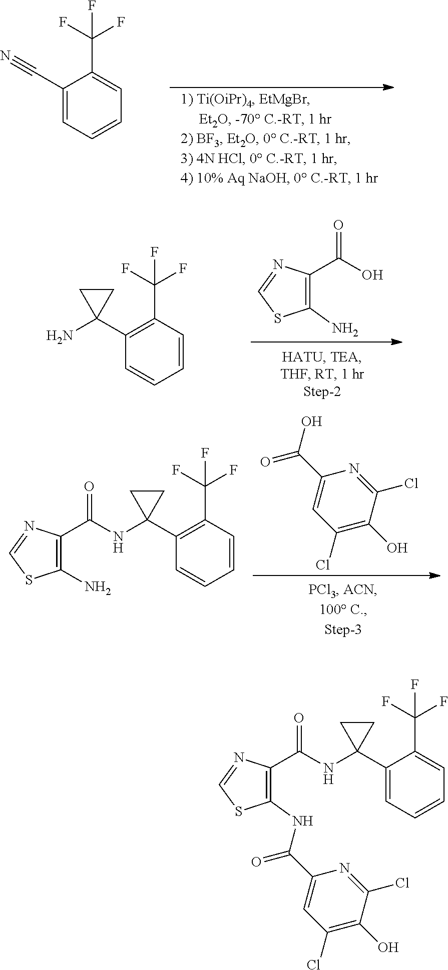

### Step 1: Synthesis of 1-(2-(trifluoromethyl)phenyl)cyclopropan-1-amine

To as stirred solution of 2-(trifluoromethyl)benzonitrile (5 g, 29.2 mmol) and titanium tetraisopropoxide (13.3 mL, 43.8 mmol) in diethyl ether (50 mL, 481 mmol) was added 1M ethylmagnesium bromide in diethyl ether (73 mL, 73 mmol) at −78° C. under nitrogen atmosphere and then reaction mixture was slowly rise to 0° C. for 1 h. To this was added boron trifluoride diethyl etherate (9.02 mL, 73 mmol) dropwise at 0° C. The reaction mixture was allowed to stir at ambient temperature for 1 hr. Then the reaction mixture was cooled to 0° C., added with 4N HCl (50 mL) and to stirred at ambient temperature for another 1 hr. Then the reaction mixture cooled to 0° C., diluted with diethyl ether (100 mL) and was added 20% Aq NaOH (80 mL). The reaction mixture was stirred at ambient temperature for 1 hr. The layers were separated, organic layer was washed with cold 4N HCl (2×25 mL). The aqueous layer was basified by using cold 20% Aq NaOH (pH=10) and extracted with diethyl ether (2×100 mL). The organic layer was dried over anhydrous sodium sulfate, filtered, and concentrated under reduced pressure at ambient temperature to afford 1-[2-(trifluoromethyl)phenyl]cyclopropan-1-amine (3 g, 14.9 mmol) as yellow liquid. LCMS (ES) m/z calcd, For C10H10F3N, 201.08; found, 202.1 (M+H).

### Step 2: Synthesis of 5-amino-N-(1-(2-(trifluoromethyl)phenyl)cyclopropyl)thiazole-4-carboxamide

To a stirred solution of 5-amino-1,3-thiazole-4-carboxylic acid (0.2 g, 1.39 mmol) and 1-[2-(trifluoromethyl)phenyl]cyclopropan-1-amine (279 mg, 1.39 mmol) and triethylamine (0.5 mL 4.16 mmol) in tetrahydrofuran (4 mL) was added HATU (633 mg, 1.66 mmol). The reaction mixture was stirred at ambient temperature for 3 h. After completion, reaction mixture was diluted with Ethyl acetate (50 mL), washed with water (2×20 mL) and brine solution (20 mL). The organic phase was dried over anhydrous sodium sulfate, filtered, and evaporated to get crude product. Which was purified through flash column chromatography, collected fractions were judged by TLC, pure fractions were combined and evaporated under reduced pressure to afford 5-amino-N-{1-[2-(trifluoromethyl)phenyl]cyclopropyl}-1,3-thiazole-4-carboxamide (0.3 g) as off-white solid. LCMS (ES) m/z calcd, For C14H12F3N3OS, 327.07; found, 328.1 (M+H).

### Step 3: Synthesis of 5-(4,6-dichloro-5-hydroxypicolinamido)-N-(1-(2-(trifluoromethyl) phenyl)cyclopropyl)thiazole-4-carboxamide

To a stirred suspension 5-amino-N-{1-[2-(trifluoromethyl)phenyl]cyclopropyl}-1,3-thiazole-4-carboxamide (0.1 g, 0.306 mmol) and 4,6-dichloro-5-hydroxypyridine-2-carboxylic acid (95.3 mg, 1.5 eq., 0.458 mmol) in acetonitrile (5 mL) at 0° C. under nitrogen atmosphere was added Phosphorus trichloride (42 mg, 0.306 mmol). The reaction mixture was heated to 100° C. for 16 hr. After completion, reaction mixture was diluted with Ethyl acetate (50 mL), washed with saturated sodium bicarbonate solution (20 mL), water (20 mL) and brine solution (20 mL). The organic phase was dried over anhydrous sodium sulfate, filtered, and evaporated to get crude product. Purification by prep HPLC afforded 4,6-dichloro-5-hydroxy-N-[4-({1-[2-(trifluoromethyl) phenyl]cyclopropyl}carbamoyl)-1,3-thiazol-5-yl]pyridine-2-carboxamide (0.05 g, 31%) as white solid. LCMS (ES) m/z calcd, For C20H13Cl2F3N4O3S, 516.00; found, 515.1 (M−H); 1H NMR (400 MHz, DMSO-d6) δ 12.49 (s, 1H), 8.67 (s, 1H), 8.34 (s, 1H), 8.12 (s, 1H), 8.05 (d, J=7.6 Hz, 1H), 7.71-7.67 (m, 2H), 7.53-7.49 (m, 1H), 1.45 (m, 2H), 1.33 (m, 2H).

### Example 25: Synthesis of 5-(4,6-dichloro-5-hydroxypicolinamido)-N-methyl-N-(2-(trifluoromethyl)benzyl)thiazole-4-carboxamide

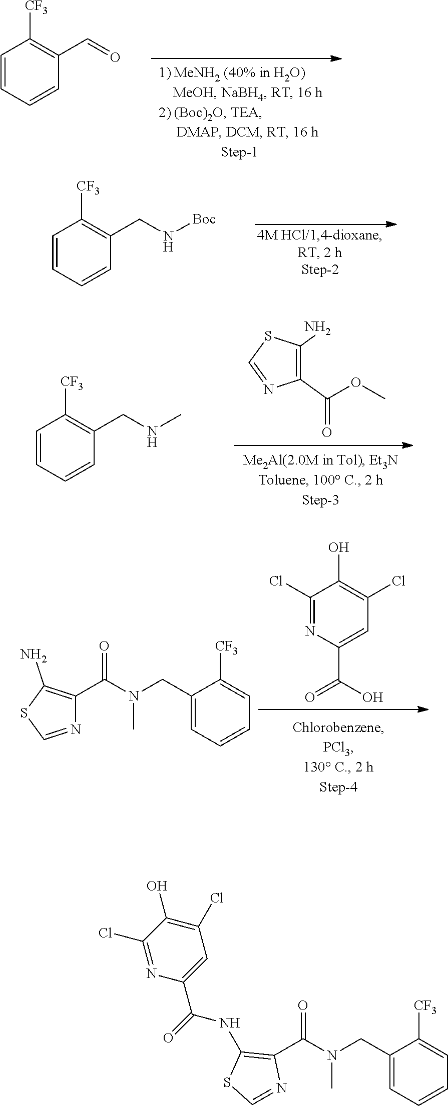

### Step 1: Synthesis of tert-butyl (2-(trifluoromethyl)benzyl)carbamate

To a stirred solution of 2-(trifluoromethyl)benzaldehyde (1 g, 5.74 mmol) in methanol (10 mL, 247 mmol) was added methanamine (3.19 mL, 28.7 mmol) and allowed to stir at room temperature for 30 min. After 30 minutes, reaction mass was cooled to 0° C. and added Sodium borohydride (0.43 g, 11.5 mmol) portion-wise and stirred at room temperature for 2 hours. To the reaction mixture was added triethylamine (2.4 mL, 17.2 mmol), N,N-dimethylpyridin-4-amine (0.14 g, 1.15 mmol) and di-tert-butyl dicarbonate (1.98 mL, 8.61 mmol) and allowed to stir at room temperature for 16 hours. The reaction mixture was concentrated under reduced pressure to remove methanol. The residue was quenched with water (50 mL) and extracted into Ethyl acetate (20 mL×2). The combined organic layer was washed with brine (20 mL), dried over sodium sulfate and evaporated under reduced pressure to result in crude tert-butyl (2-(trifluoromethyl)benzyl)carbamate (0.8 g, 48%) as a gummy solid. LCMS (ES) m/z calculated for C13H16F3NO2 is 275.11; found, 275.1 (M+H).

### Step 2: Synthesis of N-methyl-1-(2-(trifluoromethyl)phenyl)methanamine

To a stirred solution of tert-butyl (2-(trifluoromethyl)benzyl)carbamate (0.8 g, 2.77 mmol) in 4 M HCl in 1, 4 Dioxane (1.01 g, 10 eq., 27.7 mmol) was allowed to stir at room temperature for 16 hours. The reaction mixture was concentrated under reduced pressure and the residue was washed with di-ethyl ether (10 mL) to afford N-methyl-1-(2-(trifluoromethyl)phenyl)methanamine (0.6 g, 92%) as an off-white solid. LCMS (ES) m/z calculated for C9H11ClF3N is 225.05; found, 190.1 (M+H).

### Step 3: Synthesis of 5-amino-N-methyl-N-(2-(trifluoromethyl)benzyl)thiazole-4-carboxamide

To a stirred solution of methyl 5-amino-1,3-thiazole-4-carboxylate (0.25 g, 1.58 mmol), methyl({[2-(trifluoromethyl)phenyl]methyl})amine hydrochloride (0.428 g, 1.9 mmol) and triethylamine (0.22 mL, 1.58 mmol) in toluene (2 mL, 16.9 mmol). The mixture was cooled to 0° C. and added trimethylalumane (0.455 mL, 4.74 mmol). The resulting reaction mixture was heated to 100° C. for 2 h. The reaction mixture was cooled to ambient temperature and quenched with water (15 mL) and extracted into ethyl acetate (2×30 mL). The combined organics were washed with brine solution (2×10 mL), dried over sodium sulfate, filtered, and evaporated under reduced pressure to afford crude. The crude product was purified by Combi flash chromatography to afford 5-amino-N-methyl-N-(2-(trifluoromethyl)benzyl)thiazole-4-carboxamide (0.24 g, 48%) as off-white solid. LCMS (ES) m/z calculated for C13H12F3N3OS is 315.07; found, 316.1 (M+H).

### Step 4: Synthesis of 5-(4,6-dichloro-5-hydroxypicolinamido)-N-methyl-N-(2-(trifluoromethyl)benzyl)thiazole-4-carboxamide

To a stirred solution of 5-amino-N-methyl-N-{[2-(trifluoromethyl)phenyl]methyl}-1,3-thiazole-4-carboxamide (0.220 g, 0.698 mmol) and 4,6-dichloro-5-hydroxypyridine-2-carboxylic acid (0.16 g, 0.767 mmol) in chlorobenzene (4 mL) were added trichlorophosphane (0.018 mL, 0.209 mmol) dropwise to the reaction mixture at room temperature. Then the reaction was stirred at 130° C. for 2 hours. The reaction mixture was cooled to ambient temperature and quenched into ice-cold water (10 mL) and resulting mixture was extracted into ethyl acetate (5 mL×2). The combined organic layer was washed with brine (3 mL×2), dried over sodium sulfate, and evaporated under reduced pressure to result in crude. The resulting crude product was purified by prep-HPLC to afford 5-(4,6-dichloro-5-hydroxypicolinamido)-N-methyl-N-(2-(trifluoromethyl)benzyl)thiazole-4-carboxamide (0.048 g, 13%) as white solid. LCMS (ES) m/z calculated for C19H13C12F3N4O3S is 504.00; found, 505.1 (M+H). ¹H NMR (400 MHz, DMSO d6 at 90° C.) δ 12.78 (bs, 1H), 8.61 (s, 1H), 8.09 (s, 1H), 7.76 (d, J=7.6 Hz, 1H), 7.66 (t, J=7.6 Hz, 1H), 7.52-7.46 (m, 3H), 5.34 (bs, 2H), 3.05 (s, 3H).

The following compounds were synthesized as described above.

| Ex. | Name | Analytical Data |
|---|---|---|
| 26 | 5-(4,6-dichloro-5- | LCMS (ES): m/z calculated for C19H13Cl2F3N4O3S; |
| | hydroxypicolinamido)-2-methyl-N-(2- | 504.0 found, 505.1 (M + H); ¹H NMR (400 MHz, |
| | (trifluoromethyl)benzyl)thiazole- | DMSO-d₆): δ 12.60 (s, 1H), 9.07 (t, J = 6 Hz, 1H), |
| | 4-carboxamide | 8.10 (s, 1H), 7.73 (d, J = 8.4 Hz, 1H), 7.63 (t, J = 8.2 |
| | | Hz, 1H), 7.47-7.43 (m, 2H), 4.68 (s, 2H), 2.62 (s, 3H). |
| 27 | 5-(3,5-dichloro-4- | LCMS (ES): m/z calculated for C20H14Cl2F3N3O3S, |
| | hydroxybenzamido)-2-methyl-N-(2- | 503.1; found, 504.1 (M + H); ¹H NMR (400 MHz, |
| | (trifluoromethyl)benzyl)thiazole- | DMSO-d₆): δ 12.20 (s, 1H), 11.12 (s, 1 H), 9.18 (s, |
| | 4-carboxamide | 1H), 7.78 (s, 2H), 7.72 (d, J = 8.4 Hz, 1H), 7.62 (t, J = |
| | | 8.2 Hz, 1H), 7.48-7.45 (m, 2H), 4.69 (s, 2H), 2.64 (s, |
| | | 3H). |
| 28 | 2-(4,6-dichloro-5- | LCMS (ES): m/z calculated for C18H11Cl2F3N4O3S, |
| | hydroxypicolinamido)-N-(2- | 489.9; found 491.1 [M + H]; ¹H NMR (400 MHz, |
| | (trifluoromethyl) | DMSO-d₆): 11.45 (bs, 1H), 9.11 (t, J = 5.8 Hz, 1H), |
| | benzyl)thiazole-5-carboxamide | 8.21 (s, 1H), 7.96 (bs, 1H), 7.75-7.67 (m, 2H), 7.58-7.47 |
| | | (m, 2H), 4.64 (d, J = 5.6 Hz, 2H). |
| 29 | 5-(4,6-dichloro-5- | LCMS (ES) m/z calculated for C19H13Cl2F3N4O4S, |
| | hydroxypicolinamido)-3-methyl-N-(2- | 520.0; found, 521.0 (M + H); ¹H NMR (400 MHz, |
| | (trifluoromethoxy)benzyl)isothiazole- | DMSO-d₆) δ 12.36 (s, 1H), 8.34 (t, J = 6 Hz, 1H), |
| | 4-carboxamide | 8.10 (s, 1H), 7.51 (d, J = 6.8 Hz, 1H), 7.43-7.36 (m, |
| | | 3H), 4.61 (d, J = 6 Hz, 2H), 2.69-2.61 (m, 3H). |
| 30 | N-(adamantan-1-ylmethyl)-5- | LCMS (ES) m/z calculated for C21H22Cl2N4O3S, |
| | (4,6-dichloro-5- | 480.08; found 481.2 (M + H); ¹H NMR (400 MHz, |
| | hydroxypicolinamido)thiazole-4- | DMSO-d₆) δ 12.78 (s, 1H), 8.72 (s, 1H), 8.22 (t, J = |
| | carboxamide | 8.5 Hz, 1H), 8.14 (s, 1H), 3.07 (d, J = 6.4 Hz, 2H), |
| | | 1.95 (s, 3H), 1.68-1.59 (m, 6H), 1.52 (bs, 6H). |
| 31 | N-(4-(2-azaspiro[4.5]decane-2- | LCMS (ES) m/z calculated for C19H20Cl2N4O3S |
| | carbonyl)thiazol-5-yl)-4,6- | 454.06 found, 455.1 (M + H); ¹H NMR (400 MHz, |
| | dichloro-5-hydroxypicolinamide | DMSO-d₆) δ 13.13 (s, 1H), 8.68 (d, J = 11.6 Hz, 1H), |
| | | 8.12 (s, 1H), 4.05 (t, J = 6.8 1H), 3.84 (s, 1H), 3.64 |
| | | (t, J = 7.2 Hz, 1H), 2.05 (s, 2H), 1.79-1.67 (m, 2H), |
| | | 1.45-1.40 (m, 10H). |
| 32 | 5-(4,6-dichloro-5- | LCMS (ES) m/z calculated for C20H16Cl2N4O3S, |
| | hydroxypicolinamido)-N-(1- | 462.03; found, 463.1 (M + H); ¹H NMR (400 MHz, |
| | phenylcyclobutyl)thiazole-4- | DMSO-d₆ δ 12.57 (s, 1H), 9.03 (s, 1H), 8.73 (s, 1H), |
| | carboxamide | 8.12 (s, 1H), 7.54 (d, J = 7.2 Hz, 2H), 7.34 (t, J = |
| | | 7.6 Hz, 2H), 7.23-7.20 (m, 1H), 2.77-2.67 (m, 2H), |
| | | 2.60-2.58 (m, 2H), 2.06-2.00 (m, 1H), 1.89-1.84 (m, 1H). |
| 33 | 5-(4,6-dichloro-5- | LCMS (ES) m/z calculated for C17H10Cl2F2N4O3S, |
| | hydroxypicolinamido)-N-(2,6- | 457.98; found, 459.1(M + H); ¹H NMR (400 MHz, |
| | difluorobenzyl)thiazole-4- | DMSO-d₆ δ 12.77 (s, 1H), 8.89 (t, J = 8.0 Hz, 1H), |
| | carboxamide | 8.70 (s, 1H), 8.15 (s, 1H), 7.43-7.36 (m, 1H), |
| | | 7.12-7.06 (m, 2H), 4.62 (d, J = 4.0 Hz, 2H). |
| 34 | 5-(4,6-dichloro-5- | LCMS (ES)m/z calculated for C17H10Cl2F3N5O3S |
| | hydroxypicolinamido)-N-((6- | 490.98; found, 492.1 (M + H); ¹H NMR (400 MHz, |
| | (trifluoromethyl)pyridin-3- | DMSO-d₆ δ 12.73 (s, 1H), 9.36 (t, J = 6.4 Hz, 1H), 8.76 |
| | yl)methyl)thiazole-4- | (d, J = 8.8 Hz, 2H), 8.14 (s, 1H), 8.02 (d, J = 7.6 Hz, |
| | carboxamide | 1H), 7.89 (d, J = 8.0 Hz, 1H), 4.66 (d, J = 6 Hz, 2H). |
| | | LC-purity: 99.66% at 240 nm. |
| 35 | 5-(4,6-dichloro-5- | LCMS (ES)m/z calculated for C16H18Cl2N4O3S, |
| | hydroxypicolinamido)-N-(3,3- | 416.05; found 417.2 (M + H); ¹H NMR (400 MHz, |
| | dimethylbutyl)thiazole-4- | DMSO-d₆ δ 12.87 (s, 1H), 8.71 (s, 1H), 8.52 (t, J = |
| | carboxamide | 8.0 Hz, 1H), 8.16 (s, 1H), 3.32-3.38(m, 2H), 1.47-1.51 |
| | | (m, 2H), 0.94 (s, 9H). |
| 36 | 5-(4,6-dichloro-5- | LCMS (ES)m/z calculated for C14H11Cl2FN4O3S, |
| | hydroxypicolinamido)-N-((1- | 403.99; found, 405.1 (M + H); ¹H NMR (400 MHz, |
| | fluorocyclopropyl)methyl)thiazole- | DMSO-d₆ δ 12.78 (s, 1H), 8.74-8.71 (m, 2H), 8.15 |
| | 4-carboxamide | (s, 1H), 3.79-3.72 (m, 2H), 1.02-0.96 (m, 2H), |
| | | 0.89-0.86 (m, 2H). |
| 37 | 5-(4,6-dichloro-5- | LCMS (ES) m/z calculated for C18H19Cl2N5O3S, |
| | hydroxypicolinamido)-N-(2- | 455.06; found, 456.10 (M + H); ¹H NMR (400 MHz, |
| | azaspiro[3.5]nonan-7- | DMSO-d₆) δ 12.85 (s, 1H), 8.72 (d, J = 6.8 Hz, 2H), |
| | yl)thiazole-4-carboxamide | 8.22 (d, J = 8.8 Hz, 1H), 8.16 (s, 1H), 3.87 (t, J = 4.4 |
| | | Hz, 1H), 3.69 (t, J = 6.0 Hz, 2H), 3.64 (t, J = 6.0 Hz, |
| | | 2H), 2.02 (d, J = 12.4 Hz, 2H), 1.73 (d, J = 9.2 Hz, |
| | | 2H), 1.61-1.45 (m, 4H). |
| 38 | 5-(4,6-dichloro-5- | LCMS (ES) m/z calculated for C19H13Cl2F3N4O3S, |
| | hydroxypicolinamido)-3-methyl-N-(2- | 504; found, 505.1 (M + H); ¹H NMR (400 MHz, |
| | (trifluoromethyl)benzyl)isothiazole- | DMSO-d₆) δ 12.38 (s, 1H), 8.38 (t, J = 6.4 Hz, 1H), |
| | 4-carboxamide | 8.09 (s, 1H), 7.78-7.76 (m, 1H), 7.71-7.62 (m, 2H), |
| | | 7.52-7.49 (m, 1H), 4.77 (d, J = 5.6 Hz, 2H), —CH3 |
| | | protons merged with residual solvent peak. |
| 39 | 5-(4,6-dichloro-5- | LCMS (ES) m/z calculated for C15H16Cl2N4O3S, |
| | hydroxypicolinamido)-N- | 402.03; found 403.1 [M + H]; ¹H NMR (400 MHz, |
| | isopentylthiazole-4-carboxamide | DMSO-d₆) 12.87 (s, 1H), 8.71 (s, 1H), 8.56 (t, d = 6 |
| | | Hz, 1H), 8.15 (s, 1H), 3.37-3.32 (m, 2H), 1.64-1.60 |
| | | (m, 1H), 1.49-1.43 (m, 2H), 0.93-0.91(m, 6H). |
| 40 | N-((3s,5s,7s)-adamantan-1-yl)-5- | LCMS (ES): m/z calculated for C20H20Cl2N4O3S, |
| | (4,6-dichloro-5- | 466; found 467 [M + H]⁺. 1HNMR (400 MHz, DMSO- |
| | hydroxypicolinamido)thiazole-4- | d6): δ 12.64 (s, 1 H), 8.68 (s, 1H), 8.14 (s, 1 H), 7.40 |
| | carboxamide | (s, 1H), 2.15-2.11 (m, 9H), 1.70-.68 (m, 6H). |
| 41 | N-(benzo[d][1,3]dioxol-5- | LCMS (ES): m/z calculated for C18H12Cl2N4O5S, |
| | ylmethyl)-5-(4,6-dichloro-5- | 465.9; found 467 [M + H]⁺; ¹H NMR (400 MHz, |
| | hydroxypicolinamido)thiazole-4- | DMSO-d6): δ 12.81 (s, 1 H), 9.06 (t, J = 6.4 Hz, 1 H), |
| | carboxamide | 8.73 (s, 1H), 8.15 (s, 1 H), 6.94-6.92 (m, 1H), |
| | | 6.87-6.82 (m, 2H), 5.97 (s, 2H), 4.43 (d, J = 6.4 Hz, 2H). |
| 42 | 5-(4,6-dichloro-5- | LCMS (ES) m/z calculated for C18H14Cl2N4O5S2, |
| | hydroxypicolinamido)-N-(2- | 499.9; found, 501 (M + H). ¹H NMR (400 MHz, |
| | (methylsulfonyl)benzyl)thiazole- | DMSO-d6): δ 12.78 (s, 1H), 9.27 (t, J = 6.0 Hz, 1H), |
| | 4-carboxamide | 8.75 (s, 1H), 8.11 (s, 2H), 7.94 (d, J = 7.2 Hz, 1H), |
| | | 7.73-7.71 (m, 1 H), 7.63 (d, J = 7.2 Hz, 1H), 7.55 (t, |
| | | J = 8.0 Hz, 1H), 4.92 (d, J = 6.0 Hz, 2H), 3.46 (s, 3H). |
| 43 | N-(chroman-4-yl)-5-(4,6- | LCMS (ES): m/z calculated for C19H14Cl2N4O4S, |
| | dichloro-5- | 464; found, 463.1 (M − H); ¹H NMR (400 MHz, |
| | hydroxypicolinamido)thiazole-4- | DMSO-d6): δ 12.87 (s, 1H), 8.80 (d, J = 8.8 Hz, 1H), |
| | carboxamide | 8.70 (s, 1H), 8.14 (s, 1H), 7.18-7.13 (m, 2 H), 6.86 (t, |
| | | J = 7.2 Hz, 1H), 6.79 (d, J = 8.0 Hz, 1H), 5.42-5.40 (m, |
| | | 1H), 4.35-4.29 (m, 2H), 2.45-2.14 (m, 2H). |
| 44 | 4,6-dichloro-5-hydroxy-N-(4-(4- | LCMS (ES): m/z C16H13Cl2F3N4O3S calculated for |
| | (trifluoromethyl)piperidine-1- | 468.0; found, 469.0 (M + H); 1H NMR (400 MHz, |
| | carbonyl)thiazol-5- | DMSO-d6): δ 8.73 (s, 1H), 8.15 (s, 1H), 5.38-5.36 (m, |
| | yl)picolinamide | 1H), 4.72-4.68 (m, 1H), 2.89-2.80 (m, 2H), 2.79-2.70 |
| | | (m, 1H), 2.09-1.9 (m, 2H), 1.60-1.40 (m, 2H). |
| 46 | 5-(3,5-dichloro-4- | LCMS (ES): m/z C17H19Cl2N3O3S calculated for |
| | hydroxybenzamido)-N-(3,3- | 415.0; found, 414 (M − H); 1H NMR (400 MHz, |
| | dimethylbutyl)thiazole-4- | DMSO-d6): δ 12.56 (s, 1H), 11.32 (s, 1H), 8.69 (s, |
| | carboxamide | 1H), 8.64-8.61 (m, 1H), 7.86 (s, 2H), 3.38-3.30 (m, |
| | | 2H), 1.52-1.48 (m, 2H), 0.94 (s, 9H). |
| 48 | 4,6-dichloro-5-hydroxy-N-(1- | LCMS (ES): m/z calculated for C21H18Cl2F3N5O3 |
| | isopropyl-4-((2- | is 515.07; found, 516.1(M + H); 1H NMR (400 MHz, |
| | (trifluoromethyl)benzyl)carbamoyl)- | DMSO d6): δ 11.42 (s, 1H), 8.68 (t, J = 6 Hz, 1H), |
| | 1H-pyrazol-3-yl)picolinamide | 8.40 (s, 1H), 8.04 (s, 1H), 7.75 (d, J = 7.6 Hz, 1H), |
| | | 7.69-7.66 (m, 1H), 7.56 (d, J = 7.6 Hz, 1H), 7.52-7.48 |
| | | (m, 1H), 4.64 (d, J = 5.2 Hz, 2H), 4.51-4.44 (m, 1H), |
| | | 1.46 (d, J = 7.6 Hz, 6H). |
| 49 | 3-(3,5-dichloro-4- | LCMS (ES): m/z calculated for C21H14Cl2F6N4O3 |
| | hydroxybenzamido)-1-(2,2,2- | is 554.03; found, 553.1(M − H); 1H NMR (400 MHz, |
| | trifluoroethyl)-N-(2- | DMSO d6): δ 10.78 (s, 1H), 8.66 (t, J = 5.6 Hz, 1H), |
| | (trifluoromethyl)benzyl)-1H- | 8.39 (s, 1H), 8.09 (s, 2H), 7.96-7.92 (m, 1H), 7.71 (d, |
| | pyrazole-4-carboxamide | J = 7.6 Hz, 1H), 7.61-7.55 (m, 2H), 7.48-7.45 (m, |
| | | 1H), 5.26-5.19 (m, 2 H), 4.58 (d, J = 6.0 Hz, 2H). |
| 50 | 4,6-dichloro-5-hydroxy-N-(1- | LCMS (ES): m/z calculated for C20H13Cl2F6N5O3 |
| | (2,2,2-trifluoroethyl)-4-((2- | is 555.03; found, 556.1(M + H); 1H NMR (400 MHz, |
| | (trifluoromethyl)benzyl)carbamoyl)- | DMSO d6): δ 11.47 (s, 1H), 8.93 (t, J = 5.6 Hz, 1H), |
| | 1H-pyrazol-3-yl)picolinamide | 8.46 (s, 1H), 8.09 (s, 1H), 7.98 (s, 1H), 7.75 (d, J = 7.6 |
| | | Hz, 1H), 7.67 (t, J = 7.6 Hz, 1H), 7.56 (d, J = 7.6 Hz, 1H), |
| | | 7.50 (t, J = 7.6 Hz, 1H), 5.26-5.19 (m, 2 H), 4.65 (d, J = |
| | | 6.0 Hz, 2H). |
| 51 | 5-(4,6-dichloro-5- | LCMS (ES): m/z calculated for C23H14Cl2F3N5O3S |
| | hydroxypicolinamido)-2- | 567.01 found, 568.1 (M + H); 1H NMR (400 MHz, |
| | (pyridin-3-yl)-N-(2- | DMSO d6): δ 12.78 (s, 1H), 9.35 (t, J = 6.0 Hz, 1H), |
| | (trifluoromethyl)benzyl)thiazole- | 9.31-9.30 (m, 1H), 8.70-8.68 (m, 1H), 8.46-8.43 (m, |
| | 4-carboxamide | 1H), 8.18 (s, 1H) 7.77 (d, J = 7.6 Hz, 1H), 7.68-7.66 |
| | | (m, 1H), 7.61-7.57 (m, 2H), 7.50 (t, J = 7.6 Hz, 1H), |
| | | 4.79 (d, J = 6 Hz, 2H). |
| 52 | 4,6-dichloro-5-hydroxy-N-(2-((2- | LCMS (ES): m/z calculated for C20H13Cl2F3N4O4; |
| | (trifluoromethoxy)benzyl)carbamoyl)pyridin- | 500.03 found, 501.2 (M + H); ¹H NMR (400 MHz, |
| | 3-yl)picolinamide | DMSO-d₆): 13.34 (s, 1H), 9.65 (t, J = 6.4 Hz, 1H), |
| | | 9.20 (dd, J₁ = 1.2 Hz, J₂ = 7.2 Hz, 1H), 8.42 (dd, J₁ = |
| | | 1.2 Hz, J₂ = 3.2 Hz, 1H), 8.10 (s, 1H), 7.72-7.68 (m, |
| | | 1H), 7.49-7.44 (m, 1H), 7.40-7.35 (m, 3H), 4.63 (d, |
| | | J = 6.4 Hz, 2H). |
| 53 | 3-(3,5-dichloro-4- | LCMS (ES): m/z calculated for C18H22Cl2N4O3, |
| | hydroxybenzamido)-N-(3,3- | 412.11; found, 413.3 (M + H); ¹H NMR (400 MHz, |
| | dimethylbutyl)-1-methyl-1H- | DMSO-d₆): δ 10.96 (s, 1H), 10.63 (s, 1H), 8.10 (s, |
| | pyrazole-4-carboxamide | 1H), 7.92-7.91 (m, 1H), 7.87-7.82 (m, 2H), 3.87 (s, |
| | | 3H), 3.20-3.10 (m, 2H), 1.60-1.40 (m, 2H), 0.90 (s, |
| | | 9H) |
| 54 | 4,6-dichloro-N-(1-cyclopropyl-4-((2- | LCMS (ES): m/z calculated for C21H16Cl2F3N5O3, |
| | (trifluoromethyl)benzyl)carbamoyl)- | 513.06; found 514.1 [M + H]; ¹H NMR (400 MHz, |
| | 1H-pyrazol-3-yl)-5-hydroxypicolinamide | DMSO-d₆): 10.42 (s, 1H), 8.54 (t, J = 5.6 Hz, 1H), |
| | | 8.04 (s, 1H), 7.95 (s, 1H), 7.70 (d, J = 7.6 Hz, 1H), |
| | | 7.62-7.58 (m, 1H), 7.51-7.43 (m, 2H), 4.57 (d, J = |
| | | 5.6 Hz, 2H), 3.58-3.53 (m, 1H), 1.01-0.98 (m, 2H), |
| | | 0.93-088 (m, 2H). |
| 55 | 3-(3,5-dichloro-4-hydroxybenzamido)-1- | LCMS (ES) m/z calculated for C20H13Cl2F5N4O3, |
| | (difluoromethyl)-N-(2- | 522.03; found, 523.1 (M + H); ¹H NMR (400 MHz, |
| | (trifluoromethyl)benzyl)-1H- | DMSO-d₆) δ 11.07 (s, 1H), 10.77 (s, 1H), 8.90-8.87 |
| | pyrazole-4-carboxamide | (m, 1H), 8.74 (s, 1H), 8.02, 7.87 (2s, 1H, J = 60 Hz), |
| | | 7.91 (s, 2H), 7.73-7.71 (m, 1H), 7.65-7.60 (m, 2H), |
| | | 7.50-7.46 (m, 1H) 4.60 (s, 2H). |
| 56 | 4,6-dichloro-N-(1-cyclopropyl- | LCMS (ES) m/z calculated for C25H17Cl2F3N4O4, |
| | 2,4-dioxo-3-(2- | 564.06; found, 565.2 (M + H); ¹H NMR (400 MHz, |
| | (trifluoromethyl)benzyl)-1,2,3,4- | DMSO-d₆) δ 13.4 (s, 1H), 8.67 (d, J = 8 Hz, 1 H), |
| | tetrahydroquinazolin-5-yl)-5- | 7.99 (bs, 1H), 7.80-7.74 (m, 2H), 7.59-7.55 (m, 1H), |
| | hydroxypicolinamide | 7.50-7.46 (m, 1H), 7.27 (d, J = 8.4 Hz, 1H), 7.12 (d, |
| | | J = 8.4 Hz, 1H), 6.00-5.91 (m, 1H), 5.37-5.32 (m, 2 H), |
| | | 5.22-5.19 (m, 2 H), 4.8 (s, 2H). |
| 57 | 3-(3,5-dichloro-4- | LCMS (ES) m/z calculated for C27H18Cl2F3N3O4 |
| | hydroxybenzamido)-6-phenyl-N-(2- | calculated for 575.06; found, 576.1 (M + H). |
| | (trifluoromethoxy)benzyl)picolin amide | ¹H NMR (400 MHz, DMSO-d₆) δ 13.07 (s, 1H), 11.20 |
| | | (s, 1H), 9.88 (t, J = 7.5 Hz, 1H), 9.16 (d, J = 9.2 Hz, |
| | | 1H), 8.32 (d, J = 8 Hz, 3H), 7.89 (s, 2H), 7.54-7.37 |
| | | (m, 7H), 4.73 (d, J = 6.4 Hz, 2H). |
| 58 | 4,6-dichloro-5-hydroxy-N-(6-phenyl-2-((2- | LCMS (ES) m/z calculated for C26H17Cl2F3N4O4, |
| | (trifluoromethoxy)benzyl)carbamoyl)pyridin- | 576.06; found 577.6 (M + H); ¹H NMR (400 MHz, |
| | 3-yl)picolinamide | DMSO-d₆) δ 13.38 (s, 1H), 9.71 (t, J = 12.8 Hz, 1H), |
| | | 9.28 (d, J = 8.8 Hz, 1H), 8.32-8.29 (m, 3H), 8.10 s, |
| | | 1H), 7.54-7.37 (m, 7 H), 4.72 (d, J = 6.4 Hz, 2H). |

### Example 59: Synthesis of 2-cyclopropyl-5-(4,6-dichloro-5-hydroxypicolinamido)-N-(2-(trifluoromethyl)benzyl)thiazole-4-carboxamide

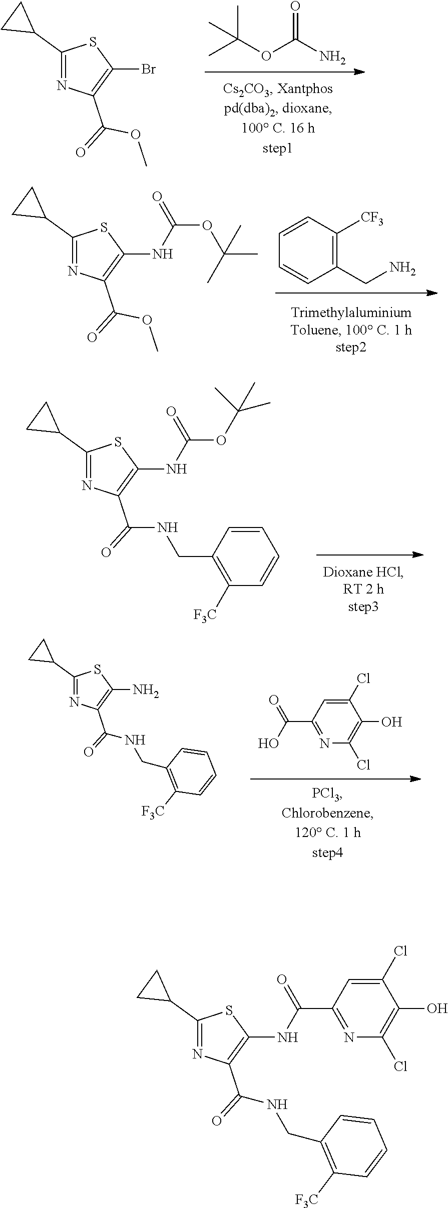

### Step 1: Synthesis of methyl 5-((tert-butoxycarbonyl)amino)-2-cyclopropylthiazole-4-carboxylate

To a stirred solution of methyl 5-bromo-2-cyclopropylthiazole-4-carboxylate (0.5 g, 1.91 mmol) and tert-butyl carbamate (0.223 g, 1.91 mmol) in 1,4-dioxane (10 mL) was added cesium carbonate (1.24 g, 3.82 mmol) and degassed with nitrogen for 10 min, followed by Pd(dba)₂ (0.054 g, 0.095 mmol), Xanthphos (0.11 g, 0.19 mmol) were added and stirred at 100° C. for 16 h. Progress of the reaction was monitored by TLC and LCMS. After completion of the reaction, the reaction mixture was filtered through a pad of celite, filtrate was diluted with ethyl acetate (50 mL), washed with water (25×2 mL), brine solution and dried over anhydrous sodium sulphate, filtered, and concentrated under vacuo to give crude. The crude product was purified by flash column chromatography to afford methyl 5-((tert-butoxycarbonyl)amino)-2-cyclopropylthiazole-4-carboxylate (0.3 g, 52% yield). LCMS (ES) m/z calcd. for; C13H18N2O4S 298.10; found, 243.1. (M-^{t}bu).

### Step 2: Synthesis of tert-butyl (2-cyclopropyl-4-((2-(trifluoromethyl)benzyl)carbamoyl) thiazol-5-yl)carbamate

To a solution of methyl 5-{[(tert-butoxy)carbonyl]amino}-2-cyclopropyl-1,3-thiazole-4-carboxylate (0.2 g, 0.670 mmol) and 1-[2-(trifluoromethyl)phenyl]methanamine (0.141 g, 0.804 mmol) in toluene (4 mL) was added 2 M trimethylaluminium in toluene (2 mL, 2 mmol) at 0° C. The reaction mixture was heated at 100° C. for 1 h. The reaction mixture was cooled to ambient temperature and quenched with ice-cold water (15 mL) and extracted with Ethyl acetate (25×2 mL). The combined organic phase was washed with brine solution (15 mL) and dried over anhydrous sodium sulphate, filtered, and evaporated in vacuo to obtain crude brown solid which was triturated with diethyl ether (10 mL) to afford tert-butyl (2-cyclopropyl-4-((2-(trifluoromethyl)benzyl)carbamoyl)thiazol-5-yl)carbamate (0.120 g, 40% yield). LCMS (ES) m/z calcd. for C20H22F3N3O3S 441.13; found, 342.1 (M-Boc).

### Step 3: Synthesis of 5-amino-2-cyclopropyl-N-(2-(trifluoromethyl)benzyl)thiazole-4-carboxamide

To a stirred solution of tert-butyl N-[2-cyclopropyl-4-({[2-(trifluoromethyl)phenyl]methyl}carbamoyl)-1,3-thiazol-5-yl]carbamate (0.120 g, 0.272 mmol) in dichloromethane (2 mL) was cooled to 0° C. and then added 4 M HCl in 1,4 dioxane (2 mL). The resulting reaction mixture was stirred at ambient temperature for 2 h. The reaction mixture was concentrated under reduced pressure and the residue obtained was triturated with diethyl ether (10 mL) to afford 5-amino-2-cyclopropyl-N-{[2-(trifluoromethyl)phenyl]methyl}-1,3-thiazole-4-carboxamide (0.090 g, 97% yield). LCMS (ES) m/z calcd. for; C15H14F3N3OS 341.08; found, 342.1 (M+H).

### Step 4: Synthesis of 2-cyclopropyl-5-(4,6-dichloro-5-hydroxypicolinamido)-N-(2-(trifluoromethyl)benzyl)thiazole-4-carboxamide

To a solution of 5-amino-2-cyclopropyl-N-{[2-(trifluoromethyl)phenyl]methyl}-1,3-thiazole-4-carboxamide (0.080 g, 0.234 mmol) and 4,6-dichloro-5-hydroxypyridine-2-carboxylic acid (0.048 g, 0.234 mmol) in chlorobenzene (1 mL) was added phosphorous trichloride (0.032 g, 0.234 mmol). The resulting reaction mixture was heated at 120° C. for 1 h. The progress of reaction was monitored by LCMS. The reaction mixture was cooled to ambient temperature and diluted with ethyl acetate (25 mL) and washed with water (10 mL×2), brine solution (10 mL). The organic phase was dried over Na₂SO₄, concentrated under reduced pressure to get crude residue which was purified by flash column chromatography to afford 4,6-dichloro-N-[2-cyclopropyl-4-({[2-(trifluoromethyl)phenyl]methyl}carbamoyl)-1,3-thiazol-5-yl]-5-hydroxypyridine-2-carboxamide (0.032 g, 25% yield) as off white solid. LCMS (ES): m/z C21H15C12F3N4O3S calcd. for 530.0; found, 531.1 (M+H); ¹H NMR (400 MHz, DMSO-d₆): δ 12.60 (s, 1H), 8.95 (t, J=6 Hz, 1H), 8.10 (s, 1H), 7.75 (d, J=7.6 1H), 7.66 (t, J=7.2 Hz, 1H) 7.50-7.46 (m, 2H), 4.71 (d, J=6 Hz, 2H), 2.38-2.33 (m, 1H), 1.20-1.14 (m, 4H).

### Example 60: Synthesis of 5-(4,6-dichloro-5-hydroxypicolinamido)-2-(2-fluorophenyl)-N-(2-(trifluoromethyl) benzyl) thiazole-4-carboxamide

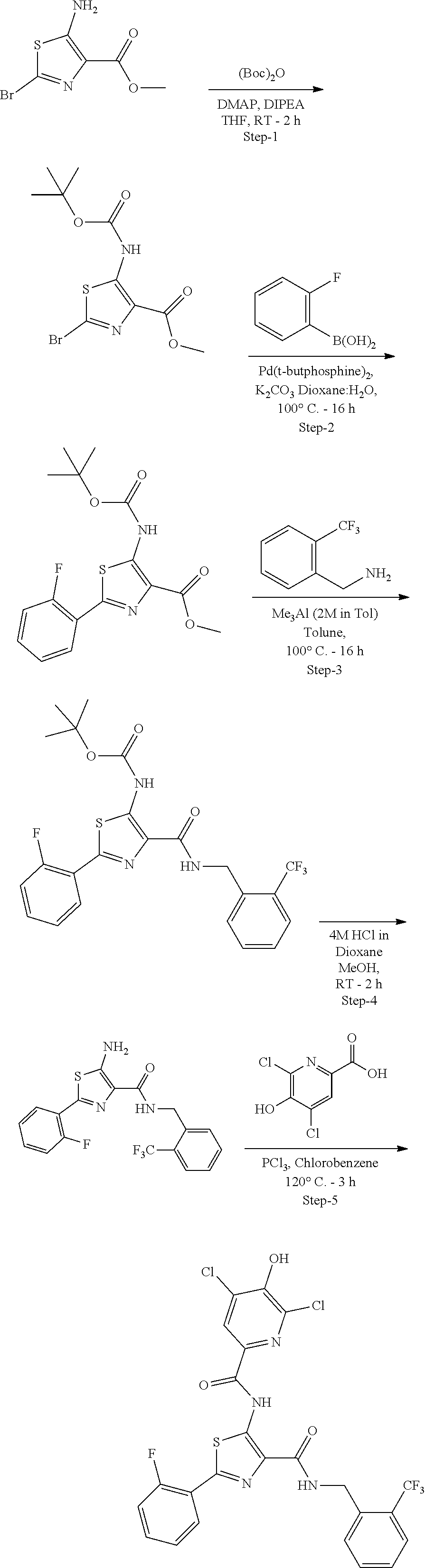

### Step 1: Preparation of methyl 2-bromo-5-((tert-butoxycarbonyl)amino)thiazole-4-carboxylate

To a stirred solution of methyl 5-amino-2-bromothiazole-4-carboxylate (1 g, 4.22 mmol) in tetrahydrofuran (20 mL, 246 mmol) was added di-tert-butyldicarbonate (1.45 mL, 6.33 mmol), DIPEA (2.2 mL, 12.7 mmol) and DMAP (0.520 g, 4.22 mmol) at ambient temperature and then stirred at same temperature for another 2 h. The reaction mixture was poured into water (40 mL) and extracted with ethyl acetate (50 mL×3). The combined organic phase was washed with water, brine and dried over anhydrous sodium sulfate, filtered, and concentrated in vacuo to get crude. The crude material was purified by flash column chromatography to afford methyl 2-bromo-5-((tert-butoxycarbonyl)amino)thiazole-4-carboxylate (0.9 g, 63%) as white solid. LCMS (ES): m/z calcd. for C10H13BrN2O4S, 335.98; found, 337.0.

### Step 2: Preparation of methyl 5-((tert-butoxycarbonyl) amino)-2-(2-fluorophenyl) thiazole-4-carboxylate

To a stirred solution of methyl 2-bromo-5-((tert-butoxycarbonyl)amino)thiazole-4-carboxylate (0.460 g, 1.36 mmol) and (2-fluorophenyl)boronic acid (0.382 g, 2.73 mmol) in 1,4-dioxane:water 8:2 (6 mL) was added and potassium carbonate (0.566 g, 4.09 mmol) and degassed with nitrogen for 10 min, followed by bis(tri-tert-butylphosphane) palladium (0.0697 g, 0.136 mmol) was added and stirred at 100° C. for 16 h. Progress of the reaction was monitored by TLC/LCMS. After completion of the reaction, the reaction mixture was filtered through a pad of celite, filtrate was diluted with ethyl acetate (50 mL), washed with water (25×2 mL), brine solution and dried over anhydrous sodium sulphate, filtered, and concentrated under vacuo to give crude. The crude product was purified by flash column chromatography to afford methyl 5-((tert-butoxycarbonyl)amino)-2-(2-fluorophenyl)thiazole-4-carboxylate (0.350 g, 73%) as brown solid. LCMS (ES): m/z calcd. for C16H17FN2O4S, 352.09; found, 353.1.

### Step 3: Preparation of tert-butyl (2-(2-fluorophenyl)-4-((2-(trifluoromethyl) benzyl) carbamoyl) thiazol-5-yl) carbamate

To a stirred solution of methyl 5-((tert-butoxycarbonyl)amino)-2-(2-fluorophenyl)thiazole-4-carboxylate (0.3 g, 0.851 mmol) in toluene (5 mL) was added 1-[2-(trifluoromethyl)phenyl]methanamine (0.119 mL, 0.851 mmol) and 2 M trimethylaluminium in toluene (0.245 mL, 2.55 mmol) at 0° C. The resulting reaction mixture was stirred at 100° C. for 16 hours. The reaction mixture was cooled to ambient temperature and quenched with ice-cold water (25 mL) and extracted with Ethyl acetate (25×2 mL). The combined organic phase was washed with brine solution (15 mL) and dried over anhydrous sodium sulphate, filtered, and evaporated in vacuo to obtain crude. The crude material was purified by flash column chromatography to afford tert-butyl (2-(2-fluorophenyl)-4-((2-(trifluoromethyl)benzyl)carbamoyl)thiazol-5-yl)carbamate (0.3 g, 71%) as off white solid. LCMS (ES): m/z calcd. C23H21F4N3O3S, 495.1; found, 496.1.

### Step 4: Preparation of 5-amino-2-(2-fluorophenyl)-N-(2-(trifluoromethyl) benzyl) thiazole-4-carboxamide

To a suspension of tert-butyl (2-(2-fluorophenyl)-4-((2-(trifluoromethyl)benzyl) carbamoyl)thiazol-5-yl)carbamate (0.250 g, 0.505 mmol) in methanol (1 mL) was added 4M HCl in dioxane (5 mL, 144 mmol) at 0° C. and then allowed to stirred at RT for 2 h. The reaction mixture was poured into water (20 mL) and extracted with ethyl acetate (20 mL×3). The organic phase was washed with water (10 ml), brine (5 ml) and dried over anhydrous sodium sulfate, filtered, and concentrated in vacuo to give 5-amino-2-(2-fluorophenyl)-N-(2-(trifluoromethyl)benzyl)thiazole-4-carboxamide (0.170 mg, 85%) as a white solid. LCMS (ES): m/z calcd. for C18H13F4N3OS, 395.07; found, 396.1.

### Step 5: Preparation of 5-(4,6-dichloro-5-hydroxypicolinamido)-2-(2-fluorophenyl)-N-(2-(trifluoromethyl) benzyl) thiazole-4-carboxamide

To a suspension of 5-amino-2-(2-fluorophenyl)-N-(2-(trifluoromethyl)benzyl)thiazole-4-carboxamide (0.175 g, 0.443 mmol) in chlorobenzene (5 mL) was added 4,6-dichloro-5-hydroxypyridine-2-carboxylic acid (0.101 g, 0.487 mmol) and phosphorous trichloride (0.0387 mL, 0.443 mmol). The resulting reaction mixture was heated at 120° C. for 3 h. The progress of reaction was monitored by LCMS. The reaction mixture was cooled to ambient temperature and diluted with ethyl acetate (25 mL) and washed with water (10 mL×2), brine solution (10 mL). The organic phase was dried over Na₂SO₄, concentrated under reduced pressure to get crude residue which was purified by flash column chromatography Na₂SO₄ to afford 5-(4,6-dichloro-5-hydroxypicolinamido)-2-(2-fluorophenyl)-N-(2-(trifluoromethyl)benzyl)thiazole-4-carboxamide (0.015 g, 6%) as a white solid. LCMS (ES): m/z C24H14C12F4N4O3S calcd. for 584.0; found, 585.1 (M−H); ¹H NMR (400 MHz, DMSO-d₆): δ 12.74 (s, 1H), 9.37 (t, J=6 Hz, 1H), 8.51-8.47 (m, 1H), 8.15 (s, 1H), 7.78 (d, J=7.6 Hz, 1H), 7.68 (t, J=7.6 Hz, 1H), 7.58-7.39 (m, 5H), 4.81 (d, J=6 Hz, 2H).

The following compounds has been synthesized using above-described procedures.

| Ex. | Spectral data |
|---|---|
| 61 | LCMS (ES) m/z calcd. for C24H15Cl2F3N4O3S, 566.02; found 567.1 (M + H) ¹H NMR |
| | (400 MHz, DMSO-d₆): δ 12.34 (s, 1H), 11.39 (br s, 1H), 9.46 (t, J = 6.0 Hz, 1H), 9.34-9.23 |
| | (m, 1H), 8.69-8.67 (m, 1H), 9.43-9.40 (m, 1H), 7.86 (s, 2H), 7.78 (t, J = 12 Hz, 1H), |
| | 7.70-7.66 (m, 1H), 7.59-7.55 (m, 2H), 7.50 (t, J = 7.6 Hz, 1H), 4.80 (s, 2H). |
| 62 | LCMS (ES) m/z calcd, for 25H16Cl2F3N3O3S, 565.02; found, 566.15 (M + H); ¹H NMR |
| | (400 MHz, DMSO-d₆) δ12.29 (s, 1H), 11.38 (bs, 1H), 9.37 (t, J = 6 Hz, 1H), 8.13-8.01 (m, |
| | 2H), 7.78-7.76 (m, 3H), 7.73-7.68 (m, 1H), 7.66-7.52 (m, 5H), 4.79 (d, J = 6 Hz, 2H). |
| 63 | LCMS (ES) m/z calcd, for C25H15Cl2F4N3O3S, 583.01; found, 582.2 (M − H); ¹H NMR |
| | (400 MHz, DMSO-d₆) δ12.3 (s, 1H), 11.37 (bs, 1H), 9.48 (t, J = 6 Hz, 1H), 8.51-8.46 (m, |
| | 1H), 7.85 (s, 2H), 7.80-7.76 (m, 1H), 7.70-7.64 (m, 1H), 7.59-7.39 (m, 5H), 4.80 (d, |
| | J = 6 Hz, 2H). |
| 64 | LCMS (ES) m/z calcd. for C25H15ClF5N3O3S 567.04; found 568.0 (M + H); ¹H NMR (400 |
| | MHz, DMSO-d₆): δ 12.33 (s, 1H), 11.65 (s, 1H), 9.49 (t, J = 6 Hz, 1H), 8.51-8.47 (m, 1H), |
| | 7.78-7.74 (m, 2H), 7.71-7.66 (m, 2H), 7.60-7.40 (m, 5H), 4.81 (d, J = 6.0 Hz, 2H). |
| 65 | LCMS (ES) m/z calcd, for C25H15Cl3F3N3O3S, 598.99; found, 600.0 (M + H); ¹H NMR |
| | (400 MHz, DMSO-d₆) δ 12.25 (s, 1H), 9.46 (bs, 1H), 8.49 (t, J = 4.8 Hz, 1H), 7.81-7.76 |
| | (m, 3H), 7.68-7.66 (m, 2H), 7.58-7.48 (m, 5H), 4.79 (d, J = 5.6 Hz, 2H). |
| 66 | LCMS (ES): m/z C22H15Cl2F3N6O3S calcd. for 570.03; found, 569.2 (M − H); ¹H NMR |
| | (400 MHz, DMSO-d₆): δ 12.69 (s, 1H), 9.03 (t, J = 6.0 Hz, 1H), 8.36 (s, 1H), 8.13 |
| | (s, 1H), 7.98 (s, 1H), 7.76 (d, J = 8.0 Hz, 1H), 7.69-7.65 (m, 1H), 7.55-7.47 (m, 2H), |
| | 4.77 (d, J = 6.0 Hz, 2H) 3.91 (s, 3H). |
| 67 | LCMS (ES) m/z calcd, for C24H14Cl3F3N4O3S, 599.98; found, 601.0 (M + H). ¹H NMR |
| | (400 MHz, DMSO-d₆) δ 12.58 (s, 1H), 9.31 (br s, 1H), 8.51-8.49 (m, 1H), 8.01 (br s, 1H), |
| | 7.77 (d, J = 7.6 Hz, 1H), 7.68-7.66 (m, 2H), 7.58-7.50 (m, 4H), 4.79 (d, J = 6 Hz, 2H). |
| 68 | LCMS (ES) m/z calcd. for C22H13Cl2F3N6O3S, 568.01; found, 569.1(M + H), ¹H NMR |
| | (400 MHz, DMSO-d₆) δ 12.76 (s, 1H), 9.46 (s, 2H), 9.42-9.41 (m, 1H), 9.28 (s, 1H), 8.16 |
| | (s, 1H), 7.79-7.77 (m, 1H), 7.68-7.66 (m, 1H), 7.58-7.56 (m, 1H), 7.52-7.50 (m, 1H), |
| | 4.79 (d, J = 6 Hz, 2H). |
| 69 | LCMS (ES) m/z calcd. for C22H15Cl2F3N6O3S 570.03; found 571.1 (M + H); ¹H NMR |
| | (400 MHz, DMSO-d₆): δ 12.77 (s, 1H), 9.14 (t, J = 6 Hz, 1H), 8.12 (s, 1H), 7.78 (d, |
| | J = 7.6 Hz, 1H), 7.68 (t, J = 7.6 Hz, 1H), 7.55-7.48 (m, 3H), 6.92 (s, 1H), 4.80 |
| | (d, J = 6 Hz, 2H), 4.27 (s, 3H). |
| 70 | LCMS (ES) m/z calcd. for C23H14Cl2F3N5O3S, 567.01; found, 568.1 (M + H). HNMR |
| | (400 MHz, DMSO-d₆) δ 12.79 (s, 1H), 9.37 (t, J = 8 Hz, 1H), 8.75 (d, J = 5.2 Hz, 2H), |
| | 8.15 (s, 1H), 8.07 (d, J = 5.2 Hz, 2H), 7.78 (d, J = 8 Hz, 1H), 7.70-7.66 (m, 1H), |
| | 7.57 (d, J = 7.6 Hz, 1H), 7.52-7.48 (m, 1H), 4.8 (d, J = 5.6 Hz, 2H). |
| 71 | LCMS (ES) m/z calcd. for C21H13Cl2F3N6O3S, 556.01; found, 557.2 (M + H); ¹H NMR |
| | (400 MHz, DMSO-d₆) δ 13.30 (s, 1H), 12.72 (s, 1H), 9.08 (br s, 1H), 8.14 (s, 1H), |
| | 7.93 (bs, 1H), 7.76 (d, J = 7.6 Hz, 1H), 7.69-7.66 (m, 1H), 7.56-7.47 (m, 2H), |
| | 6.83 (d, J = 2 Hz, 1H), 4.77 (d, J = 6 Hz, 2H). |
| 72 | LCMS (ES) m/z calcd. for. C18H11Cl2F3N4O3S, 489.99; found 491.1 (M + H), ¹H NMR |
| | (400 MHz, DMSO-d₆): δ 12.59 (s, 1H), 9.15 (t, J = 4.2 Hz, 1H), 8.12-8.06 (m, 3H), |
| | 7.56-7.51 (m, 3H), 4.17 (m, 2H). |
| 73 | LCMS (ES): m/z calcd, for C25H17Cl2F3N4O5S2, 644.00; found, 643.1 (M − H), ¹H NMR |
| | (400 MHz, DMSO-d₆): δ 12.80 (s, 1H), 9.40 (t, J = 6.2 Hz, 1H), 8.33 (d, J = 8.4 Hz, |
| | 2H), 8.16 (s, 1H), 8.08-8.02 (d, J = 8.4 Hz, 2H), 7.79-7.77 (m, 1H), 7.70-7.66 (m, |
| | 1H), 7.59-7.57 (m, 1H), 7.52-7.48 (m, 1H), 4.79 (d, J = 6 Hz, 2H), 3.30 (s, 3H). |
| 74 | LCMS (ES) m/z calcd. for C23H14Cl2F3N5O4S 583.01; found 584.1 (M + H), ¹H NMR |
| | (400 MHz, DMSO-d₆): δ 12.62 (s, 1H), 12.53 (d, J = 6.0 Hz, 1H), 9.27 (t, J = 6.4 Hz, |
| | 1H), 8.68-8.65 (m, 1H), 8.09 (s, 1H), 7.76 (d, J = 7.6 Hz, 1H), 7.67-7.64 (m, 3H), |
| | 7.54-7.45 (m, 1H), 6.52 (t, J = 13.6 Hz, 1H), 4.76 (d, J = 5.6 Hz, 2H). |
| 75 | LCMS (ES) m/z calcd, for C24H14Cl2F4N4O3S, 584.01; found, 583.1 (M − H). ¹H NMR |
| | (400 MHz, DMSO-d₆ δ 12.20 (s, 1H), 9.46 (br s, 1H), 9.14 (s, 1H), 8.69 (s, 1H), 8.41 |
| | (d, J = 9.6 Hz, 1H), 7.78-7.77 (m, 3H), 7.70-7.66 (m, 1H), 7.68-7.66 (m, 1H), 7.59-7.57 |
| | (m, 1H), 7.52.7.48 (m, 1H), 4.79 (d, J = 5.6 Hz, 2H). |
| 76 | LCMS (ES) m/z calcd, for C23H13Cl2F4N5O3S, 585.01; found, 586.0 (M + H). ¹H NMR |
| | (400 MHz, DMSO-d₆ δ 12.65 (s, 1H), 9.40-9.37 (m, 1H), 9.15 (s, 1H), 8.69 (s, 1H), |
| | 8.44-8.41 (m, 1H), 8.14-8.07 (m, 1H), 7.78-7.77 (m, 1H), 7.70-7.67 (m, 1H), 7.58-7.56 |
| | (m, 1H), 7.52-7.48 (m, 1H), 4.79 (d, J = 2 Hz, 2H). |
| 77 | LCMS (ES) m/z calcd. for C24H15Cl2F3N4O4S 582.01; found 581.1 (M − H); ¹H NMR |
| | (400 MHz, DMSO-d₆): δ 12.55 (d, J = 5.2 Hz, 1H), 12.19 (s, 1H), 9.35 (t, J = 6 Hz, 1H), |
| | 8.69-8.66 (m, 1H), 7.80-7.76 (m, 3H), 7.70-7.66 (m, 2H), 7.58-7.56 (m, 1H), 7.50 (t, |
| | J = 7.2 Hz, 1H), 6.54 (t, J = 6.8 Hz, 1H), 4.79 (d, J = 5.6 Hz, 2H). |

### Example 78: Synthesis of 5-(4,6-dichloro-5-hydroxypicolinamido)-2-(1,1-dioxidothiomorpholino)-N-(2-(trifluoromethyl)benzyl)thiazole-4-carboxamide

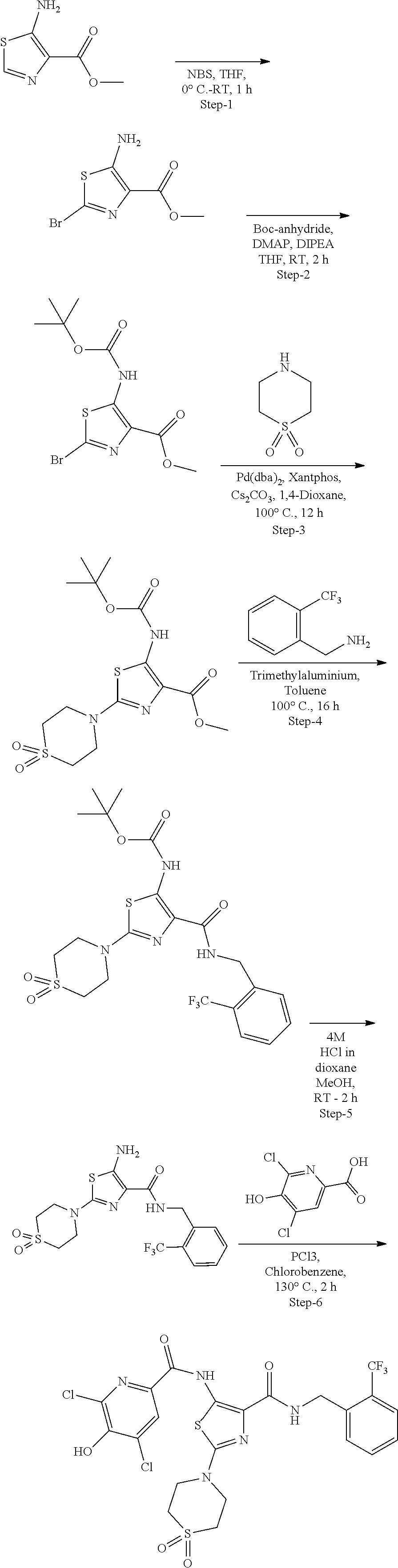

### Step 1: Synthesis of methyl 5-amino-2-bromothiazole-4-carboxylate

To a stirred solution of methyl 5-aminothiazole-4-carboxylate (0.7 g, 4.43 mmol) in tetrahydrofuran (7 mL, 86 mmol) was added N-bromosuccinimide (0.86 g, 4.87 mmol) at 0° C. and then reaction mixture was stirred at ambient temperature for 1 h. The reaction mixture was quenched with ice water (30 ml) and extracted with ethyl acetate (3×20 mL). The combined organic layer was washed with water (20 mL), brine (20 mL), dried over sodium sulfate and evaporated under reduced pressure to result in crude which was purified by flash chromatography to afford methyl 5-amino-2-bromothiazole-4-carboxylate as a brown solid (0.960 g, 92%). LCMS (ES) m/z calcd. for C5H5BrN2O2S, 235.93; found, 237.0 (M+H).

### Step 2: Synthesis of methyl 2-bromo-5-((tert-butoxycarbonyl)amino)thiazole-4-carboxylate

To a stirred solution of methyl 5-amino-2-bromothiazole-4-carboxylate (0.96 g, 4.05 mmol) in tetrahydrofuran (15 mL, 184 mmol) was added di-tert-butyldicarbonate (1.4 mL, 6.07 mmol), N,N-diisopropylethylamine (2.12 mL, 12.1 mmol) and 4-(dimethylamino)pyridine (0.5 g, 4.05 mmol) at ambient temperature. The resulting reaction mixture was stirred at same temperature for another 2 h. The progress of the reaction monitored by TLC. The reaction mixture was quenched with ice water (50 mL) and extracted with ethyl acetate (3×20 mL). The combined organic layer was washed with water (10 mL), brine (10 mL), dried over sodium sulfate and evaporated under reduced pressure to result in crude which was purified by flash column chromatography to afford methyl 2-bromo-5-((tert-butoxycarbonyl)amino)thiazole-4-carboxylate as a white solid (1.1 g, 81%). LCMS (ES) m/z calcd. C10H13BrN2O4S, 335.98; found, 337.0 (M+H).

### Step 3: Synthesis of methyl 5-((tert-butoxycarbonyl)amino)-2-(1,1-dioxidothiomorpholino)thiazole-4-carboxylate

To a stirred solution of methyl 2-bromo-5-((tert-butoxycarbonyl)amino)thiazole-4-carboxylate (0.7 g, 2.08 mmol) and thiomorpholine 1,1-dioxide (421 mg, 3.11 mmol) in 1,4-dioxane (8 mL) was added cesium carbonate (2.03 g, 6.23 mmol) and degassed with nitrogen for 10 min, followed by Pd(dba)2 (0.095 g, 0.155 mmol), Xanthphos (0.18 g, 0.311 mmol) were added and stirred at 100° C. for 12 h. Progress of the reaction was monitored by TLC and LCMS. After completion of the reaction, the reaction mixture was filtered through a pad of celite, filtrate was diluted with ethyl acetate (50 mL), washed with water (25×2 mL), brine solution and dried over anhydrous sodium sulphate, filtered, and concentrated under vacuo to give crude. The crude product was purified by flash column chromatography to afford methyl 5-((tert-butoxycarbonyl)amino)-2-(1,1-dioxidothiomorpholino)thiazole-4-carboxylate as an off-white solid (0.70 g, 87%). LCMS (ES) m/z calcd. C14H21N3O6S2, 391.09; found, 392.0 (M+H).

### Step 4: Synthesis of tert-butyl (2-(1,1-dioxidothiomorpholino)-4-((2-(trifluoromethyl) benzyl)carbamoyl)thiazol-5-yl)carbamate

To a suspension of methyl 5-((tert-butoxycarbonyl)amino)-2-(1,1-dioxidothiomorpholino)thiazole-4-carboxylate (0.7 g, 1.79 mmol) and (2-(trifluoromethyl)phenyl)methanamine (345 mg, 1.97 mmol) in toluene (7 mL) was added 2 M trimethylaluminium in toluene (1.32 mL, 2.68 mmol) at 0° C. The reaction mixture was heated at 100° C. for 16 h. The reaction mixture was cooled to ambient temperature and quenched with ice-cold water (15 mL) and extracted with ethyl acetate (25×2 mL). The combined organic phase was washed with brine solution (15 mL) and dried over anhydrous sodium sulphate, filtered, and evaporated in vacuo to obtain crude. The crude material was purified by flash column chromatography to afford tert-butyl (2-(1,1-dioxidothiomorpholino)-4-((2-(trifluoromethyl)benzyl)carbamoyl)thiazol-5-yl)carbamate as a yellow solid (0.680 g, 71%). LCMS (ES) m/z calcd. C21H25F3N4O5S2, 534.1; found, 535.1 (M+H).

### Step 5: Synthesis of 5-amino-2-(1,1-dioxidothiomorpholino)-N-(2-(trifluoromethyl) benzyl)thiazole-4-carboxamide

To a stirred solution of tert-butyl (2-(1,1-dioxidothiomorpholino)-4-((2-(trifluoromethyl)benzyl)carbamoyl)thiazol-5-yl)carbamate (680 mg, 1.27 mmol) in methanol (2 mL) was added 4M HCl in dioxane (15 mL, 60 mmol) at 0° C. and allowed to stirred at RT for 2 h. Progress of the reaction was monitored by TLC and LCMS. After completion of the reaction, the reaction mixture was evaporated under reduced pressure and obtained residue was triturated with diethyl ether to afford 5-amino-2-(1,1-dioxidothiomorpholino)-N-(2-(trifluoromethyl)benzyl)thiazole-4-carboxamide as a brown solid (0.68 g, 58%). LCMS (ES) m/z calcd. C16H17F3N4O3S2, 434.07; found, 435.0 (M+H).

### Step 6: Synthesis of 5-(4,6-dichloro-5-hydroxypicolinamido)-2-(1,1-dioxidothiomorpholino)-N-(2-(trifluoromethyl)benzyl)thiazole-4-carboxamide

To a suspension of 5-amino-2-(1,1-dioxidothiomorpholino)-N-(2-(trifluoromethyl)benzyl)thiazole-4-carboxamide (630 mg, 1.45 mmol) and 4,6-dichloro-5-hydroxypicolinic acid (332 mg, 1.6 mmol) in chlorobenzene (5 mL) was added phosphorus trichloride (0.063 mL, 0.725 mmol) dropwise under nitrogen atmosphere. The resulting reaction mixture was heated to 130° C. for 2 h. Progress of the reaction was monitored by TLC and LCMS. The reaction mixture was poured into ice-cold water (20 ml), precipitated solid was filtered and purified by flash column chromatography to afford brown color solid, which was further purified by Prep-HPLC to afford 5-(4,6-dichloro-5-hydroxypicolinamido)-2-(1,1-dioxidothiomorpholino)-N-(2-(trifluoromethyl)benzyl)thiazole-4-carboxamide as a yellow solid (0.153 g, 17%). LCMS (ES) m/z calcd. C22H18C12F3N5O5S2, 623.01; found, 622.1 (M−1); ¹H NMR (400 MHz, DMSO-d₆) δ 12.45 (s, 1H), 8.93 (t, J=6.0 Hz, 1H), 8.09 (s, 1H), 7.80 (d, J=4.0 Hz, 1H), 7.67 (t, J=4.0 Hz, 1H), 7.51-7.42 (m, 2H), 4.7 (d, J=8.0 Hz, 2H), 4.02 (br s, 4H), 3.31 (br s, 4H).

### Example 79: Synthesis of 5-(3,5-dichloro-4-hydroxybenzamido)-2-(1,1-dioxidothiomorpholino)-N-(2-(trifluoromethyl)benzyl)thiazole-4-carboxamide

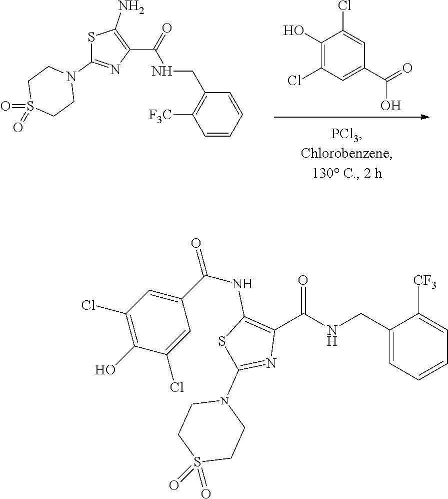 [SMILES: Nc1sc(N2CCS(=O)(=O)CC2)nc1C(=O)NCc1ccccc1C(F)(F)F ; O=C(O)c1cc(Cl)c(O)c(Cl)c1 ; O=C(Nc1sc(N2CCS(=O)(=O)CC2)nc1C(=O)NCc1ccccc1C(F)(F)F)c1cc(Cl)c(O)c(Cl)c1]

To a suspension of 5-amino-2-(1,1-dioxidothiomorpholino)-N-(2-(trifluoromethyl)benzyl) thiazole-4-carboxamide (0.5 g, 1.15 mmol) and 3,5-dichloro-4-hydroxybenzoic acid (262 mg, 1.27 mmol) in chlorobenzene (5 mL) was added phosphorus trichloride (0.050 mL, 0.575 mmol) dropwise under nitrogen atmosphere. The reaction mass was heated to 130° C. for 2 h. Progress of the reaction was monitored by TLC and LCMS. After completion of the reaction the reaction mixture was quenched with ice water (20 mL) and extracted with ethyl acetate (3×10 ml). The combined organic layer was washed with water (10 mL), brine (10 mL), dried over sodium sulfate and evaporated under reduced pressure to result in crude which was purified by flash chromatography. The column purified compound was further purified by Prep-HPLC to afford 5-(3,5-dichloro-4-hydroxybenzamido)-2-(1,1-dioxidothiomorpholino)-N-(2-(trifluoromethyl)benzyl)thiazole-4-carboxamide as a yellow solid (0.057 g, 8%). LCMS (ES) m/z calcd. C23H19C12F3N4O5S2, 622.0; found, 621.1 (M−H); ¹H NMR (400 MHz, DMSO-d₆) δ 12.0 (s, 1H), 11.28 (br s, 1H), 9.01 (t, J=8.0 Hz, 1H), 7.79 (s, 2H), 7.75 (d, J=8.0 Hz, 1H), 7.67 (t, J=8.0 Hz, 1H), 7.48 (t, J=8.0 Hz, 2H), 4.70 (d, J=4.0 Hz, 2H), 4.02 (br s, 4H), 3.28 (br s, 4H).

### Example 80: Synthesis of 5-(4,6-dichloro-5-hydroxypicolinamido)-2-morpholino-N-(2-(trifluoromethyl)benzyl)thiazole-4-carboxamide

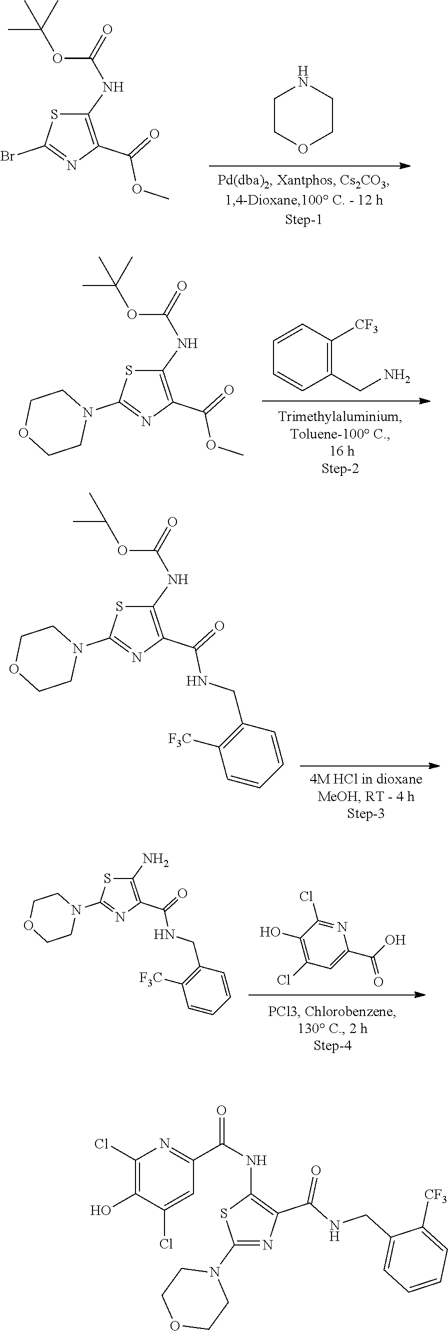

### Step 1: Synthesis of methyl 5-((tert-butoxycarbonyl)amino)-2-morpholinothiazole-4-carboxylate

To a stirred solution of methyl 2-bromo-5-((tert-butoxycarbonyl)amino)thiazole-4-carboxylate (1.65 g, 4.89 mmol) and Morpholine (0.633 mL, 7.34 mmol) in 1,4-dioxane (8 mL) was added cesium carbonate (4.78 g, 14.7 mmol) and degassed with nitrogen for 10 min, followed by Pd(dba)2 (0.14 g, 0.244 mmol), Xanthphos (0.27 g, 0.48 mmol) were added and stirred at 100° C. for 12 h. Progress of the reaction was monitored by TLC/LCMS. After completion of the reaction, the reaction mixture was filtered through a pad of celite, filtrate was diluted with ethyl acetate (50 mL), washed with water (25×2 mL), brine solution and dried over anhydrous sodium sulphate, filtered, and concentrated under vacuo to give crude. The crude product was purified by flash column chromatography to afford methyl 5-((tert-butoxycarbonyl)amino)-2-morpholinothiazole-4-carboxylate as a yellow solid (740 mg, 44%). LCMS (ES) m/z calcd. C14H21N3O5S, 343.12; found, 344.2 (M+H).

### Step 2: Synthesis of tert-butyl (2-morpholino-4-((2-(trifluoromethyl)benzyl)carbamoyl) thiazol-5-yl)carbamate

To a suspension of methyl 5-((tert-butoxycarbonyl)amino)-2-morpholinothiazole-4-carboxylate (0.7 g, 2.04 mmol) and (2-(trifluoromethyl)phenyl)methanamine (428 mg, 2.45 mmol) in toluene (7 mL) was added 2 M trimethylaluminium in toluene (1.53 mL, 3.06 mmol) at 0° C. The reaction mixture was heated at 100° C. for 16 h. The reaction mixture was cooled to ambient temperature and quenched with ice-cold water (15 mL) and extracted with ethyl acetate (25×2 mL). The combined organic phase was washed with brine solution (15 mL) and dried over anhydrous sodium sulphate, filtered, and evaporated in vacuo to obtain crude. The crude material was purified by flash column chromatography using 50% ethyl acetate in n-hexane to afford tert-butyl (2-morpholino-4-((2-(trifluoromethyl)benzyl)carbamoyl)thiazol-5-yl)carbamate as a yellow solid (430 mg, 43%). LCMS (ES) m/z calcd. C21H25F3N404S, 486.1; found, 487.1 (M+1).

### Step 3: Synthesis of 5-amino-2-morpholino-N-(2-(trifluoromethyl)benzyl)thiazole-4-carboxamide

To a stirred solution of tert-butyl (2-morpholino-4-((2-(trifluoromethyl)benzyl)carbamoyl)thiazol-5-yl)carbamate (430 mg, 0.884 mmol) in methanol (2 mL) was added HCl in dioxane (15 mL, 60 mmol) at 0° C. and allowed to stirred at RT for 4 h. Progress of the reaction was monitored by TLC and LCMS. After completion of the reaction the reaction mixture was evaporated under reduced pressure and washed with diethyl ether to afford 5-amino-2-morpholino-N-(2-(trifluoromethyl)benzyl)thiazole-4-carboxamide as a brown solid (300 mg, 26%). LCMS (ES) m/z calcd. C16H17F3N4O2S, 386.10; found, 387.0 (M+1).

### Step 4: Synthesis of 5-(4,6-dichloro-5-hydroxypicolinamido)-2-morpholino-N-(2-(trifluoromethyl)benzyl)thiazole-4-carboxamide

To a suspension of 5-amino-2-morpholino-N-(2-(trifluoromethyl)benzyl)thiazole-4-carboxamide (0.3 g, 0.776 mmol) and 4,6-dichloro-5-hydroxypicolinic acid (178 mg, 0.854 mmol) in chlorobenzene (4 mL) was added phosphorus trichloride (0.034 mL, 0.388 mmol) dropwise under nitrogen atmosphere. The reaction mass was heated to 130° C. for 2 h. Progress of the reaction was monitored by TLC and LCMS. After completion of the reaction, the reaction mixture was quenched with ice water (20 mL) and extracted with ethyl acetate (3×10 ml). The combined organic layer was washed with water (10 mL), brine (10 mL), dried over sodium sulfate and evaporated under reduced pressure to get crude which was purified by flash column chromatography to afford 5-(4,6-dichloro-5-hydroxypicolinamido)-2-morpholino-N-(2-(trifluoromethyl)benzyl)thiazole-4-carboxamide as a yellow solid (14 mg, 3%). LCMS (ES) m/z calcd. C22H18C12F3N5O4S, 575.04; found, 576.1 (M+1); ¹H NMR (400 MHz, DMSO-d₆) δ 12.25 (s, 1H), 8.69 (t, J=8.0 Hz, 1H), 7.94 (s, 1H), 7.75 (d, J=8.0 Hz, 1H), 7.67 (t, J=8.0 Hz, 1H), 7.49-7.48 (m, 2H), 4.7 (d, J=8.0 Hz, 2H), 3.73 (t, J=4.0 Hz, 4H), 3.45 (t, J=4.0 Hz, 4H).

### Example 81: Synthesis of 5-(4,6-dichloro-5-hydroxypicolinamido)-2-(tetrahydro-2H-pyran-4-yl)-N-(2-(trifluoromethyl)benzyl)thiazole-4-carboxamide

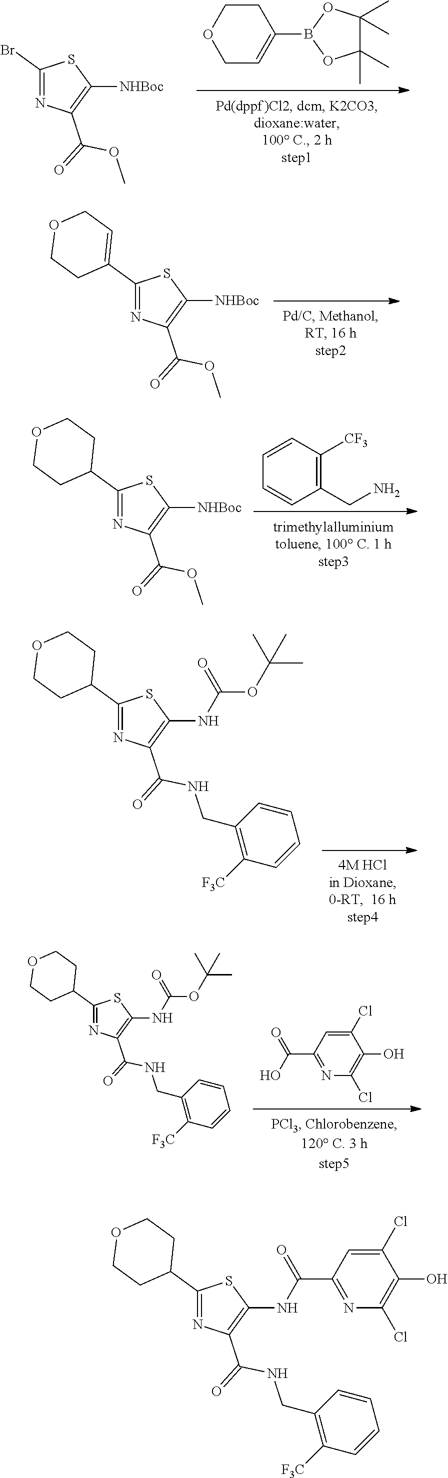

### Step 1: Synthesis of methyl 5-((tert-butoxycarbonyl)amino)-2-(3,6-dihydro-2H-pyran-4-yl)thiazole-4-carboxylate

To a stirred solution of methyl 2-bromo-5-{[(tert-butoxy)carbonyl]amino}-1,3-thiazole-4-carboxylate (1 g, 2.97 mmol) and 2-(3,6-dihydro-2H-pyran-4-yl)-4,4,5,5-tetramethyl-1,3,2-dioxaborolane (0.93 g, 4.45 mmol) in 1,4-dioxane:water (10 mL:1 mL) was added potassium carbonate (1.23 g, 8.9 mmol) and degassed with nitrogen for 10 min, followed by 1,1′-bis(diphenylphosphino)ferrocene dichloropalladium(II). DCM complex (110 mg, 0.148 mmol) was added and stirred at 100° C. for 2 h. Progress of the reaction was monitored by TLC and LCMS. After completion of the reaction, the reaction mixture was filtered through a pad of celite, filtrate was diluted with ethyl acetate (50 mL), washed with water (25×2 mL), brine solution and dried over anhydrous sodium sulphate, filtered, and concentrated under vacuo to give crude. The crude product was purified by flash column chromatography to afford methyl 5-{[(tert-butoxy)carbonyl]amino}-2-(3,6-dihydro-2H-pyran-4-yl)-1,3-thiazole-4-carboxylate (0.9 g, 89%) as white solid. LCMS (ES) m/z calcd. C₁₅H20N205S 340.11; found, 341.2 (M+1).

### Step 2: Synthesis of methyl 5-((tert-butoxycarbonyl)amino)-2-(tetrahydro-2H-pyran-4-yl)thiazole-4-carboxylate

To a stirred solution of methyl 5-{[(tert-butoxy)carbonyl]amino}-2-(3,6-dihydro-2H-pyran-4-yl)-1,3-thiazole-4-carboxylate (0.9 g, 2.64 mmol) in methanol (5 mL) and tetrahydrofuran (5 mL) was added palladium on carbon 10% w/w (0.1 g). The resulting reaction mixture was stirred under hydrogen atmosphere for 16 h at ambient temperature. The progress of the reaction was monitored by TLC and LCMS. The reaction mixture was filtered through pad of celite, and filtrate was concentrated under reduced pressure to afford methyl 5-{[(tert-butoxy)carbonyl]amino}-2-(oxan-4-yl)-1,3-thiazole-4-carboxylate (0.9 g, 99% yield) as white solid which was taken to the next step without any column purification. LCMS (ES) m/z calcd. C₁₅H22N205S 342.12; found, 343.0 (M+1).

### Step 3: Synthesis of tert-butyl (2-(tetrahydro-2H-pyran-4-yl)-4-((2-(trifluoromethyl) benzyl)carbamoyl)thiazol-5-yl)carbamate

To a suspension of methyl 5-{[(tert-butoxy)carbonyl]amino}-2-(oxan-4-yl)-1,3-thiazole-4-carboxylate (0.9 g, 2.63 mmol) and 1-[2-(trifluoromethyl)phenyl]methanamine (5) (0.691 g, 3.94 mmol) in toluene (10 mL) was added 2 M trimethylaluminium in toluene (1.97 mL 3.94 mmol) dropwise under nitrogen atmosphere at 0° C. The resulting reaction mixture was heated to 100° C. for 1 h. Progress of the reaction was monitored by TLC and LCMS. After completion of the reaction, the reaction mixture was quenched with ice water (50 mL) and extracted with ethyl acetate (2×50 mL). The combined organic layer was washed with water (50 mL), brine (50 mL), dried over sodium sulfate and evaporated under reduced pressure to get crude which was purified by flash column chromatography to afford tert-butyl (2-(tetrahydro-2H-pyran-4-yl)-4-((2-(trifluoromethyl)benzyl) carbamoyl)thiazol-5-yl)carbamate (0.65 g, 51%) as a white solid. LCMS (ES) m/z calcd. C22H26F3N3O4S 485.16; found, 486.0 (M+1).

### Step 4: Synthesis of 5-amino-2-(tetrahydro-2H-pyran-4-yl)-N-(2-(trifluoromethyl) benzyl)thiazole-4-carboxamide

To a suspension of tert-butyl N-[-(oxan-4-yl)-4-({[2-(trifluoromethyl)phenyl]methyl}carbamoyl)-1,3-thiazol-5-yl]carbamate (0.650 g, 1.34 mmol) in dichloromethane (5 mL) was added 4M HCl in dioxane (5 mL) at 0° C. and then stirred at ambient temperature for 16 h. the reaction mixture was concentrated under reduced pressure to get 5-amino-2-(tetrahydro-2H-pyran-4-yl)-N-(2-(trifluoromethyl)benzyl)thiazole-4-carboxamide (0.4 g, 77%) as off-white solid. LCMS (ES) m/z calcd. C17H18F3N3O2S 385.1; found, 386.0 (M+1).

### Step 5: Synthesis of 5-(4,6-dichloro-5-hydroxypicolinamido)-2-(tetrahydro-2H-pyran-4-yl)-N-(2-(trifluoromethyl)benzyl)thiazole-4-carboxamide

To a suspension of 5-amino-2-(tetrahydro-2H-pyran-4-yl)-N-(2-(trifluoromethyl)benzyl) thiazole-4-carboxamide (0.2 g, 0.519 mmol) and 4,6-dichloro-5-hydroxypicolinic acid (0.108 g, 0.519 mmol) in chlorobenzene (4 mL) was added phosphorus trichloride (0.071 g, 0.519 mmol) dropwise under nitrogen atmosphere. The reaction mixture was heated to 120° C. for 3 h. Progress of the reaction was monitored by TLC and LCMS. After completion of the reaction, the reaction mixture was quenched with ice water (20 ml) and extracted with ethyl acetate (3×20 ml). The combined organic layer was washed with water (10 mL), brine (10 mL), dried over sodium sulfate and evaporated under reduced pressure to get crude residue which was triturated with diethyl ether (10 mL) and filtered, the solid was dried under vacuo to afford 5-(4,6-dichloro-5-hydroxypicolinamido)-2-(tetrahydro-2H-pyran-4-yl)-N-(2-(trifluoromethyl) benzyl)thiazole-4-carboxamide (0.101 g, 33%) as pale brown solid. LCMS (ES) m/z calcd. C23H19C12F3N4O4S 574.05; found, 575.2 (M+1); ¹H NMR (400 MHz, DMSO-d₆) δ 12.23 (s, 1H), 8.81 (t, J=6.4 Hz, 1H), 7.81-7.74 (m, 2H), 7.71-7.65 (m, 1H), 7.51-7.46 (m, 2H) 4.72 (d, J=5.6 Hz, 2H), 4.00-3.93 (m, 2H), 3.49-3.44 (m, 2H), 3.25-3.17 (m, 1H), 2.09-1.99 (m, 2H), 1.85-1.75 (m, 2H).

### Example 82: Synthesis of 5-(3,5-dichloro-4-hydroxybenzamido)-2-(tetrahydro-2H-pyran-4-yl)-N-(2-(trifluoromethyl)benzyl)thiazole-4-carboxamide

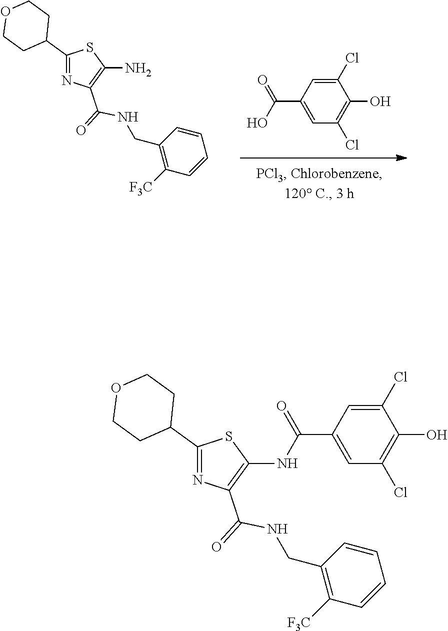 [SMILES: Nc1sc(C2CCOCC2)nc1C(=O)NCc1ccccc1C(F)(F)F ; O=C(O)c1cc(Cl)c(O)c(Cl)c1 ; O=C(Nc1sc(C2CCOCC2)nc1C(=O)NCc1ccccc1C(F)(F)F)c1cc(Cl)c(O)c(Cl)c1]

To a suspension of 5-amino-2-(tetrahydro-2H-pyran-4-yl)-N-(2-(trifluoromethyl)benzyl) thiazole-4-carboxamide (0.2 g, 0.519 mmol) and 3,5-dichloro-4-hydroxybenzoic acid (0.107 g, 0.519 mmol) in chlorobenzene (4 mL) was added phosphorus trichloride (71.3 mg, 0.519 mmol) dropwise under nitrogen atmosphere. The reaction mixture was heated to 120° C. for 3 h. Progress of the reaction was monitored by TLC and LCMS. After completion of the reaction, the reaction mixture was quenched with ice water (20 mL) and extracted with ethyl acetate (3×10 ml). The combined organic layer was washed with water (10 mL), brine (10 mL), dried over sodium sulfate and evaporated under reduced pressure to get crude which was purified by flash column chromatography to afford 5-(3,5-dichloro-4-hydroxybenzamido)-2-(tetrahydro-2H-pyran-4-yl)-N-(2-(trifluoromethyl) benzyl)thiazole-4-carboxamide as white solid (0.104 g, 34% yield). LCMS (ES) m/z calcd. C24H20Cl2F3N3O4S 573.05; found, 572.2 (M−1). ¹H NMR (400 MHz, DMSO-d₆) δ 12.24 (s, 1H), 11.32 (br s, 1H), 9.12 (t, J=6.4 Hz, 1H), 7.82 (s, 2H), 7.77-7.74 (m, 1H), 7.67 (t, J=7.6 Hz, 1H), 7.52-7.47 (m, 2H), 4.72 (d, J=6 Hz, 2H), 3.97-3.94 (m, 2H), 3.50-3.41 (m, 2H), 3.28-3.23 (m, 1H), 2.03-1.99 (m, 2H), 1.86-1.76 (m, 2H).

The following compounds has been synthesized as described procedure above.

| Ex. | Spectral data |
|---|---|
| 83 | LCMS (ES): m/z C25H22Cl2F3N3O3S calcd. for 571.07; found, 572.2 (M + H); ¹H NMR |
| | (400 MHz, DMSO-d₆): δ 11.30 (br s, 1H), 9.09 (t, J = 6.4 Hz, 1H), 7.81 (s, 2H), 7.75 (d, J = |
| | 8.0 Hz, 1H), 7.68-7.64 (m, 1H), 7.52-7.46 (m, 2H), 4.73 (d, J = 6.4 Hz, 2H) 3.01-2.94 (m, |
| | 1H), 2.11-2.09 (m, 2H), 1.82-1.79 (m, 2H), 1.71-1.68 (m, 1H), 1.60-1.50 (m, 2H), 1.46-1.36 |
| | (m, 2H), 1.32-1.22 (m, 1H). |
| 84 | LCMS (ES): m/z C24H21Cl2F3N4O3S calcd. for 572.07; found, 573.2 (M + H); ¹H NMR |
| | (400 MHz, DMSO-d₆): δ 12.66 (s, 1H), 8.98 (t, J = 6.0 Hz, 1H), 8.13 (s, 1H), 7.75 (d, J = |
| | 7.6 Hz, 1H), 7.68-7.65 (m, 1H), 7.51-7.46 (m, 2H), 4.73 (d, J = 6.4 Hz, 2H), 3.02-2.94 (m, |
| | 1H), 2.12-2.09 (m, 2H), 1.83-1.79 (m, 2H), 1.71-1.68 (m, 1H), 1.60-1.50 (m, 2H), 1.46-1.36 |
| | (m, 2H), 1.31-1.24 (m, 1H). |

### Example 85: Synthesis of 5-(4,6-dichloro-5-hydroxypicolinamido)-2-(1-methyl-1,2,3,6-tetrahydropyridin-4-yl)-N-(2-(trifluoromethyl) benzyl) thiazole-4-carboxamide

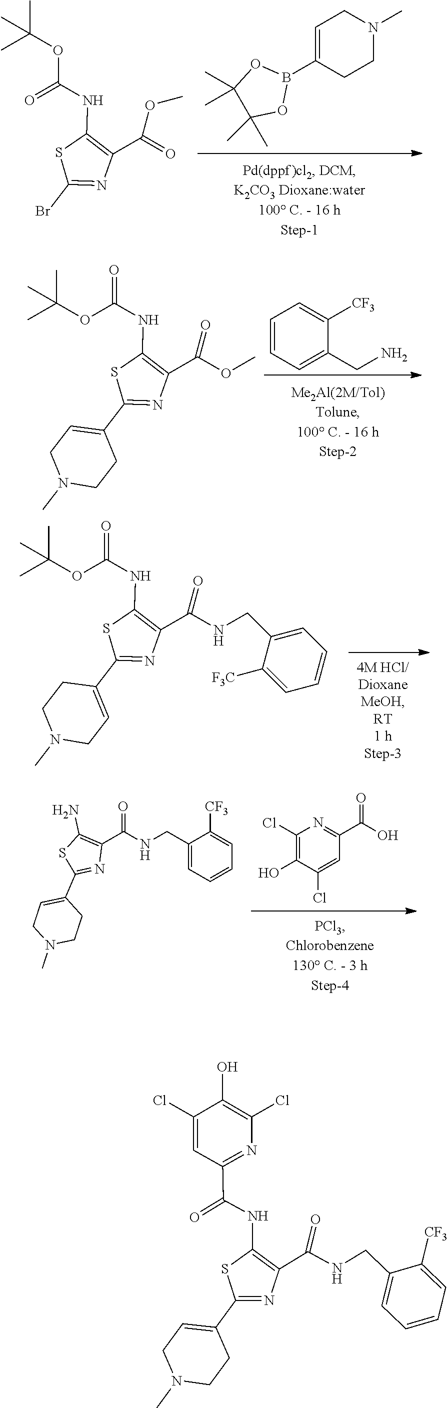

### Step 1: Preparation of methyl 5-((tert-butoxycarbonyl) amino)-2-(1-methyl-1,2,3,6-tetrahydropyridin-4-yl) thiazole-4-carboxylate

To a stirred solution of methyl 2-bromo-5-((tert-butoxycarbonyl)amino)thiazole-4-carboxylate (0.730 g, 2.16 mmol) in 1,4-dioxane (10 ml), water (1.5 ml) was added 1-methyl-4-(4,4,5,5-tetramethyl-1,3,2-dioxaborolan-2-yl)-1,2,3,6-tetrahydropyridine (0.483 g, 2.16 mmol) and potassium carbonate (0.898 g, 6.49 mmol). The reaction mixture was purged with nitrogen for 15 minutes followed by [1,1′-bis(diphenylphosphino)ferrocene]dichloropalladium(II), complex with dichloromethane (0.177 g, 0.216 mmol) was added and stirred at 100° C. for 16 h. The reaction mixture was monitored by TLC. The reaction mixture was poured into water (40 mL) and extracted with ethyl acetate (50 mL×3). The organic phase was washed with water (10 ml), brine (5 mL) and dried over anhydrous sodium sulfate, filtered, and concentrated in vacuo. The crude material was purified by flash column chromatography to afford methyl 5-((tert-butoxycarbonyl)amino)-2-(1-methyl-1,2,3,6-tetrahydropyridin-4-yl)thiazole-4-carboxylate (0.7 g, 91%) as brown solid. LCMS (ES): m/z calcd. for C16H23N3O4S 353.14; found, 354.1.

### Step 2: Preparation of tert-butyl (2-(1-methyl-1,2,3,6-tetrahydropyridin-4-yl)-4-((2-(trifluoromethyl) benzyl) carbamoyl) thiazol-5-yl) carbamate

To a stirred solution of methyl 5-((tert-butoxycarbonyl)amino)-2-(1-methyl-1,2,3,6-tetrahydropyridin-4-yl)thiazole-4-carboxylate (0.3 g, 0.849 mmol) in toluene (5 mL) was added 1-[2-(trifluoromethyl)phenyl]methanamine (0.149 g, 0.849 mmol) and 2M trimethylaluminium in toluene (0.229 mL, 2.55 mmol) drop-wise under nitrogen atmosphere at 0° C. The resulting reaction mixture was heated to 100° C. for 16 h. Progress of the reaction was monitored by TLC and LCMS. After completion of the reaction, the reaction mixture was quenched with ice-cold water (50 mL) and extracted with ethyl acetate (2×50 mL). The combined organic layer was washed with water (50 mL), brine (50 mL), dried over sodium sulfate and evaporated under reduced pressure to get crude which was purified by flash column chromatography to afford tert-butyl (2-(1-methyl-1,2,3,6-tetrahydropyridin-4-yl)-4-((2-(trifluoromethyl)benzyl)carbamoyl)thiazol-5-yl)carbamate (0.3 g, 71%) as off-white solid. LCMS (ES): m/z calcd. C23H27F3N4O3S 496.18; found, 497.2.

### Step 3: Preparation of 5-amino-2-(1-methyl-1,2,3,6-tetrahydropyridin-4-yl)-N-(2-(trifluoromethyl) benzyl) thiazole-4-carboxamide

To a suspension of tert-butyl N-[2-(1-methyl-1,2,3,6-tetrahydropyridin-4-yl)-4-({[2-(trifluoromethyl) phenyl]methyl}carbamoyl)-1,3-thiazol-5-yl]carbamate (0.280 g, 0.564 mmol) in methanol (5 mL) was added 4M HCl in dioxane (7 mL) at ambient temperature. And allowed to stir for another 1 h. The reaction mixture was concentrated under reduced pressure to give 5-amino-2-(1-methyl-1,2,3,6-tetrahydropyridin-4-yl)-N-{[2-(trifluoromethyl)phenyl]methyl}-1,3-thiazole-4-carboxamide (0.18 g, 80%) as white solid. LCMS (ES): m/z calcd. for C18H19F3N4OS 396.16; found, 397.1.

### Step 4: Preparation of 5-(4,6-dichloro-5-hydroxypicolinamido)-2-(1-methyl-1,2,3,6-tetrahydropyridin-4-yl)-N-(2-(trifluoromethyl) benzyl) thiazole-4-carboxamide

To a suspension of 5-amino-2-(1-methyl-1,2,3,6-tetrahydropyridin-4-yl)-N-{[2-(trifluoromethyl) phenyl]methyl}-1,3-thiazole-4-carboxamide (0.150 mg, 0.378 mmol) in chlorobenzene (5 mL) was added 4,6-dichloro-5-hydroxypyridine-2-carboxylic acid (0.0866 g, 0.416 mmol) and phosphorus trichloride (0.0331 mL, 0.378 mmol) at ambient temperature, the resulting reaction mixture was stirred at 130° C. for 3 h. The reaction was monitored by TLC. The reaction mixture was poured into water (20 mL) and extracted with ethyl acetate (20 mL×3). The organic phase was washed with water (10 mL), brine (3 mL) and dried over anhydrous sodium sulfate, filtered, and concentrated in vacuo. The crude material was purified by Prep HPLC to afford 4,6-dichloro-5-hydroxy-N-[2-(1-methyl-1,2,3,6-tetrahydropyridin-4-yl)-4-({[2-(trifluoromethyl) phenyl]methyl}carbamoyl)-1,3-thiazol-5-yl]pyridine-2-carboxamide. TFA salt (0.006 g, 3%) as a white solid. LCMS (ES) m/z calcd. For C24H20Cl2F3N5O3S 585.06; found 584.2 (M−H); ¹H NMR (400 MHz, DMSO-d₆): δ 12.63 (s, 1H), 9.80 (br s, 1H), 9.04 (t, J=6 Hz, 1H), 8.04 (s, 1H), 7.78 (d, J=7.6 Hz, 1H), 7.67 (t, J=8 Hz, 1H), 7.50 (t, J=7.6 Hz, 2H), 6.63 (s, 1H), 4.75 (br s, 2H), 4.05-3.88 (m, 1H), 3.76-3.64 (m, 1H), 3.36 (br s, 2H), 2.92 (s, 3H), 2.68-2.67-2.87 (m, 2H).

### Example 86: 5-(4,6-dichloro-5-hydroxypicolinamido)-2-(1-methylpiperidin-4-yl)-N-(2-(trifluoromethyl)benzyl)thiazole-4-carboxamide

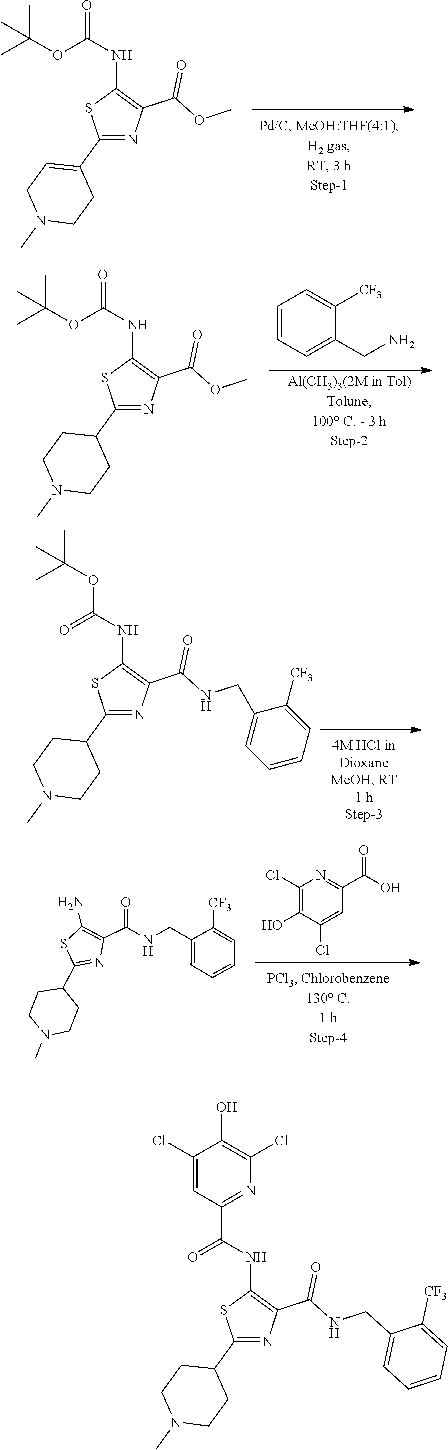

### Step 1: Synthesis of methyl 5-((tert-butoxycarbonyl)amino)-2-(1-methylpiperidin-4-yl)thiazole-4-carboxylate

To a stirred solution of 5-{[(tert-butoxy)carbonyl]amino}-2-(1-methyl-1,2,3,6-tetrahydropyridin-4-yl)-1,3-thiazole-4-carboxylate (0.250 g, 0.707 mmol) in MeOH:THF (4:1) (6 mL) was added Pd on carbon 10% (0.150 g) and stirred at room temperature for 3 h under H₂ atmosphere. The progress of the reaction was monitored by LCMS. Reaction mixture was filtered through pad of celite, filtrate was concentrated under reduced pressure to afford methyl 5-((tert-butoxycarbonyl)amino)-2-(1-methylpiperidin-4-yl)thiazole-4-carboxylate (0.250 g, 99%) as brown solid. LCMS (ES) m/z calcd. C16H25N3O4S for, 355.16; found, 356.2 (M+H).

### Step 2: Synthesis of tert-butyl (2-(1-methylpiperidin-4-yl)-4-((2-(trifluoromethyl)benzyl) carbamoyl)thiazol-5-yl)carbamate

To a stirred solution of methyl 5-((tert-butoxycarbonyl)amino)-2-(1-methylpiperidin-4-yl)thiazole-4-carboxylate (0.250 g, 0.703 mmol) in toluene (10.0 mL) was added 1-[2-(trifluoromethyl)phenyl]methanamine (0.123 g, 0.703 mmol) and trimethylaluminium (2.0 M in toluene) (0.101 g, 1.41 mmol) drop-wise at 0° C. the resulting reaction mixture was stirred at 100° C. for 3 h. reaction mixture was cooled to ambient temperature and poured into ice-cold water (25 mL), extracted with ethyl acetate (3×25 mL). the combined organic layer was dried over sodium sulphate, filtered, and concentrated to afford crude. Crude was purified using flash chromatography to afford tert-butyl (2-(1-methylpiperidin-4-yl)-4-((2-(trifluoromethyl)benzyl)carbamoyl)thiazol-5-yl)carbamate (0.150 g, 43%) as an off-white solid. LCMS (ES) m/z calcd. for. C23H29F3N4O3S, 498.19; found 499.2 (M+H).

### Step 3: Synthesis of 5-amino-2-(1-methylpiperidin-4-yl)-N-(2-(trifluoromethyl)benzyl) thiazole-4-carboxamide

To a stirred solution of tert-butyl (2-(1-methylpiperidin-4-yl)-4-((2-(trifluoromethyl)benzyl) carbamoyl)thiazol-5-yl)carbamate (0.150 g, 0.301 mmol) in DCM (3.0 mL) was added hydrogen chloride (4M in 1,4-Dioxane) (0.054 g, 1.5 mmol) and stirred at room temperature for 1 h. The reaction mixture was concentrated under reduce pressure to get crude residue which was triturated with diethylether (15 mL) and n-pentane (15 mL) to afford 5-amino-2-(1-methylpiperidin-4-yl)-N-(2-(trifluoromethyl)benzyl) thiazole-4-carboxamide (0.1 g, 83%) as brown solid. LCMS (ES) m/z calcd. for. C18H21F3N4OS, 398.14; found 399.2 (M+H).

### Step 4: Synthesis of 5-(4,6-dichloro-5-hydroxypicolinamido)-2-(1-methylpiperidin-4-yl)-N-(2-(trifluoromethyl)benzyl)thiazole-4-carboxamide

To a stirred solution of 5-amino-2-(1-methylpiperidin-4-yl)-N-{[2-(trifluoromethyl)phenyl]methyl}-1,3-thiazole-4-carboxamide (0.1 g, 0.251 mmol) in acetonitrile (10 mL, 191 mmol) was added 4,6-dichloro-5-hydroxypyridine-2-carboxylic acid (0.052 g, 0.251 mmol) and phosphorous trichloride (0.027 g, 0.201 mmol) at ambient temperature. The resulting reaction mixture was heated to 130° C. for 1 h. Reaction was cooled to room temperature and then to it was added ice-cold water (15 mL), precipitated solid was filtered to obtain crude solid. The crude compound was purified by Prep HPLC to afford 5-(4,6-dichloro-5-hydroxypicolinamido)-2-(1-methylpiperidin-4-yl)-N-(2-(trifluoromethyl)benzyl)thiazole-4-carboxamide. Formic acid salt (0.0227 g, 15.3%) as off-white solid. LCMS (ES) m/z calcd. for. C24H22C12F3N5O3S, 587.08; found 588.2 (M+H); ¹H NMR (400 MHz, DMSO-d₆): δ 12.25 (s, 1H), 9.30 (s, 1H), 8.77 (s, 1H), 7.75-7.77 (m, 1H), 7.6-7.70 (m, 1H), 7.47-7.52 (m, 2H), 4.73 (d, J=5.6 Hz, 2H), 3.46 (br s, 4H), 3.08 (br s, 2H), 2.8 (s, 3H), 2.3-2.4 (br s, 1H), 1.8-2.1 (br s, 2H).

The following compounds has been synthesized as described above.

| Ex. | Spectral data |
|---|---|
| 87 | LCMS (ES) m/z calcd. For C25H23Cl2F3N4O3S, 586.08; found, 587.2 (M + H). ¹H NMR |
| | (400 MHz, DMSO-d₆) δ 11.88 (s, 1H), 8.9 (s, 1H), 7.64-7.62 (m, 6H), 4.71 (d, J = 5.2 Hz, |
| | 2H), 3.3-2.9 (br s, 5H), 2.65 (br s, 2H), 2.3-2.1 (m, 3H), 2.0-1.7 (m, 2H). |
| 88 | LCMS (ES) m/z calcd. for. C24H19Cl2F3N4O3S, 570.05; found 571.1 (M + H); ¹H NMR |
| | (400 MHz, DMSO-d₆): δ 12.67 (s, 1H), 8.99 (t, J = 6.0 Hz, 1H), 8.11 (s, 1H), 7.77-7.75 |
| | (m, 1H), 7.69-7.65 (m, 1H), 7.52-7.47 (m, 2H), 6.67 (s, 1H), 4.74 (d, J = 5.2 Hz, 2H), |
| | 2.15-2.45 (m, 3H), 1.5-1.9 (m, 5H). |
| 89 | LCMS (ES) m/z calcd. for C25H15Cl2F4N3O3S, 583.01; found 582.2 [M − H]. ¹H NMR |
| | (400 MHz, DMSO-d₆): δ 12.33 (s, 1H), 11.65 (bs, 1H), 9.49 (t, J = 6.0 Hz, 1H), 8.50-8.48 |
| | (m, 1H), 7.78-7.66 (m, 5H), 7.58-7.48 (m, 4H), 4.79 (d, J = 5.6 Hz, 2H) |
| 90 | LCMS (ES) m/z calcd. for C24H14ClF5N4O3S, 568.04; found 567.2 [M − H]; ¹H NMR |
| | (400 MHz, DMSO-d₆): δ 12.19 (bs, 1H), 9.44 (bs, 1H), 9.13 (s, 1H), 8.67 (s, 1H), 8.40 (d, |
| | J = 7.2 Hz, 1H), 7.77 (d, J = 8 Hz, 1H), 7.70-7.58 (m, 4H), 7.52-7.48 (m, 2H), 4.80 (d, J = |
| | 5.6 Hz, 2H) |
| 91 | LCMS (ES) m/z calcd. for C26H18Cl2F3N3O5S2, 643.0; found 644.1 [M + H]⁺; ¹H NMR |
| | (400 MHz, DMSO-d₆): δ 12.35 (s, 1H), 11.4 (s, 1H), 9.26 (t, J = 6.0 Hz, 1H), 8.18-8.16 |
| | (m, 1H), 7.92-7.83 (m, 5H), 7.75 (d, J = 8 Hz, 1H), 7.69 (t, J = 7.6 Hz, 1H), 7.51-7.46 (m, |
| | 2H), 4.76 (d, J = 5.6 Hz, 2H), 3.53 (s, 3H) |
| 92 | LCMS (ES) m/z calcd. for C25H17Cl2F3N4O5S2, 644.00; found 645.1 [M + H]⁺; ¹H |
| | NMR (400 MHz, DMSO-d₆) δ 12.74 (s, 1H), 9.14 (t, J = 6.4 Hz, 1 H), 8.16 (d, J = 7.6 Hz, |
| | 1H), 8.13 (s, 1H), 7.90-7.84 (m, 3H), 7.76 (d, J = 7.6 Hz, 1H), 7.70 (t, J = 7.6 Hz, 1H), |
| | 7.50-7.48 (m, 2H), 4.75 (d, J = 6.0 Hz, 2H), 3.52 (s, 3H) |
| 93 | LCMS (ES) m/z calcd. For C₂₈H₂₂Cl₂F₄N₄O₃S is 640.07; found, 641.3 (M + H); ¹H NMR |
| | (400 MHz, DMSO-d₆) δ 12.1 (s, 1H), 9.13 (bs, 1H), 8.30 (d, J = 4.8 Hz, 1H), 7.77-7.76 |
| | (m, 1H), 7.69-7.49 (m, 7H), 4.82 (d, J = 5.6 Hz, 2H), 3.91 (s, 2H), 2.47 (bs, 6H*merged |
| | with DMSO peak) |
| 94 | LCMS (ES) m/z calcd. For C₂₈H₂₂ClF₅N₄O₃S is 624.10; found, 625.3 (M + H)⁺; ¹H NMR |
| | (400 MHz, DMSO-d₆) δ 12.23 (s, 1H), 9.25 (bs, 1H), 8.29-8.28 (m, 1H), 7.77-7.75 (m, |
| | 1H), 7.69-7.59 (m, 3H), 7.50-7.42 (m, 4H), 4.82 (d, J = 5.6 Hz, 2H), 3.71 (s, 2H), 2.38 |
| | (s, 6H) |
| 95 | LCMS (ES) m/z calcd. For C₂₆H₁₅Cl₂F₃N₄O₃S is 590.02; found, 589.2 (M − H); ¹H NMR |
| | (400 MHz, DMSO-d₆) δ 12.30 (s, 1H), 11.38 (bs, 1H), 9.50 (t, J = 5.6 Hz, 1H), 8.59-8.58 |
| | (m, 1H), 8.34-8.31 (m, 1H), 7.96-7.93 (m, 1H), 7.85 (s, 2H), 7.78-7.66 (m, 3H), |
| | 7.58-7.56 (m, 1H), 7.50-7.48 (m, 1H), 4.79 (d, J = 5.6 Hz, 2H) |
| 96 | LCMS (ES) m/z calcd. For C26H15ClF4N4O3S is 574.05; found, 573.3 (M − H), ¹H NMR |
| | (400 MHz, DMSO-d₆) δ 12.31 (s, 1H), 11.66 (bs, 1H), 9.51 (t, J = 6.0 Hz, 1H), 8.60 (s, |
| | 1H), 8.34 (d, J = 8.0 Hz, 1H), 7.95 (d, J = 7.6 Hz, 1H), 7.78-7.66 (m, 5H), 7.58-7.56 (m, |
| | 1H), 7.52-7.48 (m, 1H), 4.79 (d, J = 5.6 Hz, 2H) |
| 97 | LCMS (ES) m/z calcd. for C₂₆H₁₈Cl₂F₃N₃O₅S₂ 643.0; found, 642.1 (M − H); ¹H NMR (400 |
| | MHz, DMSO-d₆) δ 12.40 (s, 1H), 11.38 (bs, 1H), 9.51 (t, J = 8 Hz, 1H), (s, 1H), 8.38 (d, |
| | J = 8 Hz, 1H), 8.05 (d, J = 7.6 Hz, 1H), 7.87 (s, 2H), 7.84-7.77 (m, 2H), 7.70-7.66 (m, |
| | 1H), 7.59-7.57 (m, 1H), 7.52-7.48 (m, 1H), 4.81 (d, J = 8 Hz, 2H), 3.33 (s, 3H*merged |
| | with DMSO moisture peak) |
| 98 | LCMS (ES) m/z calcd. for C₂₅H₁₇Cl₂F₃N₄O₅S₂ 644.0; found, 643.1 (M − H); ¹H NMR (400 |
| | MHz, DMSO-d₆) δ 12.74 (s, 1H), 9.38 (t, J = 5.6 Hz, 1H), 8.58 (s, 1H), 8.38 (d, J = 8 Hz, |
| | 1H), 8.10 (s, 1H), 8.04 (d, J = 8 Hz, 1H), 7.84-7.77 (m, 2H), 7.68-7.66 (m, 1H), |
| | 7.58-7.56 (m, 1H), 7.52-7.50 (m, 1H), 4.80 (d, J = 5.6 Hz, 2H), 3.33 (s, 3H) |
| 99 | LCMS (ES) m/z calcd. For, C25H17Cl2F3N4O3S, 580.04; found, 581.3 (M + H); ¹H NMR |
| | (400 MHz, DMSO-d₆) δ 12.72 (s, 1H), 9.01 (d, J = 8 Hz, 1H), 8.12-8.11 (m, 3H), 8.02 (d, |
| | J = 8 Hz, 1H), 7.74-7.71 (m, 2H), 7.58-7.48 (m, 4H), 5.64-5.60 (m, 1H), 1.59 (d, J = 6.8 |
| | Hz, 3H) |
| 100 | LCMS (ES): m/z calcd. for C23H14Cl2F3N5O3S; 567.01 found, 566.2 (M − H); ¹H NMR |
| | (400 MHz, DMSO-d₆): δ 12.27 (s, 1H), 11.40 (s, 1H), 9.49 (s, 1H), 9.43 (bs, 1H), 8.73 (s, |
| | 2H), 7.79-7.77 (m, 3H), 7.71-7.67 (m, 1H), 7.60-7.59 (m, 1H), 7.51 (t, J = 7.6 Hz, 1H), |
| | 4.81 (d, J = 5.6 Hz, 2H) |
| 101 | LCMS (ES): m/z calcd. for C22H13Cl2F3N6O3S; 568.01 found, 569.2 (M + H); ¹H NMR |
| | (400 MHz, DMSO-d₆): δ 12.80 (s, 1H), 9.51 (d, J = 1.2 Hz, 1H), 9.38 (t, J = 6.4 Hz, 1H), |
| | 8.75-8.73 (m, 2H), 8.16 (s, 1H), 7.78 (d, J = 8.0 Hz, 1H), 7.68 (t, J = 7.6 Hz, 1H), 7.59 (d, |
| | J = 7.6 Hz, 1H), 7.51 (t, J = 7.6 Hz, 1H), 4.81 (d, J = 6.0 Hz, 2H) |
| 102 | LCMS (ES): m/z calcd. for C25H17Cl2F3N4O3S; 580.04 found, 579.2 (M − H); ¹H NMR |
| | (400 MHz, DMSO-d₆): δ 12.13 (s, 1H), 9.34 (s, 1H), 9.09 (bs, 1H), 8.68 (d, J = 3.6 Hz, |
| | 1H), 8.43 (d, J = 8.0 Hz, 1H), 8.01 (d, J = 7.6 Hz, 1H), 7.74-7.71 (m, 4H), 7.58-7.55 (m, |
| | 1H), 7.50-7.46 (m, 1H), 5.64-5.61 (m, 1H), 1.59 (d, J = 6.8 Hz, 3H) |
| 103 | LCMS (ES): m/z calcd. for C24H16Cl2F3N5O3S; 581.03 found, 582.2 (M + H); ¹H NMR |
| | (400 MHz, DMSO-d₆): δ 12.74 (s, 1H), 9.38 (d, J = 1.6 Hz, 1H), 9.09 (d, J = 8.0 Hz, 1H), |
| | 8.70-8.69 (m, 1H), 8.48-8.45 (m, 1H), 8.13-8.12 (m, 1H), 8.01 (d, J = 8.0 Hz, 1H), 7.74-7.71 |
| | (m, 2H), 7.60-7.57 (m, 1H), 7.50-7.46 (m, 1H), 5.66-5.59 (m, 1H), 1.59 (d, J = 7.2 Hz, 3H) |
| 104 | LCMS (ES): m/z calcd. for C26H18Cl2F3N3O5S2, 643.00; found, 642.0 (M − H), ¹H NMR |
| | (400 MHz, DMSO-d₆): δ 12.38 (s, 1H), 11.39 (bs, 1H), 9.51 (t, J = 6.4 Hz, 1H), 8.83 (d, J = |
| | 8.4 Hz, 2H), 8.07 (d, J = 8 Hz, 2H), 7.87 (s, 2H), 7.78 (d, J = 7.6 Hz, 1H), 7.68 (t, J = 7.6 |
| | Hz, 1H), 7.58 (d, J = 8 Hz, 1H), 7.50 (t, J = 7.6 Hz, 1H), 4.80 (d, J = 5.6 Hz, 2H), 3.32 (s, |
| | 3H) |

### Example 105: Synthesis of 5-(3-chloro-5-fluoro-4-hydroxybenzamido)-2-(2-fluoro-5-(pyrrolidin-1-ylmethyl)phenyl)-N-(2-(trifluoromethyl)benzyl)thiazole-4-carboxamide

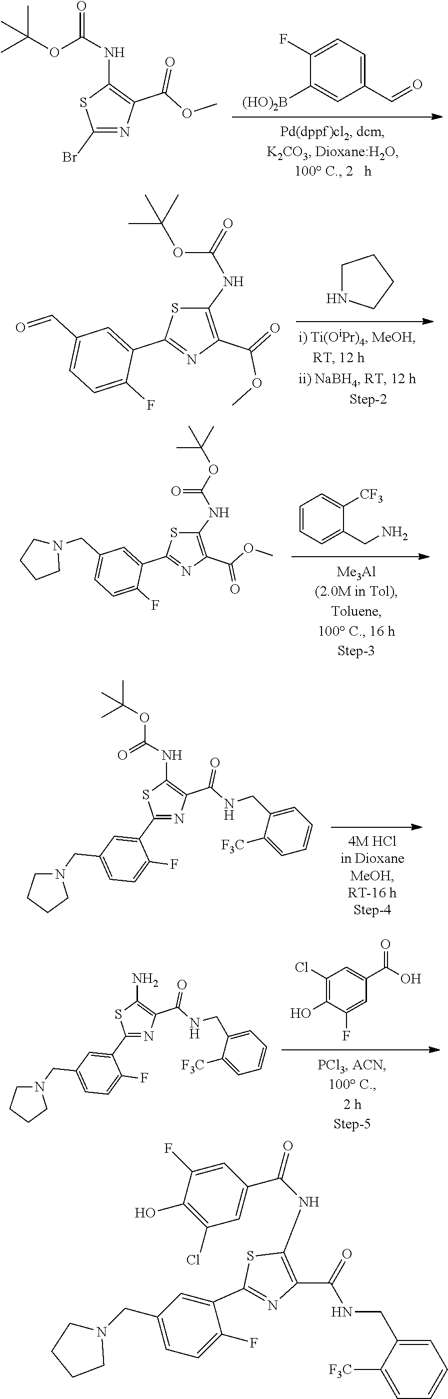

### Step1: Preparation of methyl 5-((tert-butoxycarbonyl)amino)-2-(2-fluoro-5-formylphenyl)thiazole-4-carboxylate

To a solution of methyl 2-bromo-5-{[(tert-butoxy)carbonyl]amino}-1,3-thiazole-4-carboxylate (0.9 g, 2.67 mmol) and (2-fluoro-5-formylphenyl)boronic acid (0.538 g, 3.2 mmol) dissolved in 1,4-dioxane (10 mL) was added potassium carbonate (1.11 g, 8.01 mmol) and water (1 mL). The reaction mixture was purged with nitrogen gas for 15 minutes and then added Pd(dppf)Cl₂·DCM complex (0.218 g, 0.26 mmol) at room temperature. The resulting reaction mixture was heated to 100° C. for 2 h. The progress of the reaction was monitored by TLC. The reaction mixture was filtered through pad of celite, the celite pad was washed with Ethyl acetate (25 mL), filtrate was washed with water (2×50 ml), brine solution (25 mL) and dried over Na₂SO₄, concentrated and dried to afford crude (0.85 g, 83.7% yield) as a brown solid. LCMS (ES): m/z calcd. for C17H17FN2O5S 380.08; found, 381.0 (M+H).

### Step 2: Preparation of methyl 5-((tert-butoxycarbonyl)amino)-2-(2-fluoro-5-(pyrrolidin-1-ylmethyl)phenyl)thiazole-4-carboxylate

To a stirred solution of methyl 5-{[(tert-butoxy)carbonyl]amino}-2-(2-fluoro-5-formylphenyl)-1,3-thiazole-4-carboxylate (0.6 g, 1.58 mmol) in methanol (6 mL, 148 mmol) was added pyrrolidine (1.12 g, 15.8 mmol) and Titanium isopropoxide (4.78 mL, 15.8 mmol) at room temperature and stirred for 12 h under argon atmosphere. To this was added NaBH₄ (298 mg, 7.89 mmol) at room temperature. The resulting reaction mixture was stirred at room temperature for another 2 h. Progress of the reaction was monitored by TLC. After completion, the reaction mixture was quenched with water (20 mL) and resulting residue was filtered through celite pad and washed with DCM (25 mL×2). The combined organic phase was washed with water (15), dried over sodium sulfate and evaporated under reduced pressure to result in crude compound, the crude material was triturated with 10% Ethyl acetate:Hexane (10 ml×2) and diethyl ether (10 mL×2) to afford methyl 5-((tert-butoxycarbonyl)amino)-2-(2-fluoro-5-(pyrrolidin-1-ylmethyl)phenyl)thiazole-4-carboxylate (0.205 g, 29.84%) as an off-white solid. LCMS (ES): m/z calcd. for C21H26FN3O4S, 435.16; found, 436.1 (M+H).

### Step 3: Preparation of tert-butyl (2-(2-fluoro-5-(pyrrolidin-1-ylmethyl)phenyl)-4-((2-(trifluoromethyl)benzyl)carbamoyl)thiazol-5-yl)carbamate

To a stirred solution of methyl 5-{[(tert-butoxy)carbonyl]amino}-2-{2-fluoro-4-[(pyrrolidin-1-yl)methyl]phenyl}-1,3-thiazole-4-carboxylate (0.205 g, 0.471 mmol) in Toluene (2 mL, 16.9 mmol) was added 1-[2-(trifluoromethyl)phenyl]methanamine (0.066 mL, 0.471 mmol) and cooled to 0° C. To this was added 1M trimethylaluminium in Toluene (0.136 mL, 1.41 mmol). The resulting reaction mixture was heated to 100° C. for 16 h. The Progress of the reaction was monitored by TLC. After completion of the reaction, the reaction mixture was quenched with cold water (25 mL) and extracted into EtOAc (25 mL×3). The combined organic layer was dried over sodium sulfate and evaporated under reduced pressure to give crude. The crude was purified by Combiflash chromatography using 3% Methanol: Dichloromethane as an eluent to afford tert-butyl (2-(2-fluoro-5-(pyrrolidin-1-ylmethyl)phenyl)-4-((2-(trifluoromethyl)benzyl)carbamoyl)thiazol-5-yl)carbamate (0.4 g, 77%) as off white solid. LCMS (ES): m/z calcd. for C28H30F4N4O3S, 578.20; found, 579.0 (M+H).

### Step 4: Preparation of 5-amino-2-(2-fluoro-5-(pyrrolidin-1-ylmethyl)phenyl)-N-(2-(trifluoromethyl)benzyl)thiazole-4-carboxamide

To a solution of tert-butyl N-(2-{2-fluoro-4-[(pyrrolidin-1-yl)methyl]phenyl}-4-({[2-(trifluoromethyl)phenyl]methyl}carbamoyl)-1,3-thiazol-5-yl)carbamate (0.4 g, 0.691 mmol) in methanol (4 mL) was added with 4M HCl in dioxane (0.86 mL) at 0° C. and stirred at room temperature for 16 h under argon atmosphere. Progress of the reaction was monitored by TLC. After completion, the reaction mixture was basified with sodium bicarbonate (10 mL) and extracted into Ethyl acetate (25 mL×2). The combined organic layer was washed with water (10 mL), brine solution (10 ml) dried over sodium sulfate and evaporated under reduced pressure to get 5-amino-2-(2-fluoro-5-(pyrrolidin-1-ylmethyl)phenyl)-N-(2-(trifluoromethyl)benzyl)thiazole-4-carboxamide (95 mg, 29%) as a light yellow solid. LCMS (ES): m/z calcd. for C23H22F4N4OS, 478.15; found, 479.0 (M+H).

### Step-5: Preparation of 5-(3-chloro-5-fluoro-4-hydroxybenzamido)-2-(2-fluoro-5-(pyrrolidin-1-ylmethyl)phenyl)-N-(2-(trifluoromethyl)benzyl)thiazole-4-carboxamide

To a suspension of 5-amino-2-{2-fluoro-5-[(pyrrolidin-1-yl)methyl]phenyl}-N-{[2-(trifluoromethyl)phenyl]methyl}-1,3-thiazole-4-carboxamide (0.095 g, 0.199 mmol) in acetonitrile (1 mL) was added with 3-chloro-5-fluoro-4-hydroxybenzoic acid (41.6 mg, 0.218 mmol) and Phosphorus trichloride (0.017 mL, 0.199 mmol) at room temperature and then stirred at 130° C. for 2 h. The progress of the reaction was monitored by TLC. After completion, the reaction mixture was poured into ice cold water (20 mL) and extracted with ethyl acetate (20 mL×3). The organic phase was washed with water (10 mL), brine solution (5 mL) and dried over anhydrous sodium sulfate, filtered, and concentrated in vacuo. The crude material was purified by Prep HPLC, using 0.1% TFA in Water: Acetonitrile gradient. Collected pure fractions were lyophilized to get 5-(3-chloro-5-fluoro-4-hydroxybenzamido)-2-(2-fluoro-5-(pyrrolidin-1-ylmethyl)phenyl)-N-(2-(trifluoromethyl)benzyl)thiazole-4-carboxamide (0.012 g, 9.28%). LCMS (ES): m/z calcd. for C30H24ClF5N4O3S, 650.12; found, 651.4 (M+H). 1H NMR (400 MHz, DMSO-d₆) δ 12.31 (s, 1H), 10.87 (bs, 1H), 9.27 (bs, 1H), 8.45 (d, J=6.8 Hz, 1H), 7.77 (d, J=7.6 Hz, 1H), 7.71-7.63 (m, 4H), 7.57 (d, J=6.8 Hz, 2H), 7.52-7.50 (m, 1H), 4.82 (d, J=6 Hz, 2H), 4.30 (s, 2H), 3.16 (m, 4H), 1.92 (m, 4H). LC-Purity: 99.62% at 240 nm.

### Example 106: Synthesis of N-(bicyclo[1.1.1]pentan-1-ylmethyl)-5-(3-chloro-5-fluoro-4-hydroxybenzamido)-2-(2-fluorophenyl)thiazole-4-carboxamide

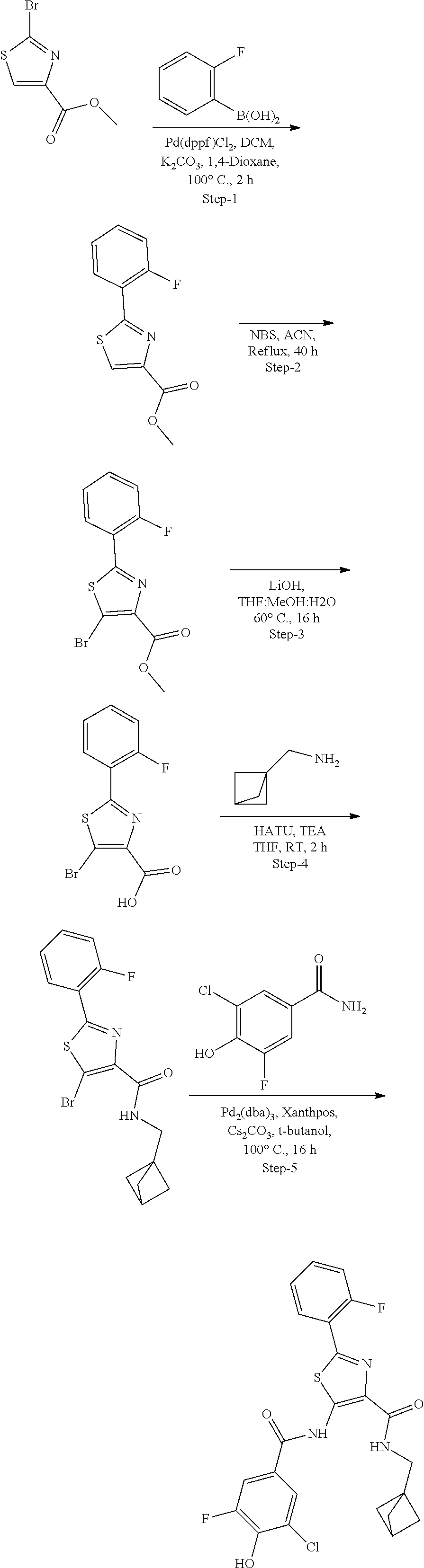

### Step1: Preparation of methyl 2-(2-fluorophenyl)thiazole-4-carboxylate

To a stirred solution of methyl 2-bromo-1,3-thiazole-4-carboxylate (50 g, 225 mmol) in 1,4-dioxane (750 mL), water (125 mL) was added (2-fluorophenyl)boronic acid (47.25 g, 337.5 mmol) and Potassium carbonate (93.5 g, 67.5 mmol). The reaction mixture was purged with nitrogen gas for 15 minutes and then Pd(dppf)Cl₂·DCM complex (9.2 g, 0.05 eq., 2.25 mmol) was added at room temperature. The resulting reaction mixture was heated to 100° C. for 2 h. The progress of the reaction was monitored by TLC. The reaction mixture was filtered through pad of celite, the celite pad was washed with EtOAc (2 L), filtrate was washed with water (2×1000 ml), brine Solution (500 mL) and dried over Na₂SO₄, concentrated and dried to afford crude. The crude material was purified by column chromatography by using 5% EtOAc: Hexane as an eluent to afford methyl 2-(2-fluorophenyl)-1,3-thiazole-4-carboxylate (41 g, 77% yield) as a white solid. LCMS (ES): m/z calcd. for C5H5BrN2O2S 237.03; found, 237.9 (M+H).

### Step 2: Preparation of methyl 5-bromo-2-(2-fluorophenyl)thiazole-4-carboxylate

To a stirred solution of methyl 2-(2-fluorophenyl)-1,3-thiazole-4-carboxylate (20 g, 84.3 mmol) in Acetonitrile was added NBS (45 g, 253 mmol) at room temperature. The resulting reaction mixture was refluxed for 24 h and then added another portion of NBS (30 g) and continued the stirring at reflux temperature for another 24 h. Progress of the reaction was monitored by TLC. After completion of the reaction. The reaction mixture was poured into ice cold water (2 L), precipitated solid was filtered and washed with water (1 L), dried to afford methyl 5-bromo-2-(2-fluorophenyl)thiazole-4-carboxylate (20 g, 75% yield) as a white fluffy solid. LCMS (ES): m/z calcd. for C11H7BrFNO2S, 314.94; found, 316.0 (M+H).

### Step 3: Preparation of 5-bromo-2-(2-fluorophenyl)thiazole-4-carboxylic acid

To a stirred solution of methyl 5-bromo-2-(2-fluorophenyl)-1,3-thiazole-4-carboxylate (16 g, 50.6 mmol) in tetrahydrofuran (200 mL), methanol (75 mL) and water (30 mL) was added LiOH·H₂O (3.64 g, 152 mmol) at room temperature. The resulting reaction mixture was stirred at 60° C. for 16 hours. Progress of the reaction was monitored by TLC and LCMS. The reaction mixture was poured into cold water and acidified to pH-2 by dil. HCl solution. Precipitated solid was filtered and washed with water (1 L), dried to afford 5-bromo-2-(2-fluorophenyl)-1,3-thiazole-4-carboxylic acid (14 g, 96%) as a white solid. LCMS (ES): m/z calcd. for C10H5BrFNO2S, 300.92; found, 301.9 (M+H).

### Step 4: Preparation of N-(bicyclo[1.1.1]pentan-1-ylmethyl)-5-bromo-2-(2-fluorophenyl)thiazole-4-carboxamide

To a stirred solution of 5-bromo-2-(2-fluorophenyl)-1,3-thiazole-4-carboxylic acid (0.2 g, 0.66 mmol) in NN-Dimethylformamide (5 ml) was added bicyclo[1.1.1]pentan-1-ylmethanamine (0.064 g, 0.66 mmol), HATU (0.3 g, 0.794 mmol) and triethylamine (0.37 mL, 2.65 mmol) at room temperature. The resulting reaction mixture was stirred at room temperature for 2 hours. The progress of the reaction was monitored by TLC and LCMS. The reaction mixture was poured into ice cold water (10 mL) and extracted with ethyl acetate (2×20 mL), the combined organic layer was washed with water, brine solution, dried over Na₂SO₄, concentrated and dried to afford crude. The crude material was purified by flash column chromatography using 5% ethyl acetate in Hexane as an eluent to afford N-(bicyclo[1.1.1]pentan-1-ylmethyl)-5-bromo-2-(2-fluorophenyl)thiazole-4-carboxamide (0.075 g, 30%) as a white solid. LCMS (ES): m/z calcd. for C16H14BrFN2OS, 380.00; found, 380.9 (M+H).

### Step-5: Preparation of N-(bicyclo[1.1.1]pentan-1-ylmethyl)-5-(3-chloro-5-fluoro-4-hydroxybenzamido)-2-(2-fluorophenyl)thiazole-4-carboxamide

To a stirred solution of N-(bicyclo[1.1.1]pentan-1-ylmethyl)-5-bromo-2-(2-fluorophenyl)thiazole-4-carboxamide (0.07 g, 0.184 mmol) and 3-chloro-5-fluoro-4-hydroxybenzamide (0.052 g, 0.275 mmol) in t-butanol (2 mL) was added cesium carbonate (0.18 g, 0.55 mmol). Then reaction mixture degassed under nitrogen gas for 10 min. Then followed by the addition of Xanthphos (11 mg, 0.018 mmol) and Pd₂(dba)₃ (15 mg, 0.018 mmol) at room temperature, the reaction mixture was heated to 100° C. for 16 hours. The progress of the reaction was monitored by LCMS and TLC. The reaction mixture was poured into water (100 ml) and extracted with ethyl acetate (3×100 ml), the combined organic layer was washed with water, brine, dried over Na2SO4 and concentrated under reduced pressure to get the crude product. The crude solid was stirred with diethyl ether (10 ml) for 30 min and then filtered the solid. The solid was further triturated with methanol, filtered, and dried to afford N-(bicyclo[1.1.1]pentan-1-ylmethyl)-5-(3-chloro-5-fluoro-4-hydroxybenzamido)-2-(2-fluorophenyl)thiazole-4-carboxamide (0.012 g, 12%). LCMS (ES): m/z calcd. for C23H18ClF2N3O3S, 489.07; found, 490.2 (M+H); 1H NMR (400 MHz, DMSO-d6): δ 12.53 (s, 1H), 11.65 (bs, 1H), 8.79 (t, J=6.0 Hz, 1H), 7.73 (s, 1H), 7.69-7.66 (m, 1H), 7.58-7.39 (m, 3H), 3.43 (d, J=6.4 Hz, 2H), 1.69 (s, 6H); LC-Purity: 99.84% at 240 nm.

### Example 107: Synthesis of 5-(4-chloro-6-fluoro-5-hydroxypicolinamido)-2-(2-fluorophenyl)-N-(2-(trifluoromethyl)benzyl)thiazole-4-carboxamide

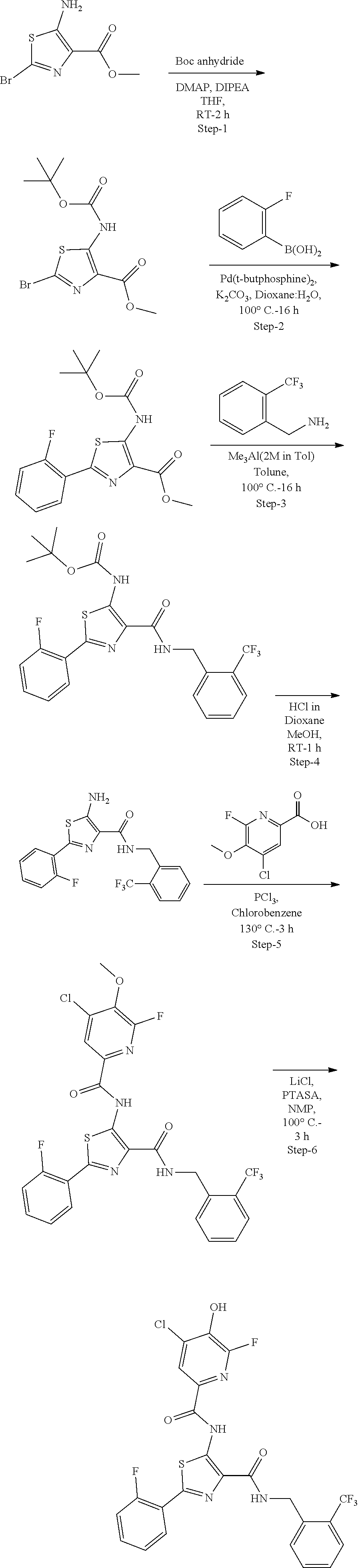

### Step-1: Preparation of methyl 2-bromo-5-((tert-butoxycarbonyl)amino)thiazole-4-carboxylate

To a stirred solution of methyl 5-amino-2-bromothiazole-4-carboxylate (1 g, 4.22 mmol) in Tetrahydrofuran (20 mL, 246 mmol) was added di-tert-butyl dicarbonate (1.45 mL, 6.33 mmol), DIPEA (2.2 mL, 12.7 mmol) and DMAP (0.520 g, 4.22 mmol) at ambient temperature and then stirred at same temperature for another 2 h. The reaction mixture was poured into water (40 mL) and extracted with ethyl acetate (50 mL×3). The combined organic phase was washed with water, brine and dried over anhydrous sodium sulfate, filtered and concentrated in vacuo to get crude. The crude material was purified by flash column chromatography using 30% Ethyl acetate in Hexane as an eluent to afford methyl 2-bromo-5-((tert-butoxycarbonyl)amino)thiazole-4-carboxylate (0.9 g, 63%) as white solid. LCMS (ES): m/z calcd. for C10H13BrN2O4S, 335.98; found, 337.0.

### Step-2: Preparation of methyl 5-((tert-butoxycarbonyl) amino)-2-(2-fluorophenyl) thiazole-4-carboxylate

To a stirred solution of methyl 2-bromo-5-((tert-butoxycarbonyl)amino)thiazole-4-carboxylate (0.460 g, 1.36 mmol) and (2-fluorophenyl)boronic acid (0.382 g, 2.73 mmol) in 1,4-dioxane:water 8:2 (6 mL) was added and potassium carbonate (0.566 g, 4.09 mmol) and degassed with nitrogen for 10 min, followed by bis(tri-tert-butylphosphane) palladium (0.0697 g, 0.136 mmol) was added and stirred at 100° C. for 16 h. Progress of the reaction was monitored by TLC/LCMS. After completion of the reaction, the reaction mixture was filtered through a pad of celite, filtrate was diluted with ethyl acetate (50 mL), washed with water (25×2 mL), brine solution and dried over anhydrous sodium sulphate, filtered and concentrated under vacuo to give crude. The crude product was purified by flash column chromatography using 20% ethyl acetate in hexane as an eluent to afford methyl 5-((tert-butoxycarbonyl)amino)-2-(2-fluorophenyl)thiazole-4-carboxylate (0.350 g, 73%) as brown solid. LCMS (ES): m/z calcd. for C16H17FN2O4S, 352.09; found, 353.1.

### Step-3: Preparation of tert-butyl (2-(2-fluorophenyl)-4-((2-(trifluoromethyl) benzyl) carbamoyl) thiazol-5-yl) carbamate

To a stirred solution of methyl 5-((tert-butoxycarbonyl)amino)-2-(2-fluorophenyl)thiazole-4-carboxylate (0.3 g, 0.851 mmol) in Toluene (5 mL) was added 1-[2-(trifluoromethyl)phenyl]methanamine (0.119 mL, 0.851 mmol) and 2 M trimethylaluminium in toluene (0.245 mL, 2.55 mmol) at 0° C. The resulting reaction mixture was stirred at 100° C. for 16 hours. The reaction mixture was cooled to ambient temperature and quenched with ice cold water (25 mL) and extracted with Ethyl acetate (25×2 mL). The combined organic phase was washed with brine solution (15 mL) and dried over anhydrous sodium sulphate, filtered, and evaporated in vacuo to obtain crude. The crude material was purified by flash column chromatography using 30% EtOAc: Hexane as the eluent to afford tert-butyl (2-(2-fluorophenyl)-4-((2-(trifluoromethyl)benzyl)carbamoyl)thiazol-5-yl)carbamate (0.3 g, 71%) as off white solid. LCMS (ES): m/z calcd. C23H21F4N3O3S, 495.12; found, 496.1.

### Step-4: Preparation of 5-amino-2-(2-fluorophenyl)-N-(2-(trifluoromethyl) benzyl) thiazole-4-carboxamide

To a suspension of tert-butyl (2-(2-fluorophenyl)-4-((2-(trifluoromethyl)benzyl)carbamoyl)thiazol-5-yl)carbamate (0.250 g, 0.505 mmol) in methanol (1 mL) was added 4M HCl in dioxane (5 mL, 144 mmol) at 0° C. and then allowed to stirred at RT for 1 h. The reaction mixture was poured into water (20 mL) and extracted with ethyl acetate (20 mL×3). The organic phase was washed with water (10 ml), brine (5 ml) and dried over anhydrous sodium sulfate, filtered and concentrated in vacuo to give 5-amino-2-(2-fluorophenyl)-N-(2-(trifluoromethyl)benzyl)thiazole-4-carboxamide (0.170 mg, 85%) as a white solid. LCMS (ES): m/z calcd. for C18H13F4N3OS, 395.07; found, 396.1.

### Step-5: Preparation of 5-(4-chloro-6-fluoro-5-methoxypicolinamido)-2-(2-fluorophenyl)-N-(2-(trifluoromethyl)benzyl)thiazole-4-carboxamide

To a suspension of 5-amino-2-(2-fluorophenyl)-N-(2-(trifluoromethyl)benzyl)thiazole-4-carboxamide (0.1 g, 0.25 mmol) in Chlorobenzene (0.5 mL) was added 4-chloro-6-fluoro-5-methoxypyridine-2-carboxylic acid (0.065 g, 0.30 mmol) and Phosphorous trichloride (0.03 mL, 0.25 mmol). The resulting reaction mixture was heated to 120° C. for 3 h. The progress of reaction was monitored by LCMS. The reaction mixture was cooled to ambient temperature and reaction mixture was quenched with ice-cold water (10 mL), solid was collected by filtration to get crude residue which was purified by flash column chromatography using 30% Ethyl acetate in Hexane as an eluent to afford 5-(4-chloro-6-fluoro-5-methoxypicolinamido)-2-(2-fluorophenyl)-N-(2-(trifluoromethyl)benzyl)thiazole-4-carboxamide (0.08 g, 54%) as an off-white solid. LCMS (ES): m/z C25H16ClF5N4O3S calcd. for 582.06; found, 583.1 (M+H); 1H NMR (400 MHz, DMSO-d6): δ 12.78 (s, 1H), 9.37 (t, J=5.6 Hz, 1H), 8.48-8.45 (m, 1H), 8.18 (s, 1H), 7.74 (d, J=8.0 Hz, 1H), 7.66-7.63 (m, 1H), 7.54-7.45 (m, 2H), 7.42-7.37 (m, 3H), 4.76 (d, J=5.6 Hz, 2H), 19F NMR (400 MHz, DMSO-d6): δ −50.05 (m, 3F), −77.37 (1F), −112.88 (1F). LC Purity: 99.1% at 254 nm.

### Step-6: Preparation of 5-(4-chloro-6-fluoro-5-hydroxypicolinamido)-2-(2-fluorophenyl)-N-(2-(trifluoromethyl)benzyl)thiazole-4-carboxamide

To a stirred solution of 4-chloro-6-fluoro-N-[2-(2-fluorophenyl)-4-({[2-(trifluoromethyl)phenyl]methyl}carbamoyl)-1,3-thiazol-5-yl]-5-methoxypyridine-2-carboxamide (80 mg, 0.137 mmol) in NMP (1 mL) was added lithium chloride (17.5 mg, 0.412 mmol) and p-TSA (78.3 mg, 0.412 mmol). The reaction mixture was heated at 100° C. for 3 h. After completion, reaction mixture was quenched with water (5 mL), solid was collected by filtration to get crude. Which was purified through flash chromatography by using 0-10% MeOH/DCM as eluent to afford 5-(4-chloro-6-fluoro-5-hydroxypicolinamido)-2-(2-fluorophenyl)-N-(2-(trifluoromethyl)benzyl)thiazole-4-carboxamide (30 mg, 38%). LCMS (ES): m/z C24H14ClF5N4O3S calcd. for 568.04; found, 569.2 (M+H); ¹H NMR (400 MHz, DMSO-d6): δ 12.68 (s, 1H), 9.39 (t, J=6.4 Hz, 1H), 8.50-8.46 (m, 1H), 8.14 (S, 1H), 7.77 (d, J=7.6 Hz, 1H), 7.69-7.65 (m, 1H), 7.58-7.38 (m, 5H), 4.78 (d, J=6.4 Hz, 2H). ¹⁹F NMR (400 MHz, DMSO-d6): δ−59.10 (3F, —CF3), −81.55 (1F, —ArF), −112.92 (1F, —ArF). LC Purity: 99.5% at 240 nm.

### Example A: Estrone Detection Assay for Evaluation of HSD17f313 Activity and Identification of Inhibitors

The liquid chromatography/mass spectrometry (LC/MS) estrone detection assay monitors the conversion of estradiol to estrone by HSD17B13. This assay was undertaken in a 96 wp format (Eppendorf deep well Plate 96/500) in an 80 μl reaction volume containing: 4 μM of Estradiol (E2; Cayman; Ser. No. 10/006,315), 6 mM NAD+(Sigma; #N0623) and 30 nM HSD17B13 enzyme (in-house; E. coli expressed His-tagged, purified, soluble protein) in a reaction containing 1M potassium phosphate buffer pH 7.4, with 0.5% vehicle (DMSO). Reactions were incubated for 2 hours at 26.5° C., and estradiol (E2) conversion to estrone (E1) was quantitated by LC-MS/MS based analyte detection for both E2 and E1 using LCMS grade reagents.

Reactions were terminated by the addition of two volumes of acetonitrile (MeCN; LCMS grade; CAS #75/05/8) containing deuterated (D4)-E1 used as internal standard (Clear Synth; #CS-T-54273; 500 ng/mL final concentration). Samples were applied to pre-prepared Bond Elut-C18 extraction cartridges (3 mL; Agilent; Ser. No. 12/102,028), washed and eluted in MeCN. Eluates were dried under nitrogen and re-suspended in 60% methanol (LCMS grade methanol; CAS #67/56/1) before submission for analysis. Aqueous linearity for E2 and E1 were included for quantification.

Analysis of samples was undertaken on a XBridge BEH C18 column (Waters; #186003033) using 0.1% Diethyl Amine in MeCN (mobile phase A; DEA CAS #109-89-7) and 0.1% Diethyl Amine in milli-Q water (mobile phase B) in a 3 min gradient allowing 25% B. Analytes were detected in negative mode using MRM analysis, with E2 having a RT of 1.85 min and E1 having a RT of 2 min. Activity of the enzyme, in the absence of NAD+, was used to evaluate specificity of conversion. Enzyme activity in the presence of test samples was expressed as a percentage of the uninhibited enzyme activity, and plotted versus inhibitor concentration. Non-linear regression was performed using a four-parameter logistic model and GraphPad Prism software (GraphPad Software, La Jolla, CA). All assessments were undertaken in duplicate evaluations and pooled during extraction process and subsequently injected as duplicates for LC-MS/MS analysis.

### Example B: Estrone Detection Assay for Evaluation of HSD17B14 Activity and Identification of Inhibitors Via Detection of Estrone Product

The liquid chromatography/mass spectrometry (LC/MS) estrone detection assay monitors the conversion of estradiol to estrone by HSD17B14. This assay was undertaken in a 96 well plates (Eppendorf deep well), in an 80 μl reaction volume containing: 8 μM of estradiol (E2; Cayman); 4 mM NAD+(Sigma); 120 nM HSD17B14 enzyme (in-house E. coli expressed His-tagged, purified, soluble protein); 1M potassium phosphate buffer pH 7.4, and 1.2% vehicle (DMSO). Reactions were incubated for 2 hours at 37° C. Estradiol (E2) conversion to estrone (E1) was quantitated by LC-MS/MS based analyte detection for E1 using LCMS grade reagents.

Reactions were terminated by the addition of two volumes of acetonitrile (MeCN; LCMS grade) containing deuterated (D4)-E1 used as internal standard (Clear Synth; 500 ng/mL final concentration). Samples were applied to pre-prepared Bond Elut-C18 extraction cartridges (3 mL; Agilent). The cartridges were washed with 1.5 mL water followed by 1.5 mL 5% aqueous methanol, and dried for 5 minutes under vacuum. They were then eluted with 4.5 mL MeCN. The eluates were dried under nitrogen and re-suspended in 200 μL of 60% methanol (LCMS grade) before submission for analysis. Standard curves of E2 and E1 in 4% DMSO were included for quantification.

Analysis of samples was performed on a XBridge BEH C18 column (Waters) using 0.1% Diethyl Amine in MeCN (mobile phase A) and 0.1% Diethyl Amine in milli-Q water (mobile phase B) in a 3 min gradient from 0-25% B. Analytes were detected in negative mode using MRM analysis, with E2 having a RT of 1.89 min and E1 having a RT of 2.07 min. Activity of the enzyme in the absence of NAD+ was used to evaluate specificity of conversion. Enzyme activity in the presence of test samples was expressed as a percentage of the uninhibited enzyme in the presence of 1.2% DMSO vehicle only, and plotted versus inhibitor concentration. Non-linear regression was performed using a four-parameter logistic model and GraphPad Prism software. All assessments were undertaken in duplicate evaluations, which were pooled during the extraction process and subsequently injected as duplicates for LC-MS/MS analysis.

### Example C: Evaluation of HSD17B2 Activity and Identification of Inhibitors Via Detection of Reduced Nicotinamide Adenine Dinucleotide (NADH) Product

The fluorescence based detection assay monitors the conversion of the co-factor NAD⁺ to NADH, which occurs coincident with the conversion of estradiol to estrone by HSD17B2. These assays were performed in 384 well plates (Greiner V-shape PP-microplate). The 20 μl reaction volume contained: 0.7 μM estradiol (E2); 6 mM NAD⁺ (Sigma); 250 nM HSD17B2 enzyme (Origene; Cat #TP303293); 0.25 M potassium phosphate buffer pH 7.4, 0.75% vehicle (DMSO). Reactions were incubated for 2 hours at 37° C., and the reaction was stopped by freezing the reaction plates at −80° C. Zero time samples were frozen immediately.

The conversion of NAD⁺ to NADH was quantitated using NAD-Glo kits (Promega; Cat #G9062) according to the manufacturers' instructions. A volume of 15 μL of enzyme reaction mixture was added to 15 μL of reconstituted Glo reagent, and the plates were incubated for 30 minutes at room temperature. Luminescence was quantitated on a Tecan Spark Reader®. A standard curve of NADH (0.1-50 μL) in potassium phosphate buffer pH 7.4/1% DMSO was run on each plate.

Activity of the enzyme in the absence of E2 was used to evaluate specificity of conversion. Enzyme activity in the presence of test samples was expressed as a percentage of the uninhibited enzyme in the presence of 1% DMSO vehicle only, and was plotted versus inhibitor concentration. Non-linear regression was performed using a four-parameter logistic model and GraphPad Prism software. All assessments were undertaken in duplicate evaluations.

The data from examples A, B, and C is shown in table 2 and 3 below:

TABLE 2

| Example | IC₅₀ with Estradiol |
|---|---|
| 1 | A |
| 2 | B |
| 3 | A |
| 4 | B |
| 5 | B |
| 6 | B |
| 7 | D |
| 8 | B |
| 9 | A |
| 10 | D |
| 11 | A |
| 12 | B |
| 13 | B |
| 14 | A |
| 15 | B |
| 16 | A |
| 17 | B |
| 18 | B |
| 19 | B |
| 20 | B |
| 21 | B |
| 22 | B |
| 23 | A |
| 24 | B |
| 25 | B |
| 26 | B |
| 27 | B |
| 28 | D |
| 29 | B |
| 30 | B |
| 31 | D |
| 32 | A |
| 33 | B |
| 34 | C |
| 35 | B |
| 36 | D |
| 37 | E |
| 38 | A |
| 39 | B |
| 40 | A |
| 41 | B |
| 42 | B |
| 43 | B |
| 44 | C |
| 46 | B |
| 48 | B |
| 49 | B |
| 50 | B |
| 51 | B |
| 52 | B |
| 53 | C |
| 54 | D |
| 55 | B |
| 56 | C |
| 57 | A |
| 58 | A |

IC₅₀ with Estradiol
A is less than or equal to 0.1 μM;
B is more than 0.1 μM and less than or equal to 0.5 μM;
C is more than 0.5 μM and less than or equal to 1.0 μM;
D is more than 1.0 μM and less than or equal to 10 μM;
E is more than 10 μM;

TABLE 3

| | HSD17B13 | | |
|---|---|---|---|
| | Inhibition_Estradiol | HSD17B2 fold | HSD17B14 fold |
| Ex. | Substrate_LCMS: Ki | selectivity | selectivity |
| 59 | A | | >331 |
| 60 | A | | >980 |
| 61 | A | 64 | >397 |
| 62 | A | 204 | |
| 63 | A | 192 | |
| 64 | A | 115 | >855 |
| 65 | A | 225 | |
| 66 | B | 36 | |
| 67 | A | 513 | |
| 68 | B | 48 | |
| 70 | A | 107 | |
| 71 | A | | |
| 72 | C | | |
| 73 | A | 175 | >646 |
| 74 | A | | |
| 75 | A | | |
| 76 | A | | |
| 77 | A | | |
| 78 | B | 10 | >180 |
| 79 | C | | |
| 80 | C | | |
| 81 | C | | |
| 82 | C | | |
| 83 | A | | |
| 84 | A | | |
| 85 | C | | |
| 86 | C | | |
| 87 | C | | |
| 88 | A | 53 | |
| 89 | A | | 688 |
| 90 | A | 107 | >797 |
| 91 | B | 24 | 76 |
| 92 | B | 44 | >215 |
| 94 | A | 1270 | >1250 |
| 95 | A | 2150 | >1640 |
| 96 | A | 985 | >2000 |
| 97 | A | | 772 |
| 98 | A | | 928 |
| 99 | A | | 253 |
| 100 | A | | >843 |
| 101 | A | | >142 |
| 102 | A | | >1230 |
| 103 | A | | >1850 |
| 104 | A | 444 | 357 |
| 105 | B | | |
| 106 | A | | |
| 107 | A | | |

HSD17B13 Inhibition_Estradiol Substrate_LCMS: Ki
A is less than or equal to 50 nM;
B is more than 50 nM and less than or equal to 100 nM;
C is more than 100 nM and less than or equal to 500 nM;

What is claimed is:

1. A compound of Formula (I), or a pharmaceutically acceptable salt, solvate, or stereoisomer thereof: 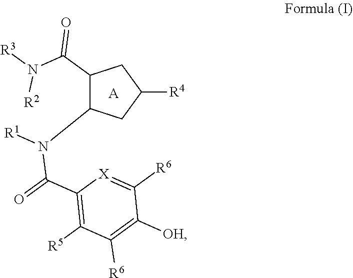
wherein:
Ring A is thiazolyl;
R¹ is hydrogen, C₁-C₆alkyl, C₁-C₆haloalkyl, C₁-C₆deuteroalkyl, C₁-C₆hydroxyalkyl, or C₁-C₆aminoalkyl;
R² is hydrogen or C₁-C₆alkyl;
R³ is C₁-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₂-C₁₀alkenyl, C₂-C₁₀alkynyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally and independently substituted with one or more R^{3a};
each R^{3a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{3a} on the same atom are taken together to form an oxo;
R⁴ is deuterium, halogen, C₁-C₆alkyl, C₂-C₆haloalkyl, C₁-C₆deuteroalkyl, C₁-C₆hydroxyalkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more with one or more R^{4a};
each R^{4a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NHS(═O)₂R^{a}, —C(═O)R^{a}, —C(═O)C(═O)R^{a}, —C(═O)OR^{b}, —C(═O)NR^{c}R^{d}, —C(═O)C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally and independently substituted with one or more R;
or two R^{4a} on the same atom are taken together to form an oxo;
or two R^{4a} on the same carbon are taken together to form a cycloalkyl or heterocycloalkyl; each optionally substituted with one or more R;
or two R^{4a} on the different atoms are taken together to form a cycloalkyl, heterocycloalkyl, aryl, or heteroaryl; each optionally substituted with one or more R;
X is N or CR^{X};
R^{X} is hydrogen, deuterium, halogen, —CN, —OH, —OR^{a}, —NR^{c}R^{d}, C₁-C₆alkyl, C₁-C₆haloalkyl, or C₁-C₆deuteroalkyl;
R⁵ is hydrogen, deuterium, halogen, —CN, —OH, —OR^{a}, —NR^{c}R^{d}, C₁-C₆alkyl, C₁-C₆haloalkyl, or C₁-C₆deuteroalkyl;
each R⁶ is independently a halogen;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom form an oxo.

2. The compound of claim 1, or a pharmaceutically acceptable salt, solvate, or stereoisomer thereof, wherein the compound is of Formula (Ia):

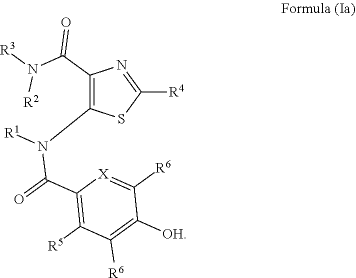

3. The compound of claim 1, or a pharmaceutically acceptable salt, solvate, or stereoisomer thereof, wherein the compound is of Formula (Ib):

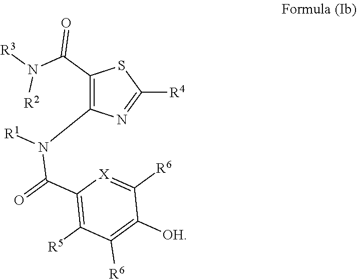

4. The compound of any one of claims 1-3, or a pharmaceutically acceptable salt, solvate, or stereoisomer thereof, wherein R¹ is hydrogen.

5. The compound of any one of claims 1-4, or a pharmaceutically acceptable salt, solvate, or stereoisomer thereof, wherein R² is hydrogen.

6. The compound of any one of claims 1-5, or a pharmaceutically acceptable salt, solvate, or stereoisomer thereof, wherein R³ is C₁-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}.

7. The compound of any one of claims 1-5, or a pharmaceutically acceptable salt, solvate, or stereoisomer thereof, wherein R³ is C₄-C₁₀alkyl, C₁-C₁₀haloalkyl, C₁-C₁₀deuteroalkyl, C₁-C₁₀hydroxyalkyl, C₁-C₁₀aminoalkyl, C₁-C₁₀heteroalkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}.

8. The compound of any one of claims 1-5, or a pharmaceutically acceptable salt, solvate, or stereoisomer thereof, wherein R³ is C₁-C₁₀alkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}.

9. The compound of any one of claims 1-5, or a pharmaceutically acceptable salt, solvate, or stereoisomer thereof, wherein R³ is C₄-C₁₀alkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{3a}.

10. The compound of any one of claims 1-5, or a pharmaceutically acceptable salt, solvate, or stereoisomer thereof, wherein R³ is C₁-C₁₀alkyl or C₁-C₆alkylene(aryl); wherein the alkyl, alkylene, and aryl is optionally and independently substituted with one or more R^{3a}.

11. The compound of any one of claims 1-5, or a pharmaceutically acceptable salt, solvate, or stereoisomer thereof, wherein R³ is C₄-C₁₀alkyl or C₂-C₆alkylene(aryl); wherein the alkyl, alkylene, and aryl is optionally and independently substituted with one or more R^{3a}.

12. The compound of any one of claims 1-5, or a pharmaceutically acceptable salt, solvate, or stereoisomer thereof, wherein R³ is C₁-C₁₀alkyl optionally and independently substituted with one or more R^{3a}.

13. The compound of any one of claims 1-5, or a pharmaceutically acceptable salt, solvate, or stereoisomer thereof, wherein R³ is C₄-C₁₀alkyl optionally and independently substituted with one or more R^{3a}.

14. The compound of any one of claims 1-5, or a pharmaceutically acceptable salt, solvate, or stereoisomer thereof, wherein R³ is C₄-C₁₀alkyl.

15. The compound of any one of claims 1-5, or a pharmaceutically acceptable salt, solvate, or stereoisomer thereof, wherein R³ is C₁-C₆alkylene(aryl); wherein the alkylene and aryl is optionally and independently substituted with one or more R^{3a}.

16. The compound of any one of claims 1-5, or a pharmaceutically acceptable salt, solvate, or stereoisomer thereof, wherein R³ is C₂-C₆alkylene(aryl); wherein the alkylene and aryl is optionally and independently substituted with one or more R^{3a}.

17. The compound of any one of claims 1-16, or a pharmaceutically acceptable salt, solvate, or stereoisomer thereof, wherein each R^{3a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; or two R^{3a} on the same atom are taken together to form an oxo.

18. The compound of any one of claims 1-16, or a pharmaceutically acceptable salt, solvate, or stereoisomer thereof, wherein each R^{3a} is independently deuterium, halogen, —OH, —OR^{a}, C₁-C₆alkyl, C₁-C₆haloalkyl, or C₁-C₆deuteroalkyl; or two R^{3a} on the same atom are taken together to form an oxo.

19. The compound of any one of claims 1-16, or a pharmaceutically acceptable salt, solvate, or stereoisomer thereof, wherein each R^{3a} is independently halogen, —OR^{a}, or C₁-C₆haloalkyl.

20. The compound of any one of claims 1-16, or a pharmaceutically acceptable salt, solvate, or stereoisomer thereof, wherein each R^{3a} is independently C₁-C₆haloalkyl.

21. The compound of any one of claims 1-20, or a pharmaceutically acceptable salt, solvate, or stereoisomer thereof, wherein X is N.

22. The compound of any one of claims 1-20, or a pharmaceutically acceptable salt, solvate, or stereoisomer thereof, wherein X is CR^{X}.

23. The compound of any one of claims 1-20 or 22, or a pharmaceutically acceptable salt, solvate, or stereoisomer thereof, wherein R^{X} is hydrogen, halogen, C₁-C₆alkyl, or C₁-C₆haloalkyl.

24. The compound of any one of claims 1-20 or 22 or 23, or a pharmaceutically acceptable salt, solvate, or stereoisomer thereof, wherein R^{X} is hydrogen or C₁-C₆alkyl.

25. The compound of any one of claims 1-20 or 22-24, or a pharmaceutically acceptable salt, solvate, or stereoisomer thereof, wherein R^{X} is hydrogen.

26. The compound of any one of claims 1-25, or a pharmaceutically acceptable salt, solvate, or stereoisomer thereof, wherein R⁵ is hydrogen, halogen, C₁-C₆alkyl, or C₁-C₆haloalkyl.

27. The compound of any one of claims 1-26, or a pharmaceutically acceptable salt, solvate, or stereoisomer thereof, wherein R⁵ is hydrogen or C₁-C₆alkyl.

28. The compound of any one of claims 1-27, or a pharmaceutically acceptable salt, solvate, or stereoisomer thereof, wherein R⁵ is hydrogen.

29. The compound of any one of claims 1-28, or a pharmaceutically acceptable salt, solvate, or stereoisomer thereof, wherein each R⁶ is chloro.

30. The compound of any one of claims 1-28, or a pharmaceutically acceptable salt, solvate, or stereoisomer thereof, wherein each R⁶ is fluoro.

31. The compound of any one of claims 1-30, or a pharmaceutically acceptable salt, solvate, or stereoisomer thereof, wherein R⁴ is cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more with one or more R^{4a}.

32. The compound of any one of claims 1-30, or a pharmaceutically acceptable salt, solvate, or stereoisomer thereof, wherein R⁴ is cycloalkyl, heterocycloalkyl, aryl, or heteroaryl; wherein the cycloalkyl, heterocycloalkyl, aryl, and heteroaryl is optionally substituted with one or more with one or more R^{4a}.

33. The compound of any one of claims 1-30, or a pharmaceutically acceptable salt, solvate, or stereoisomer thereof, wherein R⁴ is cycloalkyl optionally substituted with one or more with one or more R^{4a}.

34. The compound of any one of claims 1-30, or a pharmaceutically acceptable salt, solvate, or stereoisomer thereof, wherein R⁴ is heterocycloalkyl optionally substituted with one or more with one or more R^{4a}.

35. The compound of any one of claims 1-30, or a pharmaceutically acceptable salt, solvate, or stereoisomer thereof, wherein R⁴ is aryl or heteroaryl; wherein the aryl and heteroaryl is optionally substituted with one or more with one or more R^{4a}.

36. The compound of any one of claims 1-30, or a pharmaceutically acceptable salt, solvate, or stereoisomer thereof, wherein R⁴ is aryl optionally substituted with one or more with one or more R^{4a}.

37. The compound of any one of claims 1-30, or a pharmaceutically acceptable salt, solvate, or stereoisomer thereof, wherein R⁴ is heteroaryl optionally substituted with one or more with one or more R^{4a}.

38. The compound of any one of claims 1-37, or a pharmaceutically acceptable salt, solvate, or stereoisomer thereof, wherein each R^{4a} is independently deuterium, halogen, —CN, —OH, —OR, —S(═O)R^{a}, —NR^{c}R^{d}, C₁-C₆alkyl, or C₁-C₆haloalkyl; wherein the alkyl is optionally and independently substituted with one or more R; or two R^{4a} on the same atom are taken together to form an oxo.

39. The compound of any one of claims 1-37, or a pharmaceutically acceptable salt, solvate, or stereoisomer thereof, wherein each R^{4a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R^{4a} on the same atom are taken together to form an oxo.

40. The compound of any one of claims 1-37, or a pharmaceutically acceptable salt, solvate, or stereoisomer thereof, wherein each R^{4a} is independently deuterium, halogen, —CN, —OH, —OR^{a}, —S(═O)R^{a}, —NR^{c}R^{d}, C₁-C₆alkyl, C₁-C₆haloalkyl, C₁-C₆deuteroalkyl, cycloalkyl, or heterocycloalkyl; or two R^{4a} on the same atom are taken together to form an oxo.

41. The compound of any one of claims 1-37, or a pharmaceutically acceptable salt, solvate, or stereoisomer thereof, wherein each R^{4a} is independently halogen, —CN, —OH, —OR^{a}, —S(═O)R^{a}, —NR^{c}R^{d}, C₁-C₆alkyl, or C₁-C₆haloalkyl; or two R^{4a} on the same atom are taken together to form an oxo.

42. The compound of any one of claims 1-37, or a pharmaceutically acceptable salt, solvate, or stereoisomer thereof, wherein each R^{4a} is independently halogen, —S(═O)R^{a}, C₁-C₆alkyl, or C₁-C₆haloalkyl; or two R^{4a} on the same atom are taken together to form an oxo.

43. A compound selected from a compound found in table 1a or table 1b, or a pharmaceutically acceptable salt, solvate, or stereoisomer thereof.

44. A pharmaceutical composition comprising a compound of any one of claims 1-43, or a pharmaceutically acceptable salt, solvate, or stereoisomer thereof, and a pharmaceutically acceptable carrier.

45. A method of treating a disease in a subject in need thereof, the method comprising administering a pharmaceutically effective amount of a compound of any one of claims 1-43, or a pharmaceutically acceptable salt, solvate, or stereoisomer thereof, or a pharmaceutical composition of claim 44.

46. The method of claim 45, wherein the disease is a liver disease, a metabolic disease, or a cardiovascular disease.

47. The method of claim 45 or 46, wherein the disease is NAFLD.

48. The method of claim 45 or 46, wherein the disease is NASH.

49. The method of claim 45 or 46, wherein the disease is drug induced liver injury (DILI).

50. The method of claim 45 or 46, wherein the disease is associated with HSD17B13.

51. The method of claim 45 or 46, wherein the diseases is alcoholic liver disease.

52. The method of claim 45 or 46, wherein the disease is cirrhosis.

53. The method of claim 45 or 46, wherein the disease is decompensated portal hypertension.

54. The method of claim 45 or 46, wherein the disease is cholestatic liver disease.

55. A method for selectively inhibiting HSD17B13, the method comprising administering a pharmaceutically effective amount of a compound of any one of claims 1-43, or a pharmaceutically acceptable salt, solvate, or stereoisomer thereof.

56. The method of claim 55, wherein the compound selectively inhibit HSD17B13 over HSD17B2, HSD17B14, or any combination thereof.
